SEQUENCE LISTING (81 sequences)

SEQ ID NO: 1 - DNA, 5601 bp, Homo sapiens
ggcccggctgcgaggaggaggcggcggcggcgcaggaggatgtacttggtggcgggggacagggggttggccggctgcgggcacctcctggtctcgctgctggggctgctgctgctgctggcgcgctccggcacccgggcgctggtctgcctgccctgtgacgagtccaagtgcgaggagcccaggaactgcccggggagcatcgtgcagggcgtctgcggctgctgctacacgtgcgccagccagaggaacgagagctgcggcggcaccttcgggatttacggaacctgcgaccgggggctgcgttgtgtcatccgccccccgctcaatggcgactccctcaccgagtacgaagcgggcgtttgcgaagatgagaactggactgatgaccaactgcttggttttaaaccatgcaatgaaaaccttattgctggctgcaatataatcaatgggaaatgtgaatgtaacaccattcgaacctgcagcaatccctttgagtttccaagtcaggatatgtgcctttcagctttaaagagaattgaagaagagaagccagattgctccaaggcccgctgtgaagtccagttctctccacgttgtcctgaagattctgttctgatcgagggttatgctcctcctggggagtgctgtcccttacccagccgctgcgtgtgcaaccccgcaggctgtctgcgcaaagtctgccagccgggaaacctgaacatactagtgtcaaaagcctcagggaagccgggagagtgctgtgacctctatgagtgcaaaccagttttcggcgtggactgcaggactgtggaatgccctcctgttcagcagaccgcgtgtcccccggacagctatgaaactcaagtcagactaactgcagatggttgctgtactttgccaacaagatgcgagtgtctctctggcttatgtggtttccccgtgtgtgaggtgggatccactccccgcatagtctctcgtggcgatgggacacctggaaagtgctgtgatgtctttgaatgtgttaatgatacaaagccagcctgcgtatttaacaatgtggaatattatgatggagacatgtttcgaatggacaactgtcggttctgtcgatgccaagggggcgttgccatctgcttcactgcccagtgtggtgagataaactgcgagaggtactacgtgcccgaaggagagtgctgcccagtgtgtgaagatccagtgtatccttttaataatcccgctggctgctatgccaatggcctgatccttgcccacggagaccggtggcgggaagacgactgcacattctgccagtgcgtcaacggtgaacgccactgcgttgcgaccgtctgcggacagacctgcacaaaccctgtgaaagtgcctggggagtgttgccctgtgtgcgaagaaccaaccatcatcacagttgatccacctgcatgtggggagttatcaaactgcactctgacagggaaggactgcattaatggtttcaaacgcgatcacaatggttgtcggacctgtcagtgcataaacaccgaggaactatgttcagaacgtaaacaaggctgcaccttgaactgtcccttcggtttccttactgatgcccaaaactgtgagatctgtgagtgccgcccaaggcccaagaagtgcagacccataatctgtgacaagtattgtccacttggattgctgaagaataagcacggctgtgacatctgtcgctgtaagaaatgtccagagctctcatgcagtaagatctgccccttgggtttccagcaggacagtcacggctgtcttatctgcaagtgcagagaggcctctgcttcagctgggccacccatcctgtcgggcacttgtctcaccgtggatggtcatcatcataaaaatgaggagagctggcacgatgggtgccgggaatgctactgtctcaatggacgggaaatgtgtgccctgatcacctgcccggtgcctgcctgtggcaaccccaccattcaccctggacagtgctgcccatcatgtgcagatgactttgtggtgcagaagccagagctcagtactccctccatttgccacgcccctggaggagaatactttgtggaaggagaaacgtggaacattgactcctgtactcagtgcacctgccacagcggacgggtgctgtgtgagacagaggtgtgcccaccgctgctctgccagaacccctcacgcacccaggattcctgctgcccacagtgtacagatcaaccttttcggccttccttgtcccgcaataacagcgtacctaattactgcaaaaatgatgaaggggatatattcctggcagctgagtcctggaagcctgacgtttgtaccagctgcatctgcattgatagcgtaattagctgtttctctgagtcctgcccttctgtatcctgtgaaagacctgtcttgagaaaaggccagtgttgtccctactgcatagaagacacaattccaaagaaggtggtgtgccacttcagtgggaaggcctatgccgacgaggagcggtgggaccttgacagctgcacccactgctactgcctgcagggccagaccctctgctcgaccgtcagctgcccccctctgccctgtgttgagcccatcaacgtggaaggaagttgctgcccaatgtgtccagaaatgtatgtcccagaaccaaccaatatacccattgagaagacaaaccatcgaggagaggttgacctggaggttcccctgtggcccacgcctagtgaaaatgatatcgtccatctccctagagatatgggtcacctccaggtagattacagagataacaggctgcacccaagtgaagattcttcactggactccattgcctcagttgtggttcccataattatatgcctctctattataatagcattcctattcatcaatcagaagaaacagtggataccactgctttgctggtatcgaacaccaactaagccttcttccttaaataatcagctagtatctgtggactgcaagaaaggaaccagagtccaggtggacagttcccagagaatgctaagaattgcagaaccagatgcaagattcagtggcttctacagcatgcaaaaacagaaccatctacaggcagacaatttctaccaaacagtgtgaagaaaggcaactaggatgaggtttcaaaagacggaagacgactaaatctgctctaaaaagtaaactagaatttgtgcacttgcttagtggattgtattggattgtgacttgatgtacagcgctaagaccttactgggatgggctctgtctacagcaatgtgcagaacaagcattcccacttttcctcaagataactgaccaagtgttttcttagaaccaaagtttttaaagttgctaagatatatttgcctgtaagatagctgtagagatatttggggtggggacagtgagtttggatggggaaatgggtgggagggtggtgttgggaagaaaaattggtcagcttggctcggggagaaacctggtaacataaaagcagttcagtggcccagaggttatttttttcctattgctctgaagactgcactggttgctgcaaagctcaggcctgaatgagcaggaaacaaaaaaggccttgcgacccagctgccataaccaccttagaactaccagacgagcacatcagaaccctttgacagccatcccaggtctaaagccacaagtttcttttctatacagtcacaactgcagtaggcagtgaggaagccagagaaatgcgatagcggcatttctctaaagcgggttattaaggatatatacagttacactttttgctgcttttattttcttccaagccaatcaatcagccagttcctagcagagtcagcacatgaacaagatctaagtcatttcttgatgtgagcactggagcttttttttttttacaacgtgacaggaagaggagggagagggtgacgaacaccaggcatttccaggggctatatttcactgtttgttgttgctttgttctgttatattgttggttgttcatagtttttgttgaagctctagcttaagaagaaactttttttaaaaagactgtttggggattctttttccttattatatactgattctacaaaatagaaactacttcattttaattgtatattattcaagcacctttgttgaagctcaaaaaaaatgatgcctctttaaactttagcaattataggagtatttatgtaactatcttatgcttcaaaaaacaaaagtatttgtgtgcatgtgtatataatatatatatatacatatatatttatacacatacaatttatgttttcctgttgaatgtatttttatgagattttaaccagaacaaaggcagataaacaggcattccatagcagtgcttttgatcacttacaaattttttgaataacacaaaatctcattctacctgcagtttaattggaaagatgtgtgtgtgagagtatgtatgtgtgtgtgtgtgtgtgtgtgtgtgcgcgcgcacgcacgccttgagcagtcagcattgcacctgctatggagaagggtattcctttattaaaatcttcctcatttggatttgctttcagttggttttcaatttgctcactggccagagacattgatggcagttcttatctgcatcactaatcagctcctggattttttttttttttttttcaaacaatggtttgaaacaactactggaatattgtccacaataagctggaagtttgttgtagtatgcctcaaatataactgactgtatactatagtggtaacttttcaaacagcccttagcacttttatactaattaacccatttgtgcattgagttttcttttaaaaatgcttgttgtgaaagacacagatacccagtatgcttaacgtgaaaagaaaatgtgttctgttttgtaaaggaactttcaagtattgttgtaaatacttggacagaggttgctgaactttaaaaaaaattaatttattattataatgacctaatttattaatctgaagattaaccatttttttgtcttagaatatcaaaaagaaaaagaaaaaggtgttctagctgtttgcatcaaaggaaaaaaagatttattatcaaggggcaatatttttatcttttccaaaataaatttgttaatgatacattacaaaaatagattgacatcagcctgattagtataaattttgttggtaattaatccattcctggcataaaaagtctttatcaaaaaaaattgtagatgcttgctttttgttttttcaatcatggccatattatgaaaatactaacaggatataggacaaggtgtaaatttttttattattattttaaagatatgatttatcctgagtgctgtatctattactcttttactttggttcctgttgtgctcttgtaaaagaaaaatataatttcctgaagaataaaatagatatatggcacttggagtgcatcatagttctacagtttgtttttgttttcttcaaaaaagctgtaagagaattatctgcaacttgattcttggcaggaaataaacattttgagttgaaatcaaaaaaaaaaaaaaaaaa

SEQ ID NO: 2 - protein, 1036 aa, Homo sapiens
MYLVAGDRGLAGCGHLLVSLLGLLLLLARSGTRALVCLPCDESKCEEPRNCPGSIVQGVCGCCYTCASQRNESCGGTFGIYGTCDRGLRCVIRPPLNGDSLTEYEAGVCEDENWTDDQLLGFKPCNENLIAGCNIINGKCECNTIRTCSNPFEFPSQDMCLSALKRIEEEKPDCSKARCEVQFSPRCPEDSVLIEGYAPPGECCPLPSRCVCNPAGCLRKVCQPGNLNILVSKASGKPGECCDLYECKPVFGVDCRTVECPPVQQTACPPDSYETQVRLTADGCCTLPTRCECLSGLCGFPVCEVGSTPRIVSRGDGTPGKCCDVFECVNDTKPACVFNNVEYYDGDMFRMDNCRFCRCQGGVAICFTAQCGEINCERYYVPEGECCPVCEDPVYPFNNPAGCYANGLILAHGDRWREDDCTFCQCVNGERHCVATVCGQTCTNPVKVPGECCPVCEEPTIITVDPPACGELSNCTLTGKDCINGFKRDHNGCRTCQCINTEELCSERKQGCTLNCPFGFLTDAQNCEICECRPRPKKCRPIICDKYCPLGLLKNKHGCDICRCKKCPELSCSKICPLGFQQDSHGCLICKCREASASAGPPILSGTCLTVDGHHHKNEESWHDGCRECYCLNGREMCALITCPVPACGNPTIHPGQCCPSCADDFVVQKPELSTPSICHAPGGEYFVEGETWNIDSCTQCTCHSGRVLCETEVCPPLLCQNPSRTQDSCCPQCTDQPFRPSLSRNNSVPNYCKNDEGDIFLAAESWKPDVCTSCICIDSVISCFSESCPSVSCERPVLRKGQCCPYCIEDTIPKKVVCHFSGKAYADEERWDLDSCTHCYCLQGQTLCSTVSCPPLPCVEPINVEGSCCPMCPEMYVPEPTNIPIEKTNHRGEVDLEVPLWPTPSENDIVHLPRDMGHLQVDYRDNRLHPSEDSSLDSIASVVVPIIICLSIIIAFLFINQKKQWIPLLCWYRTPTKPSSLNNQLVSVDCKKGTRVQVDSSQRMLRIAEPDARFSGFYSMQKQNHLQADNFYQTV

SEQ ID NO: 3 - DNA, 1912 bp, Homo sapiens
ttttttttcttccctctagtgggcggggcagaggagttagccaagatgtgactttgaaaccctcagcgtctcagtgcccttttgttctaaacaaagaattttgtaattggttctaccaaagaaggatataatgaagtcactatgggaaaagatggggaggagagttgtaggattctacattaattctcttgtgcccttagcccactacttcagaatttcctgaagaaagcaagcctgaattggttttttaaattgctttaaaaattttttttaactgggttaatgcttgctgaattggaagtgaatgtccattcctttgcctcttttgcagatatacacttcagataactacaccgaggaaatgggctcaggggactatgactccatgaaggaaccctgtttccgtgaagaaaatgctaatttcaataaaatcttcctgcccaccatctactccatcatcttcttaactggcattgtgggcaatggattggtcatcctggtcatgggttaccagaagaaactgagaagcatgacggacaagtacaggctgcacctgtcagtggccgacctcctctttgtcatcacgcttcccttctgggcagttgatgccgtggcaaactggtactttgggaacttcctatgcaaggcagtccatgtcatctacacagtcaacctctacagcagtgtcctcatcctggccttcatcagtctggaccgctacctggccatcgtccacgccaccaacagtcagaggccaaggaagctgttggctgaaaaggtggtctatgttggcgtctggatccctgccctcctgctgactattcccgacttcatctttgccaacgtcagtgaggcagatgacagatatatctgtgaccgcttctaccccaatgacttgtgggtggttgtgttccagtttcagcacatcatggttggccttatcctgcctggtattgtcatcctgtcctgctattgcattatcatctccaagctgtcacactccaagggccaccagaagcgcaaggccctcaagaccacagtcatcctcatcctggctttcttcgcctgttggctgccttactacattgggatcagcatcgactccttcatcctcctggaaatcatcaagcaagggtgtgagtttgagaacactgtgcacaagtggatttccatcaccgaggccctagctttcttccactgttgtctgaaccccatcctctatgctttccttggagccaaatttaaaacctctgcccagcacgcactcacctctgtgagcagagggtccagcctcaagatcctctccaaaggaaagcgaggtggacattcatctgtttccactgagtctgagtcttcaagttttcactccagctaacacagatgtaaaagacttttttttatacgataaataacttttttttaagttacacatttttcagatataaaagactgaccaatattgtacagtttttattgcttgttggatttttgtcttgtgtttctttagtttttgtgaagtttaattgacttatttatataaattttttttgtttcatattgatgtgtgtctaggcaggacctgtggccaagttcttagttgctgtatgtctcgtggtaggactgtagaaaagggaactgaacattccagagcgtgtagtgaatcacgtaaagctagaaatgatccccagctgtttatgcatagataatctctccattcccgtggaacgtttttcctgttcttaagacgtgattttgctgtagaagatggcacttataaccaaagcccaaagtggtatagaaatgctggtttttcagttttcaggagtgggttgatttcagcacctacagtgtacagtcttgtattaagttgttaataaaagtacatgttaaacttaaaaaaaaaaaaaaaaaa

SEQ ID NO: 4 - DNA, 1691 bp, Homo sapiens
aacttcagtttgttggctgcggcagcaggtagcaaagtgacgccgagggcctgagtgctccagtagccaccgcatctggagaaccagcggttaccatggaggggatcagtatatacacttcagataactacaccgaggaaatgggctcaggggactatgactccatgaaggaaccctgtttccgtgaagaaaatgctaatttcaataaaatcttcctgcccaccatctactccatcatcttcttaactggcattgtgggcaatggattggtcatcctggtcatgggttaccagaagaaactgagaagcatgacggacaagtacaggctgcacctgtcagtggccgacctcctctttgtcatcacgcttcccttctgggcagttgatgccgtggcaaactggtactttgggaacttcctatgcaaggcagtccatgtcatctacacagtcaacctctacagcagtgtcctcatcctggccttcatcagtctggaccgctacctggccatcgtccacgccaccaacagtcagaggccaaggaagctgttggctgaaaaggtggtctatgttggcgtctggatccctgccctcctgctgactattcccgacttcatctttgccaacgtcagtgaggcagatgacagatatatctgtgaccgcttctaccccaatgacttgtgggtggttgtgttccagtttcagcacatcatggttggccttatcctgcctggtattgtcatcctgtcctgctattgcattatcatctccaagctgtcacactccaagggccaccagaagcgcaaggccctcaagaccacagtcatcctcatcctggctttcttcgcctgttggctgccttactacattgggatcagcatcgactccttcatcctcctggaaatcatcaagcaagggtgtgagtttgagaacactgtgcacaagtggatttccatcaccgaggccctagctttcttccactgttgtctgaaccccatcctctatgctttccttggagccaaatttaaaacctctgcccagcacgcactcacctctgtgagcagagggtccagcctcaagatcctctccaaaggaaagcgaggtggacattcatctgtttccactgagtctgagtcttcaagttttcactccagctaacacagatgtaaaagacttttttttatacgataaataacttttttttaagttacacatttttcagatataaaagactgaccaatattgtacagtttttattgcttgttggatttttgtcttgtgtttctttagtttttgtgaagtttaattgacttatttatataaattttttttgtttcatattgatgtgtgtctaggcaggacctgtggccaagttcttagttgctgtatgtctcgtggtaggactgtagaaaagggaactgaacattccagagcgtgtagtgaatcacgtaaagctagaaatgatccccagctgtttatgcatagataatctctccattcccgtggaacgtttttcctgttcttaagacgtgattttgctgtagaagatggcacttataaccaaagcccaaagtggtatagaaatgctggtttttcagttttcaggagtgggttgatttcagcacctacagtgtacagtcttgtattaagttgttaataaaagtacatgttaaacttaaaaaaaaaaaaaaaaaa

SEQ ID NO: 5 - protein, 356 aa, Homo sapiens
MSIPLPLLQIYTSDNYTEEMGSGDYDSMKEPCFREENANFNKIFLPTIYSIIFLTGIVGNGLVILVMGYQKKLRSMTDKYRLHLSVADLLFVITLPFWAVDAVANWYFGNFLCKAVHVIYTVNLYSSVLILAFISLDRYLAIVHATNSQRPRKLLAEKVVYVGVWIPALLLTIPDFIFANVSEADDRYICDRFYPNDLWVVVFQFQHIMVGLILPGIVILSCYCIIISKLSHSKGHQKRKALKTTVILILAFFACWLPYYIGISIDSFILLEIIKQGCEFENTVHKWISITEALAFFHCCLNPILYAFLGAKFKTSAQHALTSVSRGSSLKILSKGKRGGHSSVSTESESSSFHSS

SEQ ID NO: 6 - protein, 352 aa, Homo sapiens
MEGISIYTSDNYTEEMGSGDYDSMKEPCFREENANFNKIFLPTIYSIIFLTGIVGNGLVILVMGYQKKLRSMTDKYRLHLSVADLLFVITLPFWAVDAVANWYFGNFLCKAVHVIYTVNLYSSVLILAFISLDRYLAIVHATNSQRPRKLLAEKVVYVGVWIPALLLTIPDFIFANVSEADDRYICDRFYPNDLWVVVFQFQHIMVGLILPGIVILSCYCIIISKLSHSKGHQKRKALKTTVILILAFFACWLPYYIGISIDSFILLEIIKQGCEFENTVHKWISITEALAFFHCCLNPILYAFLGAKFKTSAQHALTSVSRGSSLKILSKGKRGGHSSVSTESESSSFHSS

SEQ ID NO: 7 - DNA, 675 bp, Homo sapiens
gacggaagcctgtcctttcttccttttggtgcgagcttgctgtggtttttgctctgggtcctctgggatggcgcctggctgtggccgcgtggtctctcacgcaggggcgccgggcgggggaacgcggccaccctgagtctggtgagtcgactgcggcggcctgtgtccgaagtgtccggggccgtgaacaagggcagcggcctggcctcaggcctgcgttcccacgtttggaaacggggagcttcgtcgatttgtgtttacatcatcgactatgccagggagttctccagataagcctggttttattttcgtcagtgaaaaggccttaccgtataactgactttatgcttgccctgcccccgtataaaataacttaaaagcagcgtgcctggttacagctgtttccacgtgcggtgctcgtcgggagtgatcacctaccctacaggtggaagatggatgcctgaagtgtagactgctgctagctgaataccatctgggagcataaaggtgacctgaaggatgtccttggtgaggattttgaaaatttgatcttcacaagagttgcctggatcatttgaaatttctgggagtctgaggagtactgacataattacctgctggagtctgtaaatacacatttaagacagtgaggatgtgaataaatatattaatgca

SEQ ID NO: 8 - DNA, 5492 bp, Homo sapiens
cttccaccaagggcccatcggtcttccccctggcgccctgctccaggagcacctctgggggcacagcggccctgggctgcctggtcaaggactacttcccagaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacacctgcaacgtgaatcacaagcccagcaacaccaaggtggacaagagagttggtgagaggccagcgcagggagggagggtgtctgctggaagccaggctcagccctcctgcctggacgcatcccggctgtgcagtcccagcccagggcaccaaggcaggccccgtctgactcctcacccggaggcctctgcccgccccactcatgctcagggagagggtcttctggctttttccaccaggctccgggcaggcacaggctggatgcccctaccccaggcccttcacacacaggggcaggtgctgcgctcagagctgccaagagccatatccaggaggaccctgcccctgacctaagcccaccccaaaggccaaactctctactcactcagctcagataccttctctcttcccagatctgagtaactcccaatcttctctctgcagagctcaaaaccccacttggtgacacaactcacacatgcccacggtgcccaggtaagccagcccaggcctcgccctccagctcaaggcgggacaagagccctagagtggcctgagtccagggacaggccccagcagggtgctgacgcatccacctccatcccagatccccgtaactcccaatcttctctctgcagagcccaaatcttgtgacacacctcccccgtgcccacggtgcccaggtaagccagcccaggcctcgccctccagctcaaggcaggacaagagccctagagtggcctgagtccagggacaggccccagcagggtgctgacgcgtccacctccatcccagatccccgtaactcccaatcttctctctgcagagcccaaatcttgtgacacacctcccccatgcccacggtgcccaggtaagccagcccaggcctcgccctccagctcaaggcgggacaagagccctagagtggcctgagtccagggacaggccccagcagggtgctgacgcatccacctccatcccagatccccgtaactcccaatcttctctctgcagagcccaaatcttgtgacacacctcccccgtgcccaaggtgcccaggtaagccagcccaggcctcgccctccagctcaaggcaggacaggtgccctagagtggcctgcatccagggacaggtcccagtcgggtgctgacacatctgcctccatctcttcctcagcacctgaactcctgggaggaccgtcagtcttcctcttccccccaaaacccaaggatacccttatgatttcccggacccctgaggtcacgtgcgtggtggtggacgtgagccacgaagaccccgaggtccagttcaagtggtacgtggacggcgtggaggtgcataatgccaagacaaagccgcgggaggagcagtacaacagcacgttccgtgtggtcagcgtcctcaccgtcctgcaccaggactggctgaacggcaaggagtacaagtgcaaggtctccaacaaagccctcccagcccccatcgagaaaaccatctccaaaaccaaaggtgggacccgcggggtatgagggccacatggacagaggccagcttgacccaccctctgccctgggagtgaccgctgtgccaacctctgtccctacaggacagccccgagaaccacaggtgtacaccctgcccccatcccgggaggagatgaccaagaaccaggtcagcctgacctgcctggtcaaaggcttctaccccagcgacatcgccgtggagtgggagagcagcgggcagccggagaacaactacaacaccacgcctcccatgctggactccgacggctccttcttcctctacagcaagctcaccgtggacaagagcaggtggcagcaggggaacatcttctcatgctccgtgatgcatgaggctctgcacaaccgcttcacgcagaagagcctctccctgtctccgggtaaatgagtgcgacggccggcaagcccccgctccccgggctctcggggtcgcgcgaggatgcttggcacgtaccccgtgtacatacttcccgggcacccagcatggaaataaagcacccagcgctgccctgggcccctgcgagactgtgatggttctttccacgggtcaggccgagtctgaggcctgagtggcatgagggaggcagagcgggtcccactgtccccacactggcccaggctgtgcaggtgtgcctgggccgcctagggtggggctcagccaggggctgccctcggcagggtgggggatttgccagcgtggccctccctccagcagcagctgccctgggctgggccacgggaagccctaggagcccctggggacagacacacagcccctgcctctgtaggagactgtcctgtcctgtgagcgccctgtcctccgacccgcatgcccactcgggggcatgcctagtccatgtgcgtagggacaggccctccctcacccatctacccccacggcactaacccctggcagccctgcccagcctcgcacccgcatggggacacaaccgactccggggacatgcactctcgggccctgtggagagactggtccagatgcccacacacacactcagcccagacccgttcaacaaaccccgcactgaggttggccggccacacggccaccacacacacacgtgcacgcctcacacacggagcctcacccgggcgaaccgcacagcacccagaccagagcaaggtcctcgcacacgtgaacactcctcggacacaggcccccacgagccccacgcggcacctcaaggcccacgagccgctcggcagcttctccacatgctgaccagctcagacaaacccagccctcctctcacaaggtgcccctgcagccgccacacacacacaggcccccacacacaggggaacacacgccacgtcgcgtccctggcactggcccacttcccaatacagcccttccctgcagctggggtcacatgaggtgtgggcttcaccatcctcctgccctctgggcctcagggagggacacgggagacggggagtgggtcctgctgagggccaggtcgctatctagggccgggtgtgtggctgagtcccggggccaaagctggtgcccagggcgggcagctgtggggagctgacctcaggacactgttggcccatcccggccgggccctacatcctgggtcctgccacagagggaatcacccccagaggcccgagcccagcaggacacagcactgaccaccctcttcctgtccagagctgcaactggaggagagctgtgcggaggcgcaggacggggagctggacgggctgtggacgaccatcaccatcttcatcacactcttcctgttaagcgtgtgctacagtgccaccgtcaccttcttcaaggtcggccgcacgttgtccccagctgtccttgacattgtcctccatgctgtcacacactgtccctgacactgtccccaggctgtccccacctgtccctgacactgtcccccacgctctcacaaactgtccctcacactgtcccccatgctgtcacaaactgtcactgacactgtcccccatgctatccccacctgtccctgacactgtccctgacactgtctctcatgctgtccccactcatctgcgacactgtaccccacgctgtccccacttgtcctcaacaatgtcccccatgctgtccccacctgtccctgatgctgtcccccacactgtcccaatctgtccccaccactctcccccacgctgtccccacctgtccctgacactgtcccccatgccatccccatctgtcccgacaatgtccccagggtgtccccagctgtccctgatgctgtcccccacactgtccccacctctccctgacgctgtcccccacgtggtccccacttgtccctgatgctgtcccccacactgtccccacctgtccctgacactgtcccccatgccatccccatctgtcccgacaatgtccctatggtgtccccagctgtccctgatgctgtcccccacactgtccccacctgtccctgacgctgtcccccacactgtccccacctccccctgacactgtcccccacactgtccccacctctccctaacactgtcccacacactgtcccctcctgtccccaacactttcccccatgctgtccccaccagtccccaacactgtacaccatgcttttcccacctgtccccaacactgtcccccatgctgtcccctcctgtccccaacaatgtcccccatgctgtttcctcctgtccccaacactgtccgccactctgtttcctcctttccctgacactgtcccccactctgtccccacctgtagccaacactatcccctacgctgtctccacctgtccctgatgctgtcccccacactgtccccactcctccctgacactgtcccctatgctgtccccaccggttcctaacactgtcccccacactgtccctacctgtccccgacactttctcccatgctgttcccacgtgtctccaacactgtcccccacacagtctccacctgtccctgacactgtcccccatgctgtcctcacccatctctgacactgtacacatactgtccccacctgtccctgatgctgtcctccatgatgtccccacctctccctgacactgtcacccatgctgtccccacctgcccctgacactctcctccacgctgttctcacctgtccccaacactctcccccacactgtctccacctgtccctgacactgtcctccacgctgtccccacctatccctgacactgtcccccatgctgtcctcacctgtccccaacactctcctccacactgtcctcacctgtccccaacactctccccccacactgtctcaacctgtccctgacactgtcccccatgctgtcctcacctgtccctgacactgtcccccatgctgtcctcacctgtctctgacactgtcccccgtgctgtccccacctgacactatcttctgtgctgtccacatgctgttgctgccctggctctgctctccatgtccaggcctcagagcaggcagtggtgaggccctggcacatgggtggcatgaggggccggataggcctcaggggcagggctgtggcctgggtggcctgaggggtgagcaggcctcgggggcagggctgtggcctcgctcacccctgtgctgtgccttgcctacaggtgaagtggatcttctcctcggtggtggacctgaagcagaccatcatccccgactataggaacatgattgggcagggggcctag

SEQ ID NO: 9 - DNA, 3530 bp, Homo sapiens
aaaacttattagagctttctcaacctgcagccctcatctccgccggcgagtagggccaggtgttgggagctcccacgtgggacaagatggtgtcttcggcgcagatgggcttcaacctgcaggctctcctggagcagctcagccaggatgagttgagcaagttcaagtatctgatcacgaccttctccctggcacacgagctccagaagatcccccacaaggaggtagacaaggctgatgggaagcaactggtagaaatcctcaccacccattgtgacagctactgggtggagatggcgagcctccaggtctttgaaaagatgcaccgaatggatctgtctgagagagcaaaggatgaagtcagagaagcagctttgaaatcctttaataaaaggaaacctctatcattagggataacacggaaagaacgaccacctctagacgtggacgaaatgctggagcgcttcaaaacagaagcacaagcgtttacagaaacgaaaggaaatgtcatctgcctgggtaaagaagtctttaaaggaaaaaagccagacaaagacaataggtgcaggtatatattgaagacgaagttccgggagatgtggaagagctggcctggagatagcaaagaggtccaggttatggctgagagatacaagatgctgatcccattcagcaaccccagggtgcttcccgggcccttctcatacacggtggtgctgtatggtcctgcaggccttgggaaaaccacgctggcccagaaactaatgctagactgggcagaggacaacctcatccacaaattcaaatatgcgttctacctcagctgcagggagctcagccgcctgggcccgtgcagttttgcagagctggtcttcagggactggcctgaattgcaggatgacattccacacatcctagcccaagcacggaaaatcttgttcgtgattgacggctttgatgagctgggagccgcacctggggcgctgatcgaggacatctgcggggactgggagaagaagaagccggtgcccgtcctcctggggagtttgctgaacagggtgatgttacccaaggccgccctgctggtcaccacgcggcccagggccctgagggacctccggatcctggcggaggagccgatctacataagggtggagggcttcctggaggaggacaggagggcctatttcctgagacactttggagacgaggaccaagccatgcgtgcctttgagctaatgaggagcaacgcggccctgttccagctgggctcggcccccgcggtgtgctggatcgtgtgcacgactctgaagctgcagatggagaagggggaggacccggtccccacctgcctcacccgcacggggctgttcctgcgtttcctctgcagccggttcccgcagggcgcacagctgcggggcgcgctgcggacgctgagcctcctggccgcgcagggcctgtgggcgcagacgtccgtgcttcaccgagaggatctggaaaggctcggggtgcaggagtccgacctccgtctgttcctggacggagacatcctccgccaggacagagtctccaaaggctgctactccttcatccacctcagcttccagcagtttctcactgccctgttctacaccctggagaaggaggaggaagaggatagggacggccacacctgggacattggggacgtacagaagctgctttccggagtagaaagactcaggaaccccgacctgatccaagcaggctactactcctttggcctcgctaacgagaagagagccaaggagttggaggccacttttggctgccggatgtcaccggacatcaaacaggaattgctgcgatgcgacataagttgtaagggtggacattcaacggtgacagacctgcaggagctcctcggctgtctgtacgagtctcaggaggaggagctggtgaaggaggtgatggctcagttcaaagaaatatccctgcacttaaatgcagtagacgttgtgccatcttcattctgcgtcaagcactgtcgaaacctgcagaaaatgtcactgcaggtaataaaggagaatctcccggagaatgtcactgcgtctgaatcagacgccgaggttgagagatcccaggatgatcagcacatgcttcctttctggacggacctttgttccatatttggatcaaataaggatctgatgggtctagcaatcaatgatagctttctcagtgcctccctagtaaggatcctgtgtgaacaaatagcctctgacacctgtcatctccagagagtggtgttcaaaaacatttccccagctgatgctcatcggaacctctgcctagctcttcgaggtcacaagactgtaacgtatctgacccttcaaggcaatgaccaggatgatatgtttcccgcattgtgtgaggtcttgagacatccagaatgtaacctgcgatatctcgggttggtgtcttgttccgctaccactcagcagtgggctgatctctccttggcccttgaagtcaaccagtccctgacgtgcgtaaacctctccgacaatgagcttctggatgagggtgctaagttgctgtacacaactttgagacaccccaagtgctttctgcagaggttgtcgttggaaaactgtcaccttacagaagccaattgcaaggaccttgctgctgtgttggttgtcagccgggagctgacacacctgtgcttggccaagaaccccattgggaatacaggggtgaagtttctgtgtgagggcttgaggtaccccgagtgtaaactgcagaccttggtgctttggaactgcgacataactagcgatggctgctgcgatctcacaaagcttctccaagaaaaatcaagcctgttgtgtttggatctggggctgaatcacataggagttaagggaatgaagttcctgtgtgaggctttgaggaaaccactgtgcaacttgagatgtctgtggttgtggggatgttccatccctccgttcagttgtgaagacctctgctctgccctcagctgcaaccagagcctcgtcactctggacctgggtcagaatcccttggggtctagtggagtgaagatgctgtttgaaaccttgacatgttccagtggcaccctccggacactcaggttgaaaatagatgactttaatgatgaactcaataagctgctggaagaaatagaagaaaaaaacccacaactgattattgatactgagaaacatcatccctgggcagaaaggccttcttctcatgacttcatgatctgaatccccccgagtcattcattctccatgaagtcatcgattttccaggtgttggtgaactgcctgtgactcctctcctccccggcccctacccctcagggataatgagttcattgctgggctagatgttttagccatgattctgcctctgttttatacctgcacacatccttatctttgttacatatgaaatatctgtatcacgggtatattgagagaaataaaggtgagagcattcacaaaaaaaaaaaaaaaaaa

SEQ ID NO: 10 - protein, 1062 aa, Homo sapiens
MVSSAQMGFNLQALLEQLSQDELSKFKYLITTFSLAHELQKIPHKEVDKADGKQLVEILTTHCDSYWVEMASLQVFEKMHRMDLSERAKDEVREAALKSFNKRKPLSLGITRKERPPLDVDEMLERFKTEAQAFTETKGNVICLGKEVFKGKKPDKDNRCRYILKTKFREMWKSWPGDSKEVQVMAERYKMLIPFSNPRVLPGPFSYTVVLYGPAGLGKTTLAQKLMLDWAEDNLIHKFKYAFYLSCRELSRLGPCSFAELVFRDWPELQDDIPHILAQARKILFVIDGFDELGAAPGALIEDICGDWEKKKPVPVLLGSLLNRVMLPKAALLVTTRPRALRDLRILAEEPIYIRVEGFLEEDRRAYFLRHFGDEDQAMRAFELMRSNAALFQLGSAPAVCWIVCTTLKLQMEKGEDPVPTCLTRTGLFLRFLCSRFPQGAQLRGALRTLSLLAAQGLWAQTSVLHREDLERLGVQESDLRLFLDGDILRQDRVSKGCYSFIHLSFQQFLTALFYTLEKEEEEDRDGHTWDIGDVQKLLSGVERLRNPDLIQAGYYSFGLANEKRAKELEATFGCRMSPDIKQELLRCDISCKGGHSTVTDLQELLGCLYESQEEELVKEVMAQFKEISLHLNAVDVVPSSFCVKHCRNLQKMSLQVIKENLPENVTASESDAEVERSQDDQHMLPFWTDLCSIFGSNKDLMGLAINDSFLSASLVRILCEQIASDTCHLQRVVFKNISPADAHRNLCLALRGHKTVTYLTLQGNDQDDMFPALCEVLRHPECNLRYLGLVSCSATTQQWADLSLALEVNQSLTCVNLSDNELLDEGAKLLYTTLRHPKCFLQRLSLENCHLTEANCKDLAAVLVVSRELTHLCLAKNPIGNTGVKFLCEGLRYPECKLQTLVLWNCDITSDGCCDLTKLLQEKSSLLCLDLGLNHIGVKGMKFLCEALRKPLCNLRCLWLWGCSIPPFSCEDLCSALSCNQSLVTLDLGQNPLGSSGVKMLFETLTCSSGTLRTLRLKIDDFNDELNKLLEEIEEKNPQLIIDTEKHHPWAERPSSHDFMI

SEQ ID NO: 11 - DNA, 2940 bp, Homo sapiens
aaacatctgcaaaagcgcaaggagaccagcccacattttagcccctcctactcaggataagactttctctaagtccggagctgaaaaaggatcctgactgaaagctagaggcattgaggagcctgaagattctcaggttttaaagacgctagagtgccaaagaagactttgaagtgtgaaaacatttcctgtaattgaaaccaaaatgtcatttatagatccttaccagcacattatagtggagcaccagtattcccacaagtttacggtagtggtgttacgtgccaccaaagtgacaaagggggcctttggtgacatgcttgatactccagatccctatgtggaactttttatctctacaacccctgacagcaggaagagaacaagacatttcaataatgacataaaccctgtgtggaatgagacctttgaatttattttggatcctaatcaggaaaatgttttggagattacgttaatggatgccaattatgtcatggatgaaactctagggacagcaacatttactgtatcttctatgaaggtgggagaaaagaaagaagttccttttattttcaaccaagtcactgaaatggttctagaaatgtctcttgaagtttgctcatgcccagacctacgatttagtatggctctgtgtgatcaggagaagactttcagacaacagagaaaagaacacataagggagagcatgaagaaactcttgggtccaaagaatagtgaaggattgcattctgcacgtgatgtgcctgtggtagccatattgggttcaggtgggggtttccgagccatggtgggattctctggtgtgatgaaggcattatacgaatcaggaattctggattgtgctacctacgttgctggtctttctggctccacctggtatatgtcaaccttgtattctcaccctgattttccagagaaagggccagaggagattaatgaagaactaatgaaaaatgttagccacaatccccttttacttctcacaccacagaaagttaaaagatatgttgagtctttatggaagaagaaaagctctggacaacctgtcacctttactgatatctttgggatgttaataggagaaacactaattcataatagaatgaatactactctgagcagtttgaaggaaaaagttaatactgcacaatgccctttacctcttttcacctgtcttcatgtcaaacctgacgtttcagagctgatgtttgcagattgggttgaatttagtccatacgaaattggcatggctaaatatggtacttttatggctcccgacttatttggaagcaaattttttatgggaacagtcgttaagaagtatgaagaaaaccccttgcatttcttaatgggtgtctggggcagtgccttttccatattgttcaacagagttttgggcgtttctggttcacaaagcagaggctccacaatggaggaagaattagaaaatattaccacaaagcatattgtgagtaatgatagctcggacagtgatgatgaatcacacgaacccaaaggcactgaaaatgaagatgctggaagtgactatcaaagtgataatcaagcaagttggattcatcgtatgataatggccttggtgagtgattcagctttattcaataccagagaaggacgtgctgggaaggtacacaacttcatgctgggcttgaatctcaatacatcttatccactgtctcctttgagtgactttgccacacaggactcctttgatgatgatgaactggatgcagctgtagcagatcctgatgaatttgagcgaatatatgagcctctggatgtcaaaagtaaaaagattcatgtagtggacagtgggctcacatttaacctgccgtatcccttgatactgagacctcagagaggggttgatctcataatctcctttgacttttctgcaaggccaagtgactctagtcctccgttcaaggaacttctacttgcagaaaagtgggctaaaatgaacaagctcccctttccaaagattgatccttatgtgtttgatcgggaagggctgaaggagtgctatgtctttaaacccaagaatcctgatatggagaaagattgcccaaccatcatccactttgttctggccaacatcaacttcagaaagtacaaggctccaggtgttccaagggaaactgaggaagagaaagaaatcgctgactttgatatttttgatgacccagaatcaccattttcaaccttcaattttcaatatccaaatcaagcattcaaaagactacatgatcttatgcacttcaatactctgaacaacattgatgtgataaaagaagccatggttgaaagcattgaatatagaagacagaatccatctcgttgctctgtttcccttagtaatgttgaggcaagaagatttttcaacaaggagtttctaagtaaacccaaagcatagttcatgtactggaaatggcagcagtttctgatgctgaggcagtttgcaatcccatgacaactggatttaaaagtacagtacagatagtcgtactgatcatgagagactggctgatactcaaagttgcagttacttagctgcatgagaataatactattataagttaggttgacaaatgatgttgattatgtaaggatatacttagctacattttcagtcagtatgaacttcctgatacaaatgtagggatatatactgtatttttaaacatttctcaccaactttcttatgtgtgttctttttaaaaattttttttcttttaaaatatttaacagttcaatctcaataagacctcgcattatgtatgaatgttattcactgactagatttattcataccatgagacaacactatttttatttatatatgcatatatatacatacatgaaataaatacatcaatataaaaataaaaaaaaaaaaaa

SEQ ID NO: 12 - protein, 749 aa, Homo sapiens
MSFIDPYQHIIVEHQYSHKFTVVVLRATKVTKGAFGDMLDTPDPYVELFISTTPDSRKRTRHFNNDINPVWNETFEFILDPNQENVLEITLMDANYVMDETLGTATFTVSSMKVGEKKEVPFIFNQVTEMVLEMSLEVCSCPDLRFSMALCDQEKTFRQQRKEHIRESMKKLLGPKNSEGLHSARDVPVVAILGSGGGFRAMVGFSGVMKALYESGILDCATYVAGLSGSTWYMSTLYSHPDFPEKGPEEINEELMKNVSHNPLLLLTPQKVKRYVESLWKKKSSGQPVTFTDIFGMLIGETLIHNRMNTTLSSLKEKVNTAQCPLPLFTCLHVKPDVSELMFADWVEFSPYEIGMAKYGTFMAPDLFGSKFFMGTVVKKYEENPLHFLMGVWGSAFSILFNRVLGVSGSQSRGSTMEEELENITTKHIVSNDSSDSDDESHEPKGTENEDAGSDYQSDNQASWIHRMIMALVSDSALFNTREGRAGKVHNFMLGLNLNTSYPLSPLSDFATQDSFDDDELDAAVADPDEFERIYEPLDVKSKKIHVVDSGLTFNLPYPLILRPQRGVDLIISFDFSARPSDSSPPFKELLLAEKWAKMNKLPFPKIDPYVFDREGLKECYVFKPKNPDMEKDCPTIIHFVLANINFRKYKAPGVPRETEEEKEIADFDIFDDPESPFSTFNFQYPNQAFKRLHDLMHFNTLNNIDVIKEAMVESIEYRRQNPSRCSVSLSNVEARRFFNKEFLSKPKA

SEQ ID NO: 13 - DNA, 38 bp, Homo sapiens
ttgggtgttcggcggagggaccaagctgaccgtcctag

SEQ ID NO: 14 - DNA, 1498 bp, Homo sapiens
gaggccagagtgccatcgaaggtaattatagagacagtaaaatccttttactctgggaaaaataaaatgctgggtgtctcacaaaatttcagaacctgatttcaaacggatcataacaaagaggagatcaaatttagcatggtggactgctcgacaggatatatttgtcaatggaatgtttccacatattataccaccaacatgagaaaaaaatgatcattgtttatttgaagcttgatgatattctaacgctgccttttctcttctcattttagagaaaaatgagcaggcggaattgttggatttgtaagatgtgcagagatgaatctaagaggcccccttcaaaccttactttggaggaagtattacagtgggcccagtcttttgaaaatttaatggctacaaaatatggtccagtagtctatgcagcatatttaaaaatggagcacagtgacgagaatattcaattctggatggcatgtgaaacctataagaaaattgcctcacggtggagcagaatttctagggcaaagaagctttataagatttacatccagccacagtcccctagagagattaacattgacagttcgacaagagagactatcatcaggaacattcaggaacccactgaaacatgttttgaagaagctcagaaaatagtctatatgcatatggaaagggattcctaccccagatttctaaagtcagaaatgtaccaaaaacttttgaaaactatgcagtccaacaacagtttctgactacaactcaaaagtttaaatagaaaacagtatattgaaagtggtgggtttgatctttttatttagaaacccacaaaatcagaaacacagtacaaataaaacagaaatcaaactataagttgacttttagttcctaaaaagaaacatatttcaaaagcaatggaatctagaattcttataacatgaataacaaaatgtacagcaagcctatgtagttcaattaatatataaggaaaaggaaggtctttcttcatgatacaagcattataaagtttttactgtagtagtcaattaatggatatttccttgttaataaaattttgtgtcataatttacaaattagttctttaaaaattgttgttatatgaattgtgtttctagcatgaatgttctatagagtactctaaataacttgaatttatagacaaatgctactcacagtacaatcaattgtattataccatgagaaaatcaaaaaggtgttcttcagagacattttatctataaaattttcctactattatgttcattaacaaacttctttatcacatgtatcttctacatgtaaaacatttctgatgattttttaacaaaaaatatatgaatttcttcatttgctcttgcatctacattgctataaggatataaaatgtggtttctatattttgagatgttttttccttacaatgtgaactcatcgtgatcttggaaatcaataaagtcaaatatcaactaaa

SEQ ID NO: 15 - DNA, 1458 bp, Homo sapiens
gaggccagagtgccatcgaaggtaattatagagacagtaaaatccttttactctgggaaaaataaaatgctgggtgtctcacaaaatttcagaacctgatttcaaacggatcataacaaagaggagatcaaatttagcatggtggactgctcgacaggatatatttgtcaatggaatgtttccacatattataccaccaacatgagaaaaaaatgatcattgtttatttgaagcttgaaaaatgagcaggcggaattgttggatttgtaagatgtgcagagatgaatctaagaggcccccttcaaaccttactttggaggaagtattacagtgggcccagtcttttgaaaatttaatggctacaaaatatggtccagtagtctatgcagcatatttaaaaatggagcacagtgacgagaatattcaattctggatggcatgtgaaacctataagaaaattgcctcacggtggagcagaatttctagggcaaagaagctttataagatttacatccagccacagtcccctagagagattaacattgacagttcgacaagagagactatcatcaggaacattcaggaacccactgaaacatgttttgaagaagctcagaaaatagtctatatgcatatggaaagggattcctaccccagatttctaaagtcagaaatgtaccaaaaacttttgaaaactatgcagtccaacaacagtttctgactacaactcaaaagtttaaatagaaaacagtatattgaaagtggtgggtttgatctttttatttagaaacccacaaaatcagaaacacagtacaaataaaacagaaatcaaactataagttgacttttagttcctaaaaagaaacatatttcaaaagcaatggaatctagaattcttataacatgaataacaaaatgtacagcaagcctatgtagttcaattaatatataaggaaaaggaaggtctttcttcatgatacaagcattataaagtttttactgtagtagtcaattaatggatatttccttgttaataaaattttgtgtcataatttacaaattagttctttaaaaattgttgttatatgaattgtgtttctagcatgaatgttctatagagtactctaaataacttgaatttatagacaaatgctactcacagtacaatcaattgtattataccatgagaaaatcaaaaaggtgttcttcagagacattttatctataaaattttcctactattatgttcattaacaaacttctttatcacatgtatcttctacatgtaaaacatttctgatgattttttaacaaaaaatatatgaatttcttcatttgctcttgcatctacattgctataaggatataaaatgtggtttctatattttgagatgttttttccttacaatgtgaactcatcgtgatcttggaaatcaataaagtcaaatatcaactaaa

SEQ ID NO: 16 - protein, 159 aa, Homo sapiens
MSRRNCWICKMCRDESKRPPSNLTLEEVLQWAQSFENLMATKYGPVVYAAYLKMEHSDENIQFWMACETYKKIASRWSRISRAKKLYKIYIQPQSPREINIDSSTRETIIRNIQEPTETCFEEAQKIVYMHMERDSYPRFLKSEMYQKLLKTMQSNNSF

SEQ ID NO: 17 - protein, 159 aa, Homo sapiens; identical to SEQ ID NO: 16

SEQ ID NO: 18 - DNA, 1216 bp, Homo sapiens
ttcggcacttgggagaagatgtttgaaaaaactgactctgctaatgagcctggactcagagctcaagtctgaactctacctccagacagaatgaagttcatctcgacatctctgcttctcatgctgctggtcagcagcctctctccagtccaaggtgttctggaggtctattacacaagcttgaggtgtagatgtgtccaagagagctcagtctttatccctagacgcttcattgatcgaattcaaatcttgccccgtgggaatggttgtccaagaaaagaaatcatagtctggaagaagaacaagtcaattgtgtgtgtggaccctcaagctgaatggatacaaagaatgatggaagtattgagaaaaagaagttcttcaactctaccagttccagtgtttaagagaaagattccctgatgctgatatttccactaagaacacctgcattcttcccttatccctgctctggattttagttttgtgcttagttaaatcttttccagggagaaagaacttccccatacaaataaggcatgaggactatgtgaaaaataaccttgcaggagctgatggggcaaactcaagcttcttcactcacagcaccctatatacacttggagtttgcattcttattcatcagggaggaaagtttctttgaaaatagttattcagttataagtaatacaggattattttgattatatacttgttgtttaatgtttaaaatttcttagaaaacaatggaatgagaatttaagcctcaaatttgaacatgtggcttgaattaagaagaaaattatggcatatattaaaagcaggcttctatgaaagactcaaaaagctgcctgggaggcagatggaacttgagcctgtcaagaggcaaaggaatccatgtagtagatatcctctgcttaaaaactcactacggaggagaattaagtcctacttttaaagaatttctttataaaatttactgtctaagattaatagcattcgaagatccccagacttcatagaatactcagggaaagcatttaaagggtgatgtacacatgtatcctttcacacatttgccttgacaaacttctttcactcacatctttttcactgactttttttgtgggggcggggccggggggactctggtatctaattctttaatgattcctataaatctaatgacattcaataaagttgagcaaacattttactt

SEQ ID NO: 19 - protein, 109 aa, Homo sapiens
MKFISTSLLLMLLVSSLSPVQGVLEVYYTSLRCRCVQESSVFIPRRFIDRIQILPRGNGCPRKEIIVWKKNKSIVCVDPQAEWIQRMMEVLRKRSSSTLPVPVFKRKIP

SEQ ID NO: 20 - DNA, 5817 bp, Homo sapiens
gcggagaaggaggcggagggagcgattgtggccccggccgcggtggccggcgcggcctgccctttgtgaccgcagctcgcgccccacgccccgcgcccatggccgccgtgccgggctccctggccacgcgtgcccgcccgcggacctgagccccgcgcctgggatgccggggatgcgcgtcccccggccctgcggctgctccgggctgggcgcggggcgatggacctgagcatgaagaagttcgccgtgcgcaggttcttctctgtgtacctgcgcaggaagtcgcgctccaagagctccagcctgagccggctcgaggaagaaggcgtcgtgaaggagatagacatcagccatcatgtgaaggagggctttgagaaggcagatccttcccagtttgagctgctgaaggttttaggacaaggatcctatggaaaggtgttcctggtgaggaaggtgaaggggtccgacgctgggcagctctacgccatgaaggtccttaagaaagccaccctaaaagttcgggaccgagtgagatcgaagatggagagagacatcttggcagaagtgaatcaccccttcattgtgaagcttcattatgcctttcagacggaaggaaagctctacctgatcctggacttcctgcggggaggggacctcttcacccggctctccaaagaggtcatgttcacggaggaggatgtcaagttctacctggctgagctggccttggctttagaccatctccacagcctggggatcatctacagagatctgaagcctgagaacatcctcctggatgaagaggggcacattaagatcacagatttcggcctgagtaaggaggccattgaccacgacaagagagcgtactccttctgcgggacgatcgagtacatggcgcccgaggtggtgaaccggcgaggacacacgcagagtgccgactggtggtccttcggcgtgctcatgtttgagatgctcacggggtccctgccgttccaggggaaggacaggaaggagaccatggctctcatcctcaaagccaagctggggatgccgcagttcctcagtggggaggcacagagtttgctgcgagctctcttcaaacggaacccctgcaaccggctgggtgctggcattgacggagtggaggaaattaagcgccatcccttctttgtgaccatagactggaacacgctgtaccggaaggagatcaagccaccgttcaaaccagcagtgggcaggcctgaggacaccttccactttgaccccgagttcacagcgcggacgcccacagactctcctggcgtccccccgagtgcaaacgctcatcacctgtttagaggattcagctttgtggcctcaagcctgatccaggagccctcacagcaagatctgcacaaagtcccagttcacccaatcgtgcagcagttacacgggaacaacatccacttcaccgatggctacgagatcaaggaggacatcggggtgggctcctactcagtgtgcaagcgatgtgtgcataaagccacagacaccgagtatgccgtgaagatcattgataagagcaagagagacccctcggaagagattgagatcctcctgcggtacggccagcacccgaacatcatcaccctcaaggatgtctatgatgatggcaagtttgtgtacctggtaatggagctgatgcgtggtggggagctcctggaccgcatcctccggcagagatacttctcggagcgcgaagccagtgacgtcctgtgcaccatcaccaagaccatggactacctccattcccagggggttgttcatcgagacctgaagccgagtaacatcctgtacagggatgagtcggggagcccagaatccatccgagtctgcgacttcggctttgccaagcagctgcgcgcggggaacgggctgctcatgacaccctgctacacggccaatttcgtggccccggaggtcctgaagcgtcaaggctatgatgcggcgtgtgacatctggagtttggggatcctgttgtacaccatgctggcaggatttaccccttttgcaaatgggccagacgatacccctgaggagattctggcgcggatcggcagtgggaagtatgccctttctgggggaaactgggactcgatatctgacgcagctaaagacgtcgtgtccaagatgctccacgtggaccctcatcagcgcctgacggcgatgcaagtgctcaaacacccgtgggtggtcaacagagagtacctgtccccaaaccagctcagccgacaggacgtgcacctggtgaagggcgcgatggccgccacctactttgctctaaacagaacacctcaggccccgcggctggagcccgtgctgtcatccaacctggctcagcgcagaggcatgaagagactcacgtccacgcggctgtagcgggtgggaccctggccccagcgtcccctgccagcatcctcgtgggctcacagaccccggcctcggagcccgtctggcacccagagtgaccacaagtccagcagggaggcggcgcccgccctcgccgtgtccgtgttttctttttcagccccggagagggtcctgacctgggggcttctccaagcctcactgcgccagcctccccgcccgctctcttttctcccaagcgaaaccaaatgcgccccttcacctcgcgtgcccgtgcgaggccgggggcttctttcagagcccgcgggtcctctcatacatggcttctgtttctgccgagagatctgttttccaattatgaagccggtcggtttggtcagactcccgacacccacgtcccaggtacccggtgggaaagtggcagtgcgagggcgcagccattggtggttgcagggccccagagggctggggtgacctggcatcccggggctccccacgggctggatgacggggttggcactgtggcgtccaggaggagatgcctggttctgcccaaaataatccaaagagccgtttcctcctcgcccttcagtttttgcctgaggtgctgggtagcccatcctttcctctgtcccagattcaaatgaggagtaagagcccagacgagaggaaggcaggctggatctttgccttgagagctccgtgtcaccaggatggaagggggtgcctctcggaggagcctgtgtccacctccagtctcggctttccccggggggccaagcgcactgggctgccgtctgtccccagctcccgtggccacacagctatctggaggctttgcagggagtcgtgggttctcgcacctgctcagccctgtgtcggcttcctgtgtgctcacctaaagctgtggttttgctgtgttcacttcgatttttctggtctgtggagaaactgtgaattggagaaatggagctctgtggcttcccacccaaaccttctcagtccagctggaggctggagggagacacaggccccacccagcagactgaggggcagaggcacaggtgggagggcagcggagatcagcgtggacaggagcgatgcactttgtagatgctgtggctttgtgttgcgttttgtgtctctgttgcacagatctgttttttcacactgatccgtattcccctgggtgtgcacacagggcgggtgtggggcatttaggccatgctgtgctctacttcattgagtaaaatcgagtgagaggttccgggcagcaggatcgacgcccagtccagccggcagagggaacacacgggtccttcattgtcctgtaagggtgttgaagatgctccctggcggcccccaagcagactagatgggaggaggcgccgctcagcccctcaccctgcatcactgaagagcggcgcctctgcagcaagcagggcttcaggaggtgcccgctggccacagccaggttttccctaagaagatgttattttgttgggttttgttccccctccatctcgattctcgtacccaactaaaaaaaaaaaaataaagaaaaaatgtgctgcgttctgaaaaataactccttagcttggtctgattgttttcagaccttaaaatataaacttgtttcacaagctttaatccatgtggattttttttttcttagagaaccacaaaacataaaaggagcaagtcggactgaatacctgtttccatagtgcccacagggtattcctcacattttctccatagaagatgctttttcccaaggctagaacgacttccaccatgatgaatttgctttttaggtcttaattatttcacttctttttagaaacttaggaagaagtggataatcctgaggtcacacaatctgtcctcccagaaatgaacaaaagtcatcaccttttctgcttgctacacaggcaacgattcccccatcagctgcccggaccctttggcctggcttggtgtgcaggcctgtctgtttgcttaaagtcagtgggttctggtgcagggagtgagaagtgggggaagtgaaagggaaagcatccgtgagaaagcggccacggttttccctccttgtgtgcccatggggcaccagctcatggtctttttcagtcatcccagtttgtacagacttagcttctgaactctaagaatgccaaagggaccgacgagactccccatcacagcgagctctgtccttacatgtatttgatgtgcatcagcggaggagaacactggcttggccctgctccgctgagtgtctgtgaaatacctctactttccctcccatatccagaacaaaatgatacttgacatccttccacaaaagtcagcctaaagaagttatggtatcatatgttaaactaagctttcaaaaaccttagtgaaatagcaagtgactgctttcaagcagcagtcgacatgtaaatgaaggtgttcttagaattcgcattttgccagctcagcgcacctccacaacgaatgaaatgctccgtatgatttgcacaaatgacatagacctccccaaaagttaactggctctccttcctcacacagttcatcataacccaaccccccacccccgggtcatgaaaatcacagaacttataaacacattgaaccctagatctcaggcttcctgacctaccgccagtggccccttgctggccaccctatagggtcctccttccctggcagccccccatgtgggagaaatacctgattctcccaatctgcagtgggagagctttgctgaattccatcccaaagtcaaacatgggcaagaggtgaggatttcacttttaccctcaagtccgatttgtctgtgattttaaactaactgtgtatgtattgatgtttggaagattgtttgaattttaaagtgataatagtacttaatgttatccagtattgttcattaaatggtgttatcctaaagctgcacttgggatttttacctaacgctttactgattctctcaagcacatggcaaagtttgatttgcactccgttcatttctgacacgttttgctgcctcctacctttctaagcgtcatgcaaattcgagaatggagaaggacgctgccggtccctgagcggtgtggagagggcggaaggtggactccagcgcagcttgaggggctgaggacggaggctgcagcatctgtgtcgttctactgagcacgcttctctgcctcgctcctgactcagcactttgttcactggctcagcagttatgtttacacatcatttttatgttcctgctttgtaattcatgtttgagatgggtggccactgtacagatatttattacgctttccagactttctgaatagatttttttgaataaacatggttttatgaagtgtaatctttttctagcctaacaat

SEQ ID NO: 21 - DNA, 5735 bp, Homo sapiens
gcgtcccttggcttccgacatcccgtctggccgtccccctgtgccggtccgagcctctgtttatttcctttcctactatcaatactcgaccagcagaaaaggaaagtttaaaaatgccaatcgcacagttgctggaactatggaaaaagatcgaggtggagcctatggaaatagagaccacagaggaggatctcaacctggatgtggagcccaccacagaagacactgcagaagaagaagaaggcgtcgtgaaggagatagacatcagccatcatgtgaaggagggctttgagaaggcagatccttcccagtttgagctgctgaaggttttaggacaaggatcctatggaaaggtgttcctggtgaggaaggtgaaggggtccgacgctgggcagctctacgccatgaaggtccttaagaaagccaccctaaaagttcgggaccgagtgagatcgaagatggagagagacatcttggcagaagtgaatcaccccttcattgtgaagcttcattatgcctttcagacggaaggaaagctctacctgatcctggacttcctgcggggaggggacctcttcacccggctctccaaagaggtcatgttcacggaggaggatgtcaagttctacctggctgagctggccttggctttagaccatctccacagcctggggatcatctacagagatctgaagcctgagaacatcctcctggatgaagaggggcacattaagatcacagatttcggcctgagtaaggaggccattgaccacgacaagagagcgtactccttctgcgggacgatcgagtacatggcgcccgaggtggtgaaccggcgaggacacacgcagagtgccgactggtggtccttcggcgtgctcatgtttgagatgctcacggggtccctgccgttccaggggaaggacaggaaggagaccatggctctcatcctcaaagccaagctggggatgccgcagttcctcagtggggaggcacagagtttgctgcgagctctcttcaaacggaacccctgcaaccggctgggtgctggcattgacggagtggaggaaattaagcgccatcccttctttgtgaccatagactggaacacgctgtaccggaaggagatcaagccaccgttcaaaccagcagtgggcaggcctgaggacaccttccactttgaccccgagttcacagcgcggacgcccacagactctcctggcgtccccccgagtgcaaacgctcatcacctgtttagaggattcagctttgtggcctcaagcctgatccaggagccctcacagcaagatctgcacaaagtcccagttcacccaatcgtgcagcagttacacgggaacaacatccacttcaccgatggctacgagatcaaggaggacatcggggtgggctcctactcagtgtgcaagcgatgtgtgcataaagccacagacaccgagtatgccgtgaagatcattgataagagcaagagagacccctcggaagagattgagatcctcctgcggtacggccagcacccgaacatcatcaccctcaaggatgtctatgatgatggcaagtttgtgtacctggtaatggagctgatgcgtggtggggagctcctggaccgcatcctccggcagagatacttctcggagcgcgaagccagtgacgtcctgtgcaccatcaccaagaccatggactacctccattcccagggggttgttcatcgagacctgaagccgagtaacatcctgtacagggatgagtcggggagcccagaatccatccgagtctgcgacttcggctttgccaagcagctgcgcgcggggaacgggctgctcatgacaccctgctacacggccaatttcgtggccccggaggtcctgaagcgtcaaggctatgatgcggcgtgtgacatctggagtttggggatcctgttgtacaccatgctggcaggatttaccccttttgcaaatgggccagacgatacccctgaggagattctggcgcggatcggcagtgggaagtatgccctttctgggggaaactgggactcgatatctgacgcagctaaagacgtcgtgtccaagatgctccacgtggaccctcatcagcgcctgacggcgatgcaagtgctcaaacacccgtgggtggtcaacagagagtacctgtccccaaaccagctcagccgacaggacgtgcacctggtgaagggcgcgatggccgccacctactttgctctaaacagaacacctcaggccccgcggctggagcccgtgctgtcatccaacctggctcagcgcagaggcatgaagagactcacgtccacgcggctgtagcgggtgggaccctggccccagcgtcccctgccagcatcctcgtgggctcacagaccccggcctcggagcccgtctggcacccagagtgaccacaagtccagcagggaggcggcgcccgccctcgccgtgtccgtgttttctttttcagccccggagagggtcctgacctgggggcttctccaagcctcactgcgccagcctccccgcccgctctcttttctcccaagcgaaaccaaatgcgccccttcacctcgcgtgcccgtgcgaggccgggggcttctttcagagcccgcgggtcctctcatacatggcttctgtttctgccgagagatctgttttccaattatgaagccggtcggtttggtcagactcccgacacccacgtcccaggtacccggtgggaaagtggcagtgcgagggcgcagccattggtggttgcagggccccagagggctggggtgacctggcatcccggggctccccacgggctggatgacggggttggcactgtggcgtccaggaggagatgcctggttctgcccaaaataatccaaagagccgtttcctcctcgcccttcagtttttgcctgaggtgctgggtagcccatcctttcctctgtcccagattcaaatgaggagtaagagcccagacgagaggaaggcaggctggatctttgccttgagagctccgtgtcaccaggatggaagggggtgcctctcggaggagcctgtgtccacctccagtctcggctttccccggggggccaagcgcactgggctgccgtctgtccccagctcccgtggccacacagctatctggaggctttgcagggagtcgtgggttctcgcacctgctcagccctgtgtcggcttcctgtgtgctcacctaaagctgtggttttgctgtgttcacttcgatttttctggtctgtggagaaactgtgaattggagaaatggagctctgtggcttcccacccaaaccttctcagtccagctggaggctggagggagacacaggccccacccagcagactgaggggcagaggcacaggtgggagggcagcggagatcagcgtggacaggagcgatgcactttgtagatgctgtggctttgtgttgcgttttgtgtctctgttgcacagatctgttttttcacactgatccgtattcccctgggtgtgcacacagggcgggtgtggggcatttaggccatgctgtgctctacttcattgagtaaaatcgagtgagaggttccgggcagcaggatcgacgcccagtccagccggcagagggaacacacgggtccttcattgtcctgtaagggtgttgaagatgctccctggcggcccccaagcagactagatgggaggaggcgccgctcagcccctcaccctgcatcactgaagagcggcgcctctgcagcaagcagggcttcaggaggtgcccgctggccacagccaggttttccctaagaagatgttattttgttgggttttgttccccctccatctcgattctcgtacccaactaaaaaaaaaaaaataaagaaaaaatgtgctgcgttctgaaaaataactccttagcttggtctgattgttttcagaccttaaaatataaacttgtttcacaagctttaatccatgtggattttttttttcttagagaaccacaaaacataaaaggagcaagtcggactgaatacctgtttccatagtgcccacagggtattcctcacattttctccatagaagatgctttttcccaaggctagaacgacttccaccatgatgaatttgctttttaggtcttaattatttcacttctttttagaaacttaggaagaagtggataatcctgaggtcacacaatctgtcctcccagaaatgaacaaaagtcatcaccttttctgcttgctacacaggcaacgattcccccatcagctgcccggaccctttggcctggcttggtgtgcaggcctgtctgtttgcttaaagtcagtgggttctggtgcagggagtgagaagtgggggaagtgaaagggaaagcatccgtgagaaagcggccacggttttccctccttgtgtgcccatggggcaccagctcatggtctttttcagtcatcccagtttgtacagacttagcttctgaactctaagaatgccaaagggaccgacgagactccccatcacagcgagctctgtccttacatgtatttgatgtgcatcagcggaggagaacactggcttggccctgctccgctgagtgtctgtgaaatacctctactttccctcccatatccagaacaaaatgatacttgacatccttccacaaaagtcagcctaaagaagttatggtatcatatgttaaactaagctttcaaaaaccttagtgaaatagcaagtgactgctttcaagcagcagtcgacatgtaaatgaaggtgttcttagaattcgcattttgccagctcagcgcacctccacaacgaatgaaatgctccgtatgatttgcacaaatgacatagacctccccaaaagttaactggctctccttcctcacacagttcatcataacccaaccccccacccccgggtcatgaaaatcacagaacttataaacacattgaaccctagatctcaggcttcctgacctaccgccagtggccccttgctggccaccctatagggtcctccttccctggcagccccccatgtgggagaaatacctgattctcccaatctgcagtgggagagctttgctgaattccatcccaaagtcaaacatgggcaagaggtgaggatttcacttttaccctcaagtccgatttgtctgtgattttaaactaactgtgtatgtattgatgtttggaagattgtttgaattttaaagtgataatagtacttaatgttatccagtattgttcattaaatggtgttatcctaaagctgcacttgggatttttacctaacgctttactgattctctcaagcacatggcaaagtttgatttgcactccgttcatttctgacacgttttgctgcctcctacctttctaagcgtcatgcaaattcgagaatggagaaggacgctgccggtccctgagcggtgtggagagggcggaaggtggactccagcgcagcttgaggggctgaggacggaggctgcagcatctgtgtcgttctactgagcacgcttctctgcctcgctcctgactcagcactttgttcactggctcagcagttatgtttacacatcatttttatgttcctgctttgtaattcatgtttgagatgggtggccactgtacagatatttattacgctttccagactttctgaatagatttttttgaataaacatggttttatgaagtgtaatctttttctagcctaacaat

SEQ ID NO: 22 - protein, 733 aa, Homo sapiens
MDLSMKKFAVRRFFSVYLRRKSRSKSSSLSRLEEEGVVKEIDISHHVKEGFEKADPSQFELLKVLGQGSYGKVFLVRKVKGSDAGQLYAMKVLKKATLKVRDRVRSKMERDILAEVNHPFIVKLHYAFQTEGKLYLILDFLRGGDLFTRLSKEVMFTEEDVKFYLAELALALDHLHSLGIIYRDLKPENILLDEEGHIKITDFGLSKEAIDHDKRAYSFCGTIEYMAPEVVNRRGHTQSADWWSFGVLMFEMLTGSLPFQGKDRKETMALILKAKLGMPQFLSGEAQSLLRALFKRNPCNRLGAGIDGVEEIKRHPFFVTIDWNTLYRKEIKPPFKPAVGRPEDTFHFDPEFTARTPTDSPGVPPSANAHHLFRGFSFVASSLIQEPSQQDLHKVPVHPIVQQLHGNNIHFTDGYEIKEDIGVGSYSVCKRCVHKATDTEYAVKIIDKSKRDPSEEIEILLRYGQHPNIITLKDVYDDGKFVYLVMELMRGGELLDRILRQRYFSEREASDVLCTITKTMDYLHSQGVVHRDLKPSNILYRDESGSPESIRVCDFGFAKQLRAGNGLLMTPCYTANFVAPEVLKRQGYDAACDIWSLGILLYTMLAGFTPFANGPDDTPEEILARIGSGKYALSGGNWDSISDAAKDVVSKMLHVDPHQRLTAMQVLKHPWVVNREYLSPNQLSRQDVHLVKGAMAATYFALNRTPQAPRLEPVLSSNLAQRRGMKRLTSTRL

SEQ ID NO: 23 - protein, 741 aa, Homo sapiens
MPIAQLLELWKKIEVEPMEIETTEEDLNLDVEPTTEDTAEEEEGVVKEIDISHHVKEGFEKADPSQFELLKVLGQGSYGKVFLVRKVKGSDAGQLYAMKVLKKATLKVRDRVRSKMERDILAEVNHPFIVKLHYAFQTEGKLYLILDFLRGGDLFTRLSKEVMFTEEDVKFYLAELALALDHLHSLGIIYRDLKPENILLDEEGHIKITDFGLSKEAIDHDKRAYSFCGTIEYMAPEVVNRRGHTQSADWWSFGVLMFEMLTGSLPFQGKDRKETMALILKAKLGMPQFLSGEAQSLLRALFKRNPCNRLGAGIDGVEEIKRHPFFVTIDWNTLYRKEIKPPFKPAVGRPEDTFHFDPEFTARTPTDSPGVPPSANAHHLFRGFSFVASSLIQEPSQQDLHKVPVHPIVQQLHGNNIHFTDGYEIKEDIGVGSYSVCKRCVHKATDTEYAVKIIDKSKRDPSEEIEILLRYGQHPNIITLKDVYDDGKFVYLVMELMRGGELLDRILRQRYFSEREASDVLCTITKTMDYLHSQGVVHRDLKPSNILYRDESGSPESIRVCDFGFAKQLRAGNGLLMTPCYTANFVAPEVLKRQGYDAACDIWSLGILLYTMLAGFTPFANGPDDTPEEILARIGSGKYALSGGNWDSISDAAKDVVSKMLHVDPHQRLTAMQVLKHPWVVNREYLSPNQLSRQDVHLVKGAMAATYFALNRTPQAPRLEPVLSSNLAQRRGMKRLTSTRL

SEQ ID NO: 24 - DNA, 1147 bp, Homo sapiens
accaaatcaaccataggtccaagaacaattgtctctggacggcagctatgcgactcaccgtgctgtgtgctgtgtgcctgctgcctggcagcctggccctgccgctgcctcaggaggcgggaggcatgagtgagctacagtgggaacaggctcaggactatctcaagagattttatctctatgactcagaaacaaaaaatgccaacagtttagaagccaaactcaaggagatgcaaaaattctttggcctacctataactggaatgttaaactcccgcgtcatagaaataatgcagaagcccagatgtggagtgccagatgttgcagaatactcactatttccaaatagcccaaaatggacttccaaagtggtcacctacaggatcgtatcatatactcgagacttaccgcatattacagtggatcgattagtgtcaaaggctttaaacatgtggggcaaagagatccccctgcatttcaggaaagttgtatggggaactgctgacatcatgattggctttgcgcgaggagctcatggggactcctacccatttgatgggccaggaaacacgctggctcatgcctttgcgcctgggacaggtctcggaggagatgctcacttcgatgaggatgaacgctggacggatggtagcagtctagggattaacttcctgtatgctgcaactcatgaacttggccattctttgggtatgggacattcctctgatcctaatgcagtgatgtatccaacctatggaaatggagatccccaaaattttaaactttcccaggatgatattaaaggcattcagaaactatatggaaagagaagtaattcaagaaagaaatagaaacttcaggcagaacatccattcattcattcattggattgtatatcattgttgcacaatcagaattgataagcactgttcctccactccatttagcaattatgtcacccttttttattgcagttggtttttgaatgtctttcactccttttaaggataaactcctttatggtgtgactgtgtcttattcatctatacttgcagtgggtagatgtcaataaatgttacatacacaaataaataaaatgtttattccatggtaaatttaaaaaaaaaaaaaaaaaaaaaaaaaaaaa

SEQ ID NO: 25 - protein, 267 aa, Homo sapiens
MRLTVLCAVCLLPGSLALPLPQEAGGMSELQWEQAQDYLKRFYLYDSETKNANSLEAKLKEMQKFFGLPITGMLNSRVIEIMQKPRCGVPDVAEYSLFPNSPKWTSKVVTYRIVSYTRDLPHITVDRLVSKALNMWGKEIPLHFRKVVWGTADIMIGFARGAHGDSYPFDGPGNTLAHAFAPGTGLGGDAHFDEDERWTDGSSLGINFLYAATHELGHSLGMGHSSDPNAVMYPTYGNGDPQNFKLSQDDIKGIQKLYGKRSNSRKK

SEQ ID NO: 26 - DNA, 1847 bp, Homo sapiens
ggtaccatagagttgctctgaaaacagaagatagagggagtctcggagctcgccatctccagcgatctctacattgggaaaaaacatggagtcagctccggcagcccccgaccccgccgccagcgagccaggcagcagcggcgcggacgcggccgccggctccagggagaccccgctgaaccaggaatccgcccgcaagagcgagccgcctgccccggtgcgcagacagagctattccagcaccagcagaggtatctcagtaacgaagaagacacatacatctcaaattgaaattattccatgcaagatctgtggagacaaatcatcaggaatccattatggtgtcattacatgtgaaggctgcaagggctttttcaggagaagtcagcaaagcaatgccacctactcctgtcctcgtcagaagaactgtttgattgatcgaaccagtagaaaccgctgccaacactgtcgattacagaaatgccttgccgtagggatgtctcgagatgctgtaaaatttggccgaatgtcaaaaaagcagagagacagcttgtatgcagaagtacagaaacaccggatgcagcagcagcagcgcgaccaccagcagcagcctggagaggctgagccgctgacgcccacctacaacatctcggccaacgggctgacggaacttcacgacgacctcagtaactacattgacgggcacacccctgaggggagtaaggcagactccgccgtcagcagcttctacctggacatacagccttccccagaccagtcaggtcttgatatcaatggaatcaaaccagaaccaatatgtgactacacaccagcatcaggcttctttccctactgttcgttcaccaacggcgagacttccccaactgtgtccatggcagaattagaacaccttgcacagaatatatctaaatcgcatctggaaacctgccaatacttgagagaagagctccagcagataacgtggcagacctttttacaggaagaaattgagaactatcaaaacaagcagcgggaggtgatgtggcaattgtgtgccatcaaaattacagaagctatacagtatgtggtggagtttgccaaacgcattgatggatttatggaactgtgtcaaaatgatcaaattgtgcttctaaaagcaggttctctagaggtggtgtttatcagaatgtgccgtgcctttgactctcagaacaacaccgtgtactttgatgggaagtatgccagccccgacgtcttcaaatccttaggttgtgaagactttattagctttgtgtttgaatttggaaagagtttatgttctatgcacctgactgaagatgaaattgcattattttctgcatttgtactgatgtcagcagatcgctcatggctgcaagaaaaggtaaaaattgaaaaactgcaacagaaaattcagctagctcttcaacacgtcctacagaagaatcaccgagaagatggaatactaacaaagttaatatgcaaggtgtctacattaagagccttatgtggacgacatacagaaaagctaatggcatttaaagcaatatacccagacattgtgcgacttcattttcctccattatacaaggagttgttcacttcagaatttgagccagcaatgcaaattgatgggtaaatgttatcacctaagcacttctagaatgtctgaagtacaaacatgaaaaacaaacaaaaaaattaaccgagacactttatatggccctgcacagacctggagcgccacacactgcacatcttttggtgatcggggtcaggcaaaggaggggaaacaatgaaaacaaataaagttgaacttgtttttctca

SEQ ID NO: 27 - DNA, 2020 bp, Homo sapiens
gcagattcacagggcctctgagcattatcccccatactcctccccatcattctccacccagctgttggagccatctgtctgatcaccttggactccatagtacactggggcaaagcacagccccagtttctggaggcagatgggtaaccaggaaaaggcatgaatgagggggccccaggagacagtgacttagagactgaggcaagagtgccgtggtcaatcatgggtcattgtcttcgaactggacaggccagaatgtctgccacacccacacctgcaggtgaaggagccagaagggatgaactttttgggattctccaaatactccatcagtgtatcctgtcttcaggtgatgcttttgttcttactggcgtctgttgttcctggaggcagaatggcaagccaccatattcacaaaaggaagataaggaagtacaaactggatacatgaatgctcaaattgaaattattccatgcaagatctgtggagacaaatcatcaggaatccattatggtgtcattacatgtgaaggctgcaagggctttttcaggagaagtcagcaaagcaatgccacctactcctgtcctcgtcagaagaactgtttgattgatcgaaccagtagaaaccgctgccaacactgtcgattacagaaatgccttgccgtagggatgtctcgagatgctgtaaaatttggccgaatgtcaaaaaagcagagagacagcttgtatgcagaagtacagaaacaccggatgcagcagcagcagcgcgaccaccagcagcagcctggagaggctgagccgctgacgcccacctacaacatctcggccaacgggctgacggaacttcacgacgacctcagtaactacattgacgggcacacccctgaggggagtaaggcagactccgccgtcagcagcttctacctggacatacagccttccccagaccagtcaggtcttgatatcaatggaatcaaaccagaaccaatatgtgactacacaccagcatcaggcttctttccctactgttcgttcaccaacggcgagacttccccaactgtgtccatggcagaattagaacaccttgcacagaatatatctaaatcgcatctggaaacctgccaatacttgagagaagagctccagcagataacgtggcagacctttttacaggaagaaattgagaactatcaaaacaagcagcgggaggtgatgtggcaattgtgtgccatcaaaattacagaagctatacagtatgtggtggagtttgccaaacgcattgatggatttatggaactgtgtcaaaatgatcaaattgtgcttctaaaagcaggttctctagaggtggtgtttatcagaatgtgccgtgcctttgactctcagaacaacaccgtgtactttgatgggaagtatgccagccccgacgtcttcaaatccttaggttgtgaagactttattagctttgtgtttgaatttggaaagagtttatgttctatgcacctgactgaagatgaaattgcattattttctgcatttgtactgatgtcagcagatcgctcatggctgcaagaaaaggtaaaaattgaaaaactgcaacagaaaattcagctagctcttcaacacgtcctacagaagaatcaccgagaagatggaatactaacaaagttaatatgcaaggtgtctacattaagagccttatgtggacgacatacagaaaagctaatggcatttaaagcaatatacccagacattgtgcgacttcattttcctccattatacaaggagttgttcacttcagaatttgagccagcaatgcaaattgatgggtaaatgttatcacctaagcacttctagaatgtctgaagtacaaacatgaaaaacaaacaaaaaaattaaccgagacactttatatggccctgcacagacctggagcgccacacactgcacatcttttggtgatcggggtcaggcaaaggaggggaaacaatgaaaacaaataaagttgaacttgtttttctca

SEQ ID NO: 28 - DNA, 1996 bp, Homo sapiens
gcagattcacagggcctctgagcattatcccccatactcctccccatcattctccacccagctgttggagccatctgtctgatcaccttggactccatagtacactggggcaaagcacagccccagtttctggaggcagatgggtaaccaggaaaaggcatgaatgagggggccccaggagacagtgacttagagactgaggcaagagtgccgtggtcaatcatgggtcattgtcttcgaactggacaggccagaatgtctgccacacccacacctgcaggtgaaggagccagaagctcttcaacctgtagctccctgagcaggctgttctggtctcaacttgagcacataaactgggatggagccacagccaagaactttattaatttaagggagttcttctcttttctgctccctgcattgagaaaagctcaaattgaaattattccatgcaagatctgtggagacaaatcatcaggaatccattatggtgtcattacatgtgaaggctgcaagggctttttcaggagaagtcagcaaagcaatgccacctactcctgtcctcgtcagaagaactgtttgattgatcgaaccagtagaaaccgctgccaacactgtcgattacagaaatgccttgccgtagggatgtctcgagatgctgtaaaatttggccgaatgtcaaaaaagcagagagacagcttgtatgcagaagtacagaaacaccggatgcagcagcagcagcgcgaccaccagcagcagcctggagaggctgagccgctgacgcccacctacaacatctcggccaacgggctgacggaacttcacgacgacctcagtaactacattgacgggcacacccctgaggggagtaaggcagactccgccgtcagcagcttctacctggacatacagccttccccagaccagtcaggtcttgatatcaatggaatcaaaccagaaccaatatgtgactacacaccagcatcaggcttctttccctactgttcgttcaccaacggcgagacttccccaactgtgtccatggcagaattagaacaccttgcacagaatatatctaaatcgcatctggaaacctgccaatacttgagagaagagctccagcagataacgtggcagacctttttacaggaagaaattgagaactatcaaaacaagcagcgggaggtgatgtggcaattgtgtgccatcaaaattacagaagctatacagtatgtggtggagtttgccaaacgcattgatggatttatggaactgtgtcaaaatgatcaaattgtgcttctaaaagcaggttctctagaggtggtgtttatcagaatgtgccgtgcctttgactctcagaacaacaccgtgtactttgatgggaagtatgccagccccgacgtcttcaaatccttaggttgtgaagactttattagctttgtgtttgaatttggaaagagtttatgttctatgcacctgactgaagatgaaattgcattattttctgcatttgtactgatgtcagcagatcgctcatggctgcaagaaaaggtaaaaattgaaaaactgcaacagaaaattcagctagctcttcaacacgtcctacagaagaatcaccgagaagatggaatactaacaaagttaatatgcaaggtgtctacattaagagccttatgtggacgacatacagaaaagctaatggcatttaaagcaatatacccagacattgtgcgacttcattttcctccattatacaaggagttgttcacttcagaatttgagccagcaatgcaaattgatgggtaaatgttatcacctaagcacttctagaatgtctgaagtacaaacatgaaaaacaaacaaaaaaattaaccgagacactttatatggccctgcacagacctggagcgccacacactgcacatcttttggtgatcggggtcaggcaaaggaggggaaacaatgaaaacaaataaagttgaacttgtttttctca

SEQ ID NO: 29 - DNA, 1687 bp, Homo sapiens
tgtggctcgggcggcggcggcgcggcggcggcagagggggctccggggtcggaccatccgctctccctgcgctctccgcaccgcgcttaaatgatgtattttgtgatcgcagcgatgaaagctcaaattgaaattattccatgcaagatctgtggagacaaatcatcaggaatccattatggtgtcattacatgtgaaggctgcaagggctttttcaggagaagtcagcaaagcaatgccacctactcctgtcctcgtcagaagaactgtttgattgatcgaaccagtagaaaccgctgccaacactgtcgattacagaaatgccttgccgtagggatgtctcgagatgctgtaaaatttggccgaatgtcaaaaaagcagagagacagcttgtatgcagaagtacagaaacaccggatgcagcagcagcagcgcgaccaccagcagcagcctggagaggctgagccgctgacgcccacctacaacatctcggccaacgggctgacggaacttcacgacgacctcagtaactacattgacgggcacacccctgaggggagtaaggcagactccgccgtcagcagcttctacctggacatacagccttccccagaccagtcaggtcttgatatcaatggaatcaaaccagaaccaatatgtgactacacaccagcatcaggcttctttccctactgttcgttcaccaacggcgagacttccccaactgtgtccatggcagaattagaacaccttgcacagaatatatctaaatcgcatctggaaacctgccaatacttgagagaagagctccagcagataacgtggcagacctttttacaggaagaaattgagaactatcaaaacaagcagcgggaggtgatgtggcaattgtgtgccatcaaaattacagaagctatacagtatgtggtggagtttgccaaacgcattgatggatttatggaactgtgtcaaaatgatcaaattgtgcttctaaaagcaggttctctagaggtggtgtttatcagaatgtgccgtgcctttgactctcagaacaacaccgtgtactttgatgggaagtatgccagccccgacgtcttcaaatccttaggttgtgaagactttattagctttgtgtttgaatttggaaagagtttatgttctatgcacctgactgaagatgaaattgcattattttctgcatttgtactgatgtcagcagatcgctcatggctgcaagaaaaggtaaaaattgaaaaactgcaacagaaaattcagctagctcttcaacacgtcctacagaagaatcaccgagaagatggaatactaacaaagttaatatgcaaggtgtctacattaagagccttatgtggacgacatacagaaaagctaatggcatttaaagcaatatacccagacattgtgcgacttcattttcctccattatacaaggagttgttcacttcagaatttgagccagcaatgcaaattgatgggtaaatgttatcacctaagcacttctagaatgtctgaagtacaaacatgaaaaacaaacaaaaaaattaaccgagacactttatatggccctgcacagacctggagcgccacacactgcacatcttttggtgatcggggtcaggcaaaggaggggaaacaatgaaaacaaataaagttgaacttgtttttctca

SEQ ID NO: 30 - protein, 523 aa, Homo sapiens
MESAPAAPDPAASEPGSSGADAAAGSRETPLNQESARKSEPPAPVRRQSYSSTSRGISVTKKTHTSQIEIIPCKICGDKSSGIHYGVITCEGCKGFFRRSQQSNATYSCPRQKNCLIDRTSRNRCQHCRLQKCLAVGMSRDAVKFGRMSKKQRDSLYAEVQKHRMQQQQRDHQQQPGEAEPLTPTYNISANGLTELHDDLSNYIDGHTPEGSKADSAVSSFYLDIQPSPDQSGLDINGIKPEPICDYTPASGFFPYCSFTNGETSPTVSMAELEHLAQNISKSHLETCQYLREELQQITWQTFLQEEIENYQNKQREVMWQLCAIKITEAIQYVVEFAKRIDGFMELCQNDQIVLLKAGSLEVVFIRMCRAFDSQNNTVYFDGKYASPDVFKSLGCEDFISFVFEFGKSLCSMHLTEDEIALFSAFVLMSADRSWLQEKVKIEKLQQKIQLALQHVLQKNHREDGILTKLICKVSTLRALCGRHTEKLMAFKAIYPDIVRLHFPPLYKELFTSEFEPAMQIDG

SEQ ID NO: 31 - protein, 556 aa, Homo sapiens
MNEGAPGDSDLETEARVPWSIMGHCLRTGQARMSATPTPAGEGARRDELFGILQILHQCILSSGDAFVLTGVCCSWRQNGKPPYSQKEDKEVQTGYMNAQIEIIPCKICGDKSSGIHYGVITCEGCKGFFRRSQQSNATYSCPRQKNCLIDRTSRNRCQHCRLQKCLAVGMSRDAVKFGRMSKKQRDSLYAEVQKHRMQQQQRDHQQQPGEAEPLTPTYNISANGLTELHDDLSNYIDGHTPEGSKADSAVSSFYLDIQPSPDQSGLDINGIKPEPICDYTPASGFFPYCSFTNGETSPTVSMAELEHLAQNISKSHLETCQYLREELQQITWQTFLQEEIENYQNKQREVMWQLCAIKITEAIQYVVEFAKRIDGFMELCQNDQIVLLKAGSLEVVFIRMCRAFDSQNNTVYFDGKYASPDVFKSLGCEDFISFVFEFGKSLCSMHLTEDEIALFSAFVLMSADRSWLQEKVKIEKLQQKIQLALQHVLQKNHREDGILTKLICKVSTLRALCGRHTEKLMAFKAIYPDIVRLHFPPLYKELFTSEFEPAMQIDG

SEQ ID NO: 32 - protein, 548 aa, Homo sapiens
MNEGAPGDSDLETEARVPWSIMGHCLRTGQARMSATPTPAGEGARSSSTCSSLSRLFWSQLEHINWDGATAKNFINLREFFSFLLPALRKAQIEIIPCKICGDKSSGIHYGVITCEGCKGFFRRSQQSNATYSCPRQKNCLIDRTSRNRCQHCRLQKCLAVGMSRDAVKFGRMSKKQRDSLYAEVQKHRMQQQQRDHQQQPGEAEPLTPTYNISANGLTELHDDLSNYIDGHTPEGSKADSAVSSFYLDIQPSPDQSGLDINGIKPEPICDYTPASGFFPYCSFTNGETSPTVSMAELEHLAQNISKSHLETCQYLREELQQITWQTFLQEEIENYQNKQREVMWQLCAIKITEAIQYVVEFAKRIDGFMELCQNDQIVLLKAGSLEVVFIRMCRAFDSQNNTVYFDGKYASPDVFKSLGCEDFISFVFEFGKSLCSMHLTEDEIALFSAFVLMSADRSWLQEKVKIEKLQQKIQLALQHVLQKNHREDGILTKLICKVSTLRALCGRHTEKLMAFKAIYPDIVRLHFPPLYKELFTSEFEPAMQIDG

SEQ ID NO: 33 - protein, 468 aa, Homo sapiens
MMYFVIAAMKAQIEIIPCKICGDKSSGIHYGVITCEGCKGFFRRSQQSNATYSCPRQKNCLIDRTSRNRCQHCRLQKCLAVGMSRDAVKFGRMSKKQRDSLYAEVQKHRMQQQQRDHQQQPGEAEPLTPTYNISANGLTELHDDLSNYIDGHTPEGSKADSAVSSFYLDIQPSPDQSGLDINGIKPEPICDYTPASGFFPYCSFTNGETSPTVSMAELEHLAQNISKSHLETCQYLREELQQITWQTFLQEEIENYQNKQREVMWQLCAIKITEAIQYVVEFAKRIDGFMELCQNDQIVLLKAGSLEVVFIRMCRAFDSQNNTVYFDGKYASPDVFKSLGCEDFISFVFEFGKSLCSMHLTEDEIALFSAFVLMSADRSWLQEKVKIEKLQQKIQLALQHVLQKNHREDGILTKLICKVSTLRALCGRHTEKLMAFKAIYPDIVRLHFPPLYKELFTSEFEPAMQIDG

SEQ ID NO: 34 - DNA, 2943 bp, Homo sapiens
accaggcaacaccattgaaggctcatatgtaaaaatccatgccttcctttctcccaatctccattcccaaacttagccactggcttctggctgaggccttacgcatacctcccggggcttgcacacaccttcttctacagaagacacaccttgggcatatcctacagaagaccaggcttctctctggtccttggtagagggctactttactgtaacagggccagggtggagagttctctcctgaagctccatcccctctataggaaatgtgttgacaatattcagaagagtaagaggatcaagacttctttgtgctcaaataccactgttctcttctctaccctgccctaaccaggagcttgtcaccccaaactctgaggtgatttatgccttaatcaagcaaacttccctcttcagaaaagatggctcattttccctcaaaagttgccaggagctgccaagtattctgccaattcaccctggagcacaatcaacaaattcagccagaacacaactacagctactattagaactattattattaataaattcctctccaaatctagccccttgacttcggatttcacgatttctcccttcctcctagaaacttgataagtttcccgcgcttccctttttctaagactacatgtttgtcatcttataaagcaaaggggtgaataaatgaaccaaatcaataacttctggaatatctgcaaacaacaataatatcagctatgccatctttcactattttagccagtatcgagttgaatgaacatagaaaaatacaaaactgaattcttccctgtaaattccccgttttgacgacgcacttgtagccacgtagccacgcctacttaagacaattacaaaaggcgaagaagactgactcaggcttaagctgccagccagagagggagtcatttcattggcgtttgagtcagcaaagaagtcaagatggccaaagttccagacatgtttgaagacctgaagaactgttacagtgaaaatgaagaagacagttcctccattgatcatctgtctctgaatcagaaatccttctatcatgtaagctatggcccactccatgaaggctgcatggatcaatctgtgtctctgagtatctctgaaacctctaaaacatccaagcttaccttcaaggagagcatggtggtagtagcaaccaacgggaaggttctgaagaagagacggttgagtttaagccaatccatcactgatgatgacctggaggccatcgccaatgactcagaggaagaaatcatcaagcctaggtcagcaccttttagcttcctgagcaatgtgaaatacaactttatgaggatcatcaaatacgaattcatcctgaatgacgccctcaatcaaagtataattcgagccaatgatcagtacctcacggctgctgcattacataatctggatgaagcagtgaaatttgacatgggtgcttataagtcatcaaaggatgatgctaaaattaccgtgattctaagaatctcaaaaactcaattgtatgtgactgcccaagatgaagaccaaccagtgctgctgaaggagatgcctgagatacccaaaaccatcacaggtagtgagaccaacctcctcttcttctgggaaactcacggcactaagaactatttcacatcagttgcccatccaaacttgtttattgccacaaagcaagactactgggtgtgcttggcaggggggccaccctctatcactgactttcagatactggaaaaccaggcgtaggtctggagtctcacttgtctcacttgtgcagtgttgacagttcatatgtaccatgtacatgaagaagctaaatcctttactgttagtcatttgctgagcatgtactgagccttgtaattctaaatgaatgtttacactctttgtaagagtggaaccaacactaacatataatgttgttatttaaagaacaccctatattttgcatagtaccaatcattttaattattattcttcataacaattttaggaggaccagagctactgactatggctaccaaaaagactctacccatattacagatgggcaaattaaggcataagaaaactaagaaatatgcacaatagcagttgaaacaagaagccacagacctaggatttcatgatttcatttcaactgtttgccttctacttttaagttgctgatgaactcttaatcaaatagcataagtttctgggacctcagttttatcattttcaaaatggagggaataatacctaagccttcctgccgcaacagttttttatgctaatcagggaggtcattttggtaaaatacttcttgaagccgagcctcaagatgaaggcaaagcacgaaatgttattttttaattattatttatatatgtatttataaatatatttaagataattataatatactatatttatgggaaccccttcatcctctgagtgtgaccaggcatcctccacaatagcagacagtgttttctgggataagtaagtttgatttcattaatacagggcattttggtccaagttgtgcttatcccatagccaggaaactctgcattctagtacttgggagacctgtaatcatataataaatgtacattaattaccttgagccagtaattggtccgatctttgactcttttgccattaaacttacctgggcattcttgtttcaattccacctgcaatcaagtcctacaagctaaaattagatgaactcaactttgacaaccatgagaccactgttatcaaaactttcttttctggaatgtaatcaatgtttcttctaggttctaaaaattgtgatcagaccataatgttacattattatcaacaatagtgattgatagagtgttatcagtcataactaaataaagcttgcaacaaaattctctgacaaaaaaaaaaaaaaaa

SEQ ID NO: 35 - protein, 271 aa, Homo sapiens
MAKVPDMFEDLKNCYSENEEDSSSIDHLSLNQKSFYHVSYGPLHEGCMDQSVSLSISETSKTSKLTFKESMVVVATNGKVLKKRRLSLSQSITDDDLEAIANDSEEEIIKPRSAPFSFLSNVKYNFMRIIKYEFILNDALNQSIIRANDQYLTAAALHNLDEAVKFDMGAYKSSKDDAKITVILRISKTQLYVTAQDEDQPVLLKEMPEIPKTITGSETNLLFFWETHGTKNYFTSVAHPNLFIATKQDYWVCLAGGPPSITDFQILENQA

SEQ ID NO: 36 - DNA, 10412 bp, Homo sapiens
gtaattgcgagcgagagtgagtggggccgggacccgcagagccgagccgacccttctctcccgggctgcggcagggcagggcggggagctccgcgcaccaacagagccggttctcagggcgctttgctccttgttttttccccggttctgttttctccccttctccggaaggcttgtcaaggggtaggagaaagagacgcaaacacaaaagtggaaaacagttaatgaccagccacggcgtccctgctgtgagctctggccgctgccttccagggctcccgagccacacgctgggggtgctggctgagggaacatggcttgttggcctcagctgaggttgctgctgtggaagaacctcactttcagaagaagacaaacatgtcagctgctgctggaagtggcctggcctctatttatcttcctgatcctgatctctgttcggctgagctacccaccctatgaacaacatgaatgccattttccaaataaagccatgccctctgcaggaacacttccttgggttcaggggattatctgtaatgccaacaacccctgtttccgttacccgactcctggggaggctcccggagttgttggaaactttaacaaatccattgtggctcgcctgttctcagatgctcggaggcttcttttatacagccagaaagacaccagcatgaaggacatgcgcaaagttctgagaacattacagcagatcaagaaatccagctcaaacttgaagcttcaagatttcctggtggacaatgaaaccttctctgggttcctgtatcacaacctctctctcccaaagtctactgtggacaagatgctgagggctgatgtcattctccacaaggtatttttgcaaggctaccagttacatttgacaagtctgtgcaatggatcaaaatcagaagagatgattcaacttggtgaccaagaagtttctgagctttgtggcctaccaagggagaaactggctgcagcagagcgagtacttcgttccaacatggacatcctgaagccaatcctgagaacactaaactctacatctcccttcccgagcaaggagctggctgaagccacaaaaacattgctgcatagtcttgggactctggcccaggagctgttcagcatgagaagctggagtgacatgcgacaggaggtgatgtttctgaccaatgtgaacagctccagctcctccacccaaatctaccaggctgtgtctcgtattgtctgcgggcatcccgagggaggggggctgaagatcaagtctctcaactggtatgaggacaacaactacaaagccctctttggaggcaatggcactgaggaagatgctgaaaccttctatgacaactctacaactccttactgcaatgatttgatgaagaatttggagtctagtcctctttcccgcattatctggaaagctctgaagccgctgctcgttgggaagatcctgtatacacctgacactccagccacaaggcaggtcatggctgaggtgaacaagaccttccaggaactggctgtgttccatgatctggaaggcatgtgggaggaactcagccccaagatctggaccttcatggagaacagccaagaaatggaccttgtccggatgctgttggacagcagggacaatgaccacttttgggaacagcagttggatggcttagattggacagcccaagacatcgtggcgtttttggccaagcacccagaggatgtccagtccagtaatggttctgtgtacacctggagagaagctttcaacgagactaaccaggcaatccggaccatatctcgcttcatggagtgtgtcaacctgaacaagctagaacccatagcaacagaagtctggctcatcaacaagtccatggagctgctggatgagaggaagttctgggctggtattgtgttcactggaattactccaggcagcattgagctgccccatcatgtcaagtacaagatccgaatggacattgacaatgtggagaggacaaataaaatcaaggatgggtactgggaccctggtcctcgagctgacccctttgaggacatgcggtacgtctgggggggcttcgcctacttgcaggatgtggtggagcaggcaatcatcagggtgctgacgggcaccgagaagaaaactggtgtctatatgcaacagatgccctatccctgttacgttgatgacatctttctgcgggtgatgagccggtcaatgcccctcttcatgacgctggcctggatttactcagtggctgtgatcatcaagggcatcgtgtatgagaaggaggcacggctgaaagagaccatgcggatcatgggcctggacaacagcatcctctggtttagctggttcattagtagcctcattcctcttcttgtgagcgctggcctgctagtggtcatcctgaagttaggaaacctgctgccctacagtgatcccagcgtggtgtttgtcttcctgtccgtgtttgctgtggtgacaatcctgcagtgcttcctgattagcacactcttctccagagccaacctggcagcagcctgtgggggcatcatctacttcacgctgtacctgccctacgtcctgtgtgtggcatggcaggactacgtgggcttcacactcaagatcttcgctagcctgctgtctcctgtggcttttgggtttggctgtgagtactttgccctttttgaggagcagggcattggagtgcagtgggacaacctgtttgagagtcctgtggaggaagatggcttcaatctcaccacttcggtctccatgatgctgtttgacaccttcctctatggggtgatgacctggtacattgaggctgtctttccaggccagtacggaattcccaggccctggtattttccttgcaccaagtcctactggtttggcgaggaaagtgatgagaagagccaccctggttccaaccagaagagaatatcagaaatctgcatggaggaggaacccacccacttgaagctgggcgtgtccattcagaacctggtaaaagtctaccgagatgggatgaaggtggctgtcgatggcctggcactgaatttttatgagggccagatcacctccttcctgggccacaatggagcggggaagacgaccaccatgtcaatcctgaccgggttgttccccccgacctcgggcaccgcctacatcctgggaaaagacattcgctctgagatgagcaccatccggcagaacctgggggtctgtccccagcataacgtgctgtttgacatgctgactgtcgaagaacacatctggttctatgcccgcttgaaagggctctctgagaagcacgtgaaggcggagatggagcagatggccctggatgttggtttgccatcaagcaagctgaaaagcaaaacaagccagctgtcaggtggaatgcagagaaagctatctgtggccttggcctttgtcgggggatctaaggttgtcattctggatgaacccacagctggtgtggacccttactcccgcaggggaatatgggagctgctgctgaaataccgacaaggccgcaccattattctctctacacaccacatggatgaagcggacgtcctgggggacaggattgccatcatctcccatgggaagctgtgctgtgtgggctcctccctgtttctgaagaaccagctgggaacaggctactacctgaccttggtcaagaaagatgtggaatcctccctcagttcctgcagaaacagtagtagcactgtgtcatacctgaaaaaggaggacagtgtttctcagagcagttctgatgctggcctgggcagcgaccatgagagtgacacgctgaccatcgatgtctctgctatctccaacctcatcaggaagcatgtgtctgaagcccggctggtggaagacatagggcatgagctgacctatgtgctgccatatgaagctgctaaggagggagcctttgtggaactctttcatgagattgatgaccggctctcagacctgggcatttctagttatggcatctcagagacgaccctggaagaaatattcctcaaggtggccgaagagagtggggtggatgctgagacctcagatggtaccttgccagcaagacgaaacaggcgggccttcggggacaagcagagctgtcttcgcccgttcactgaagatgatgctgctgatccaaatgattctgacatagacccagaatccagagagacagacttgctcagtgggatggatggcaaagggtcctaccaggtgaaaggctggaaacttacacagcaacagtttgtggcccttttgtggaagagactgctaattgccagacggagtcggaaaggattttttgctcagattgtcttgccagctgtgtttgtctgcattgcccttgtgttcagcctgatcgtgccaccctttggcaagtaccccagcctggaacttcagccctggatgtacaacgaacagtacacatttgtcagcaatgatgctcctgaggacacgggaaccctggaactcttaaacgccctcaccaaagaccctggcttcgggacccgctgtatggaaggaaacccaatcccagacacgccctgccaggcaggggaggaagagtggaccactgccccagttccccagaccatcatggacctcttccagaatgggaactggacaatgcagaacccttcacctgcatgccagtgtagcagcgacaaaatcaagaagatgctgcctgtgtgtcccccaggggcaggggggctgcctcctccacaaagaaaacaaaacactgcagatatccttcaggacctgacaggaagaaacatttcggattatctggtgaagacgtatgtgcagatcatagccaaaagcttaaagaacaagatctgggtgaatgagtttaggtatggcggcttttccctgggtgtcagtaatactcaagcacttcctccgagtcaagaagttaatgatgccatcaaacaaatgaagaaacacctaaagctggccaaggacagttctgcagatcgatttctcaacagcttgggaagatttatgacaggactggacaccaaaaataatgtcaaggtgtggttcaataacaagggctggcatgcaatcagctctttcctgaatgtcatcaacaatgccattctccgggccaacctgcaaaagggagagaaccctagccattatggaattactgctttcaatcatcccctgaatctcaccaagcagcagctctcagaggtggctctgatgaccacatcagtggatgtccttgtgtccatctgtgtcatctttgcaatgtccttcgtcccagccagctttgtcgtattcctgatccaggagcgggtcagcaaagcaaaacacctgcagttcatcagtggagtgaagcctgtcatctactggctctctaattttgtctgggatatgtgcaattacgttgtccctgccacactggtcattatcatcttcatctgcttccagcagaagtcctatgtgtcctccaccaatctgcctgtgctagcccttctacttttgctgtatgggtggtcaatcacacctctcatgtacccagcctcctttgtgttcaagatccccagcacagcctatgtggtgctcaccagcgtgaacctcttcattggcattaatggcagcgtggccacctttgtgctggagctgttcaccgacaataagctgaataatatcaatgatatcctgaagtccgtgttcttgatcttcccacatttttgcctgggacgagggctcatcgacatggtgaaaaaccaggcaatggctgatgccctggaaaggtttggggagaatcgctttgtgtcaccattatcttgggacttggtgggacgaaacctcttcgccatggccgtggaaggggtggtgttcttcctcattactgttctgatccagtacagattcttcatcaggcccagacctgtaaatgcaaagctatctcctctgaatgatgaagatgaagatgtgaggcgggaaagacagagaattcttgatggtggaggccagaatgacatcttagaaatcaaggagttgacgaagatatatagaaggaagcggaagcctgctgttgacaggatttgcgtgggcattcctcctggtgagtgctttgggctcctgggagttaatggggctggaaaatcatcaactttcaagatgttaacaggagataccactgttaccagaggagatgctttccttaacaaaaatagtatcttatcaaacatccatgaagtacatcagaacatgggctactgccctcagtttgatgccatcacagagctgttgactgggagagaacacgtggagttctttgcccttttgagaggagtcccagagaaagaagttggcaaggttggtgagtgggcgattcggaaactgggcctcgtgaagtatggagaaaaatatgctggtaactatagtggaggcaacaaacgcaagctctctacagccatggctttgatcggcgggcctcctgtggtgtttctggatgaacccaccacaggcatggatcccaaagcccggcggttcttgtggaattgtgccctaagtgttgtcaaggaggggagatcagtagtgcttacatctcatagtatggaagaatgtgaagctctttgcactaggatggcaatcatggtcaatggaaggttcaggtgccttggcagtgtccagcatctaaaaaataggtttggagatggttatacaatagttgtacgaatagcagggtccaacccggacctgaagcctgtccaggatttctttggacttgcatttcctggaagtgttctaaaagagaaacaccggaacatgctacaataccagcttccatcttcattatcttctctggccaggatattcagcatcctctcccagagcaaaaagcgactccacatagaagactactctgtttctcagacaacacttgaccaagtatttgtgaactttgccaaggaccaaagtgatgatgaccacttaaaagacctctcattacacaaaaaccagacagtagtggacgttgcagttctcacatcttttctacaggatgagaaagtgaaagaaagctatgtatgaagaatcctgttcatacggggtggctgaaagtaaagaggaactagactttcctttgcaccatgtgaagtgttgtggagaaaagagccagaagttgatgtgggaagaagtaaactggatactgtactgatactattcaatgcaatgcaattcaatgcaatgaaaacaaaattccattacaggggcagtgcctttgtagcctatgtcttgtatggctctcaagtgaaagacttgaatttagttttttacctatacctatgtgaaactctattatggaacccaatggacatatgggtttgaactcacacttttttttttttttttgttcctgtgtattctcattggggttgcaacaataattcatcaagtaatcatggccagcgattattgatcaaaatcaaaaggtaatgcacatcctcattcactaagccatgccatgcccaggagactggtttcccggtgacacatccattgctggcaatgagtgtgccagagttattagtgccaagtttttcagaaagtttgaagcaccatggtgtgtcatgctcacttttgtgaaagctgctctgctcagagtctatcaacattgaatatcagttgacagaatggtgccatgcgtggctaacatcctgctttgattccctctgataagctgttctggtggcagtaacatgcaacaaaaatgtgggtgtctccaggcacgggaaacttggttccattgttatattgtcctatgcttcgagccatgggtctacagggtcatccttatgagactcttaaatatacttagatcctggtaagaggcaaagaatcaacagccaaactgctggggctgcaagctgctgaagccagggcatgggattaaagagattgtgcgttcaaacctagggaagcctgtgcccatttgtcctgactgtctgctaacatggtacactgcatctcaagatgtttatctgacacaagtgtattatttctggctttttgaattaatctagaaaatgaaaagatggagttgtattttgacaaaaatgtttgtactttttaatgttatttggaattttaagttctatcagtgacttctgaatccttagaatggcctctttgtagaaccctgtggtatagaggagtatggccactgccccactatttttattttcttatgtaagtttgcatatcagtcatgactagtgcctagaaagcaatgtgatggtcaggatctcatgacattatatttgagtttctttcagatcatttaggatactcttaatctcacttcatcaatcaaatattttttgagtgtatgctgtagctgaaagagtatgtacgtacgtataagactagagagatattaagtctcagtacacttcctgtgccatgttattcagctcactggtttacaaatataggttgtcttgtggttgtaggagcccactgtaacaatactgggcagcctttttttttttttttttaattgcaacaatgcaaaagccaagaaagtataagggtcacaagtctaaacaatgaattcttcaacagggaaaacagctagcttgaaaacttgctgaaaaacacaacttgtgtttatggcatttagtaccttcaaataattggctttgcagatattggataccccattaaatctgacagtctcaaatttttcatctcttcaatcactagtcaagaaaaatataaaaacaacaaatacttccatatggagcatttttcagagttttctaacccagtcttatttttctagtcagtaaacatttgtaaaaatactgtttcactaatacttactgttaactgtcttgagagaaaagaaaaatatgagagaactattgtttggggaagttcaagtgatctttcaatatcattactaacttcttccactttttccagaatttgaatattaacgctaaaggtgtaagacttcagatttcaaattaatctttctatattttttaaatttacagaatattatataacccactgctgaaaaagaaaaaaatgattgttttagaagttaaagtcaatattgattttaaatataagtaatgaaggcatatttccaataactagtgatatggcatcgttgcattttacagtatcttcaaaaatacagaatttatagaataatttctcctcatttaatatttttcaaaatcaaagttatggtttcctcattttactaaaatcgtattctaattcttcattatagtaaatctatgagcaactccttacttcggttcctctgatttcaaggccatattttaaaaaatcaaaaggcactgtgaactattttgaagaaaacacaacattttaatacagattgaaaggacctcttctgaagctagaaacaatctatagttatacatcttcattaatactgtgttaccttttaaaatagtaattttttacattttcctgtgtaaacctaattgtggtagaaatttttaccaactctatactcaatcaagcaaaatttctgtatattccctgtggaatgtacctatgtgagtttcagaaattctcaaaatacgtgttcaaaaatttctgcttttgcatctttgggacacctcagaaaacttattaacaactgtgaatatgagaaatacagaagaaaataataagccctctatacataaatgcccagcacaattcattgttaaaaaacaaccaaacctcacactactgtatttcattatctgtactgaaagcaaatgctttgtgactattaaatgttgcacatcattcattcactgtatagtaatcattgactaaagccatttgtctgtgttttcttcttgtggttgtatatatcaggtaaaatattttccaaagagccatgtgtcatgtaatactgaaccactttgatattgagacattaatttgtacccttgttattatctactagtaataatgtaatactgtagaaatattgctctaattcttttcaaaattgttgcatcccccttagaatgtttctatttccataaggatttaggtatgctattatcccttcttataccctaagatgaagctgtttttgtgctctttgttcatcattggccctcattccaagcactttacgctgtctgtaatgggatctatttttgcactggaatatctgagaattgcaaaactagacaaaagtttcacaacagatttctaagttaaatcattttcattaaaaggaaaaaagaaaaaaaattttgtatgtcaataactttatatgaagtattaaaatgcatatttctatgttgtaatataatgagtcacaaaataaagctgtgacagttctgttggtctacagaaa

SEQ ID NO: 37 - protein, 2261 aa, Homo sapiens
MACWPQLRLLLWKNLTFRRRQTCQLLLEVAWPLFIFLILISVRLSYPPYEQHECHFPNKAMPSAGTLPWVQGIICNANNPCFRYPTPGEAPGVVGNFNKSIVARLFSDARRLLLYSQKDTSMKDMRKVLRTLQQIKKSSSNLKLQDFLVDNETFSGFLYHNLSLPKSTVDKMLRADVILHKVFLQGYQLHLTSLCNGSKSEEMIQLGDQEVSELCGLPREKLAAAERVLRSNMDILKPILRTLNSTSPFPSKELAEATKTLLHSLGTLAQELFSMRSWSDMRQEVMFLTNVNSSSSSTQIYQAVSRIVCGHPEGGGLKIKSLNWYEDNNYKALFGGNGTEEDAETFYDNSTTPYCNDLMKNLESSPLSRIIWKALKPLLVGKILYTPDTPATRQVMAEVNKTFQELAVFHDLEGMWEELSPKIWTFMENSQEMDLVRMLLDSRDNDHFWEQQLDGLDWTAQDIVAFLAKHPEDVQSSNGSVYTWREAFNETNQAIRTISRFMECVNLNKLEPIATEVWLINKSMELLDERKFWAGIVFTGITPGSIELPHHVKYKIRMDIDNVERTNKIKDGYWDPGPRADPFEDMRYVWGGFAYLQDVVEQAIIRVLTGTEKKTGVYMQQMPYPCYVDDIFLRVMSRSMPLFMTLAWIYSVAVIIKGIVYEKEARLKETMRIMGLDNSILWFSWFISSLIPLLVSAGLLVVILKLGNLLPYSDPSVVFVFLSVFAVVTILQCFLISTLFSRANLAAACGGIIYFTLYLPYVLCVAWQDYVGFTLKIFASLLSPVAFGFGCEYFALFEEQGIGVQWDNLFESPVEEDGFNLTTSVSMMLFDTFLYGVMTWYIEAVFPGQYGIPRPWYFPCTKSYWFGEESDEKSHPGSNQKRISEICMEEEPTHLKLGVSIQNLVKVYRDGMKVAVDGLALNFYEGQITSFLGHNGAGKTTTMSILTGLFPPTSGTAYILGKDIRSEMSTIRQNLGVCPQHNVLFDMLTVEEHIWFYARLKGLSEKHVKAEMEQMALDVGLPSSKLKSKTSQLSGGMQRKLSVALAFVGGSKVVILDEPTAGVDPYSRRGIWELLLKYRQGRTIILSTHHMDEADVLGDRIAIISHGKLCCVGSSLFLKNQLGTGYYLTLVKKDVESSLSSCRNSSSTVSYLKKEDSVSQSSSDAGLGSDHESDTLTIDVSAISNLIRKHVSEARLVEDIGHELTYVLPYEAAKEGAFVELFHEIDDRLSDLGISSYGISETTLEEIFLKVAEESGVDAETSDGTLPARRNRRAFGDKQSCLRPFTEDDAADPNDSDIDPESRETDLLSGMDGKGSYQVKGWKLTQQQFVALLWKRLLIARRSRKGFFAQIVLPAVFVCIALVFSLIVPPFGKYPSLELQPWMYNEQYTFVSNDAPEDTGTLELLNALTKDPGFGTRCMEGNPIPDTPCQAGEEEWTTAPVPQTIMDLFQNGNWTMQNPSPACQCSSDKIKKMLPVCPPGAGGLPPPQRKQNTADILQDLTGRNISDYLVKTYVQIIAKSLKNKIWVNEFRYGGFSLGVSNTQALPPSQEVNDAIKQMKKHLKLAKDSSADRFLNSLGRFMTGLDTKNNVKVWFNNKGWHAISSFLNVINNAILRANLQKGENPSHYGITAFNHPLNLTKQQLSEVALMTTSVDVLVSICVIFAMSFVPASFVVFLIQERVSKAKHLQFISGVKPVIYWLSNFVWDMCNYVVPATLVIIIFICFQQKSYVSSTNLPVLALLLLLYGWSITPLMYPASFVFKIPSTAYVVLTSVNLFIGINGSVATFVLELFTDNKLNNINDILKSVFLIFPHFCLGRGLIDMVKNQAMADALERFGENRFVSPLSWDLVGRNLFAMAVEGVVFFLITVLIQYRFFIRPRPVNAKLSPLNDEDEDVRRERQRILDGGGQNDILEIKELTKIYRRKRKPAVDRICVGIPPGECFGLLGVNGAGKSSTFKMLTGDTTVTRGDAFLNKNSILSNIHEVHQNMGYCPQFDAITELLTGREHVEFFALLRGVPEKEVGKVGEWAIRKLGLVKYGEKYAGNYSGGNKRKLSTAMALIGGPPVVFLDEPTTGMDPKARRFLWNCALSVVKEGRSVVLTSHSMEECEALCTRMAIMVNGRFRCLGSVQHLKNRFGDGYTIVVRIAGSNPDLKPVQDFFGLAFPGSVLKEKHRNMLQYQLPSSLSSLARIFSILSQSKKRLHIEDYSVSQTTLDQVFVNFAKDQSDDDHLKDLSLHKNQTVVDVAVLTSFLQDEKVKESYV

SEQ ID NO: 38 - DNA, 12057 bp, Homo sapiens
acagtgatataatgatgatgggtgtcacaacccgcatttgaacttgcaggcgagctgccccgagcctttctggggaagaactccaggcgtgcggacgcaacagccgagaacattaggtgttgtggacaggagctgggaccaagatcttcggccagccccgcatcctcccgcatcttccagcaccgtcccgcaccctccgcatccttccccgggccaccacgcttcctatgtgacccgcctgggcaacgccgaacccagtcgcgcagcgctgcagtgaattttccccccaaactgcaataagccgccttccaaggccaagatgttcataaatataaagagcatcttatggatgtgttcaaccttaatagtaacccatgcgctacataaagtcaaagtgggaaaaagcccaccggtgaggggctccctctctggaaaagtcagcctaccttgtcatttttcaacgatgcctactttgccacccagttacaacaccagtgaatttctccgcatcaaatggtctaagattgaagtggacaaaaatggaaaagatttgaaagagactactgtccttgtggcccaaaatggaaatatcaagattggtcaggactacaaagggagagtgtctgtgcccacacatcccgaggctgtgggcgatgcctccctcactgtggtcaagctgctggcaagtgatgcgggtctttaccgctgtgacgtcatgtacgggattgaagacacacaagacacggtgtcactgactgtggatggggttgtgtttcactacagggcggcaaccagcaggtacacactgaattttgaggctgctcagaaggcttgtttggacgttggggcagtcatagcaactccagagcagctctttgctgcctatgaagatggatttgagcagtgtgacgcaggctggctggctgatcagactgtcagatatcccatccgggctcccagagtaggctgttatggagataagatgggaaaggcaggagtcaggacttatggattccgttctccccaggaaacttacgatgtgtattgttatgtggatcatctggatggtgatgtgttccacctcactgtccccagtaaattcaccttcgaggaggctgcaaaagagtgtgaaaaccaggatgccaggctggcaacagtgggggaactccaggcggcatggaggaacggctttgaccagtgcgattacgggtggctgtcggatgccagcgtgcgccaccctgtgactgtggccagggcccagtgtggaggtggtctacttggggtgagaaccctgtatcgttttgagaaccagacaggcttccctccccctgatagcagatttgatgcctactgctttaaacctaaagaggctacaaccatcgatttgagtatcctcgcagaaactgcatcacccagtttatccaaagaaccacaaatggtttctgatagaactacaccaatcatccctttagttgatgaattacctgtcattccaacagagttccctcccgtgggaaatattgtcagttttgaacagaaagccacagtccaacctcaggctatcacagatagtttagccaccaaattacccacacctactggcagtaccaagaagccctgggatatggatgactactcaccttctgcttcaggacctcttggaaagctagacatatcagaaattaaggaagaagtgctccagagtacaactggcgtctctcattatgctacggattcatgggatggtgtcgtggaagataaacaaacacaagaatcggttacacagattgaacaaatagaagtgggtcctttggtaacatctatggaaatcttaaagcacattccttccaaggaattccctgtaactgaaacaccattggtaactgcaagaatgatcctggaatccaaaactgaaaagaaaatggtaagcactgtttctgaattggtaaccacaggtcactatggattcaccttgggagaagaggatgatgaagacagaacacttacagttggatctgatgagagcaccttgatctttgaccaaattcctgaagtcattacggtgtcaaagacttcagaagacaccatccacactcatttagaagacttggagtcagtctcagcatccacaactgtttcccctttaattatgcctgataataatggatcatccatggatgactgggaagagagacaaactagtggtaggataacggaagagtttcttggcaaatatctgtctactacaccttttccatcacagcatcgtacagaaatagaattgtttccttattctggtgataaaatattagtagagggaatttccacagttatttatccttctctacaaacagaaatgacacatagaagagaaagaacagaaacactaataccagagatgagaacagatacttatacagatgaaatacaagaagagatcactaaaagtccatttatgggaaaaacagaagaagaagtcttctctgggatgaaactctctacatctctctcagagccaattcatgttacagagtcttctgtggaaatgaccaagtcttttgatttcccaacattgataacaaagttaagtgcagagccaacagaagtaagagatatggaggaagactttacagcaactccaggtactacaaaatatgatgaaaatattacaacagtgcttttggcccatggtactttaagtgttgaagcagccactgtatcaaaatggtcatgggatgaagataatacaacatccaagcctttagagtctacagaaccttcagcctcttcaaaattgccccctgccttactcacaactgtggggatgaatggaaaggataaagacatcccaagtttcactgaagatggagcagatgaatttactcttattccagatagtactcaaaagcagttagaggaggttactgatgaagacatagcagcccatggaaaattcacaattagatttcagccaactacatcaactggtattgcagaaaagtcaactttgagagattctacaactgaagaaaaagttccacctatcacaagcactgaaggccaagtttatgcaaccatggaaggaagtgctttgggtgaagtagaagatgtggacctctctaagccagtatctactgttccccaatttgcacacacttcagaggtggaaggattagcatttgttagttatagtagcacccaagagcctactacttatgtagactcttcccataccattcctctttctgtaattcccaagacagactggggagtgttagtaccttctgttccatcagaagatgaagttctaggtgaaccctctcaagacatacttgtcattgatcagactcgccttgaagcgactatttctccagaaactatgagaacaacaaaaatcacagagggaacaactcaggaagaattcccttggaaagaacagactgcagagaaaccagttcctgctctcagttctacagcttggactcccaaggaggcagtaacaccactggatgaacaagagggcgatggatcagcatatacagtctctgaagatgaattgttgacaggttctgagagggtcccagttttagaaacaactccagttggaaaaattgatcacagtgtgtcttatccaccaggtgctgtaactgagcacaaagtgaaaacagatgaagtggtaacactaacaccacgcattgggccaaaagtatctttaagtccagggcctgaacaaaaatatgaaacagaaggtagtagtacaacaggatttacatcatctttgagtccttttagtacccacattacccagcttatggaagaaaccactactgagaaaacatccctagaggatattgatttaggctcaggattatttgaaaagcccaaagccacagaactcatagaattttcaacaatcaaagtcacagttccaagtgatattaccactgccttcagttcagtagacagacttcacacaacttcagcattcaagccatcttccgcgatcactaagaaaccacctctcatcgacagggaacctggtgaagaaacaaccagtgacatggtaatcattggagaatcaacatctcatgttcctcccactacccttgaagatattgtagccaaggaaacagaaaccgatattgatagagagtatttcacgacttcaagtcctcctgctacacagccaacaagaccacccactgtggaagacaaagaggcctttggacctcaggcgctttctacgccacagcccccagcaagcacaaaatttcaccctgacattaatgtttatattattgaggtcagagaaaataagacaggtcgaatgagtgatttgagtgtaattggtcatccaatagattcagaatctaaagaagatgaaccttgtagtgaagaaacagatccagtgcatgatctaatggctgaaattttacctgaattccctgacataattgaaatagacctataccacagtgaagaaaatgaagaagaagaagaagagtgtgcaaatgctactgatgtgacaaccaccccatctgtgcagtacataaatgggaagcatctcgttaccactgtgcccaaggacccagaagctgcagaagctaggcgtggccagtttgaaagtgttgcaccttctcagaatttctcggacagctctgaaagtgatactcatccatttgtaatagccaaaacggaattgtctactgctgtgcaacctaatgaatctacagaaacaactgagtctcttgaagttacatggaagcctgagacttaccctgaaacatcagaacatttttcaggtggtgagcctgatgttttccccacagtcccattccatgaggaatttgaaagtggaacagccaaaaaaggggcagaatcagtcacagagagagatactgaagttggtcatcaggcacatgaacatactgaacctgtatctctgtttcctgaagagtcttcaggagagattgccattgaccaagaatctcagaaaatagcctttgcaagggctacagaagtaacatttggtgaagaggtagaaaaaagtacttctgtcacatacactcccactatagttccaagttctgcatcagcatatgtttcagaggaagaagcagttaccctaataggaaatccttggccagatgacctgttgtctaccaaagaaagctgggtagaagcaactcctagacaagttgtagagctctcagggagttcttcgattccaattacagaaggctctggagaagcagaagaagatgaagatacaatgttcaccatggtaactgatttatcacagagaaatactactgatacactcattactttagacactagcaggataatcacagaaagcttttttgaggttcctgcaaccaccatttatccagtttctgaacaaccttctgcaaaagtggtgcctaccaagtttgtaagtgaaacagacacttctgagtggatttccagtaccactgttgaggaaaagaaaaggaaggaggaggagggaactacaggtacggcttctacatttgaggtatattcatctacacagagatcggatcaattaattttaccctttgaattagaaagtccaaatgtagctacatctagtgattcaggtaccaggaaaagttttatgtccttgacaacaccaacacagtctgaaagggaaatgacagattctactcctgtctttacagaaacaaatacattagaaaatttgggggcacagaccactgagcacagcagtatccatcaacctggggttcaggaagggctgaccactctcccacgtagtcctgcctctgtctttatggagcagggctctggagaagctgctgccgacccagaaaccaccactgtttcttcattttcattaaacgtagagtatgcaattcaagccgaaaaggaagtagctggcactttgtctccgcatgtggaaactacattctccactgagccaacaggactggttttgagtacagtaatggacagagtagttgctgaaaatataacccaaacatccagggaaatagtgatttcagagcgattaggagaaccaaattatggggcagaaataaggggcttttccacaggttttcctttggaggaagatttcagtggtgactttagagaatactcaacagtgtctcatcccatagcaaaagaagaaacggtaatgatggaaggctctggagatgcagcatttagggacacccagacttcaccatctacagtacctacttcagttcacatcagtcacatatctgactcagaaggacccagtagcaccatggtcagcacttcagccttcccctgggaagagtttacatcctcagctgagggctcaggtgagcaactggtcacagtcagcagctctgttgttccagtgcttcccagtgctgtgcaaaagttttctggtacagcttcctccattatcgacgaaggattgggagaagtgggtactgtcaatgaaattgatagaagatccaccattttaccaacagcagaagtggaaggtacgaaagctccagtagagaaggaggaagtaaaggtcagtggcacagtttcaacaaactttccccaaactatagagccagccaaattatggtctaggcaagaagtcaaccctgtaagacaagaaattgaaagtgaaacaacatcagaggaacaaattcaagaagaaaagtcatttgaatcccctcaaaactctcctgcaacagaacaaacaatctttgattcacagacatttactgaaactgaactcaaaaccacagattattctgtactaacaacaaagaaaacttacagtgatgataaagaaatgaaggaggaagacacttctttagttaacatgtctactccagatccagatgcaaatggcttggaatcttacacaactctccctgaagctactgaaaagtcacattttttcttagctactgcattagtaactgaatctataccagctgaacatgtagtcacagattcaccaatcaaaaaggaagaaagtacaaaacattttccgaaaggcatgagaccaacaattcaagagtcagatactgagctcttattctctggactgggatcaggagaagaagttttacctactctaccaacagagtcagtgaattttactgaagtggaacaaatcaataacacattatatccccacacttctcaagtggaaagtacctcaagtgacaaaattgaagactttaacagaatggaaaatgtggcaaaagaagttggaccactcgtatctcaaacagacatctttgaaggtagtgggtcagtaaccagcacaacattaatagaaattttaagtgacactggagcagaaggacccacggtggcacctctccctttctccacggacatcggacatcctcaaaatcagactgtcaggtgggcagaagaaatccagactagtagaccacaaaccataactgaacaagactctaacaagaattcttcaacagcagaaattaacgaaacaacaacctcatctactgattttctggctagagcttatggttttgaaatggccaaagaatttgttacatcagcaccaaaaccatctgacttgtattatgaaccttctggagaaggatctggagaagtggatattgttgattcatttcacacttctgcaactactcaggcaaccagacaagaaagcagcaccacatttgtttctgatgggtccctggaaaaacatcctgaggtgccaagcgctaaagctgttactgctgatggattcccaacagtttcagtgatgctgcctcttcattcagagcagaacaaaagctcccctgatccaactagcacactgtcaaatacagtgtcatatgagaggtccacagacggtagtttccaagaccgtttcagggaattcgaggattccaccttaaaacctaacagaaaaaaacccactgaaaatattatcatagacctggacaaagaggacaaggatttaatattgacaattacagagagtaccatccttgaaattctacctgagctgacatcggataaaaatactatcatagatattgatcatactaaacctgtgtatgaagacattcttggaatgcaaacagatatagatacagaggtaccatcagaaccacatgacagtaatgatgaaagtaatgatgacagcactcaagttcaagagatctatgaggcagctgtcaacctttctttaactgaggaaacatttgagggctctgctgatgttctggctagctacactcaggcaacacatgatgaatcaatgacttatgaagatagaagccaactagatcacatgggctttcacttcacaactgggatccctgctcctagcacagaaacagaattagacgttttacttcccacggcaacatccctgccaattcctcgtaagtctgccacagttattccagagattgaaggaataaaagctgaagcaaaagccctggatgacatgtttgaatcaagcactttgtctgatggtcaagctattgcagaccaaagtgaaataataccaacattgggccaatttgaaaggactcaggaggagtatgaagacaaaaaacatgctggtccttcttttcagccagaattctcttcaggagctgaggaggcattagtagaccatactccctatctaagtattgctactacccaccttatggatcagagtgtaacagaggtgcctgatgtgatggaaggatccaatcccccatattacactgatacaacattagcagtttcaacatttgcgaagttgtcttctcagacaccatcatctcccctcactatctactcaggcagtgaagcctctggacacacagagatcccccagcccagtgctctgccaggaatagacgtcggctcatctgtaatgtccccacaggattcttttaaggaaattcatgtaaatattgaagcgactttcaaaccatcaagtgaggaataccttcacataactgagcctccctctttatctcctgacacaaaattagaaccttcagaagatgatggtaaacctgagttattagaagaaatggaagcttctcccacagaacttattgctgtggaaggaactgagattctccaagatttccaaaacaaaaccgatggtcaagtttctggagaagcaatcaagatgtttcccaccattaaaacacctgaggctggaactgttattacaactgccgatgaaattgaattagaaggtgctacacagtggccacactctacttctgcttctgccacctatggggtcgaggcaggtgtggtgccttggctaagtccacagacttctgagaggcccacgctttcttcttctccagaaataaaccctgaaactcaagcagctttaatcagagggcaggattccacgatagcagcatcagaacagcaagtggcagcgagaattcttgattccaatgatcaggcaacagtaaaccctgtggaatttaatactgaggttgcaacaccaccattttcccttctggagacttctaatgaaacagatttcctgattggcattaatgaagagtcagtggaaggcacggcaatctatttaccaggacctgatcgctgcaaaatgaacccgtgccttaacggaggcacctgttatcctactgaaacttcctacgtatgcacctgtgtgccaggatacagcggagaccagtgtgaacttgattttgatgaatgtcactctaatccctgtcgtaatggagccacttgtgttgatggttttaacacattcaggtgcctctgccttccaagttatgttggtgcactttgtgagcaagataccgagacatgtgactatggctggcacaaattccaagggcagtgctacaaatactttgcccatcgacgcacatgggatgcagctgaacgggaatgccgtctgcagggtgcccatctcacaagcatcctgtctcacgaagaacaaatgtttgttaatcgtgtgggccatgattatcagtggataggcctcaatgacaagatgtttgagcatgacttccgttggactgatggcagcacactgcaatacgagaattggagacccaaccagccagacagcttcttttctgctggagaagactgtgttgtaatcatttggcatgagaatggccagtggaatgatgttccctgcaattaccatctcacctatacgtgcaagaaaggaacagtcgcttgcggccagccccctgttgtagaaaatgccaagacctttggaaagatgaaacctcgttatgaaatcaactccctgattagataccactgcaaagatggtttcattcaacgtcaccttccaactatccggtgcttaggaaatggaagatgggctatacctaaaattacctgcatgaacccatctgcataccaaaggacttattctatgaaatactttaaaaattcctcatcagcaaaggacaattcaataaatacatccaaacatgatcatcgttggagccggaggtggcaggagtcgaggcgctgatccctaaaatggcgaacatgtgttttcatcatttcagccaaagtcctaacttcctgtgcctttcctatcacctcgagaagtaattatcagttggtttggatttttggaccaccgttcagtcattttgggttgccgtgctcccaaaacattttaaatgaaagtattggcattcaaaaagacagcagacaaaatgaaagaaaatgagagcagaaagtaagcatttccagcctatctaatttctttagttttctatttgcctccagtgcagtccatttcctaatgtataccagcctactgtactatttaaaatgctcaatttcagcaccgatggccatgtaaataagatgatttaatgttgattttaatcctgtatataaaataaaaagtcacaatgagtttgggcatatttaatgatgattatggagccttagaggtctttaatcattggttcggctgcttttatgtagtttaggctggaaatggtttcacttgctctttgactgtcagcaagactgaagatggcttttcctggacagctagaaaacacaaaatcttgtaggtcattgcacctatctcagccataggtgcagtttgcttctacatgatgctaaaggctgcgaatgggatcctgatggaactaaggactccaatgtcgaactcttctttgctgcattcctttttcttcacttacaagaaaggcctgaatggaggacttttctgtaaccaggaacattttttaggggtcaaagtgctaataattaactcaaccaggtctactttttaatggctttcataacactaactcataaggttaccgatcaatgcatttcatacggatatagacctagggctctggagggtgggggattgttaaaacacatgcaaaaaaaaaaaaaaaaaaaaaaaaagaaattttgtatatataaccattttaatcttttataaagttttgaatgttcatgtatgaatgctgcagctgtgaagcatacataaataaatgaagtaagccatactgatttaatttattggatgttattttccctaagacctgaaaatgaacatagtatgctagttatttttcagtgttagccttttactttcctcacacaatttggaatcatataatataggtactttgtccctgattaaataatgtgacggatagaatgcatcaagtgtttattatgaaaagagtggaaaagtatatagcttttagcaaaaggtgtttgcccattctaagaaatgagcgaatatatagaaatagtgtgggcatttcttcctgttaggtggagtgtatgtgttgacatttctccccatctcttcccactctgttttctccccattatttgaataaagtgactgctgaagatgactttgaatccttatccacttaatttaatgtttaaagaaaaacctgtaatggaaagtaagactccttccctaatttcagtttagagcaacttgaagaagagtagacaaaaaataaaatgcacatagaaaaagagaaaaagggcacaaagggattggcccaatattgattctttttt

SEQ ID NO: 39 - protein, 3396 aa, Homo sapiens
MFINIKSILWMCSTLIVTHALHKVKVGKSPPVRGSLSGKVSLPCHFSTMPTLPPSYNTSEFLRIKWSKIEVDKNGKDLKETTVLVAQNGNIKIGQDYKGRVSVPTHPEAVGDASLTVVKLLASDAGLYRCDVMYGIEDTQDTVSLTVDGVVFHYRAATSRYTLNFEAAQKACLDVGAVIATPEQLFAAYEDGFEQCDAGWLADQTVRYPIRAPRVGCYGDKMGKAGVRTYGFRSPQETYDVYCYVDHLDGDVFHLTVPSKFTFEEAAKECENQDARLATVGELQAAWRNGFDQCDYGWLSDASVRHPVTVARAQCGGGLLGVRTLYRFENQTGFPPPDSRFDAYCFKPKEATTIDLSILAETASPSLSKEPQMVSDRTTPIIPLVDELPVIPTEFPPVGNIVSFEQKATVQPQAITDSLATKLPTPTGSTKKPWDMDDYSPSASGPLGKLDISEIKEEVLQSTTGVSHYATDSWDGVVEDKQTQESVTQIEQIEVGPLVTSMEILKHIPSKEFPVTETPLVTARMILESKTEKKMVSTVSELVTTGHYGFTLGEEDDEDRTLTVGSDESTLIFDQIPEVITVSKTSEDTIHTHLEDLESVSASTTVSPLIMPDNNGSSMDDWEERQTSGRITEEFLGKYLSTTPFPSQHRTEIELFPYSGDKILVEGISTVIYPSLQTEMTHRRERTETLIPEMRTDTYTDEIQEEITKSPFMGKTEEEVFSGMKLSTSLSEPIHVTESSVEMTKSFDFPTLITKLSAEPTEVRDMEEDFTATPGTTKYDENITTVLLAHGTLSVEAATVSKWSWDEDNTTSKPLESTEPSASSKLPPALLTTVGMNGKDKDIPSFTEDGADEFTLIPDSTQKQLEEVTDEDIAAHGKFTIRFQPTTSTGIAEKSTLRDSTTEEKVPPITSTEGQVYATMEGSALGEVEDVDLSKPVSTVPQFAHTSEVEGLAFVSYSSTQEPTTYVDSSHTIPLSVIPKTDWGVLVPSVPSEDEVLGEPSQDILVIDQTRLEATISPETMRTTKITEGTTQEEFPWKEQTAEKPVPALSSTAWTPKEAVTPLDEQEGDGSAYTVSEDELLTGSERVPVLETTPVGKIDHSVSYPPGAVTEHKVKTDEVVTLTPRIGPKVSLSPGPEQKYETEGSSTTGFTSSLSPFSTHITQLMEETTTEKTSLEDIDLGSGLFEKPKATELIEFSTIKVTVPSDITTAFSSVDRLHTTSAFKPSSAITKKPPLIDREPGEETTSDMVIIGESTSHVPPTTLEDIVAKETETDIDREYFTTSSPPATQPTRPPTVEDKEAFGPQALSTPQPPASTKFHPDINVYIIEVRENKTGRMSDLSVIGHPIDSESKEDEPCSEETDPVHDLMAEILPEFPDIIEIDLYHSEENEEEEEECANATDVTTTPSVQYINGKHLVTTVPKDPEAAEARRGQFESVAPSQNFSDSSESDTHPFVIAKTELSTAVQPNESTETTESLEVTWKPETYPETSEHFSGGEPDVFPTVPFHEEFESGTAKKGAESVTERDTEVGHQAHEHTEPVSLFPEESSGEIAIDQESQKIAFARATEVTFGEEVEKSTSVTYTPTIVPSSASAYVSEEEAVTLIGNPWPDDLLSTKESWVEATPRQVVELSGSSSIPITEGSGEAEEDEDTMFTMVTDLSQRNTTDTLITLDTSRIITESFFEVPATTIYPVSEQPSAKVVPTKFVSETDTSEWISSTTVEEKKRKEEEGTTGTASTFEVYSSTQRSDQLILPFELESPNVATSSDSGTRKSFMSLTTPTQSEREMTDSTPVFTETNTLENLGAQTTEHSSIHQPGVQEGLTTLPRSPASVFMEQGSGEAAADPETTTVSSFSLNVEYAIQAEKEVAGTLSPHVETTFSTEPTGLVLSTVMDRVVAENITQTSREIVISERLGEPNYGAEIRGFSTGFPLEEDFSGDFREYSTVSHPIAKEETVMMEGSGDAAFRDTQTSPSTVPTSVHISHISDSEGPSSTMVSTSAFPWEEFTSSAEGSGEQLVTVSSSVVPVLPSAVQKFSGTASSIIDEGLGEVGTVNEIDRRSTILPTAEVEGTKAPVEKEEVKVSGTVSTNFPQTIEPAKLWSRQEVNPVRQEIESETTSEEQIQEEKSFESPQNSPATEQTIFDSQTFTETELKTTDYSVLTTKKTYSDDKEMKEEDTSLVNMSTPDPDANGLESYTTLPEATEKSHFFLATALVTESIPAEHVVTDSPIKKEESTKHFPKGMRPTIQESDTELLFSGLGSGEEVLPTLPTESVNFTEVEQINNTLYPHTSQVESTSSDKIEDFNRMENVAKEVGPLVSQTDIFEGSGSVTSTTLIEILSDTGAEGPTVAPLPFSTDIGHPQNQTVRWAEEIQTSRPQTITEQDSNKNSSTAEINETTTSSTDFLARAYGFEMAKEFVTSAPKPSDLYYEPSGEGSGEVDIVDSFHTSATTQATRQESSTTFVSDGSLEKHPEVPSAKAVTADGFPTVSVMLPLHSEQNKSSPDPTSTLSNTVSYERSTDGSFQDRFREFEDSTLKPNRKKPTENIIIDLDKEDKDLILTITESTILEILPELTSDKNTIIDIDHTKPVYEDILGMQTDIDTEVPSEPHDSNDESNDDSTQVQEIYEAAVNLSLTEETFEGSADVLASYTQATHDESMTYEDRSQLDHMGFHFTTGIPAPSTETELDVLLPTATSLPIPRKSATVIPEIEGIKAEAKALDDMFESSTLSDGQAIADQSEIIPTLGQFERTQEEYEDKKHAGPSFQPEFSSGAEEALVDHTPYLSIATTHLMDQSVTEVPDVMEGSNPPYYTDTTLAVSTFAKLSSQTPSSPLTIYSGSEASGHTEIPQPSALPGIDVGSSVMSPQDSFKEIHVNIEATFKPSSEEYLHITEPPSLSPDTKLEPSEDDGKPELLEEMEASPTELIAVEGTEILQDFQNKTDGQVSGEAIKMFPTIKTPEAGTVITTADEIELEGATQWPHSTSASATYGVEAGVVPWLSPQTSERPTLSSSPEINPETQAALIRGQDSTIAASEQQVAARILDSNDQATVNPVEFNTEVATPPFSLLETSNETDFLIGINEESVEGTAIYLPGPDRCKMNPCLNGGTCYPTETSYVCTCVPGYSGDQCELDFDECHSNPCRNGATCVDGFNTFRCLCLPSYVGALCEQDTETCDYGWHKFQGQCYKYFAHRRTWDAAERECRLQGAHLTSILSHEEQMFVNRVGHDYQWIGLNDKMFEHDFRWTDGSTLQYENWRPNQPDSFFSAGEDCVVIIWHENGQWNDVPCNYHLTYTCKKGTVACGQPPVVENAKTFGKMKPRYEINSLIRYHCKDGFIQRHLPTIRCLGNGRWAIPKITCMNPSAYQRTYSMKYFKNSSSAKDNSINTSKHDHRWSRRWQESRR

SEQ ID NO: 40 - DNA, 3834 bp, Homo sapiens
acagtgatataatgatgatgggtgtcacaacccgcatttgaacttgcaggcgagctgccccgagcctttctggggaagaactccaggcgtgcggacgcaacagccgagaacattaggtgttgtggacaggagctgggaccaagatcttcggccagccccgcatcctcccgcatcttccagcaccgtcccgcaccctccgcatccttccccgggccaccacgcttcctatgtgacccgcctgggcaacgccgaacccagtcgcgcagcgctgcagtgaattttccccccaaactgcaataagccgccttccaaggccaagatgttcataaatataaagagcatcttatggatgtgttcaaccttaatagtaacccatgcgctacataaagtcaaagtgggaaaaagcccaccggtgaggggctccctctctggaaaagtcagcctaccttgtcatttttcaacgatgcctactttgccacccagttacaacaccagtgaatttctccgcatcaaatggtctaagattgaagtggacaaaaatggaaaagatttgaaagagactactgtccttgtggcccaaaatggaaatatcaagattggtcaggactacaaagggagagtgtctgtgcccacacatcccgaggctgtgggcgatgcctccctcactgtggtcaagctgctggcaagtgatgcgggtctttaccgctgtgacgtcatgtacgggattgaagacacacaagacacggtgtcactgactgtggatggggttgtgtttcactacagggcggcaaccagcaggtacacactgaattttgaggctgctcagaaggcttgtttggacgttggggcagtcatagcaactccagagcagctctttgctgcctatgaagatggatttgagcagtgtgacgcaggctggctggctgatcagactgtcagatatcccatccgggctcccagagtaggctgttatggagataagatgggaaaggcaggagtcaggacttatggattccgttctccccaggaaacttacgatgtgtattgttatgtggatcatctggatggtgatgtgttccacctcactgtccccagtaaattcaccttcgaggaggctgcaaaagagtgtgaaaaccaggatgccaggctggcaacagtgggggaactccaggcggcatggaggaacggctttgaccagtgcgattacgggtggctgtcggatgccagcgtgcgccaccctgtgactgtggccagggcccagtgtggaggtggtctacttggggtgagaaccctgtatcgttttgagaaccagacaggcttccctccccctgatagcagatttgatgcctactgctttaaacgacctgatcgctgcaaaatgaacccgtgccttaacggaggcacctgttatcctactgaaacttcctacgtatgcacctgtgtgccaggatacagcggagaccagtgtgaacttgattttgatgaatgtcactctaatccctgtcgtaatggagccacttgtgttgatggttttaacacattcaggtgcctctgccttccaagttatgttggtgcactttgtgagcaagataccgagacatgtgactatggctggcacaaattccaagggcagtgctacaaatactttgcccatcgacgcacatgggatgcagctgaacgggaatgccgtctgcagggtgcccatctcacaagcatcctgtctcacgaagaacaaatgtttgttaatcgtgtgggccatgattatcagtggataggcctcaatgacaagatgtttgagcatgacttccgttggactgatggcagcacactgcaatacgagaattggagacccaaccagccagacagcttcttttctgctggagaagactgtgttgtaatcatttggcatgagaatggccagtggaatgatgttccctgcaattaccatctcacctatacgtgcaagaaaggaacagtcgcttgcggccagccccctgttgtagaaaatgccaagacctttggaaagatgaaacctcgttatgaaatcaactccctgattagataccactgcaaagatggtttcattcaacgtcaccttccaactatccggtgcttaggaaatggaagatgggctatacctaaaattacctgcatgaacccatctgcataccaaaggacttattctatgaaatactttaaaaattcctcatcagcaaaggacaattcaataaatacatccaaacatgatcatcgttggagccggaggtggcaggagtcgaggcgctgatccctaaaatggcgaacatgtgttttcatcatttcagccaaagtcctaacttcctgtgcctttcctatcacctcgagaagtaattatcagttggtttggatttttggaccaccgttcagtcattttgggttgccgtgctcccaaaacattttaaatgaaagtattggcattcaaaaagacagcagacaaaatgaaagaaaatgagagcagaaagtaagcatttccagcctatctaatttctttagttttctatttgcctccagtgcagtccatttcctaatgtataccagcctactgtactatttaaaatgctcaatttcagcaccgatggccatgtaaataagatgatttaatgttgattttaatcctgtatataaaataaaaagtcacaatgagtttgggcatatttaatgatgattatggagccttagaggtctttaatcattggttcggctgcttttatgtagtttaggctggaaatggtttcacttgctctttgactgtcagcaagactgaagatggcttttcctggacagctagaaaacacaaaatcttgtaggtcattgcacctatctcagccataggtgcagtttgcttctacatgatgctaaaggctgcgaatgggatcctgatggaactaaggactccaatgtcgaactcttctttgctgcattcctttttcttcacttacaagaaaggcctgaatggaggacttttctgtaaccaggaacattttttaggggtcaaagtgctaataattaactcaaccaggtctactttttaatggctttcataacactaactcataaggttaccgatcaatgcatttcatacggatatagacctagggctctggagggtgggggattgttaaaacacatgcaaaaaaaaaaaaaaaaaaaaaaaaagaaattttgtatatataaccattttaatcttttataaagttttgaatgttcatgtatgaatgctgcagctgtgaagcatacataaataaatgaagtaagccatactgatttaatttattggatgttattttccctaagacctgaaaatgaacatagtatgctagttatttttcagtgttagccttttactttcctcacacaatttggaatcatataatataggtactttgtccctgattaaataatgtgacggatagaatgcatcaagtgtttattatgaaaagagtggaaaagtatatagcttttagcaaaaggtgtttgcccattctaagaaatgagcgaatatatagaaatagtgtgggcatttcttcctgttaggtggagtgtatgtgttgacatttctccccatctcttcccactctgttttctccccattatttgaataaagtgactgctgaagatgactttgaatccttatccacttaatttaatgtttaaagaaaaacctgtaatggaaagtaagactccttccctaatttcagtttagagcaacttgaagaagagtagacaaaaaataaaatgcacatagaaaaagagaaaaagggcacaaagggattggcccaatattgattctttttt

SEQ ID NO: 41 - protein, 655 aa, Homo sapiens
MFINIKSILWMCSTLIVTHALHKVKVGKSPPVRGSLSGKVSLPCHFSTMPTLPPSYNTSEFLRIKWSKIEVDKNGKDLKETTVLVAQNGNIKIGQDYKGRVSVPTHPEAVGDASLTVVKLLASDAGLYRCDVMYGIEDTQDTVSLTVDGVVFHYRAATSRYTLNFEAAQKACLDVGAVIATPEQLFAAYEDGFEQCDAGWLADQTVRYPIRAPRVGCYGDKMGKAGVRTYGFRSPQETYDVYCYVDHLDGDVFHLTVPSKFTFEEAAKECENQDARLATVGELQAAWRNGFDQCDYGWLSDASVRHPVTVARAQCGGGLLGVRTLYRFENQTGFPPPDSRFDAYCFKRPDRCKMNPCLNGGTCYPTETSYVCTCVPGYSGDQCELDFDECHSNPCRNGATCVDGFNTFRCLCLPSYVGALCEQDTETCDYGWHKFQGQCYKYFAHRRTWDAAERECRLQGAHLTSILSHEEQMFVNRVGHDYQWIGLNDKMFEHDFRWTDGSTLQYENWRPNQPDSFFSAGEDCVVIIWHENGQWNDVPCNYHLTYTCKKGTVACGQPPVVENAKTFGKMKPRYEINSLIRYHCKDGFIQRHLPTIRCLGNGRWAIPKITCMNPSAYQRTYSMKYFKNSSSAKDNSINTSKHDHRWSRRWQESRR

SEQ ID NO: 42 - DNA, 2861 bp, Homo sapiens
gtccctcacaccgagagttcctgcgcgtggggagttggagagtttgcgtggcgggaacgcggcggcagtgagagcgagcggcgccggcccttgcgtccggtgcggcgatgctgaccccggcgttcgacctcagccaggatccggacttcctgactatcgccatccgcgtgccctacgcccgggtctccgagttcgacgtctacttcgaggggtctgacttcaagttctacgccaagccatactttctcagattaacccttcctggaagaattgtagaaaatggaagtgagcaagggtcctatgatgcagataaaggaatttttaccattcgcctgcccaaagaaacccctggccagcattttgaggggctgaacatgttaactgctcttctggcaccaagaaaatccaggacagcaaaaccacttgtggaagaaataggtgcttctgagattcctgaggaagtagttgacgatgaagagtttgattgggaaattgagcagacaccctgtgaagaggtatcagaaagtgctttgaatccgcagtgccactatggatttggaaacttacgatcaggagtgttgcaacggttacaggatgaactgagtgatgttattgatattaaggatccagatttcacccctgcagctgaacgaagacagaagcgcctggccgctgagctggccaagtttgatcctgatcattatctagctgacttttttgaagatgaggcgattgaacagattttgaagtataatccttggtggactgacaaatattcaaaaatgatggcctttttggaaaagagtcaggaacaagaaaatcatgctacattagtgtctttttctgaagaagagaagtatcagctacgaaaatttgtcaataaatcttatctgctggacaagagagcctgtcgtcaagtgtgctacagtttgattgatatccttctggcatattgctatgaaacccgtgtcactgaaggagagaagaatgttgaatctgcatggaatatcaggaaactgagtccaacactatgctggtttgagacttggactaacgttcatgatatcatggtgtcttttggaagaagggtgttgtgttacccactctatcgccatttcaagctggtgatgaaggcctacagggacactataaagatattgcaactgggtaaaagtgcagttttaaagtgtctcctggatattcacaaaatttttcaggaaaatgacccagcgtacatactgaatgatctctacatctcagactactgtgtgtggattcagaaagtcaaatccaaaaagttggcagctcttgcagaagccttaaaggaagtctcccttacaaaggcccagctggggttagaactggaagaactagaagcagcagcactgcttgtccaggaggaagaaactgcattaaaagcagcccattcagtttctgggcagcagacactttgctccagctctgaggcaagtgattcggaggactcagacagcagcgtgtcatctggaaacgaagactcaggctcagattcagaacaagatgaactcaaagatagtccatctgagacagtcagttctttgcaaggtccctttcttgaagaaagcagtgcctttcttattgttgatggtggagtacgcagaaacacagccatccaggagtctgatgccagtcagggaaagccacttgcctcttcctggcctcttggagtgtctgggcctctgatagaggagcttggggaacaactgaagactacagttcaggtttctgaacccaagggcaccactgctgtaaaccgcagcaatattcaggagagagacggctgtcagacaccaaataattgactcttaggtggttttattcattgttgagaaatatggtagattgggtttcatttaccgaatgagaattcttcattttcactttgtaatttttcttagtatatagtcagcccactgtatttgtgtgttccacatctgtggattcaaccaactgcagatcaaaaatattgaagaaaaaatcgcatctgtaccaaacatgtacagacttttttcttgttattattctctaaataatacagtatgacaactatttccacagcatttacattgtattgggtaatataagtaatctagtgatgatttaaactgtgcaggaggatgtgggtaggttatatgcaaatactgcaccattttttatcaggggcttgagcatctgaggattttagtatcctcaggagtcctggaaccaaccccccacagatacggggacaactttatgacattgtttttcaaccaatgaatgtttataccttttgttttccttgccgcgactgtgaagataaagttcaaaagtatttttaccaaagtgtagctaatatttcaagctgaaaataatagttctactgcccgtgtctccagaatgtagagcccatcaatatttttattttaggaggtgtacttgacacccaataaactgcacgtatcaaaagtgtacaatgtgttggcgtatgtataccctgcgtaaaactatcaccacaactgaagtgaggaacggacccatcattcccaagtgcccttgtgccctgttattagtttgcatgtcctagaattctatgtaaattgaatcatacaatatgtatcccttttttggtcaggcatattttactcagtatagttattttgagattcatccatgtggtagcatgtgtcacgagtttttgtttttatgtttttcattgctgagtactgcttcattgaatagatttgttcattgacctatcgatggacacctgggttgtttccagtttggggctgttataaataaagctgttatgaacatttgtgtacaaaaaaaaaaaaaaa

SEQ ID NO: 43 - protein, 577 aa, Homo sapiens
MLTPAFDLSQDPDFLTIAIRVPYARVSEFDVYFEGSDFKFYAKPYFLRLTLPGRIVENGSEQGSYDADKGIFTIRLPKETPGQHFEGLNMLTALLAPRKSRTAKPLVEEIGASEIPEEVVDDEEFDWEIEQTPCEEVSESALNPQCHYGFGNLRSGVLQRLQDELSDVIDIKDPDFTPAAERRQKRLAAELAKFDPDHYLADFFEDEAIEQILKYNPWWTDKYSKMMAFLEKSQEQENHATLVSFSEEEKYQLRKFVNKSYLLDKRACRQVCYSLIDILLAYCYETRVTEGEKNVESAWNIRKLSPTLCWFETWTNVHDIMVSFGRRVLCYPLYRHFKLVMKAYRDTIKILQLGKSAVLKCLLDIHKIFQENDPAYILNDLYISDYCVWIQKVKSKKLAALAEALKEVSLTKAQLGLELEELEAAALLVQEEETALKAAHSVSGQQTLCSSSEASDSEDSDSSVSSGNEDSGSDSEQDELKDSPSETVSSLQGPFLEESSAFLIVDGGVRRNTAIQESDASQGKPLASSWPLGVSGPLIEELGEQLKTTVQVSEPKGTTAVNRSNIQERDGCQTPNN

SEQ ID NO: 44 - DNA, 1110 bp, Homo sapiens
cctgggcggctccgctagctgtttttcgtcttccctaggctatttctgccgggcgctccgcgaagatgcagctcaagccgatggagatcaaccccgagatgctgaacaaagtgctgtcccggctgggggtcgccggccagtggcgcttcgtggacgtgctggggctggaagaggagtctctgggctcggtgccagcgcctgcctgcgcgctgctgctgctgtttcccctcacggcccagcatgagaacttcaggaaaaagcagattgaagagctgaagggacaagaagttagtcctaaagtgtacttcatgaagcagaccattgggaattcctgtggcacaatcggacttattcacgcagtggccaataatcaagacaaactgggatttgaggatggatcagttctgaaacagtttctttctgaaacagagaaaatgtcccctgaagacagagcaaaatgctttgaaaagaatgaggccatacaggcagcccatgatgccgtggcacaggaaggccaatgtcgggtagatgacaaggtgaatttccattttattctgtttaacaacgtggatggccacctctatgaacttgatggacgaatgccttttccggtgaaccatggcgccagttcagaggacaccctgctgaaggacgctgccaaggtctgcagagaattcaccgagcgtgagcaaggagaagtccgcttctctgccgtggctctctgcaaggcagcctaatgctctgtgggagggactttgctgatttcccctcttcccttcaacatgaaaatatatacccccccatgcagtctaaaatgcttcagtacttgtgaaacacagctgttcttctgttctgcagacacgccttcccctcagccacacccaggcacttaagcacaagcagagtgcacagctgtccactgggccattgtggtgtgagcttcagatggtgaagcattctccccagtgtatgtcttgtatccgatatctaacgctttaaatggctactttggtttctgtctgtaagttaagaccttggatgtggtttaattgtttgtcctcaaaaggaataaaacttttctgctgataagataaaaaaaaaaaaaaaaaa

SEQ ID NO: 45 - protein, 223 aa, Homo sapiens [sequence omitted]

SEQ ID NO: 46 - DNA, 7644 bp, Homo sapiens
agcggaggctttgctgtggcagctgctggagcggcggccgcctcgggagccggaggagaggcagccgcggagcgccgagctggcctcgccccgaggcccggccctgggtgtggggaaccgcgctgaggagctggaaactttcccggcaggagctgactgctgggcaggaacgtctctcaggagaaagagtggaagagaaaattgtgaactaaggccccctgcccccttttcctggtgcatgtgaagttatcaaaaacagaaacaagtgttgaaaatgttaaaggctgtgctgaagaagagccgagagggaggaaagggaggcaagaaggaagcaggaagtgactttggtccagagacttctccagtcctgcaccttgaccacagtgctgactctcctgtgagcagtcttcccacagcagaggacacctatagggtgagcttggccaaaggtgtctcgatgtctctgccttcctcacctttgctgcctcgacagtctcacttggtgcaatcaagagtgaacaaaaaatccccaggtcccgtcaggaagcccaagtatgtggaaagccccagagtgcctggagatgcagttataatgccattcagagaagtagccaagccaacagagcctgatgagcatgaagcaaaggccgataatgaaccgagctgttcgccggcagctcaagaactgttgacaaggctgggatttttactgggagaagggatcccaagtgccacacacataaccattgaagacaaaaatgaaaccatgtgcacagctctgagtcaaggcatcagtccttgctccacactaacaagcagcaccgcatctcctagcaccgatagcccctgctcaaccttgaatagctgtgtcagcaagacggcagccaacaaaagtccctgtgagaccattagcagccctagttccaccctggaaagcaaggacagtggaattatagccacaattacaagttcatccgaaaatgatgaccggagtggctccagtttggaatggaataaagatggaagcctaagattaggggttcagaagggagtgcttcatgaccgcagggcagataactgctccccagtggcagaagaggagaccaccgggtcagcagagagcacgctgcccaaagcagaatcctcagctggagatggtccagtcccttattctcagggctccagctcactaataatgccacggcccaactcagttgcagcaacaagctcaaccaaattggaagatctgagttatttagacgggcagagaaatgctcctctacggacgtcaattagattaccatggcacaatacggccggaggtagggcacaggaagttaaagcacgatttgctccctacaagccacaagacattttgttgaaacccttgttgtttgaagtaccaagcataacaacagactctgtgtttgtgggaagggattggctctttcaccagatagaagaaaacttgaggaacacagaactggcagaaaacagaggcgcggtggtggttggcaatgtgggatttgggaagacggcaatcatttccaagttggtggccctgagctgccacggaagccgcatgaggcagattgcttccaacagcccgggttcatcacctaaaacctctgaccccactcaggatcttcatttcactccgttgctttcaccgagttcttccacaagtgcttccagcacagctaaaacacctcttgggtctatcagtgctgaaaaccagagaccaagagaggatgcagtgaaatatcttgcttctaaggtggtggcctaccactactgccaggctgacaacacgtacacttgcctggtgcccgagtttgtgcacagcatcgcagctttgctctgccggtcccatcagctggccgcctacagagaccttctgataaaggagccccaactacagagcatgctgagcctccgatcctgtgtgcaggacccggtggcagctttcaagaggggagtgctggagccactcacaaacctgagaaatgagcagaaaattcctgaagaagaatacattattttgatagatggcttaaatgaagctgagtttcataaacctgattatggagatacgctttcttcatttattaccaaaattatttctaaatttcctgcctggttgaagttgattgtgactgtaagagcaaattttcaggaaatcataagtgcgctgccatttgtcaagctttccttagatgacttcccagacaacaaagacatccacagtgacctgcacgcctacgtccagcacagggtgcacagcagccaggacatcctcagcaacatctccctgaacggcaaggccgatgccacactcattggaaaagtgagcagccacctggtgctgcggagcctcggctcctacctgtacctcaagctcaccctggaccttttccagaggggccacttggtcattaagagtgccagctacaaggtggtgcccgtgtctctctctgagctctatttgcttcagtgcaacatgaagttcatgacccagtccgcctttgagagggcacttccgattctcaacgtggccctcgcatccctccaccccatgacagacgagcagatctttcaggctattaatgctggccacatccagggggagcagggatgggaagactttcagcagaggatggacgccctctcctgcttcctcattaagaggcgagacaaaacccgcatgttctgccacccgtccttcagggagtggcttgtatggagagcagacggggaaaacacggccttcctgtgtgagcccaggaacgggcacgcgctcttggcattcatgttctcgcgtcaggagggcaagttgaaccgccagcagaccatggagcttggccaccacatcctgaaggcgcacattttcaagggcctcagtaagaagacgggaatttcttcaagccatctccaagccctgtggatcggctacagcaccgaggggctgtccgccgccctggcctctctcaggaatctctatactcccaacgtgaaggtgagccgtctcctgattttgggaggggccaacgtgaactacaggacagaagtgttaaataatgccccaatcctgtgcgtccagtctcaccttggccacgaggaagttgtcactctgctcctggaatttggtgcctgcctggacggaacgtcagagaacggcatgactgccctctgttacgcagcagctgctggccacatgaagctggtgtgtctgctgaccaagaagggagtgagagtggaccacttggataagaagggccagtgtgcgcttgtccacagtgccctacggggccacggtgacattctccagtacctgctgacttgtgagtggtcgccgggtcctccccagccaggcaccctgaggaagagccacgccctgcagcaggcgctgaccgcggcggccagcatgggccacagctcggtggtccagtgcttgctggggatggagaaggaacatgaagtagaagtcaatggcaccgacacattgtggggagaaacagccctgactgccgccgcaggaagagggaagctggaggtctgtgagctgctgctggggcatggagctgctgtgtcgcggacaaacaggagaggggttccacctttgttttgtgcagcacgccaggggcattggcagattgttagactgctgttggaacgcggctgtgatgtgaacctaagtgacaagcaaggccggacgcccctcatggtggctgcttgtgaagggcacttgagcaccgtggaattcctcctttcaaaaggtgcagccctttcttctctagacaaagagggtctgtcagcattaagctgggcttgtctgaaaggtcacagggcagtggtccagtatctggttgaagaaggagctgcaatagaccagacagacaagaatggccgcacacccttggacctggctgccttctatggcgatgccgagactgtgctgtacctggtggagaagggagccgtgatcgagcatgtggaccacagcgggatgcggcccttggacagagccatcggctgccggaacacatctgtagtggtggcgctactcagaaagggagccaagttaggaaatgctgcttgggcgatggccacttccaaacctgatatcttgattatacttttacagaaattaatggaggaaggaaatgtgatgtacaaaaaagggaaaatgaaagaggcagcccagaggtaccagtatgccttaagaaagtttcctcgagaaggattcggagaggacatgagacccttcaatgaattaagggtttccctctatctcaatttgtcgcgatgccgaagaaaaacaaatgactttggcatggcagaggaatttgcttccaaggctctcgaattgaagcccaagtcctatgaagccttttatgccagagcaagagcgaagagaaatagcaggcaattcgtggcagctctggctgacctgcaagaggctgtgaaactctgtcccaccaatcaggaagtcaagaggcttctggcccgcgtagaagaggagtgcaaacaactccagaggagtcaacagcaaaaacagcagggcccgctaccagctccactcaacgactccgagaacgaagaggacaccccaacccctggcttaagtgaccactttcactctgaggagactgaagaggaagaaacttctccccaggaagaatctgtttccccaactcccaggtcccagccatcctcatctgtcccttcctcatacatccgaaaccttcaagaagggttacagtccaaaggaaggccggtatcgccacagagcagggcaggaatcggcaagtccctgagagagcctgtggcccagccagggctgctcctgcagccctccaagcaggcccagatcgtgaaaaccagccagcacctgggctctggccagtcggcagtgagaaatggcagtatgaaagttcagatctcttctcagaaccctcctccaagtcccatgccagggagaatcgctgccactcctgctgggagcagaacccagcatttagagggaacaggtactttcactacaagagctggttgtggccactttggggatcggctgggccccagccagaatgtccgcctgcagtgtggtgagaatggccctgcacaccctttaccaagtaagacgaaaaccacagagaggcttctgtctcattcctccgtggctgtggacgcagcccctccaaaccaaggtgggctggcgacctgcagcgacgtgcgacacccagcttccctcaccagctcaggctcttctggttctccatccagcagcataaagatgtcaagttcaaccagtagtttgacttcgagcagcagtttttcagatggcttcaaggtccaaggaccagatactagaattaaagacaaggttgtaacccacgttcagagcggtacagctgagcacagaccccgcaacacgccgttcatgggcatcatggataagactgcgaggttccaacagcagagcaatcctccaagccgcagctggcactgtccggcaccagaggggctgctgacaaacacgtcttctgcagctggcctgcagtctgctaacactgagaagccctctctcatgcaagtgggaggatataataaccaagccaaaacctgttctgtttctaccctgagtgcaagtgtccacaatggggcacaagtgaaggagctagaagaaagcaagtgccaaattccagtccactctcaagagaacaggataactaagactgtttctcatctgtaccaggaaagtatctccaaacagcagcctcatattagtaatgaagcccacaggagccacctcactgcagccaaaccaaagcgatcatttatagagtcaaatgtgtgaaccttaagaaatccccatttgtggaatttggaaacgtgtgttgactcctggtggtaaattaaatagtttttttcatcagaaaaattattttttagccattttttttctttggggtggatctgatgccattgatatatctaaaatgtgggataaaacttctttaatagctagaaatcaccataaataagaatgctaaacagaattgaaaattatatcaacttaaaattttaagacagcccagaagacattaatgactctcacttatgaaattgtttggcttttgccactttcttccttgcctttgctatatggtagaatcacagaacttacttagagaataaatatgtctattgttcaagagtaacaggtttaactcatgaccaagtgatgtacatccaagtgatgtattctggaaacgatggaattttacagttacagttccattgagtcaaatcccattttatatatacataaaaattaagttctgagtgagttctagctaaatataagtgcgactgtaaacgcagccaatttttttaagcagaatatgagaacacctaagtattctcttcatagcagttcctataaagggattaaacacttatttctgtgttatggttcttattcatatatttttatagcacttttggaacctatatttgtgcttgaaggtgtttttgatatttggaaacagtataagccatttggagtcatgattggtggtcaagtggattcaagctaaaatactaagaccagcattcttagtggcgcttataaattagctctcacctggtttccaaactgcttttaacaatggtagtgctcctggaacaatccttccaagctcctctaaggacaatatttaattcagatactaaaggtaagactggttgttacttttgttttgttgtacaattagtactttatagtcacatgttgtatatattaaatagcccagttttattcagacttgtaaatagaactatttcaatgtagttaatctaaaaacaaaaaagaaaaccccagtcacgatttgcatgttctctgtaagcttcatccatgctggttattgcactgaatgatatattattagggcatgttaacagtataccagtaacagcactttatctcatttatatgaacacctttgaggtgctacttaagtccaagctctgatgtattattcatttgtaaagataaggtacaggaatgaaccttggtttaaaggtatttttatatgaaaatggtgtgttattggaagatgttaaaatgctaatttgagagaagtaggagtgtatctgttttatatgttgggatgtgaaatttattttctaaaattgaggagaaggaagttatatatttgcagaatgttttaaagtgaattgttgtaatgaagttcctgtgaacatcattatggttttgtacaaataggaacctctgatgtcattcttcaacgtttgttcctgtgtgtacaattgtactttgtatgaacagctttatcatttttataggctttccatgagttttgctgtaactactatggcttatttattttctttaatatttgtgaaagtcttactcctttgttagttttgtttctgcacaactactgtacttttccatatggaataaagactattaatagaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 47 - protein, 1861 aa, Homo sapiens
MLKAVLKKSREGGKGGKKEAGSDFGPETSPVLHLDHSADSPVSSLPTAEDTYRVSLAKGVSMSLPSSPLLPRQSHLVQSRVNKKSPGPVRKPKYVESPRVPGDAVIMPFREVAKPTEPDEHEAKADNEPSCSPAAQELLTRLGFLLGEGIPSATHITIEDKNETMCTALSQGISPCSTLTSSTASPSTDSPCSTLNSCVSKTAANKSPCETISSPSSTLESKDSGIIATITSSSENDDRSGSSLEWNKDGSLRLGVQKGVLHDRRADNCSPVAEEETTGSAESTLPKAESSAGDGPVPYSQGSSSLIMPRPNSVAATSSTKLEDLSYLDGQRNAPLRTSIRLPWHNTAGGRAQEVKARFAPYKPQDILLKPLLFEVPSITTDSVFVGRDWLFHQIEENLRNTELAENRGAVVVGNVGFGKTAIISKLVALSCHGSRMRQIASNSPGSSPKTSDPTQDLHFTPLLSPSSSTSASSTAKTPLGSISAENQRPREDAVKYLASKVVAYHYCQADNTYTCLVPEFVHSIAALLCRSHQLAAYRDLLIKEPQLQSMLSLRSCVQDPVAAFKRGVLEPLTNLRNEQKIPEEEYIILIDGLNEAEFHKPDYGDTLSSFITKIISKFPAWLKLIVTVRANFQEIISALPFVKLSLDDFPDNKDIHSDLHAYVQHRVHSSQDILSNISLNGKADATLIGKVSSHLVLRSLGSYLYLKLTLDLFQRGHLVIKSASYKVVPVSLSELYLLQCNMKFMTQSAFERALPILNVALASLHPMTDEQIFQAINAGHIQGEQGWEDFQQRMDALSCFLIKRRDKTRMFCHPSFREWLVWRADGENTAFLCEPRNGHALLAFMFSRQEGKLNRQQTMELGHHILKAHIFKGLSKKTGISSSHLQALWIGYSTEGLSAALASLRNLYTPNVKVSRLLILGGANVNYRTEVLNNAPILCVQSHLGHEEVVTLLLEFGACLDGTSENGMTALCYAAAAGHMKLVCLLTKKGVRVDHLDKKGQCALVHSALRGHGDILQYLLTCEWSPGPPQPGTLRKSHALQQALTAAASMGHSSVVQCLLGMEKEHEVEVNGTDTLWGETALTAAAGRGKLEVCELLLGHGAAVSRTNRRGVPPLFCAARQGHWQIVRLLLERGCDVNLSDKQGRTPLMVAACEGHLSTVEFLLSKGAALSSLDKEGLSALSWACLKGHRAVVQYLVEEGAAIDQTDKNGRTPLDLAAFYGDAETVLYLVEKGAVIEHVDHSGMRPLDRAIGCRNTSVVVALLRKGAKLGNAAWAMATSKPDILIILLQKLMEEGNVMYKKGKMKEAAQRYQYALRKFPREGFGEDMRPFNELRVSLYLNLSRCRRKTNDFGMAEEFASKALELKPKSYEAFYARARAKRNSRQFVAALADLQEAVKLCPTNQEVKRLLARVEEECKQLQRSQQQKQQGPLPAPLNDSENEEDTPTPGLSDHFHSEETEEEETSPQEESVSPTPRSQPSSSVPSSYIRNLQEGLQSKGRPVSPQSRAGIGKSLREPVAQPGLLLQPSKQAQIVKTSQHLGSGQSAVRNGSMKVQISSQNPPPSPMPGRIAATPAGSRTQHLEGTGTFTTRAGCGHFGDRLGPSQNVRLQCGENGPAHPLPSKTKTTERLLSHSSVAVDAAPPNQGGLATCSDVRHPASLTSSGSSGSPSSSIKMSSSTSSLTSSSSFSDGFKVQGPDTRIKDKVVTHVQSGTAEHRPRNTPFMGIMDKTARFQQQSNPPSRSWHCPAPEGLLTNTSSAAGLQSANTEKPSLMQVGGYNNQAKTCSVSTLSASVHNGAQVKELEESKCQIPVHSQENRITKTVSHLYQESISKQQPHISNEAHRSHLTAAKPKRSFIESNV

SEQ ID NO: 48 - DNA, 4307 bp, Homo sapiens
aggggcggggccggactcgagcggggcggggctcgcgccagcgcccccagctccgtggcggcttcgcccgcgagtccagaggcaggcgagcagctcggtcgcccccaccggccccatggcagcccccggcgccccagctgagtacggctacatccggaccgtcctgggccagcagatcctgggacaactggacagctccagcctggcgctgccctccgaggccaagctgaagctggcggggagcagcggccgcggcggccagacagtcaagagcctgcggatccaggagcaggtgcagcagaccctcgcccggaagggccgcagctccgtgggcaacggaaatcttcaccgaaccagcagtgttcctgagtatgtctacaacctacacttggttgaaaatgattttgttggaggccgttcccctgttcctaaaacctatgacatgctaaaggctggcacaactgccacttatgaaggtcgctggggaagaggaacagcacagtacagctcccagaagtccgtggaagaaaggtccttgaggcatcctctgaggagactggagatttctcctgacagcagcccggagagggctcactacacgcacagcgattaccagtacagccagagaagccaggctgggcacaccctgcaccaccaagaaagcaggcgggccgccctcctagtgccaccgagatatgctcgttccgagatcgtgggggtcagccgtgctggcaccacaagcaggcagcgccactttgacacataccacagacagtaccagcatggctctgttagcgacaccgtttttgacagcatccctgccaacccggccctgctcacgtaccccaggccagggaccagccgcagcatgggcaacctcttggagaaggagaactacctgacggcagggctcactgtcgggcaggtcaggccgctggtgcccctgcagcccgtcactcagaacagggcttccaggtcctcctggcatcagagctccttccacagcacccgcacgctgagggaagctgggcccagtgtcgccgtggattccagcgggaggagagcgcacttgactgtcggccaggcggccgcagggggaagtgggaatctgctcactgagagaagcactttcactgactcccagctggggaatgcagacatggagatgactctggagcgagcagtgagtatgctcgaggcagaccacatgctgccatccaggatttctgctgcagctactttcatacagcacgagtgcttccagaaatctgaagctcggaagagggttaaccagcttcgtggcatcctcaagcttctgcagctcctaaaagttcagaatgaagacgttcagcgagctgtgtgtggggccttgagaaacttagtatttgaagacaatgacaacaaattggaggtggctgaactaaatggggtacctcggctgctccaggtgctgaagcaaaccagagacttggagactaaaaaacaaataacaggtttgctgtggaatttgtcatctaatgacaaactcaagaatctcatgataacagaagcattgcttacgctgacggagaatatcatcatccccttttctgggtggcctgaaggagactacccaaaagcaaatggtttgctcgattttgacatattctacaacgtcactggatgcctaagaaacatgagttctgctggcgctgatgggagaaaagcgatgagaagatgtgacggactcattgactcactggtccattatgtcagaggaaccattgcagattaccagccagatgacaaggccacggagaattgtgtgtgcattcttcataacctctcctaccagctggaggcagagctcccagagaaatattcccagaatatctatattcaaaaccggaatatccagactgacaacaacaaaagtattggatgttttggcagtcgaagcaggaaagtaaaagagcaataccaggacgtgccgatgccggaggaaaagagcaaccccaagggcgtggagtggctgtggcattccattgttataaggatgtatctgtccttgatcgccaaaagtgtccgcaactacacacaagaagcatccttaggagctctgcagaacctcacggccggaagtggaccaatgccgacatcagtggctcagacagttgtccagaaggaaagtggcctgcagcacacccgaaagatgctgcatgttggtgacccaagtgtgaaaaagacagccatctcgctgctgaggaatctgtcccggaatctttctctgcagaatgaaattgccaaagaaactctccctgatttggtttccatcattcctgacacagtcccgagtactgaccttctcattgaaactacagcctctgcctgttacacattgaacaacataatccaaaacagttaccagaatgcacgcgaccttctaaacaccgggggcatccagaaaattatggccattagtgcaggcgatgcctatgcctccaacaaagcaagtaaagctgcttccgtccttctgtattctctgtgggcacacacggaactgcatcatgcctacaagaaggctcagtttaagaagacagattttgtcaacagccggactgccaaagcctaccactcccttaaagactgaggaaaatgacaaagtattctcggctgcaaaaatccccaaaggaaaacacctatttttctactacccagcccaagaaacctcaaaagcatgccttgtttctatccttctctatttccgtggtcccctgaatccagaaaacaaatagaacataattttatgagtcttccagaagacctttgcaagtttgccaccagtagataccggccacaggctcgacaaatagtggtctttgttattagggcttatggtacatggcttcctggaatcaaaatgtgaattcatgtggaagggacattaatccaataaataaggaaagaagctgttgcattactgggattttaaaagtttgatttacatttatattccttttctggttcccatgttttgtcactcatgtgcacattgcttcgccattgggcctccagtgtattgttctgcagtgttgaaacagaatggaaatgacaagaaatatctgcagttatccaggagaaagtataatggcaaaattattggtttctttctttactttgtgcttgtttttatccccttgggttgtttttctctgatttttaaataaacttaagaaatttagattacagagtatgcatgactgtaagaaaaagaaattgagaggaagtgatcatagcaaattaaagaagtcttttcctcccagaacttaaagtaaaataaaaaataaataaataaataaaatcttttccacagagaaaggcaactgtgatgataaaatttaacgttcccccaaacactgagtcaatgagatttttctcaggagatactttacctataacaacgccgttaaatccaaatctcttctaaacgatggcattctatgtaatgcctttcctggacttttttggccactgccctggactagtgaaagaatggactctatctttatctgcaagaggaactaaggccttctctcagactgcctggccagcctggggcactgaaaatacggctcatgttaatgagttacattatcagccagcccagccttgcccaccatttaagaaatatcacagagccactagatctcatatgatcttcttcaagccattattttaactcaagaaaactctagagaagaaaagtgaagaagtcatgttgaagaagatgtaagaatgtgtcaagaccatccagaaatgatatgagaaatactgatattttaaatggttgacatcatccagcgaaatgaatctacattaaatgttgttttaactgcgctatgattaaaaccattcatatagagttagtctttacaactactattctgttatttttttttttaatctgacaacatttgtcctaagtaagataagcaaaaaaattcttcaactccttttggcaagaaaactgtaacagaaaataaattttgaatgtgtacttaagtctttattatatttgaagcaattttttttcaattttaaaagctgaatgaagacaacttaggttgctaacctagttcaaaatgaaattatttagataccaatttttaaaatactggagagaatttatatgtctttttccagagttctgatgataagcatttggagtgcatttattcctccagataataaatgtgtgttcagaactttttgtgttttttaaggcattaataaagccttcgataatattaaatacaaaatgagaccaa

SEQ ID NO: 49 - DNA, 4439 bp, Homo sapiens
aggggcggggccggactcgagcggggcggggctcgcgccagcgcccccagctccgtggcggcttcgcccgcgagtccagaggcaggcgagcagctcggtcgcccccaccggccccatggcagcccccggcgccccagctgagtacggctacatccggaccgtcctgggccagcagatcctgggacaactggacagctccagcctggcgctgccctccgaggccaagctgaagctggcggggagcagcggccgcggcggccagacagtcaagagcctgcggatccaggagcaggtgcagcagaccctcgcccggaagggccgcagctccgtgggcaacggaaatcttcaccgaaccagcagtgttcctgagtatgtctacaacctacacttggttgaaaatgattttgttggaggccgttcccctgttcctaaaacctatgacatgctaaaggctggcacaactgccacttatgaaggtcgctggggaagaggaacagcacagtacagctcccagaagtccgtggaagaaaggtccttgaggcatcctctgaggagactggagatttctcctgacagcagcccggagagggctcactacacgcacagcgattaccagtacagccagagaagccaggctgggcacaccctgcaccaccaagaaagcaggcgggccgccctcctagtgccaccgagatatgctcgttccgagatcgtgggggtcagccgtgctggcaccacaagcaggcagcgccactttgacacataccacagacagtaccagcatggctctgttagcgacaccgtttttgacagcatccctgccaacccggccctgctcacgtaccccaggccagggaccagccgcagcatgggcaacctcttggagaaggagaactacctgacggcagggctcactgtcgggcaggtcaggccgctggtgcccctgcagcccgtcactcagaacagggcttccaggtcctcctggcatcagagctccttccacagcacccgcacgctgagggaagctgggcccagtgtcgccgtggattccagcgggaggagagcgcacttgactgtcggccaggcggccgcagggggaagtgggaatctgctcactgagagaagcactttcactgactcccagctggggaatgcagacatggagatgactctggagcgagcagtgagtatgctcgaggcagaccacatgctgccatccaggatttctgctgcagctactttcatacagcacgagtgcttccagaaatctgaagctcggaagagggttaaccagcttcgtggcatcctcaagcttctgcagctcctaaaagttcagaatgaagacgttcagcgagctgtgtgtggggccttgagaaacttagtatttgaagacaatgacaacaaattggaggtggctgaactaaatggggtacctcggctgctccaggtgctgaagcaaaccagagacttggagactaaaaaacaaataacagaccatacagtcaatttaagaagtaggaatggctggccgggcgcggtggctcacgcctgtaatcccagcactttgggaggccaaggcgggcggatcacgaggtcaggagttcgagaccagcctgaccaacatggtttgctgtggaatttgtcatctaatgacaaactcaagaatctcatgataacagaagcattgcttacgctgacggagaatatcatcatccccttttctgggtggcctgaaggagactacccaaaagcaaatggtttgctcgattttgacatattctacaacgtcactggatgcctaagaaacatgagttctgctggcgctgatgggagaaaagcgatgagaagatgtgacggactcattgactcactggtccattatgtcagaggaaccattgcagattaccagccagatgacaaggccacggagaattgtgtgtgcattcttcataacctctcctaccagctggaggcagagctcccagagaaatattcccagaatatctatattcaaaaccggaatatccagactgacaacaacaaaagtattggatgttttggcagtcgaagcaggaaagtaaaagagcaataccaggacgtgccgatgccggaggaaaagagcaaccccaagggcgtggagtggctgtggcattccattgttataaggatgtatctgtccttgatcgccaaaagtgtccgcaactacacacaagaagcatccttaggagctctgcagaacctcacggccggaagtggaccaatgccgacatcagtggctcagacagttgtccagaaggaaagtggcctgcagcacacccgaaagatgctgcatgttggtgacccaagtgtgaaaaagacagccatctcgctgctgaggaatctgtcccggaatctttctctgcagaatgaaattgccaaagaaactctccctgatttggtttccatcattcctgacacagtcccgagtactgaccttctcattgaaactacagcctctgcctgttacacattgaacaacataatccaaaacagttaccagaatgcacgcgaccttctaaacaccgggggcatccagaaaattatggccattagtgcaggcgatgcctatgcctccaacaaagcaagtaaagctgcttccgtccttctgtattctctgtgggcacacacggaactgcatcatgcctacaagaaggctcagtttaagaagacagattttgtcaacagccggactgccaaagcctaccactcccttaaagactgaggaaaatgacaaagtattctcggctgcaaaaatccccaaaggaaaacacctatttttctactacccagcccaagaaacctcaaaagcatgccttgtttctatccttctctatttccgtggtcccctgaatccagaaaacaaatagaacataattttatgagtcttccagaagacctttgcaagtttgccaccagtagataccggccacaggctcgacaaatagtggtctttgttattagggcttatggtacatggcttcctggaatcaaaatgtgaattcatgtggaagggacattaatccaataaataaggaaagaagctgttgcattactgggattttaaaagtttgatttacatttatattccttttctggttcccatgttttgtcactcatgtgcacattgcttcgccattgggcctccagtgtattgttctgcagtgttgaaacagaatggaaatgacaagaaatatctgcagttatccaggagaaagtataatggcaaaattattggtttctttctttactttgtgcttgtttttatccccttgggttgtttttctctgatttttaaataaacttaagaaatttagattacagagtatgcatgactgtaagaaaaagaaattgagaggaagtgatcatagcaaattaaagaagtcttttcctcccagaacttaaagtaaaataaaaaataaataaataaataaaatcttttccacagagaaaggcaactgtgatgataaaatttaacgttcccccaaacactgagtcaatgagatttttctcaggagatactttacctataacaacgccgttaaatccaaatctcttctaaacgatggcattctatgtaatgcctttcctggacttttttggccactgccctggactagtgaaagaatggactctatctttatctgcaagaggaactaaggccttctctcagactgcctggccagcctggggcactgaaaatacggctcatgttaatgagttacattatcagccagcccagccttgcccaccatttaagaaatatcacagagccactagatctcatatgatcttcttcaagccattattttaactcaagaaaactctagagaagaaaagtgaagaagtcatgttgaagaagatgtaagaatgtgtcaagaccatccagaaatgatatgagaaatactgatattttaaatggttgacatcatccagcgaaatgaatctacattaaatgttgttttaactgcgctatgattaaaaccattcatatagagttagtctttacaactactattctgttatttttttttttaatctgacaacatttgtcctaagtaagataagcaaaaaaattcttcaactccttttggcaagaaaactgtaacagaaaataaattttgaatgtgtacttaagtctttattatatttgaagcaattttttttcaattttaaaagctgaatgaagacaacttaggttgctaacctagttcaaaatgaaattatttagataccaatttttaaaatactggagagaatttatatgtctttttccagagttctgatgataagcatttggagtgcatttattcctccagataataaatgtgtgttcagaactttttgtgttttttaaggcattaataaagccttcgataatattaaatacaaaatgagaccaa

SEQ ID NO: 50 - protein, 837 aa, Homo sapiens
MAAPGAPAEYGYIRTVLGQQILGQLDSSSLALPSEAKLKLAGSSGRGGQTVKSLRIQEQVQQTLARKGRSSVGNGNLHRTSSVPEYVYNLHLVENDFVGGRSPVPKTYDMLKAGTTATYEGRWGRGTAQYSSQKSVEERSLRHPLRRLEISPDSSPERAHYTHSDYQYSQRSQAGHTLHHQESRRAALLVPPRYARSEIVGVSRAGTTSRQRHFDTYHRQYQHGSVSDTVFDSIPANPALLTYPRPGTSRSMGNLLEKENYLTAGLTVGQVRPLVPLQPVTQNRASRSSWHQSSFHSTRTLREAGPSVAVDSSGRRAHLTVGQAAAGGSGNLLTERSTFTDSQLGNADMEMTLERAVSMLEADHMLPSRISAAATFIQHECFQKSEARKRVNQLRGILKLLQLLKVQNEDVQRAVCGALRNLVFEDNDNKLEVAELNGVPRLLQVLKQTRDLETKKQITGLLWNLSSNDKLKNLMITEALLTLTENIIIPFSGWPEGDYPKANGLLDFDIFYNVTGCLRNMSSAGADGRKAMRRCDGLIDSLVHYVRGTIADYQPDDKATENCVCILHNLSYQLEAELPEKYSQNIYIQNRNIQTDNNKSIGCFGSRSRKVKEQYQDVPMPEEKSNPKGVEWLWHSIVIRMYLSLIAKSVRNYTQEASLGALQNLTAGSGPMPTSVAQTVVQKESGLQHTRKMLHVGDPSVKKTAISLLRNLSRNLSLQNEIAKETLPDLVSIIPDTVPSTDLLIETTASACYTLNNIIQNSYQNARDLLNTGGIQKIMAISAGDAYASNKASKAASVLLYSLWAHTELHHAYKKAQFKKTDFVNSRTAKAYHSLKD

SEQ ID NO: 51 - protein, 881 aa, Homo sapiens
MAAPGAPAEYGYIRTVLGQQILGQLDSSSLALPSEAKLKLAGSSGRGGQTVKSLRIQEQVQQTLARKGRSSVGNGNLHRTSSVPEYVYNLHLVENDFVGGRSPVPKTYDMLKAGTTATYEGRWGRGTAQYSSQKSVEERSLRHPLRRLEISPDSSPERAHYTHSDYQYSQRSQAGHTLHHQESRRAALLVPPRYARSEIVGVSRAGTTSRQRHFDTYHRQYQHGSVSDTVFDSIPANPALLTYPRPGTSRSMGNLLEKENYLTAGLTVGQVRPLVPLQPVTQNRASRSSWHQSSFHSTRTLREAGPSVAVDSSGRRAHLTVGQAAAGGSGNLLTERSTFTDSQLGNADMEMTLERAVSMLEADHMLPSRISAAATFIQHECFQKSEARKRVNQLRGILKLLQLLKVQNEDVQRAVCGALRNLVFEDNDNKLEVAELNGVPRLLQVLKQTRDLETKKQITDHTVNLRSRNGWPGAVAHACNPSTLGGQGGRITRSGVRDQPDQHGLLWNLSSNDKLKNLMITEALLTLTENIIIPFSGWPEGDYPKANGLLDFDIFYNVTGCLRNMSSAGADGRKAMRRCDGLIDSLVHYVRGTIADYQPDDKATENCVCILHNLSYQLEAELPEKYSQNIYIQNRNIQTDNNKSIGCFGSRSRKVKEQYQDVPMPEEKSNPKGVEWLWHSIVIRMYLSLIAKSVRNYTQEASLGALQNLTAGSGPMPTSVAQTVVQKESGLQHTRKMLHVGDPSVKKTAISLLRNLSRNLSLQNEIAKETLPDLVSIIPDTVPSTDLLIETTASACYTLNNIIQNSYQNARDLLNTGGIQKIMAISAGDAYASNKASKAASVLLYSLWAHTELHHAYKKAQFKKTDFVNSRTAKAYHSLKD

SEQ ID NO: 52 - DNA, 5750 bp, Homo sapiens
ataaccccgcacaccgacttgcatgcaattatcatagcccgagtgctcctccgttgagagacttcgcccccgagaccgctgactgtgaatgacaaatcaaaagtcagggttgcagaatcagccggactttcctgctcatttgcagcagagggaggaagcagagaatgaaagattctgaaaataaaggtgcctcatctccagacatggagcccagctatgggggaggtctctttgacatggtaaaaggaggtgcagggaggctctttagtaacctaaaggacaacttgaaagacaccctcaaagacacatcttctagagtgatacaatctgtgaccagctacacaaagggagatttagacttcacttatgttacctccagaattattgtgatgtcctttcctctggacaatgttgacataggattcaggaatcaggttgatgacattcgaagctttttggattccagacatcttgaccactacacagtatacaatctgtcacctaagtcttatcgaactgccaagtttcacagccgggtctcagaatgcagttggcccattaggcaggctcccagtctgcacaacctttttgctgtgtgtcggaatatgtataactggctactgcagaatcccaaaaatgtctgtgttgtccactgcttggatggacgggcggcatcatcaattctggttggtgctatgttcattttctgtaatctctactctactcctggcccagccattcgattgctatatgcaaagcgaccaggaattggactttcaccatcccataggagatacctgggctatatgtgtgacctactggcagacaagccctaccgccctcacttcaagcctctcacaattaagtcgatcactgtcagtccaataccctttttcaacaaacagaggaatggatgtcgcccttactgtgatgtactcattggagaaaccaaaatatattcgacttgcacagattttgaacgaatgaaagaatatcgtgtccaagatggaaaaatcttcattcccttgaacatcactgtgcaaggagacgtggttgtttccatgtatcacttgaggtcaaccattgggagccggctacaggctaaggtgaccaacacacagatattccagcttcagtttcacactggattcataccactggacacaacagttttaaagttcaccaagcctgagttagatgcatgtgatgtaccagaaaaatatcctcagctatttcaggtgacactggatgtagaactacagccccatgacaaagtaatagacttaactccaccatgggaacattactgcacaaaagatgtcaatcccagcatcctcttctcttctcaccaggaacatcaagatacgctggccttaggaggacaggctccaatagatatccctccagacaaccccaggcattacggacaaagtggtttctttgcctctctctgttggcaagatcagaaatcggagaagtcattctgtgaggaggaccacgctgccctagtgaatcaggaaagtgagcaatcagatgatgaacttctgacactttccagtccgcatggcaatgccaatggtgacaagcctcatggagtcaagaagcccagcaaaaagcagcaggagccagcagcccctccaccccctgaggatgtggaccttttgggcctggaagggtctgcaatgagtaacagcttctctccgccagcggctcctcccaccaattctgaactactgagtgacctgtttgggggtggaggtgcagctggtcccacccaggctggacagtcaggagtggaagatgtgtttcatcctagtggacctgcgtctacccagtcaacaccacgccgctctgccacctccacctctgcgtctccaaccctaagagtgggagaaggtgccacctttgacccatttggagcaccttctaaaccatcaggtcaggatttgctgggttcttttctgaacacatccagtgcttccagtgacccctttctccagccaacaagaagtccttcgcccacagtacatgcttctagtacgcctgctgtgaacattcagccagatgtttctggaggttgggactggcatgctaaaccaggaggctttggaatgggaagcaagtcagctgccaccagcccaaccggatcctcgcatggtactcccacccatcaaagcaaaccccagactctggatccttttgccgaccttgggacactaggtagttcttcctttgccagcaaacccaccacaccaactggattgggtggaggattcccgcctctcagctcgccacagaaggcgtctccccagcctatgggtggcgggtggcagcagggaggtgcctacaactggcagcagccacagcctaagcctcagcccagcatgccccactcctctccccagaaccgacccaactacaacgtgagcttctcagccatgcctgggggccagaacgaacgtgggaaaggatcaagtaatttggaagggaaacaaaaagcagctgattttgaagacctactctctggtcaaggtttcaatgctcacaaagacaaaaaggggcctcggacaatagctgagatgagaaaggaggaaatggccaaggaaatggatcctgagaaattaaagattctggaatggattgaaggcaaagaaagaaatatcagagcccttctttccacgatgcataccgtactatgggctggggagaccaagtggaaaccagttggcatggcagacctggtaacaccagagcaggtgaagaaggtgtacaggaaggctgtcctggtggtgcacccagataaagctactgggcaaccctatgaacaatacgcaaagatgattttcatggagctcaatgatgcctggtctgaatttgaaaaccaaggccaaaagcccttatattaatttatgagcttttccatctctgctgcagacctgtgctaatgcttagtgtgtgtcacaattctgaggttttcgcagatgaaccaaaaactccagtaacatgttttcagtactaaaccgttaagttactcatgaattaatttctcattgataaggaatgtggatgtttggtttctccaaagttcccaccataaaagatccaaagcatgagaggaacttctagtcagatgaccttgcagaaccaccgcattccaccctgccctttggggagcctactcagcattctacctggggaaatggaaaacagaggccaccacccatgaaggcataacacccatcacattgtctgagaataggattaatgagcaaaagttatgataatagagttatatgatggtttaatggtttctagatttttcttggaaagatataatttgagcagtggctttatgcagactccaagaacaaaaccaccaggtatttttttttaagtaaaaatttactaaggcctttagcataatactgtaaatgtcttttggtcaagagaactaacaaatataaaggaacttgaaatgtggggagtgataatagtccagtgattaaactacagtccagtgattaccaccaactttctgactaagctaaaaatgaatgaaaatggtggctaaagaaaatagggactctgaagatgtcgaaatctttattcctggttaggaaggtgggccgctacccttctttccctttagaaaactgtgattaattaaataaaaagtcttactactataacaactctaaacctggtttaaatgaaacatctaattttgctgtaaggcttaatgctcttcttttgtttagccaagaaaacattcttagttggaaacaggtggtaaagtagtttggcctgttcacaacttatcaggaccaatatctagtgtggactcagtgttctctaggtttgtgagcagttgtggtaagagaatggaaggcaacactgaataggagttaagggaatggcacgatcacagtgagagagctgcactcacctgcagacccagtcagatggcttgtcagtatctgctctgaggaaccaggtcccacctccccacaaggcaaatctagacccattaaattattacagtgggcttttttgttttaaaagtggttggggatccacaggaacgacattcatacagggacatttgtgaaagcaaagcaagaatgaatgctttcccgatctcagactggctggattcagatcagtgtgttggctggttctcattttaaggggtaagcagtttgctattctgtgactttagtagatctcttttgtgtgcacatatacaatgtgcattatacatatatattaaatatatccacaaagcagatgggcaaaaagattttcatgctaatcttcaaaggttcatgctcaatattgtgaaaagcttttaaaagttgttttgaaaagacaatcattttttgtttttgttttaactttcttgtggcaagctataccggaaaagcggcatgtcaaaaatactttaggttctacatagatctctttctctcttttgttttgtttgtttgtttgttaaagttcattatttattttaccttttactgacaatgtgaaactcgaagaaaatgttctctttttaaatatacagtcataaactgttcaaggtaaccataacaaactaggtgttatttattaacgtattataaaataatgtttgatccagtatgtgcttgtcttatttaaaattggaatgtgagacatgttgctgtgacctgtttttctttctcattcacatttgtagatattgtgtgaactacagtatataatgataacaattaaaaggatattctgtggatgtcacgtattttgaaatgatagaactacattagctttgtatcatgtttggataattcatcaatgttcacagtttaaaacatcattaaacattatgtaattacaatgagaaagaatcttacttaaatttggagattttcccccacatctcttttccggatacattataattctggacccctatttatctcaaaactcttaatatatgcagaccaacaggtctttgcattccttttaaataactggttgtgacaaagcttgttgttgatcagattcactgtttcatcatatttattgtaatatattttttgttttgtaaatatgttacaacaaaatgtgattggtctaaaatatttgtaatgtatattaaaaggttcaaaaatatttgtataaatctaagttatttgggtacttgtaggaatacaatgaggagcttgaatgccaccttctgacatgatttacttttaaggaaagttatgaggagagaatacaaaaagcatgcaccaaaatgtaatctgacaggatttctggatttatacgtaatcactcctgccccaacacacacacacacacacacacacacacacacacacatgcacgcacacatgattttgattatgctacatgatatatgtttcaaaaagaattagcatagaaatcctggtctcctagccaaaaaaatcaaaggattttcaaaaaaacgaatctgtatgttgaggcaaaaggattgaacctggaagtctgggactttatcatagaaacaaagtctcagatattttagttctttggaaacaaatgctgtaattcaaaagcatttgacctgtcactgtactatctacatgtggaagaatgttcaagttgaatcctaatgccgtgaatgaaacacagtctgtgtagggaatgagcaaaaaagttgaattccaattgctttttggccttttggctaaaataaatgtagcatctaattttatcagttt

SEQ ID NO: 53 - protein, 913 aa, Homo sapiens
MKDSENKGASSPDMEPSYGGGLFDMVKGGAGRLFSNLKDNLKDTLKDTSSRVIQSVTSYTKGDLDFTYVTSRIIVMSFPLDNVDIGFRNQVDDIRSFLDSRHLDHYTVYNLSPKSYRTAKFHSRVSECSWPIRQAPSLHNLFAVCRNMYNWLLQNPKNVCVVHCLDGRAASSILVGAMFIFCNLYSTPGPAIRLLYAKRPGIGLSPSHRRYLGYMCDLLADKPYRPHFKPLTIKSITVSPIPFFNKQRNGCRPYCDVLIGETKIYSTCTDFERMKEYRVQDGKIFIPLNITVQGDVVVSMYHLRSTIGSRLQAKVTNTQIFQLQFHTGFIPLDTTVLKFTKPELDACDVPEKYPQLFQVTLDVELQPHDKVIDLTPPWEHYCTKDVNPSILFSSHQEHQDTLALGGQAPIDIPPDNPRHYGQSGFFASLCWQDQKSEKSFCEEDHAALVNQESEQSDDELLTLSSPHGNANGDKPHGVKKPSKKQQEPAAPPPPEDVDLLGLEGSAMSNSFSPPAAPPTNSELLSDLFGGGGAAGPTQAGQSGVEDVFHPSGPASTQSTPRRSATSTSASPTLRVGEGATFDPFGAPSKPSGQDLLGSFLNTSSASSDPFLQPTRSPSPTVHASSTPAVNIQPDVSGGWDWHAKPGGFGMGSKSAATSPTGSSHGTPTHQSKPQTLDPFADLGTLGSSSFASKPTTPTGLGGGFPPLSSPQKASPQPMGGGWQQGGAYNWQQPQPKPQPSMPHSSPQNRPNYNVSFSAMPGGQNERGKGSSNLEGKQKAADFEDLLSGQGFNAHKDKKGPRTIAEMRKEEMAKEMDPEKLKILEWIEGKERNIRALLSTMHTVLWAGETKWKPVGMADLVTPEQVKKVYRKAVLVVHPDKATGQPYEQYAKMIFMELNDAWSEFENQGQKPLY

SEQ ID NO: 54 - DNA, 5942 bp, Homo sapiens
ggagaactccaccgggctatgcgaacagaatcctgcgaagaggatggcattccctgtggatatgctggaaaattgcagccatgaggaattggaaaattctgctgaagattacatgtcagatttaaggtgtggggaccctgaaaatccagagtgtttttctcttctcaatattacgattcctattagcctgtcaaatgtaggctttgtacctctttatggtggagatcagacccagaaaattcttgctctctttgcacctgaagattcactgacagctgtggcactttaccttgctgatcagtggtgggctattgatgatattgtgaaaacatctgttccttcaagagaggggcttaagcaggtgagcactcttggggagagagtggttctgtatgttctgaatcgaattatttatagaaaacaggaaatggagagaaatgagatcccattcctgtgtcatagcagtactgattatgctaagattctgtggaagaaaggagaggccattgggttttattcagttaagcctacaggaagcatatgtgcctcttttcttacccaaagttaccaattgccagttcttgatacaatgtttctaagaaagaaatacagaggtaaagattttgggcttcacatgctggaggactttgttgattcctttacagaagatgcgcttggcttgcggtatccactgtcttctctcatgtatacagcttgcaagcaatactttgagaagtatccaggagaccatgaactcctttgggaagttgaaggtgttggacactggtaccagcgaataccagtcaccagagcattacaaagagaagcacttaaaattctagcactttctcaaaatgaacctaaaagacctatgtctggagaatatggtcctgcatctgttccagaatacgaagcaagaactgaagacaatcagtctagtgagatgcagctaactattgattctctaaaagatgcctttgcaagcacttccgaaggtcatgataaaacatctgtttccactcatactcgaagtggtaatctaaagcggccaaagattggaaagcggtttcaggattctgaatttagcagttctcaaggtgaagatgaaaagacctcccagacttcacttacagcttcaataaacaaattggagtctactgcacgcccatcagagagctcagaagaattcctggaagaagaacctgaacagagagggattgaatttgaggatgaaagcagtgatagagatgcacggccagcactggaaacccagccacagcaagagaagcaagatggtgaaaaggaatctgaattagagcctatgaatggtgagataatggacgattctcttaagacctcacttataacagaagaggaagactccactagtgaagttttagatgaagaattaaaattgcagccttttaattccagtgaagactctacaaatcttgttccactggtggtagaatcttcaaaaccccctgaggtagatgcaccagataagaccccacgtatacctgactcagaaatgttgatggatgaaggcacatctgatgaaaaggggcacatggaagagaaattgtccctacttccaagaaagaaagcacatcttgggagttcagacaatgttgctactatgtcaaatgaagaacgatctgatggtggttttccaaactctgtgatagctgaattttccgaagaaccggtctctgagaatttgtctcctaatactacttcctcattggaagaccagggtgaggagggggtatctgagccccaggaaacatctactgctcttcctcagagttctttgatagaggttgaacttgaagacgtgccattttcacagaatgcaggacagaagaatcagtcagaggagcagtctgaagcatcttccgagcaactggatcagtttacacaatcggcagaaaaagctgtggatagcagctcagaggaaatagaagtggaagtgcctgtggtagacaggcggaatttaagaagaaaggccaaagggcataaaggacctgctaagaagaaagctaagctgacctgaaggaagaagaaagtggatgataaatcctcttctttgtaacatagttgttgtttttaaaatatggtaattaataaacagcatggggcacaggacaaaaatttccaaaatttcaatttgaacttacttactatgcagttttttgcttccctttagaacgtagaattcaccattgtttttaattttccaggctatttttggtaaatgcaattttttatttttattaaccatgtttcaattttgggaaaccagatcatattatattttctttatcaggtggtacatctgaccattatttcaaaaatataattaaaaagctgtgaaagtagtaggatttccattacttataaggatcacagaaatcttttatactaagggttttaatagtaaaacttggtgagggttctagaagttttaaatttcaaaactaatcaccaattttttaaaatgtaggcatgcctaaacaaaaataagtgtcaataaattagaaaaaatataacttcaagtaaatgagagcacaaaaacagaaacttacggtcaaaggttcatgaagatattttggttttggcctcttaatcctagatatagatgcagttttttcctttgacttcactatctatggaaaggtacacataaaatctgtttctgattcattctatcatctgatgttaacagtactgtcagggatttaactgtggcatgtggggactaattatttaaaatttcatttgaagtattagtttgctgtaattttttttgtctgcatatgtgcagacgcctagaagtttagactttcacctttaatcaggtaaaaatcacaactatactttggagacatggtttaatttggacttctggtcctgatcagatagataaattaagctggagattttacttttatcccaacccagttaacatctatatttcagaagtatgaggctccttcctaaggcatctccttaaatcttgccacctccctaattctacagtcttatttatattcccccacagctttcatctacttctgtaactcatggagtgattctccaatcaacagatcttcctgcctgccggcttatgcccttgatgtccatttttgggtaggtttggaggtgcaccccacaatgttagacctatatggcaggagtaaaactgcccccagtgttagacctatatggcaggggtaaactgcctttgggggagctgtggttacaagggcttcaatcatattggaggcttcaatcatcctttgatttgtactgagtactggttgcaagctggcctgttaagtatatattgtgttgccataacaaaggcctaggaaaatttgaagaaaaaaaacactgaactgttattttttgtaatttatttaaaaatggacaaaatggttattagaatccaaagataatctaaaaacagttctactctttggaaatattatgtgtactcaaatttgcacttaagttggagaatgttttgagaccatttaaattgttttaaaaatagcttgatcttgatccttatgcaaaagaaactaagactttgtgtcattatttgaggtagctaaggactgatgttgaaagtgtattcagtctatagtaagctgatgtcaggtcagtaaattttgtttggggatgttggtattttcgatcaagttaaaatcttaaagcagacaaaaatagtaaacgctcctatttgtgttagattttgaacaattgtattaccctatattttctttaattagtcaaaggtaagccagattatttcattctaaatgaaaattaaagcactttggccatttattttttcaacaaatgctaatactaagtaaaaaaaaaaaagtatgactactattagtgaatctaccaaacatttatgctgttttatacattttatgagaaacttctgtcaatatcacaaagggaaatactctgttaagattcagtaaattcttaatctttacatgtttcatttcttagatttgaaaacacttgtagatattttatttttacttggatttgtttaaaatactttcttttcaaattattttaaacatggaaacgcttcatgaatttgtgtcatccttgcacaggggccatgctaattttccctgtattgttctaattttagtatatgtgctgccgaagtgagcactaaaagactttctttagacaagtgtagttttttaaagattgagctcactggctcaactcttgatgtgtgaatatagttgagtttacaaaaaatgtttattaatcatatatgaccattgggggctaacagtataaacttattcttgttaaacttgtttgggttaaatagtaagtgaattgtaaacaatttagaagttttttgttcagcacacaagaacacttctgttacagattctctctgacagaaattgttattttggggtttatagattatagagggaaaagttagatcacttggaaaaacctaactctcatgttcagtaacttgagtatagaatttataccacaatttatcttcaataaggaacctacatgaacttaatcagttatctgtagtttttgcaaggcattagaaaaaatttcacaattacagaggactgaaaatgtgatttcaaccggctagtctagttgttgaatgtccagtcacattacacatagtcctttgtgaacttatttgtgaagggagttcactttttatgtacatacgtgtatatttccttgtgtaatataaagcagatggttattagtattttagttctttcaagtttaaattattcaattgctagacatcacagaaattagtatatttaagaatttattataaagctcctctacaacttttaaaggtgagagggtctattatgatttttttataaaatcaaacagcatgatttagaacgtcagtcttctacttaaaatgtgtctatattcatggtatttccattcagtgtagatggtaaggaaaacatctagtcattaaagatttacttatagcagactccagaacactacctacaatggggtttcagtgactagaaatcttgaaggtttatgattgcatcaaattgagcctgtactaatatctaaatatctgctcttatgttctgctttatcccttgggattaccttctaaggtttggtcacaccagcaaactgaaacagatcttaatgtacataaaaaatttatactaatattactcatctgtgttacagtattttgaaaaatgtttcaggtgtcatctagtcgtctttcatgggtgtgattagttacaaatgttatgcttatgttttcatacttaatatcaagctttctttgtaccttacaagatagtatttttggttctaaaaaaaacaagcaaacttcatttttgtagtatccagaaattacctggagtcagtatttttattcgccttctagaacttgtgatccacacatcacccatttatattagtggtaaaattatctgctaaaatctaattgtaagaaaggcttaccttctgtcatcaagtgattgtatcatcctggatcgtcatttccaaggaactagcctttcttttcctaagcgtctgtatgtgttctaaaacttccagtatatatttatagaacttagaaaaaatgttacattattcagagtagcaagtcttactggagtacctatgtttgttttcttggtttttatttttttttaagtttaaaagtagtaattaaacctatattttgtgattgtttcctggtctgtgtttttaaattcctttccttcagttttcctcatgaagatgtttcagatactgaatttgtttagacattgaatgactttgttaaaggcacaattaatcacattggttgtactctgaagacagacttctttaaaaaaaaaataaacaatttaaaacaa

SEQ ID NO: 55 - protein, 670 aa, Homo sapiens
MAFPVDMLENCSHEELENSAEDYMSDLRCGDPENPECFSLLNITIPISLSNVGFVPLYGGDQTQKILALFAPEDSLTAVALYLADQWWAIDDIVKTSVPSREGLKQVSTLGERVVLYVLNRIIYRKQEMERNEIPFLCHSSTDYAKILWKKGEAIGFYSVKPTGSICASFLTQSYQLPVLDTMFLRKKYRGKDFGLHMLEDFVDSFTEDALGLRYPLSSLMYTACKQYFEKYPGDHELLWEVEGVGHWYQRIPVTRALQREALKILALSQNEPKRPMSGEYGPASVPEYEARTEDNQSSEMQLTIDSLKDAFASTSEGHDKTSVSTHTRSGNLKRPKIGKRFQDSEFSSSQGEDEKTSQTSLTASINKLESTARPSESSEEFLEEEPEQRGIEFEDESSDRDARPALETQPQQEKQDGEKESELEPMNGEIMDDSLKTSLITEEEDSTSEVLDEELKLQPFNSSEDSTNLVPLVVESSKPPEVDAPDKTPRIPDSEMLMDEGTSDEKGHMEEKLSLLPRKKAHLGSSDNVATMSNEERSDGGFPNSVIAEFSEEPVSENLSPNTTSSLEDQGEEGVSEPQETSTALPQSSLIEVELEDVPFSQNAGQKNQSEEQSEASSEQLDQFTQSAEKAVDSSSEEIEVEVPVVDRRNLRRKAKGHKGPAKKKAKLT

SEQ ID NO: 56 - DNA, 3276 bp, Homo sapiens
aatagactaaacccagagcctcaaagcagtgcactccgtgaaggcaaagagaacacgctgcaaaaggctttccaagaatcctcgacatggcaaggaggagctcgttccagtcgtgtcagataatatccctgttcacttttgccgttggagtcaatatctgcttaggattcactgcacatcgaattaagagagcagaaggatgggaggaaggtcctcctacagtgctatcagactccccctggaccaacatctccggatcttgcaagggcaggtgctttgaacttcaagaggctggacctcctgattgtcgctgtgacaacttgtgtaagagctataccagttgctgccatgactttgatgagctgtgtttgaagacagcccgtggctgggagtgtactaaggacagatgtggagaagtcagaaatgaagaaaatgcctgtcactgctcagaggactgcttggccaggggagactgctgtaccaattaccaagtggtttgcaaaggagagtcgcattgggttgatgatgactgtgaggaaataaaggccgcagaatgccctgcagggtttgttcgccctccattaatcatcttctccgtggatggcttccgtgcatcatacatgaagaaaggcagcaaagtcatgcctaatattgaaaaactaaggtcttgtggcacacactctccctacatgaggccggtgtacccaactaaaacctttcctaacttatacactttggccactgggctatatccagaatcacatggaattgttggcaattcaatgtatgatcctgtatttgatgccacttttcatctgcgagggcgagagaaatttaatcatagatggtggggaggtcaaccgctatggattacagccaccaagcaaggggtgaaagctggaacattcttttggtctgttgtcatccctcacgagcggagaatattaaccatattgcagtggctcaccctgccagatcatgagaggccttcggtctatgccttctattctgagcaacctgatttctctggacacaaatatggccctttcggccctgaggagagtagttatggctcaccttttactccggctaagagacctaagaggaaagttgcccctaagaggagacaggaaagaccagttgctcctccaaagaaaagaagaagaaaaatacataggatggatcattatgctgcggaaactcgtcaggacaaaatgacaaatcctctgagggaaatcgacaaaattgtggggcaattaatggatggactgaaacaactaaaactgcatcggtgtgtcaacgtcatctttgtcggagaccatggaatggaagatgtcacatgtgatagaactgagttcttgagtaattacctaactaatgtggatgatattactttagtgcctggaactctaggaagaattcgatccaaatttagcaacaatgctaaatatgaccccaaagccattattgccaatctcacgtgtaaaaaaccagatcagcactttaagccttacttgaaacagcaccttcccaaacgtttgcactatgccaacaacagaagaattgaggatatccatttattggtggaacgcagatggcatgttgcaaggaaacctttggatgtttataagaaaccatcaggaaaatgctttttccagggagaccacggatttgataacaaggtcaacagcatgcagactgtttttgtaggttatggctcaacatttaagtacaagactaaagtgcctccatttgaaaacattgaactttacaatgttatgtgtgatctcctgggattgaagccagctcctaataatgggacccatggaagtttgaatcatctcctgcgcactaataccttcaggccaaccatgccagaggaagttaccagacccaattatccagggattatgtaccttcagtctgattttgacctgggctgcacttgtgatgataaggtagagccaaagaacaagttggatgaactcaacaaacggcttcatacaaaagggtctacagaagagagacacctcctctatgggcgacctgcagtgctttatcggactagatatgatatcttatatcacactgactttgaaagtggttatagtgaaatattcctaatgccactctggacatcatatactgtttccaaacaggctgaggtttccagcgttcctgaccatctgaccagttgcgtccggcctgatgtccgtgtttctccgagtttcagtcagaactgtttggcctacaaaaatgataagcagatgtcctacggattcctctttcctccttatctgagctcttcaccagaggctaaatatgatgcattccttgtaaccaatatggttccaatgtatcctgctttcaaacgggtctggaattatttccaaagggtattggtgaagaaatatgcttcggaaagaaatggagttaacgtgataagtggaccaatcttcgactatgactatgatggcttacatgacacagaagacaaaataaaacagtacgtggaaggcagttccattcctgttccaactcactactacagcatcatcaccagctgtctggatttcactcagcctgccgacaagtgtgacggccctctctctgtgtcctccttcatcctgcctcaccggcctgacaacgaggagagctgcaatagctcagaggacgaatcaaaatgggtagaagaactcatgaagatgcacacagctagggtgcgtgacattgaacatctcaccagcctggacttcttccgaaagaccagccgcagctacccagaaatcctgacactcaagacatacctgcatacatatgagagcgagatttaactttctgagcatctgcagtacagtcttatcaactggttgtatatttttatattgtttttgtatttattaatttgaaaccaggacattaaaaatgttagtattttaatcctgtaccaaatctgacatattatgcctgaatgactccactgtttttctctaatgcttgatttaggtagccttgtgttctgagtagagcttgtaataaatactgcagcttgagtttttagtggaagcttctaaatggtgctgcagatttgatatttgcattgaggaaatattaattttccaatgcacagttgccacatttagtcctgtactgtatggaaacactgattttgtaaagttgcctttatttgctgttaactgttaactatgacagatatatttaagccttataaaccaatcttaaacataataaatcacacattcagttttttctggt

SEQ ID NO: 57 - DNA, 3120 bp, Homo sapiens
aatagactaaacccagagcctcaaagcagtgcactccgtgaaggcaaagagaacacgctgcaaaaggctttccaagaatcctcgacatggcaaggaggagctcgttccagtcgtgtcagataatatccctgttcacttttgccgttggagtcaatatctgcttaggattcactgcacatcgaattaagagagcagaaggatgggaggaaggtcctcctacagtgctatcagactccccctggaccaacatctccggatcttgcaagggcaggtgctttgaacttcaagaggctggacctcctgattgtcgctgtgacaacttgtgtaagagctataccagttgctgccatgactttgatgagctgtgtttgaagacagcccgtggctgggagtgtactaaggacagatgtggagaagtcagaaatgaagaaaatgcctgtcactgctcagaggactgcttggccaggggagactgctgtaccaattaccaagtggtttgcaaaggagagtcgcattgggttgatgatgactgtgaggaaataaaggccgcagaatgccctgcagggtttgttcgccctccattaatcatcttctccgtggatggcttccgtgcatcatacatgaagaaaggcagcaaagtcatgcctaatattgaaaaactaaggtcttgtggcacacactctccctacatgaggccggtgtacccaactaaaacctttcctaacttatacactttggccactgggctatatccagaatcacatggaattgttggcaattcaatgtatgatcctgtatttgatgccacttttcatctgcgagggcgagagaaatttaatcatagatggtggggaggtcaaccgctatggattacagccaccaagcaaggggtgaaagctggaacattcttttggtctgttgtcatccctcacgagcggagaatattaaccatattgcagtggctcaccctgccagatcatgagaggccttcggtctatgccttctattctgagcaacctgatttctctggacacaaatatggccctttcggccctgagatgacaaatcctctgagggaaatcgacaaaattgtggggcaattaatggatggactgaaacaactaaaactgcatcggtgtgtcaacgtcatctttgtcggagaccatggaatggaagatgtcacatgtgatagaactgagttcttgagtaattacctaactaatgtggatgatattactttagtgcctggaactctaggaagaattcgatccaaatttagcaacaatgctaaatatgaccccaaagccattattgccaatctcacgtgtaaaaaaccagatcagcactttaagccttacttgaaacagcaccttcccaaacgtttgcactatgccaacaacagaagaattgaggatatccatttattggtggaacgcagatggcatgttgcaaggaaacctttggatgtttataagaaaccatcaggaaaatgctttttccagggagaccacggatttgataacaaggtcaacagcatgcagactgtttttgtaggttatggctcaacatttaagtacaagactaaagtgcctccatttgaaaacattgaactttacaatgttatgtgtgatctcctgggattgaagccagctcctaataatgggacccatggaagtttgaatcatctcctgcgcactaataccttcaggccaaccatgccagaggaagttaccagacccaattatccagggattatgtaccttcagtctgattttgacctgggctgcacttgtgatgataaggtagagccaaagaacaagttggatgaactcaacaaacggcttcatacaaaagggtctacagaagagagacacctcctctatgggcgacctgcagtgctttatcggactagatatgatatcttatatcacactgactttgaaagtggttatagtgaaatattcctaatgccactctggacatcatatactgtttccaaacaggctgaggtttccagcgttcctgaccatctgaccagttgcgtccggcctgatgtccgtgtttctccgagtttcagtcagaactgtttggcctacaaaaatgataagcagatgtcctacggattcctctttcctccttatctgagctcttcaccagaggctaaatatgatgcattccttgtaaccaatatggttccaatgtatcctgctttcaaacgggtctggaattatttccaaagggtattggtgaagaaatatgcttcggaaagaaatggagttaacgtgataagtggaccaatcttcgactatgactatgatggcttacatgacacagaagacaaaataaaacagtacgtggaaggcagttccattcctgttccaactcactactacagcatcatcaccagctgtctggatttcactcagcctgccgacaagtgtgacggccctctctctgtgtcctccttcatcctgcctcaccggcctgacaacgaggagagctgcaatagctcagaggacgaatcaaaatgggtagaagaactcatgaagatgcacacagctagggtgcgtgacattgaacatctcaccagcctggacttcttccgaaagaccagccgcagctacccagaaatcctgacactcaagacatacctgcatacatatgagagcgagatttaactttctgagcatctgcagtacagtcttatcaactggttgtatatttttatattgtttttgtatttattaatttgaaaccaggacattaaaaatgttagtattttaatcctgtaccaaatctgacatattatgcctgaatgactccactgtttttctctaatgcttgatttaggtagccttgtgttctgagtagagcttgtaataaatactgcagcttgagtttttagtggaagcttctaaatggtgctgcagatttgatatttgcattgaggaaatattaattttccaatgcacagttgccacatttagtcctgtactgtatggaaacactgattttgtaaagttgcctttatttgctgttaactgttaactatgacagatatatttaagccttataaaccaatcttaaacataataaatcacacattcagttttttctggt

SEQ ID NO: 58 - DNA, 3212 bp, Homo sapiens
aatagactaaacccagagcctcaaagcagtgcactccgtgaaggcaaagagaacacgctgcaaaaggctttccaagaatcctcgacatggcaaggaggagctcgttccagtcgtgtcagataatatccctgttcacttttgccgttggagtcaatatctgcttaggattcactgcacatcgaattaagagagcagaaggatgggaggaaggtcctcctacagtgctatcagactccccctggaccaacatctccggatcttgcaagggcaggtgctttgaacttcaagaggctggacctcctgattgtcgctgtgacaacttgtgtaagagctataccagttgctgccatgactttgatgagctgtgtttgaagacagcccgtggctgggagtgtactaaggacagatgtggagaagtcagaaatgaagaaaatgcctgtcactgctcagaggactgcttggccaggggagactgctgtaccaattaccaagtggtttgcaaaggagagtcgcattgggttgatgatgactgtgaggaaataaaggccgcagaatgccctgcagggtttgttcgccctccattaatcatcttctccgtggatggcttccgtgcatcatacatgaagaaaggcagcaaagtcatgcctaatattgaaaaactaaggtcttgtggcacacactctccctacatgaggccggtgtacccaactaaaacctttcctaacttatacactttggccactgggctatatccagaatcacatggaattgttggcaattcaatgtatgatcctgtatttgatgccacttttcatctgcgagggcgagagaaatttaatcatagatggtggggaggtcaaccgctatggattacagccaccaagcaaggggtgaaagctggaacattcttttggtctgttgtcatccctcacgagcggagaatattaaccatattgcagtggctcaccctgccagatcatgagaggccttcggtctatgccttctattctgagcaacctgatttctctggacacaaatatggccctttcggccctgagatgacaaatcctctgagggaaatcgacaaaattgtggggcaattaatggatggactgaaacaactaaaactgcatcggtgtgtcaacgtcatctttgtcggagaccatggaatggaagatgtcacatgtgatagaactgagttcttgagtaattacctaactaatgtggatgatattactttagtgcctggaactctaggaagaattcgatccaaatttagcaacaatgctaaatatgaccccaaagccattattgccaatctcacgtgtaaaaaaccagatcagcactttaagccttacttgaaacagcaccttcccaaacgtttgcactatgccaacaacagaagaattgaggatatccatttattggtggaacgcagatggcatgttgcaaggaaacctttggatgtttataagaaaccatcaggaaaatgctttttccagggagaccacggatttgataacaaggtcaacagcatgcagactgtttttgtaggttatggctcaacatttaagtacaagactaaagtgcctccatttgaaaacattgaactttacaatgttatgtgtgatctcctgggattgaagccagctcctaataatgggacccatggaagtttgaatcatctcctgcgcactaataccttcaggccaaccatgccagaggaagttaccagacccaattatccagggattatgtaccttcagtctgattttgacctgggctgcacttgtgatgataaggtagagccaaagaacaagttggatgaactcaacaaacggcttcatacaaaagggtctacagaagctgaaaccaggaaattcagaggcagcagaaatgaaaacaaggaaaacattaatggaaattttgaacctagaaaagagagacacctcctctatgggcgacctgcagtgctttatcggactagatatgatatcttatatcacactgactttgaaagtggttatagtgaaatattcctaatgccactctggacatcatatactgtttccaaacaggctgaggtttccagcgttcctgaccatctgaccagttgcgtccggcctgatgtccgtgtttctccgagtttcagtcagaactgtttggcctacaaaaatgataagcagatgtcctacggattcctctttcctccttatctgagctcttcaccagaggctaaatatgatgcattccttgtaaccaatatggttccaatgtatcctgctttcaaacgggtctggaattatttccaaagggtattggtgaagaaatatgcttcggaaagaaatggagttaacgtgataagtggaccaatcttcgactatgactatgatggcttacatgacacagaagacaaaataaaacagtacgtggaaggcagttccattcctgttccaactcactactacagcatcatcaccagctgtctggatttcactcagcctgccgacaagtgtgacggccctctctctgtgtcctccttcatcctgcctcaccggcctgacaacgaggagagctgcaatagctcagaggacgaatcaaaatgggtagaagaactcatgaagatgcacacagctagggtgcgtgacattgaacatctcaccagcctggacttcttccgaaagaccagccgcagctacccagaaatcctgacactcaagacatacctgcatacatatgagagcgagatttaactttctgagcatctgcagtacagtcttatcaactggttgtatatttttatattgtttttgtatttattaatttgaaaccaggacattaaaaatgttagtattttaatcctgtaccaaatctgacatattatgcctgaatgactccactgtttttctctaatgcttgatttaggtagccttgtgttctgagtagagcttgtaataaatactgcagcttgagtttttagtggaagcttctaaatggtgctgcagatttgatatttgcattgaggaaatattaattttccaatgcacagttgccacatttagtcctgtactgtatggaaacactgattttgtaaagttgcctttatttgctgttaactgttaactatgacagatatatttaagccttataaaccaatcttaaacataataaatcacacattcagttttttctggaaaaaaaaaaaaaaaaaa

SEQ ID NO: 59 - protein, 915 aa, Homo sapiens
MARRSSFQSCQIISLFTFAVGVNICLGFTAHRIKRAEGWEEGPPTVLSDSPWTNISGSCKGRCFELQEAGPPDCRCDNLCKSYTSCCHDFDELCLKTARGWECTKDRCGEVRNEENACHCSEDCLARGDCCTNYQVVCKGESHWVDDDCEEIKAAECPAGFVRPPLIIFSVDGFRASYMKKGSKVMPNIEKLRSCGTHSPYMRPVYPTKTFPNLYTLATGLYPESHGIVGNSMYDPVFDATFHLRGREKFNHRWWGGQPLWITATKQGVKAGTFFWSVVIPHERRILTILQWLTLPDHERPSVYAFYSEQPDFSGHKYGPFGPEESSYGSPFTPAKRPKRKVAPKRRQERPVAPPKKRRRKIHRMDHYAAETRQDKMTNPLREIDKIVGQLMDGLKQLKLHRCVNVIFVGDHGMEDVTCDRTEFLSNYLTNVDDITLVPGTLGRIRSKFSNNAKYDPKAIIANLTCKKPDQHFKPYLKQHLPKRLHYANNRRIEDIHLLVERRWHVARKPLDVYKKPSGKCFFQGDHGFDNKVNSMQTVFVGYGSTFKYKTKVPPFENIELYNVMCDLLGLKPAPNNGTHGSLNHLLRTNTFRPTMPEEVTRPNYPGIMYLQSDFDLGCTCDDKVEPKNKLDELNKRLHTKGSTEERHLLYGRPAVLYRTRYDILYHTDFESGYSEIFLMPLWTSYTVSKQAEVSSVPDHLTSCVRPDVRVSPSFSQNCLAYKNDKQMSYGFLFPPYLSSSPEAKYDAFLVTNMVPMYPAFKRVWNYFQRVLVKKYASERNGVNVISGPIFDYDYDGLHDTEDKIKQYVEGSSIPVPTHYYSIITSCLDFTQPADKCDGPLSVSSFILPHRPDNEESCNSSEDESKWVEELMKMHTARVRDIEHLTSLDFFRKTSRSYPEILTLKTYLHTYESEI

SEQ ID NO: 60 - protein, 863 aa, Homo sapiens
MARRSSFQSCQIISLFTFAVGVNICLGFTAHRIKRAEGWEEGPPTVLSDSPWTNISGSCKGRCFELQEAGPPDCRCDNLCKSYTSCCHDFDELCLKTARGWECTKDRCGEVRNEENACHCSEDCLARGDCCTNYQVVCKGESHWVDDDCEEIKAAECPAGFVRPPLIIFSVDGFRASYMKKGSKVMPNIEKLRSCGTHSPYMRPVYPTKTFPNLYTLATGLYPESHGIVGNSMYDPVFDATFHLRGREKFNHRWWGGQPLWITATKQGVKAGTFFWSVVIPHERRILTILQWLTLPDHERPSVYAFYSEQPDFSGHKYGPFGPEMTNPLREIDKIVGQLMDGLKQLKLHRCVNVIFVGDHGMEDVTCDRTEFLSNYLTNVDDITLVPGTLGRIRSKFSNNAKYDPKAIIANLTCKKPDQHFKPYLKQHLPKRLHYANNRRIEDIHLLVERRWHVARKPLDVYKKPSGKCFFQGDHGFDNKVNSMQTVFVGYGSTFKYKTKVPPFENIELYNVMCDLLGLKPAPNNGTHGSLNHLLRTNTFRPTMPEEVTRPNYPGIMYLQSDFDLGCTCDDKVEPKNKLDELNKRLHTKGSTEERHLLYGRPAVLYRTRYDILYHTDFESGYSEIFLMPLWTSYTVSKQAEVSSVPDHLTSCVRPDVRVSPSFSQNCLAYKNDKQMSYGFLFPPYLSSSPEAKYDAFLVTNMVPMYPAFKRVWNYFQRVLVKKYASERNGVNVISGPIFDYDYDGLHDTEDKIKQYVEGSSIPVPTHYYSIITSCLDFTQPADKCDGPLSVSSFILPHRPDNEESCNSSEDESKWVEELMKMHTARVRDIEHLTSLDFFRKTSRSYPEILTLKTYLHTYESEI

SEQ ID NO: 61 - protein, 888 aa, Homo sapiens
MARRSSFQSCQIISLFTFAVGVNICLGFTAHRIKRAEGWEEGPPTVLSDSPWTNISGSCKGRCFELQEAGPPDCRCDNLCKSYTSCCHDFDELCLKTARGWECTKDRCGEVRNEENACHCSEDCLARGDCCTNYQVVCKGESHWVDDDCEEIKAAECPAGFVRPPLIIFSVDGFRASYMKKGSKVMPNIEKLRSCGTHSPYMRPVYPTKTFPNLYTLATGLYPESHGIVGNSMYDPVFDATFHLRGREKFNHRWWGGQPLWITATKQGVKAGTFFWSVVIPHERRILTILQWLTLPDHERPSVYAFYSEQPDFSGHKYGPFGPEMTNPLREIDKIVGQLMDGLKQLKLHRCVNVIFVGDHGMEDVTCDRTEFLSNYLTNVDDITLVPGTLGRIRSKFSNNAKYDPKAIIANLTCKKPDQHFKPYLKQHLPKRLHYANNRRIEDIHLLVERRWHVARKPLDVYKKPSGKCFFQGDHGFDNKVNSMQTVFVGYGSTFKYKTKVPPFENIELYNVMCDLLGLKPAPNNGTHGSLNHLLRTNTFRPTMPEEVTRPNYPGIMYLQSDFDLGCTCDDKVEPKNKLDELNKRLHTKGSTEAETRKFRGSRNENKENINGNFEPRKERHLLYGRPAVLYRTRYDILYHTDFESGYSEIFLMPLWTSYTVSKQAEVSSVPDHLTSCVRPDVRVSPSFSQNCLAYKNDKQMSYGFLFPPYLSSSPEAKYDAFLVTNMVPMYPAFKRVWNYFQRVLVKKYASERNGVNVISGPIFDYDYDGLHDTEDKIKQYVEGSSIPVPTHYYSIITSCLDFTQPADKCDGPLSVSSFILPHRPDNEESCNSSEDESKWVEELMKMHTARVRDIEHLTSLDFFRKTSRSYPEILTLKTYLHTYESEI

SEQ ID NO: 62 - DNA, 2329 bp, Homo sapiens
caaaagggaactttatatggaaaagcttcaagaacatttaatcaaagcaaaagcctttaccataaagaagacgctggagatctatgtgcccatcaaacagttcttttacaacctcatccacccggagtatagcgccgtgactgacgtgtatgtactcatgttcctggctgacactgtggacttcatcatcattgtcttcggcttttgggcctttgggaaacactcagcagctgcagacatcacctcttcactgtcagaggaccaggtcccggggccgtttttggtgatggtcctcattcagtttggaaccatggtggtggaccgagccctctacctcaggaagactgtactgggaaaggtcatcttccaggtcattcttgtgttcggaattcacttctggatgttcttcatcttacctggtgtgactgagaggaaattcagccagaacctggttgcccagctttggtactttgtgaaatgtgtttacttcgggttgtctgcttaccagatccgttgtggctacccaacgcgagtcctggggaacttcctcaccaagagctacaattacgtcaacctcttcttattccaagggtttcgcctcgtgccctttttgactgagctgagggcagtgatggactgggtgtggacggacacaactttgagcctgtccagctggatctgtgtggaggacatctatgctcacatattcatcctgaagtgttggcgggagtcggagaagcctattttcacaatgagtgcccaacaaagccagttgaaagttatggaccagcagagctttaacaaatttatacaagctttttctagggacaccggtgctatgcaatttctggaaaattatgaaaaagaagacataacagtagcagaactggaaggaaactcaaattctttgtggaccatcagcccacccagtaagcagaaaatgatacacgaactcctggaccccaatagtagcttctctgttgttttttcatggagtattcagagaaacttaagtctgggtgcaaaatcggaaatagcaacagataagctttcttttcctcttaaaaatattactcgaaagaatatcgctaaaatgatagcaggcaacagcacagaaagttcaaaaacaccagtgaccatagaaaagatttatccatattatgtgaaagcacctagtgattctaactcaaaacctataaagcaacttttatctgaaaataatttcatggatattaccatcattttgtccagagacaatacaactaaatataacagtgagtggtgggttctcaacctgactggaaacagaatatacaatccgaactctcaggccctggaactggtggtcttcaatgacaaagtcagtcccccaagtctggggttcctggctggctatggtattatgggattatatgcttcagttgtccttgtgattgggaaatttgtccgtgaattcttcagtgggatttctcactccatcatgtttgaagagcttccaaatgtggatcgaattttgaagttgtgcacagatatttttttagttcgagagacaggagaactggagctagaagaagatctctatgccaaattaatattcctatatcgctcaccagagacaatgatcaaatggactagagaaaaaacaaattgaaaccttagaacacagactgcaaataatgttaacatttgaattttttttaaaagcacaatattctcataagagctaagcatttctagttcgacggaaatggtttgtttctcttctgataggtagacaaaaggagctgatatccttctgcagtaaaagctacctggcaagttaaggcactgttgaaaatgttatttgtaactccatttctctgaaatcagggctacttgctttatgttttagtcaacagtgtctcgcattctgattgatcatgtgaaggaatcatttatgggccccgtccctaagagaaacagaagaggagtcagaaagaaagatgcctgtgttttcctctgtggggcccgtgcacttcctggagagatgctacaatgcaatatacagcgctccatccccactggggaagctgctgtgatgagactagatgagccttcaacacactcagaaaatgcaacagcaatagggggcagacagctcctacctgtgtttctaggagcaaaagagagggaactaattgcccgtgaagacgccagtggaaggatcagcctcattctaagcaaaaacatagtattagtgatactcttactgccttatcttaaccaaggactaataggatacctttccattaaacaccagtgacttctcaggaaaaaaaaaaaaaaaaaa

SEQ ID NO: 63 - protein, 544 aa, Homo sapiens
MEKLQEHLIKAKAFTIKKTLEIYVPIKQFFYNLIHPEYSAVTDVYVLMFLADTVDFIIIVFGFWAFGKHSAAADITSSLSEDQVPGPFLVMVLIQFGTMVVDRALYLRKTVLGKVIFQVILVFGIHFWMFFILPGVTERKFSQNLVAQLWYFVKCVYFGLSAYQIRCGYPTRVLGNFLTKSYNYVNLFLFQGFRLVPFLTELRAVMDWVWTDTTLSLSSWICVEDIYAHIFILKCWRESEKPIFTMSAQQSQLKVMDQQSFNKFIQAFSRDTGAMQFLENYEKEDITVAELEGNSNSLWTISPPSKQKMIHELLDPNSSFSVVFSWSIQRNLSLGAKSEIATDKLSFPLKNITRKNIAKMIAGNSTESSKTPVTIEKIYPYYVKAPSDSNSKPIKQLLSENNFMDITIILSRDNTTKYNSEWWVLNLTGNRIYNPNSQALELVVFNDKVSPPSLGFLAGYGIMGLYASVVLVIGKFVREFFSGISHSIMFEELPNVDRILKLCTDIFLVRETGELELEEDLYAKLIFLYRSPETMIKWTREKTN

SEQ ID NO: 64 - DNA, 1866 bp, Homo sapiens
gcattgctacctgcccttctcaaagcccacatgttgtattttatgcaaatctacagattatctatcattatctaaatgcaggcatctgaaaaccagcagtaatcctgcctctgaagtttatcaggaaaggagcttaaaagagaaccaaattcagcctgtgttggaactctcagtcccagaggggtgtggtttgtagctctccggcctgctgttggacttaggctgtgacccacagaaggacgccagaaagtactcaagacattcacggtgccccggtcagcactcgccatgacgaagacttctacatgcatataccacttccttgttctgagctggtatactttcctcaattattacatctcacaggaaggaaaagacgaggtgaaacccaaaatcttggcaaatggtgcaaggtggaaatatatgacgctgcttaatctgctcaagaacaggactgctgggtttgacatctaccagccaggaagctttaggcagctcttgcagaccattttctacggggtcacctgcctggatgatgtgctgaaaagaaccaaagggggaaaagacattaagttcctaactgccttcagagacctgcttttcaccactctggcttttcctgtatccacgtttgtatttttggcattctggatcctctttctctacaatcgagatctcatttaccccaaggtcctagatactgtcatccccgtgtggctgaatcatgcaatgcacactttcatattccccatcacattggctgaagtcgtcctcaggcctcactcctatccatcaaagaagacaggactcaccttgctggctgctgccagcattgcttacatcagccgcatcctatggctctactttgagacgggtacctgggtgtatcctgtgtttgccaaactcagcctcttgggtctagcagctttcttctctctcagctacgtcttcatcgccagcatctacctacttggagagaagctcaaccactggaaatggggtgacatgaggcagccacggaagaagaggaagtaattgcacaccattttccaagaaccaagaaagaagaaaacacaagagatttttctcatcttttttttttttttctggtggagggaggtggtggaggaacatagcaaagtaggagggacagagagtgatacttaaatttaataagaggttcgtgaaggtagcttaacttgagaactcttggttttttgaaaggttgactgcacatgccaaaaacaactcctgctatctcagaattaattatctttgaccttcgtggaggatggtctctggttaaaatctggccaaagaaactcacataaacttggtgttagagaacatctagagagagagagaggaacttagagtcatttaaactcttcagtttacagagaaggatgctgaggacctagatgagaagttacctgcaaaaggcaaaagggttacttagtgtcagaaccaaggcaatgacttctctctcccagatctcctagctactggtcctgggccatttttttttttttaaataatcccaactttctttaaaagacaagcatttcagtaagctagttattttcatgggttgctcatccatttttttcagtgatctaaaaatgtagggagatggctactactgaagttgtctgtctacttgggataatagcaaattaattgaagacaatgggaaagtaagttataaaaaatactgggaaatctgtttctcttctgagcaagcattcagggcaggtataaacatcaaacatagtgacattgtcaaaacctcttccatttgaacattgattaattcatcaaataaatggtatagtaataaattttgcttgcagaagaaaaaaaaaa

SEQ ID NO: 65 - DNA, 1812 bp, Homo sapiens
gcattgctacctgcccttctcaaagcccacatgttgtattttatgcaaatctacagattatctatcattatctaaatgcaggcatctgaaaaccagcagtaatcctgcctctgaagtttatcaggaaaggagcttaaaagagaaccaaattcagcctgtgttggaactctcagtcccagaggggtgtggtttgtagctctccggcctgctgttggacttaggctgtgacccacagaaggacgccagaaagtactcaagacattcacggtgccccggtcagcactcgccatgacgaagacttctacatgcatataccacttccttgttctgagctggtatactttcctcaattattacatctcacaggaaggaaaagacgaggtgaaacccaaaatcttggcaaatggtgcaaggtggaaatatatgacgctgcttaatctgctcttgcagaccattttctacggggtcacctgcctggatgatgtgctgaaaagaaccaaagggggaaaagacattaagttcctaactgccttcagagacctgcttttcaccactctggcttttcctgtatccacgtttgtatttttggcattctggatcctctttctctacaatcgagatctcatttaccccaaggtcctagatactgtcatccccgtgtggctgaatcatgcaatgcacactttcatattccccatcacattggctgaagtcgtcctcaggcctcactcctatccatcaaagaagacaggactcaccttgctggctgctgccagcattgcttacatcagccgcatcctatggctctactttgagacgggtacctgggtgtatcctgtgtttgccaaactcagcctcttgggtctagcagctttcttctctctcagctacgtcttcatcgccagcatctacctacttggagagaagctcaaccactggaaatggggtgacatgaggcagccacggaagaagaggaagtaattgcacaccattttccaagaaccaagaaagaagaaaacacaagagatttttctcatcttttttttttttttctggtggagggaggtggtggaggaacatagcaaagtaggagggacagagagtgatacttaaatttaataagaggttcgtgaaggtagcttaacttgagaactcttggttttttgaaaggttgactgcacatgccaaaaacaactcctgctatctcagaattaattatctttgaccttcgtggaggatggtctctggttaaaatctggccaaagaaactcacataaacttggtgttagagaacatctagagagagagagaggaacttagagtcatttaaactcttcagtttacagagaaggatgctgaggacctagatgagaagttacctgcaaaaggcaaaagggttacttagtgtcagaaccaaggcaatgacttctctctcccagatctcctagctactggtcctgggccatttttttttttttaaataatcccaactttctttaaaagacaagcatttcagtaagctagttattttcatgggttgctcatccatttttttcagtgatctaaaaatgtagggagatggctactactgaagttgtctgtctacttgggataatagcaaattaattgaagacaatgggaaagtaagttataaaaaatactgggaaatctgtttctcttctgagcaagcattcagggcaggtataaacatcaaacatagtgacattgtcaaaacctcttccatttgaacattgattaattcatcaaataaatggtatagtaataaattttgcttgcagaagaaaaaaaaaa

SEQ ID NO: 66 - protein, 248 aa, Homo sapiens
MTKTSTCIYHFLVLSWYTFLNYYISQEGKDEVKPKILANGARWKYMTLLNLLKNRTAGFDIYQPGSFRQLLQTIFYGVTCLDDVLKRTKGGKDIKFLTAFRDLLFTTLAFPVSTFVFLAFWILFLYNRDLIYPKVLDTVIPVWLNHAMHTFIFPITLAEVVLRPHSYPSKKTGLTLLAAASIAYISRILWLYFETGTWVYPVFAKLSLLGLAAFFSLSYVFIASIYLLGEKLNHWKWGDMRQPRKKRK

SEQ ID NO: 67 - protein, 230 aa, Homo sapiens
MTKTSTCIYHFLVLSWYTFLNYYISQEGKDEVKPKILANGARWKYMTLLNLLLQTIFYGVTCLDDVLKRTKGGKDIKFLTAFRDLLFTTLAFPVSTFVFLAFWILFLYNRDLIYPKVLDTVIPVWLNHAMHTFIFPITLAEVVLRPHSYPSKKTGLTLLAAASIAYISRILWLYFETGTWVYPVFAKLSLLGLAAFFSLSYVFIASIYLLGEKLNHWKWGDMRQPRKKRK

SEQ ID NO: 68 - DNA, 5623 bp, Homo sapiens
cttgttgactaggcgctgttcttgctggctggtgccccagggcctggagaggtctgaagaaacctgggagccagcagcccggggctccactctgggttctgaaagcccattccctgctctgcggctcctcccaccccacctcttctcagccttgcagctcaagggttgatctcaggagtccaggacccaggagagggaagaatctgaggaacacagaacagtgagcgttgcccacaccccatctcccgtcaccacatctcccctcaccctcaccctccctgcctggccctggaccccatcccaggacctccctatcagctgacttcttccagtgtcttgcaggcccctctgggctcctccctcccctggcttttcctaccactccccctctatcggcgtctatctgtaggtgccctgggatttataaaactgggttccgaatgctgaataagagacggtaagagccaaggcaaaggacagcactgttctctgcctgcctgataccctcaccacctgggaacatcccccagacaccctcttaactccgggacagagatggctggcggagcctggggccgcctggcctgttacttggagttcctgaagaaggaggagctgaaggagttccagcttctgctcgccaataaagcgcactccaggagctcttcgggtgagacacccgctcagccagagaagacgagtggcatggaggtggcctcgtacctggtggctcagtatggggagcagcgggcctgggacctagccctccatacctgggagcagatggggctgaggtcactgtgcgcccaagcccaggaaggggcaggccactctccctcattcccctacagcccaagtgaaccccacctggggtctcccagccaacccacctccaccgcagtgctaatgccctggatccatgaattgccggcggggtgcacccagggctcagagagaagggttttgagacagctgcctgacacatctggacgccgctggagagaaatctctgcctcactcctctaccaagctcttccaagctccccagaccatgagtctccaagccaggagtcacccaacgcccccacatccacagcagtgctggggagctggggatccccacctcagcccagcctagcacccagagagcaggaggctcctgggacccaatggcctctggatgaaacgtcaggaatttactacacagaaatcagagaaagagagagagagaaatcagagaaaggcaggcccccatgggcagcggtggtaggaacgcccccacaggcgcacaccagcctacagccccaccaccacccatgggagccttctgtgagagagagcctctgttccacatggccctggaaaaatgaggattttaaccaaaaattcacacagctgctacttctacaaagacctcaccccagaagccaagatcccctggtcaagagaagctggcctgattatgtggaggagaatcgaggacatttaattgagatcagagacttatttggcccaggcctggatacccaagaacctcgcatagtcatactgcagggggctgctggaattgggaagtcaacactggccaggcaggtgaaggaagcctgggggagaggccagctgtatggggaccgcttccagcatgtcttctacttcagctgcagagagctggcccagtccaaggtggtgagtctcgctgagctcatcggaaaagatgggacagccactccggctcccattagacagatcctgtctaggccagagcggctgctcttcatcctcgatggtgtagatgagccaggatgggtcttgcaggagccgagttctgagctctgtctgcactggagccagccacagccggcggatgcactgctgggcagtttgctggggaaaactatacttcccgaggcatccttcctgatcacggctcggaccacagctctgcagaacctcattccttctttggagcaggcacgttgggtagaggtcctggggttctctgagtccagcaggaaggaatatttctacagatatttcacagatgaaaggcaagcaattagagcctttaggttggtcaaatcaaacaaagagctctgggccctgtgtcttgtgccctgggtgtcctggctggcctgcacttgcctgatgcagcagatgaagcggaaggaaaaactcacactgacttccaagaccaccacaaccctctgtctacattaccttgcccaggctctccaagctcagccattgggaccccagctcagagacctctgctctctggctgctgagggcatctggcaaaaaaagacccttttcagtccagatgacctcaggaagcatgggttagatggggccatcatctccaccttcttgaagatgggtattcttcaagagcaccccatccctctgagctacagcttcattcacctctgtttccaagagttctttgcagcaatgtcctatgtcttggaggatgagaaggggagaggtaaacattctaattgcatcatagatttggaaaagacgctagaagcatatggaatacatggcctgtttggggcatcaaccacacgtttcctattgggcctgttaagtgatgagggggagagagagatggagaacatctttcactgccggctgtctcaggggaggaacctgatgcagtgggtcccgtccctgcagctgctgctgcagccacactctctggagtccctccactgcttgtacgagactcggaacaaaacgttcctgacacaagtgatggcccatttcgaagaaatgggcatgtgtgtagaaacagacatggagctcttagtgtgcactttctgcattaaattcagccgccacgtgaagaagcttcagctgattgagggcaggcagcacagatcaacatggagccccaccatggtagtcctgttcaggtgggtcccagtcacagatgcctattggcagattctcttctccgtcctcaaggtcaccagaaacctgaaggagctggacctaagtggaaactcgctgagccactctgcagtgaagagtctttgtaagaccctgagacgccctcgctgcctcctggagaccctgcggttggctggctgtggcctcacagctgaggactgcaaggaccttgcctttgggctgagagccaaccagaccctgaccgagctggacctgagcttcaatgtgctcacggatgctggagccaaacacctttgccagagactgagacagccgagctgcaagctacagcgactgcagctggtcagctgtggcctcacgtctgactgctgccaggacctggcctctgtgcttagtgccagccccagcctgaaggagctagacctgcagcagaacaacctggatgacgttggcgtgcgactgctctgtgaggggctcaggcatcctgcctgcaaactcatacgcctggggctggaccagacaactctgagtgatgagatgaggcaggaactgagggccctggagcaggagaaacctcagctgctcatcttcagcagacggaaaccaagtgtgatgacccctactgagggcctggatacgggagagatgagtaatagcacatcctcactcaagcggcagagactcggatcagagagggcggcttcccatgttgctcaggctaatctcaaactcctggacgtgagcaagatcttcccaattgctgagattgcagaggaaagctccccagaggtagtaccggtggaactcttgtgcgtgccttctcctgcctctcaaggggacctgcatacgaagcctttggggactgacgatgacttctggggccccacggggcctgtggctactgaggtagttgacaaagaaaagaacttgtaccgagttcacttccctgtagctggctcctaccgctggcccaacacgggtctctgctttgtgatgagagaagcggtgaccgttgagattgaattctgtgtgtgggaccagttcctgggtgagatcaacccacagcacagctggatggtggcagggcctctgctggacatcaaggctgagcctggagctgtggaagctgtgcacctccctcactttgtggctctccaagggggccatgtggacacatccctgttccaaatggcccactttaaagaggaggggatgctcctggagaagccagccagggtggagctgcatcacatagttctggaaaaccccagcttctcccccttgggagtcctcctgaaaatgatccataatgccctgcgcttcattcccgtcacctctgtggtgttgctttaccaccgcgtccatcctgaggaagtcaccttccacctctacctgatcccaagtgactgctccattcggaaggccatagatgatctagaaatgaaattccagtttgtgcgaatccacaagccacccccgctgaccccactttatatgggctgtcgttacactgtgtctgggtctggttcagggatgctggaaatactccccaaggaactggagctctgctatcgaagccctggagaagaccagctgttctcggagttctacgttggccacttgggatcagggatcaggctgcaagtgaaagacaagaaagatgagactctggtgtgggaggccttggtgaaaccaggagatctcatgcctgcaactactctgatccctccagcccgcatagccgtaccttcacctctggatgccccgcagttgctgcactttgtggaccagtatcgagagcagctgatagcccgagtgacatcggtggaggttgtcttggacaaactgcatggacaggtgctgagccaggagcagtacgagagggtgctggctgagaacacgaggcccagccagatgcggaagctgttcagcttgagccagtcctgggaccggaagtgcaaagatggactctaccaagccctgaaggagacccatcctcacctcattatggaactctgggagaagggcagcaaaaagggactcctgccactcagcagctgaagtatcaacaccagcccttgacccttgagtcctggctttggctgacccttctttgggtctcagtttctttctctgcaaacaagttgccatctggtttgccttccagcactaaagtaatggaactttgatgatgcctttgctgggcattatgtgtccatgccagggatgccacagggggccccagtccaggtggcctaacagcatctcagggaatgtccatctggagctggcaagacccctgcagacctcatagagcctcatctggtggccacagcagccaagcctagagccctccggatcccatccaggcgcaaagaggaataggagggacatggaaccatttgcctctggctgtgtcacagggtgagccccaaaattggggttcagcgtgggaggccacgtggattcttggctttgtacaggaagatctacaagagcaagccaacagagtaaagtggaaggaagtttattcagaaaataaaggagtatcacagctcttttagaatttgtctagcaggctttccagtttttaccagaaaacccctataaattaaaaattttttacttaaatttaagaattaaaaaaatacaaaaaagaaaaaatgaaaataaaggaataagaagttacctactccaaaaaaaaaaa

SEQ ID NO: 69 - DNA, 5491 bp, Homo sapiens
cttgttgactaggcgctgttcttgctggctggtgccccagggcctggagaggtctgaagaaacctgggagccagcagcccggggctccactctgggttctgaaagcccattccctgctctgcggctcctcccaccccacctcttctcagccttgcagctcaagggttgatctcaggagtccaggacccaggagagggaagaatctgaggaacacagaacagtgagcgttgcccacaccccatctcccgtcaccacatctcccctcaccctcaccctccctgcctggccctggaccccatcccaggacctccctatcagctgacttcttccagtgtcttgcaggcccctctgggctcctccctcccctggcttttcctaccactccccctctatcggcgtctatctgtaggtgccctgggatttataaaactgggttccgaatgctgaataagagacggtaagagccaaggcaaaggacagcactgttctctgcctgcctgataccctcaccacctgggaacatcccccagacaccctcttaactccgggacagagatggctggcggagcctggggccgcctggcctgttacttggagttcctgaagaaggaggagctgaaggagttccagcttctgctcgccaataaagcgcactccaggagctcttcgggtgagacacccgctcagccagagaagacgagtggcatggaggtggcctcgtacctggtggctcagtatggggagcagcgggcctgggacctagccctccatacctgggagcagatggggctgaggtcactgtgcgcccaagcccaggaaggggcaggccactctccctcattcccctacagcccaagtgaaccccacctggggtctcccagccaacccacctccaccgcagtgctaatgccctggatccatgaattgccggcggggtgcacccagggctcagagagaagggttttgagacagctgcctgacacatctggacgccgctggagagaaatctctgcctcactcctctaccaagctcttccaagctccccagaccatgagtctccaagccaggagtcacccaacgcccccacatccacagcagtgctggggagctggggatccccacctcagcccagcctagcacccagagagcaggaggctcctgggacccaatggcctctggatgaaacgtcaggaatttactacacagaaatcagagaaagagagagagagaaatcagagaaaggcaggcccccatgggcagcggtggtaggaacgcccccacaggcgcacaccagcctacagccccaccaccacccatgggagccttctgtgagagagagcctctgttccacatggccctggaaaaatgaggattttaaccaaaaattcacacagctgctacttctacaaagacctcaccccagaagccaagatcccctggtcaagagaagctggcctgattatgtggaggagaatcgaggacatttaattgagatcagagacttatttggcccaggcctggatacccaagaacctcgcatagtcatactgcagggggctgctggaattgggaagtcaacactggccaggcaggtgaaggaagcctgggggagaggccagctgtatggggaccgcttccagcatgtcttctacttcagctgcagagagctggcccagtccaaggtggtgagtctcgctgagctcatcggaaaagatgggacagccactccggctcccattagacagatcctgtctaggccagagcggctgctcttcatcctcgatggtgtagatgagccaggatgggtcttgcaggagccgagttctgagctctgtctgcactggagccagccacagccggcggatgcactgctgggcagtttgctggggaaaactatacttcccgaggcatccttcctgatcacggctcggaccacagctctgcagaacctcattccttctttggagcaggcacgttgggtagaggtcctggggttctctgagtccagcaggaaggaatatttctacagatatttcacagatgaaaggcaagcaattagagcctttaggttggtcaaatcaaacaaagagctctgggccctgtgtcttgtgccctgggtgtcctggctggcctgcacttgcctgatgcagcagatgaagcggaaggaaaaactcacactgacttccaagaccaccacaaccctctgtctacattaccttgcccaggctctccaagctcagccattgggaccccagctcagagacctctgctctctggctgctgagggcatctggcaaaaaaagacccttttcagtccagatgacctcaggaagcatgggttagatggggccatcatctccaccttcttgaagatgggtattcttcaagagcaccccatccctctgagctacagcttcattcacctctgtttccaagagttctttgcagcaatgtcctatgtcttggaggatgagaaggggagaggtaaacattctaattgcatcatagatttggaaaagacgctagaagcatatggaatacatggcctgtttggggcatcaaccacacgtttcctattgggcctgttaagtgatgagggggagagagagatggagaacatctttcactgccggctgtctcaggggaggaacctgatgcagtgggtcccgtccctgcagctgctgctgcagccacactctctggagtccctccactgcttgtacgagactcggaacaaaacgttcctgacacaagtgatggcccatttcgaagaaatgggcatgtgtgtagaaacagacatggagctcttagtgtgcactttctgcattaaattcagccgccacgtgaagaagcttcagctgattgagggcaggcagcacagatcaacatggagccccaccatggtagtcctgttcaggtgggtcccagtcacagatgcctattggcagattctcttctccgtcctcaaggtcaccagaaacctgaaggagctggacctaagtggaaactcgctgagccactctgcagtgaagagtctttgtaagaccctgagacgccctcgctgcctcctggagaccctgcggttggctggctgtggcctcacagctgaggactgcaaggaccttgcctttgggctgagagccaaccagaccctgaccgagctggacctgagcttcaatgtgctcacggatgctggagccaaacacctttgccagagactgagacagccgagctgcaagctacagcgactgcagctggtcagctgtggcctcacgtctgactgctgccaggacctggcctctgtgcttagtgccagccccagcctgaaggagctagacctgcagcagaacaacctggatgacgttggcgtgcgactgctctgtgaggggctcaggcatcctgcctgcaaactcatacgcctggggctggaccagacaactctgagtgatgagatgaggcaggaactgagggccctggagcaggagaaacctcagctgctcatcttcagcagacggaaaccaagtgtgatgacccctactgagggcctggatacgggagagatgagtaatagcacatcctcactcaagcggcagagactcggatcagagagggcggcttcccatgttgctcaggctaatctcaaactcctggacgtgagcaagatcttcccaattgctgagattgcagaggaaagctccccagaggtagtaccggtggaactcttgtgcgtgccttctcctgcctctcaaggggacctgcatacgaagcctttggggactgacgatgacttctggggccccacggggcctgtggctactgaggtagttgacaaagaaaagaacttgtaccgagttcacttccctgtagctggctcctaccgctggcccaacacgggtctctgctttgtgatgagagaagcggtgaccgttgagattgaattctgtgtgtgggaccagttcctgggtgagatcaacccacagcacagctggatggtggcagggcctctgctggacatcaaggctgagcctggagctgtggaagctgtgcacctccctcactttgtggctctccaagggggccatgtggacacatccctgttccaaatggcccactttaaagaggaggggatgctcctggagaagccagccagggtggagctgcatcacatagttctggaaaaccccagcttctcccccttgggagtcctcctgaaaatgatccataatgccctgcgcttcattcccgtcacctctgtggtgttgctttaccaccgcgtccatcctgaggaagtcaccttccacctctacctgatcccaagtgactgctccattcggaaggaactggagctctgctatcgaagccctggagaagaccagctgttctcggagttctacgttggccacttgggatcagggatcaggctgcaagtgaaagacaagaaagatgagactctggtgtgggaggccttggtgaaaccaggagatctcatgcctgcaactactctgatccctccagcccgcatagccgtaccttcacctctggatgccccgcagttgctgcactttgtggaccagtatcgagagcagctgatagcccgagtgacatcggtggaggttgtcttggacaaactgcatggacaggtgctgagccaggagcagtacgagagggtgctggctgagaacacgaggcccagccagatgcggaagctgttcagcttgagccagtcctgggaccggaagtgcaaagatggactctaccaagccctgaaggagacccatcctcacctcattatggaactctgggagaagggcagcaaaaagggactcctgccactcagcagctgaagtatcaacaccagcccttgacccttgagtcctggctttggctgacccttctttgggtctcagtttctttctctgcaaacaagttgccatctggtttgccttccagcactaaagtaatggaactttgatgatgcctttgctgggcattatgtgtccatgccagggatgccacagggggccccagtccaggtggcctaacagcatctcagggaatgtccatctggagctggcaagacccctgcagacctcatagagcctcatctggtggccacagcagccaagcctagagccctccggatcccatccaggcgcaaagaggaataggagggacatggaaccatttgcctctggctgtgtcacagggtgagccccaaaattggggttcagcgtgggaggccacgtggattcttggctttgtacaggaagatctacaagagcaagccaacagagtaaagtggaaggaagtttattcagaaaataaaggagtatcacagctcttttagaatttgtctagcaggctttccagtttttaccagaaaacccctataaattaaaaattttttacttaaatttaagaattaaaaaaatacaaaaaagaaaaaatgaaaataaaggaataagaagttacctactccaaaaaaaaaaa

SEQ ID NO: 70 - DNA, 5533 bp, Homo sapiens
cttgttgactaggcgctgttcttgctggctggtgccccagggcctggagaggtctgaagaaacctgggagccagcagcccggggctccactctgggttctgaaagcccattccctgctctgcggctcctcccaccccacctcttctcagccttgcagctcaagggttgatctcaggagtccaggacccaggagagggaagaatctgaggaacacagaacagtgagcgttgcccacaccccatctcccgtcaccacatctcccctcaccctcaccctccctgcctggccctggaccccatcccaggacctccctatcagctgacttcttccagtgtcttgcaggcccctctgggctcctccctcccctggcttttcctaccactccccctctatcggcgtctatctgtaggtgccctgggatttataaaactgggttccgaatgctgaataagagacggtaagagccaaggcaaaggacagcactgttctctgcctgcctgataccctcaccacctgggaacatcccccagacaccctcttaactccgggacagagatggctggcggagcctggggccgcctggcctgttacttggagttcctgaagaaggaggagctgaaggagttccagcttctgctcgccaataaagcgcactccaggagctcttcgggtgagacacccgctcagccagagaagacgagtggcatggaggtggcctcgtacctggtggctcagtatggggagcagcgggcctgggacctagccctccatacctgggagcagatggggctgaggtcactgtgcgcccaagcccaggaaggggcaggccactctccctcattcccctacagcccaagtgaaccccacctggggtctcccagccaacccacctccaccgcagtgctaatgccctggatccatgaattgccggcggggtgcacccagggctcagagagaagggttttgagacagctgcctgacacatctggacgccgctggagagaaatctctgcctcactcctctaccaagctcttccaagctccccagaccatgagtctccaagccaggagtcacccaacgcccccacatccacagcagtgctggggagctggggatccccacctcagcccagcctagcacccagagagcaggaggctcctgggacccaatggcctctggatgaaacgtcaggaatttactacacagaaatcagagaaagagagagagagaaatcagagaaaggcaggcccccatgggcagcggtggtaggaacgcccccacaggcgcacaccagcctacagccccaccaccacccatgggagccttctgtgagagagagcctctgttccacatggccctggaaaaatgaggattttaaccaaaaattcacacagctgctacttctacaaagacctcaccccagaagccaagatcccctggtcaagagaagctggcctgattatgtggaggagaatcgaggacatttaattgagatcagagacttatttggcccaggcctggatacccaagaacctcgcatagtcatactgcagggggctgctggaattgggaagtcaacactggccaggcaggtgaaggaagcctgggggagaggccagctgtatggggaccgcttccagcatgtcttctacttcagctgcagagagctggcccagtccaaggtggtgagtctcgctgagctcatcggaaaagatgggacagccactccggctcccattagacagatcctgtctaggccagagcggctgctcttcatcctcgatggtgtagatgagccaggatgggtcttgcaggagccgagttctgagctctgtctgcactggagccagccacagccggcggatgcactgctgggcagtttgctggggaaaactatacttcccgaggcatccttcctgatcacggctcggaccacagctctgcagaacctcattccttctttggagcaggcacgttgggtagaggtcctggggttctctgagtccagcaggaaggaatatttctacagatatttcacagatgaaaggcaagcaattagagcctttaggttggtcaaatcaaacaaagagctctgggccctgtgtcttgtgccctgggtgtcctggctggcctgcacttgcctgatgcagcagatgaagcggaaggaaaaactcacactgacttccaagaccaccacaaccctctgtctacattaccttgcccaggctctccaagctcagccattgggaccccagctcagagacctctgctctctggctgctgagggcatctggcaaaaaaagacccttttcagtccagatgacctcaggaagcatgggttagatggggccatcatctccaccttcttgaagatgggtattcttcaagagcaccccatccctctgagctacagcttcattcacctctgtttccaagagttctttgcagcaatgtcctatgtcttggaggatgagaaggggagaggtaaacattctaattgcatcatagatttggaaaagacgctagaagcatatggaatacatggcctgtttggggcatcaaccacacgtttcctattgggcctgttaagtgatgagggggagagagagatggagaacatctttcactgccggctgtctcaggggaggaacctgatgcagtgggtcccgtccctgcagctgctgctgcagccacactctctggagtccctccactgcttgtacgagactcggaacaaaacgttcctgacacaagtgatggcccatttcgaagaaatgggcatgtgtgtagaaacagacatggagctcttagtgtgcactttctgcattaaattcagccgccacgtgaagaagcttcagctgattgagggcaggcagcacagatcaacatggagccccaccatggtagtcctgttcaggtgggtcccagtcacagatgcctattggcagattctcttctccgtcctcaaggtcaccagaaacctgaaggagctggacctaagtggaaactcgctgagccactctgcagtgaagagtctttgtaagaccctgagacgccctcgctgcctcctggagaccctgcggttggctggctgtggcctcacagctgaggactgcaaggaccttgcctttgggctgagagccaaccagaccctgaccgagctggacctgagcttcaatgtgctcacggatgctggagccaaacacctttgccagagactgagacagccgagctgcaagctacagcgactgcagctggtcagctgtggcctcacgtctgactgctgccaggacctggcctctgtgcttagtgccagccccagcctgaaggagctagacctgcagcagaacaacctggatgacgttggcgtgcgactgctctgtgaggggctcaggcatcctgcctgcaaactcatacgcctggggaaaccaagtgtgatgacccctactgagggcctggatacgggagagatgagtaatagcacatcctcactcaagcggcagagactcggatcagagagggcggcttcccatgttgctcaggctaatctcaaactcctggacgtgagcaagatcttcccaattgctgagattgcagaggaaagctccccagaggtagtaccggtggaactcttgtgcgtgccttctcctgcctctcaaggggacctgcatacgaagcctttggggactgacgatgacttctggggccccacggggcctgtggctactgaggtagttgacaaagaaaagaacttgtaccgagttcacttccctgtagctggctcctaccgctggcccaacacgggtctctgctttgtgatgagagaagcggtgaccgttgagattgaattctgtgtgtgggaccagttcctgggtgagatcaacccacagcacagctggatggtggcagggcctctgctggacatcaaggctgagcctggagctgtggaagctgtgcacctccctcactttgtggctctccaagggggccatgtggacacatccctgttccaaatggcccactttaaagaggaggggatgctcctggagaagccagccagggtggagctgcatcacatagttctggaaaaccccagcttctcccccttgggagtcctcctgaaaatgatccataatgccctgcgcttcattcccgtcacctctgtggtgttgctttaccaccgcgtccatcctgaggaagtcaccttccacctctacctgatcccaagtgactgctccattcggaaggccatagatgatctagaaatgaaattccagtttgtgcgaatccacaagccacccccgctgaccccactttatatgggctgtcgttacactgtgtctgggtctggttcagggatgctggaaatactccccaaggaactggagctctgctatcgaagccctggagaagaccagctgttctcggagttctacgttggccacttgggatcagggatcaggctgcaagtgaaagacaagaaagatgagactctggtgtgggaggccttggtgaaaccaggagatctcatgcctgcaactactctgatccctccagcccgcatagccgtaccttcacctctggatgccccgcagttgctgcactttgtggaccagtatcgagagcagctgatagcccgagtgacatcggtggaggttgtcttggacaaactgcatggacaggtgctgagccaggagcagtacgagagggtgctggctgagaacacgaggcccagccagatgcggaagctgttcagcttgagccagtcctgggaccggaagtgcaaagatggactctaccaagccctgaaggagacccatcctcacctcattatggaactctgggagaagggcagcaaaaagggactcctgccactcagcagctgaagtatcaacaccagcccttgacccttgagtcctggctttggctgacccttctttgggtctcagtttctttctctgcaaacaagttgccatctggtttgccttccagcactaaagtaatggaactttgatgatgcctttgctgggcattatgtgtccatgccagggatgccacagggggccccagtccaggtggcctaacagcatctcagggaatgtccatctggagctggcaagacccctgcagacctcatagagcctcatctggtggccacagcagccaagcctagagccctccggatcccatccaggcgcaaagaggaataggagggacatggaaccatttgcctctggctgtgtcacagggtgagccccaaaattggggttcagcgtgggaggccacgtggattcttggctttgtacaggaagatctacaagagcaagccaacagagtaaagtggaaggaagtttattcagaaaataaaggagtatcacagctcttttagaatttgtctagcaggctttccagtttttaccagaaaacccctataaattaaaaattttttacttaaatttaagaattaaaaaaatacaaaaaagaaaaaatgaaaataaaggaataagaagttacctactccaaaaaaaaaaa

SEQ ID NO: 71 - DNA, 5401 bp, Homo sapiens
cttgttgactaggcgctgttcttgctggctggtgccccagggcctggagaggtctgaagaaacctgggagccagcagcccggggctccactctgggttctgaaagcccattccctgctctgcggctcctcccaccccacctcttctcagccttgcagctcaagggttgatctcaggagtccaggacccaggagagggaagaatctgaggaacacagaacagtgagcgttgcccacaccccatctcccgtcaccacatctcccctcaccctcaccctccctgcctggccctggaccccatcccaggacctccctatcagctgacttcttccagtgtcttgcaggcccctctgggctcctccctcccctggcttttcctaccactccccctctatcggcgtctatctgtaggtgccctgggatttataaaactgggttccgaatgctgaataagagacggtaagagccaaggcaaaggacagcactgttctctgcctgcctgataccctcaccacctgggaacatcccccagacaccctcttaactccgggacagagatggctggcggagcctggggccgcctggcctgttacttggagttcctgaagaaggaggagctgaaggagttccagcttctgctcgccaataaagcgcactccaggagctcttcgggtgagacacccgctcagccagagaagacgagtggcatggaggtggcctcgtacctggtggctcagtatggggagcagcgggcctgggacctagccctccatacctgggagcagatggggctgaggtcactgtgcgcccaagcccaggaaggggcaggccactctccctcattcccctacagcccaagtgaaccccacctggggtctcccagccaacccacctccaccgcagtgctaatgccctggatccatgaattgccggcggggtgcacccagggctcagagagaagggttttgagacagctgcctgacacatctggacgccgctggagagaaatctctgcctcactcctctaccaagctcttccaagctccccagaccatgagtctccaagccaggagtcacccaacgcccccacatccacagcagtgctggggagctggggatccccacctcagcccagcctagcacccagagagcaggaggctcctgggacccaatggcctctggatgaaacgtcaggaatttactacacagaaatcagagaaagagagagagagaaatcagagaaaggcaggcccccatgggcagcggtggtaggaacgcccccacaggcgcacaccagcctacagccccaccaccacccatgggagccttctgtgagagagagcctctgttccacatggccctggaaaaatgaggattttaaccaaaaattcacacagctgctacttctacaaagacctcaccccagaagccaagatcccctggtcaagagaagctggcctgattatgtggaggagaatcgaggacatttaattgagatcagagacttatttggcccaggcctggatacccaagaacctcgcatagtcatactgcagggggctgctggaattgggaagtcaacactggccaggcaggtgaaggaagcctgggggagaggccagctgtatggggaccgcttccagcatgtcttctacttcagctgcagagagctggcccagtccaaggtggtgagtctcgctgagctcatcggaaaagatgggacagccactccggctcccattagacagatcctgtctaggccagagcggctgctcttcatcctcgatggtgtagatgagccaggatgggtcttgcaggagccgagttctgagctctgtctgcactggagccagccacagccggcggatgcactgctgggcagtttgctggggaaaactatacttcccgaggcatccttcctgatcacggctcggaccacagctctgcagaacctcattccttctttggagcaggcacgttgggtagaggtcctggggttctctgagtccagcaggaaggaatatttctacagatatttcacagatgaaaggcaagcaattagagcctttaggttggtcaaatcaaacaaagagctctgggccctgtgtcttgtgccctgggtgtcctggctggcctgcacttgcctgatgcagcagatgaagcggaaggaaaaactcacactgacttccaagaccaccacaaccctctgtctacattaccttgcccaggctctccaagctcagccattgggaccccagctcagagacctctgctctctggctgctgagggcatctggcaaaaaaagacccttttcagtccagatgacctcaggaagcatgggttagatggggccatcatctccaccttcttgaagatgggtattcttcaagagcaccccatccctctgagctacagcttcattcacctctgtttccaagagttctttgcagcaatgtcctatgtcttggaggatgagaaggggagaggtaaacattctaattgcatcatagatttggaaaagacgctagaagcatatggaatacatggcctgtttggggcatcaaccacacgtttcctattgggcctgttaagtgatgagggggagagagagatggagaacatctttcactgccggctgtctcaggggaggaacctgatgcagtgggtcccgtccctgcagctgctgctgcagccacactctctggagtccctccactgcttgtacgagactcggaacaaaacgttcctgacacaagtgatggcccatttcgaagaaatgggcatgtgtgtagaaacagacatggagctcttagtgtgcactttctgcattaaattcagccgccacgtgaagaagcttcagctgattgagggcaggcagcacagatcaacatggagccccaccatggtagtcctgttcaggtgggtcccagtcacagatgcctattggcagattctcttctccgtcctcaaggtcaccagaaacctgaaggagctggacctaagtggaaactcgctgagccactctgcagtgaagagtctttgtaagaccctgagacgccctcgctgcctcctggagaccctgcggttggctggctgtggcctcacagctgaggactgcaaggaccttgcctttgggctgagagccaaccagaccctgaccgagctggacctgagcttcaatgtgctcacggatgctggagccaaacacctttgccagagactgagacagccgagctgcaagctacagcgactgcagctggtcagctgtggcctcacgtctgactgctgccaggacctggcctctgtgcttagtgccagccccagcctgaaggagctagacctgcagcagaacaacctggatgacgttggcgtgcgactgctctgtgaggggctcaggcatcctgcctgcaaactcatacgcctggggaaaccaagtgtgatgacccctactgagggcctggatacgggagagatgagtaatagcacatcctcactcaagcggcagagactcggatcagagagggcggcttcccatgttgctcaggctaatctcaaactcctggacgtgagcaagatcttcccaattgctgagattgcagaggaaagctccccagaggtagtaccggtggaactcttgtgcgtgccttctcctgcctctcaaggggacctgcatacgaagcctttggggactgacgatgacttctggggccccacggggcctgtggctactgaggtagttgacaaagaaaagaacttgtaccgagttcacttccctgtagctggctcctaccgctggcccaacacgggtctctgctttgtgatgagagaagcggtgaccgttgagattgaattctgtgtgtgggaccagttcctgggtgagatcaacccacagcacagctggatggtggcagggcctctgctggacatcaaggctgagcctggagctgtggaagctgtgcacctccctcactttgtggctctccaagggggccatgtggacacatccctgttccaaatggcccactttaaagaggaggggatgctcctggagaagccagccagggtggagctgcatcacatagttctggaaaaccccagcttctcccccttgggagtcctcctgaaaatgatccataatgccctgcgcttcattcccgtcacctctgtggtgttgctttaccaccgcgtccatcctgaggaagtcaccttccacctctacctgatcccaagtgactgctccattcggaaggaactggagctctgctatcgaagccctggagaagaccagctgttctcggagttctacgttggccacttgggatcagggatcaggctgcaagtgaaagacaagaaagatgagactctggtgtgggaggccttggtgaaaccaggagatctcatgcctgcaactactctgatccctccagcccgcatagccgtaccttcacctctggatgccccgcagttgctgcactttgtggaccagtatcgagagcagctgatagcccgagtgacatcggtggaggttgtcttggacaaactgcatggacaggtgctgagccaggagcagtacgagagggtgctggctgagaacacgaggcccagccagatgcggaagctgttcagcttgagccagtcctgggaccggaagtgcaaagatggactctaccaagccctgaaggagacccatcctcacctcattatggaactctgggagaagggcagcaaaaagggactcctgccactcagcagctgaagtatcaacaccagcccttgacccttgagtcctggctttggctgacccttctttgggtctcagtttctttctctgcaaacaagttgccatctggtttgccttccagcactaaagtaatggaactttgatgatgcctttgctgggcattatgtgtccatgccagggatgccacagggggccccagtccaggtggcctaacagcatctcagggaatgtccatctggagctggcaagacccctgcagacctcatagagcctcatctggtggccacagcagccaagcctagagccctccggatcccatccaggcgcaaagaggaataggagggacatggaaccatttgcctctggctgtgtcacagggtgagccccaaaattggggttcagcgtgggaggccacgtggattcttggctttgtacaggaagatctacaagagcaagccaacagagtaaagtggaaggaagtttattcagaaaataaaggagtatcacagctcttttagaatttgtctagcaggctttccagtttttaccagaaaacccctataaattaaaaattttttacttaaatttaagaattaaaaaaatacaaaaaagaaaaaatgaaaataaaggaataagaagttacctactccaaaaaaaaaaa

SEQ ID NO: 72 - DNA, 5100 bp, Homo sapiens
cttgttgactaggcgctgttcttgctggctggtgccccagggcctggagaggtctgaagaaacctgggagccagcagcccggggctccactctgggttctgaaagcccattccctgctctgcggctcctcccaccccacctcttctcagccttgcagctcaagggttgatctcaggagtccaggacccaggagagggaagaatctgaggaacacagaacagtgagcgttgcccacaccccatctcccgtcaccacatctcccctcaccctcaccctccctgcctggccctggaccccatcccaggacctccctatcagctgacttcttccagtgtcttgcaggcccctctgggctcctccctcccctggcttttcctaccactccccctctatcggcgtctatctgtaggtgccctgggatttataaaactgggttccgaatgctgaataagagacggtaagagccaaggcaaaggacagcactgttctctgcctgcctgataccctcaccacctgggaacatcccccagacaccctcttaactccgggacagagatggctggcggagcctggggccgcctggcctgttacttggagttcctgaagaaggaggagctgaaggagttccagcttctgctcgccaataaagcgcactccaggagctcttcgggtgagacacccgctcagccagagaagacgagtggcatggaggtggcctcgtacctggtggctcagtatggggagcagcgggcctgggacctagccctccatacctgggagcagatggggctgaggtcactgtgcgcccaagcccaggaaggggcaggccactctccctcattcccctacagcccaagtgaaccccacctggggtctcccagccaacccacctccaccgcagtgctaatgccctggatccatgaattgccggcggggtgcacccagggctcagagagaagggttttgagacagctgcctgacacatctggacgccgctggagagaaatctctgcctcactcctctaccaagctcttccaagctccccagaccatgagtctccaagccaggagtcacccaacgcccccacatccacagcagtgctggggagctggggatccccacctcagcccagcctagcacccagagagcaggaggctcctgggacccaatggcctctggatgaaacgtcaggaatttactacacagaaatcagagaaagagagagagagaaatcagagaaaggcaggcccccatgggcagcggtggtaggaacgcccccacaggcgcacaccagcctacagccccaccaccacccatgggagccttctgtgagagagagcctctgttccacatggccctggaaaaatgaggattttaaccaaaaattcacacagctgctacttctacaaagacctcaccccagaagccaagatcccctggtcaagagaagctggcctgattatgtggaggagaatcgaggacatttaattgagatcagagacttatttggcccaggcctggatacccaagaacctcgcatagtcatactgcagggggctgctggaattgggaagtcaacactggccaggcaggtgaaggaagcctgggggagaggccagctgtatggggaccgcttccagcatgtcttctacttcagctgcagagagctggcccagtccaaggtggtgagtctcgctgagctcatcggaaaagatgggacagccactccggctcccattagacagatcctgtctaggccagagcggctgctcttcatcctcgatggtgtagatgagccaggatgggtcttgcaggagccgagttctgagctctgtctgcactggagccagccacagccggcggatgcactgctgggcagtttgctggggaaaactatacttcccgaggcatccttcctgatcacggctcggaccacagctctgcagaacctcattccttctttggagcaggcacgttgggtagaggtcctggggttctctgagtccagcaggaaggaatatttctacagatatttcacagatgaaaggcaagcaattagagcctttaggttggtcaaatcaaacaaagagctctgggccctgtgtcttgtgccctgggtgtcctggctggcctgcacttgcctgatgcagcagatgaagcggaaggaaaaactcacactgacttccaagaccaccacaaccctctgtctacattaccttgcccaggctctccaagctcagccattgggaccccagctcagagacctctgctctctggctgctgagggcatctggcaaaaaaagacccttttcagtccagatgacctcaggaagcatgggttagatggggccatcatctccaccttcttgaagatgggtattcttcaagagcaccccatccctctgagctacagcttcattcacctctgtttccaagagttctttgcagcaatgtcctatgtcttggaggatgagaaggggagaggtaaacattctaattgcatcatagatttggaaaagacgctagaagcatatggaatacatggcctgtttggggcatcaaccacacgtttcctattgggcctgttaagtgatgagggggagagagagatggagaacatctttcactgccggctgtctcaggggaggaacctgatgcagtgggtcccgtccctgcagctgctgctgcagccacactctctggagtccctccactgcttgtacgagactcggaacaaaacgttcctgacacaagtgatggcccatttcgaagaaatgggcatgtgtgtagaaacagacatggagctcttagtgtgcactttctgcattaaattcagccgccacgtgaagaagcttcagctgattgagggcaggcagcacagatcaacatggagccccaccatggtagtcctgttcaggtgggtcccagtcacagatgcctattggcagattctcttctccgtcctcaaggtcaccagaaacctgaaggagctggacctaagtggaaactcgctgagccactctgcagtgaagagtctttgtaagaccctgagacgccctcgctgcctcctggagaccctgcggttggctggctgtggcctcacagctgaggactgcaaggaccttgcctttgggctgagagccaaccagaccctgaccgagctggacctgagcttcaatgtgctcacggatgctggagccaaacacctttgccagagactgagacagccgagctgcaagctacagcgactgcagctggtcagctgtggcctcacgtctgactgctgccaggacctggcctctgtgcttagtgccagccccagcctgaaggagctagacctgcagcagaacaacctggatgacgttggcgtgcgactgctctgtgaggggctcaggcatcctgcctgcaaactcatacgcctggggctggaccagacaactctgagtgatgagatgaggcaggaactgagggccctggagcaggagaaacctcagctgctcatcttcagcagacggaaaccaagtgtgatgacccctactgagggcctggatacgggagagatgagtaatagcacatcctcactcaagcggcagagactcggatcagagagggcggcttcccatgttgctcaggctaatctcaaactcctggacgtgagcaagatcttcccaattgctgagattgcaggcaagagccacgaggaaagctccccagaggtagtaccggtggaactcttgtgcgtgccttctcctgcctctcaaggggacctgcatacgaagcctttggggactgacgatgacttctggggccccacggggcctgtggctactgaggtagttgacaaagaaaagaacttgtaccgagttcacttccctgtagctggctcctaccgctggcccaacacgggtctctgctttgtgatgagagaagcggtgaccgttgagattgaattctgtgtgtgggaccagttcctgggtgagatcaacccacagcacagctggatggtggcagggcctctgctggacatcaaggctgagcctggagctgtggaagctgtgcacctccctcactttgtggctctccaagggggccatgtggacacatccctgttccaaatggcccactttaaagaggaggggatgctcctggagaagccagccagggtggagctgcatcacatagttctggaaaaccccagcttctcccccttgggagtcctcctgaaaatgatccataatgccctgcgcttcattcccgtcacctctgtggtgttgctttaccaccgcgtccatcctgaggaagtcaccttccacctctacctgatcccaagtgactgctccattcggaaggccatagatgatctagaaatgaaattccagtttgtgcgaatccacaagccacccccgctgaccccactttatatgggctgtcgttacactgtgtctgggtctggttcagggatgctggaaatactccccaaggaactggagctctgctatcgaagccctggagaagaccagctgttctcggagttctacgttggccacttgggatcagggatcaggctgcaagtgaaagacaagaaagatgagactctggtgtgggaggccttggtgaaaccaggaaggaacaccagccagccgtggaacctcaggtgcaacagagacgccaggagatactagtgcccagcagcctgcggcagtaccaatgaagccagagagggcttggtggatgacaaggaggcctgagtagaccgcaggtgggtctgagaaatgggcttaggtgaggcaggtctttgaaggatttgttcttaatcatatgcgagatgctcaaaaggctggatgcctgcttttgtgggtgaagagcaagaagagaaaacaggttgtacacatacagatgcagatggagagacagagaaaaaaaaggaagaaggcagagaaatgcaccaattcttgagctgtattatctctggaccttgggattgtgggaggctttattttactactgattttgcctacactgttttctcaatttctagttttctacaaagatgatgtgttagctttttcacgcattaagattaaaatttaaaacagaaa

SEQ ID NO: 73 - protein, 1473 aa, Homo sapiens
MAGGAWGRLACYLEFLKKEELKEFQLLLANKAHSRSSSGETPAQPEKTSGMEVASYLVAQYGEQRAWDLALHTWEQMGLRSLCAQAQEGAGHSPSFPYSPSEPHLGSPSQPTSTAVLMPWIHELPAGCTQGSERRVLRQLPDTSGRRWREISASLLYQALPSSPDHESPSQESPNAPTSTAVLGSWGSPPQPSLAPREQEAPGTQWPLDETSGIYYTEIREREREKSEKGRPPWAAVVGTPPQAHTSLQPHHHPWEPSVRESLCSTWPWKNEDFNQKFTQLLLLQRPHPRSQDPLVKRSWPDYVEENRGHLIEIRDLFGPGLDTQEPRIVILQGAAGIGKSTLARQVKEAWGRGQLYGDRFQHVFYFSCRELAQSKVVSLAELIGKDGTATPAPIRQILSRPERLLFILDGVDEPGWVLQEPSSELCLHWSQPQPADALLGSLLGKTILPEASFLITARTTALQNLIPSLEQARWVEVLGFSESSRKEYFYRYFTDERQAIRAFRLVKSNKELWALCLVPWVSWLACTCLMQQMKRKEKLTLTSKTTTTLCLHYLAQALQAQPLGPQLRDLCSLAAEGIWQKKTLFSPDDLRKHGLDGAIISTFLKMGILQEHPIPLSYSFIHLCFQEFFAAMSYVLEDEKGRGKHSNCIIDLEKTLEAYGIHGLFGASTTRFLLGLLSDEGEREMENIFHCRLSQGRNLMQWVPSLQLLLQPHSLESLHCLYETRNKTFLTQVMAHFEEMGMCVETDMELLVCTFCIKFSRHVKKLQLIEGRQHRSTWSPTMVVLFRWVPVTDAYWQILFSVLKVTRNLKELDLSGNSLSHSAVKSLCKTLRRPRCLLETLRLAGCGLTAEDCKDLAFGLRANQTLTELDLSFNVLTDAGAKHLCQRLRQPSCKLQRLQLVSCGLTSDCCQDLASVLSASPSLKELDLQQNNLDDVGVRLLCEGLRHPACKLIRLGLDQTTLSDEMRQELRALEQEKPQLLIFSRRKPSVMTPTEGLDTGEMSNSTSSLKRQRLGSERAASHVAQANLKLLDVSKIFPIAEIAEESSPEVVPVELLCVPSPASQGDLHTKPLGTDDDFWGPTGPVATEVVDKEKNLYRVHFPVAGSYRWPNTGLCFVMREAVTVEIEFCVWDQFLGEINPQHSWMVAGPLLDIKAEPGAVEAVHLPHFVALQGGHVDTSLFQMAHFKEEGMLLEKPARVELHHIVLENPSFSPLGVLLKMIHNALRFIPVTSVVLLYHRVHPEEVTFHLYLIPSDCSIRKAIDDLEMKFQFVRIHKPPPLTPLYMGCRYTVSGSGSGMLEILPKELELCYRSPGEDQLFSEFYVGHLGSGIRLQVKDKKDETLVWEALVKPGDLMPATTLIPPARIAVPSPLDAPQLLHFVDQYREQLIARVTSVEVVLDKLHGQVLSQEQYERVLAENTRPSQMRKLFSLSQSWDRKCKDGLYQALKETHPHLIMELWEKGSKKGLLPLSS

SEQ ID NO: 74 - protein, 1429 aa, Homo sapiens
MAGGAWGRLACYLEFLKKEELKEFQLLLANKAHSRSSSGETPAQPEKTSGMEVASYLVAQYGEQRAWDLALHTWEQMGLRSLCAQAQEGAGHSPSFPYSPSEPHLGSPSQPTSTAVLMPWIHELPAGCTQGSERRVLRQLPDTSGRRWREISASLLYQALPSSPDHESPSQESPNAPTSTAVLGSWGSPPQPSLAPREQEAPGTQWPLDETSGIYYTEIREREREKSEKGRPPWAAVVGTPPQAHTSLQPHHHPWEPSVRESLCSTWPWKNEDFNQKFTQLLLLQRPHPRSQDPLVKRSWPDYVEENRGHLIEIRDLFGPGLDTQEPRIVILQGAAGIGKSTLARQVKEAWGRGQLYGDRFQHVFYFSCRELAQSKVVSLAELIGKDGTATPAPIRQILSRPERLLFILDGVDEPGWVLQEPSSELCLHWSQPQPADALLGSLLGKTILPEASFLITARTTALQNLIPSLEQARWVEVLGFSESSRKEYFYRYFTDERQAIRAFRLVKSNKELWALCLVPWVSWLACTCLMQQMKRKEKLTLTSKTTTTLCLHYLAQALQAQPLGPQLRDLCSLAAEGIWQKKTLFSPDDLRKHGLDGAIISTFLKMGILQEHPIPLSYSFIHLCFQEFFAAMSYVLEDEKGRGKHSNCIIDLEKTLEAYGIHGLFGASTTRFLLGLLSDEGEREMENIFHCRLSQGRNLMQWVPSLQLLLQPHSLESLHCLYETRNKTFLTQVMAHFEEMGMCVETDMELLVCTFCIKFSRHVKKLQLIEGRQHRSTWSPTMVVLFRWVPVTDAYWQILFSVLKVTRNLKELDLSGNSLSHSAVKSLCKTLRRPRCLLETLRLAGCGLTAEDCKDLAFGLRANQTLTELDLSFNVLTDAGAKHLCQRLRQPSCKLQRLQLVSCGLTSDCCQDLASVLSASPSLKELDLQQNNLDDVGVRLLCEGLRHPACKLIRLGLDQTTLSDEMRQELRALEQEKPQLLIFSRRKPSVMTPTEGLDTGEMSNSTSSLKRQRLGSERAASHVAQANLKLLDVSKIFPIAEIAEESSPEVVPVELLCVPSPASQGDLHTKPLGTDDDFWGPTGPVATEVVDKEKNLYRVHFPVAGSYRWPNTGLCFVMREAVTVEIEFCVWDQFLGEINPQHSWMVAGPLLDIKAEPGAVEAVHLPHFVALQGGHVDTSLFQMAHFKEEGMLLEKPARVELHHIVLENPSFSPLGVLLKMIHNALRFIPVTSVVLLYHRVHPEEVTFHLYLIPSDCSIRKELELCYRSPGEDQLFSEFYVGHLGSGIRLQVKDKKDETLVWEALVKPGDLMPATTLIPPARIAVPSPLDAPQLLHFVDQYREQLIARVTSVEVVLDKLHGQVLSQEQYERVLAENTRPSQMRKLFSLSQSWDRKCKDGLYQALKETHPHLIMELWEKGSKKGLLPLSS

SEQ ID NO: 75 - protein, 1443 aa, Homo sapiens
MAGGAWGRLACYLEFLKKEELKEFQLLLANKAHSRSSSGETPAQPEKTSGMEVASYLVAQYGEQRAWDLALHTWEQMGLRSLCAQAQEGAGHSPSFPYSPSEPHLGSPSQPTSTAVLMPWIHELPAGCTQGSERRVLRQLPDTSGRRWREISASLLYQALPSSPDHESPSQESPNAPTSTAVLGSWGSPPQPSLAPREQEAPGTQWPLDETSGIYYTEIREREREKSEKGRPPWAAVVGTPPQAHTSLQPHHHPWEPSVRESLCSTWPWKNEDFNQKFTQLLLLQRPHPRSQDPLVKRSWPDYVEENRGHLIEIRDLFGPGLDTQEPRIVILQGAAGIGKSTLARQVKEAWGRGQLYGDRFQHVFYFSCRELAQSKVVSLAELIGKDGTATPAPIRQILSRPERLLFILDGVDEPGWVLQEPSSELCLHWSQPQPADALLGSLLGKTILPEASFLITARTTALQNLIPSLEQARWVEVLGFSESSRKEYFYRYFTDERQAIRAFRLVKSNKELWALCLVPWVSWLACTCLMQQMKRKEKLTLTSKTTTTLCLHYLAQALQAQPLGPQLRDLCSLAAEGIWQKKTLFSPDDLRKHGLDGAIISTFLKMGILQEHPIPLSYSFIHLCFQEFFAAMSYVLEDEKGRGKHSNCIIDLEKTLEAYGIHGLFGASTTRFLLGLLSDEGEREMENIFHCRLSQGRNLMQWVPSLQLLLQPHSLESLHCLYETRNKTFLTQVMAHFEEMGMCVETDMELLVCTFCIKFSRHVKKLQLIEGRQHRSTWSPTMVVLFRWVPVTDAYWQILFSVLKVTRNLKELDLSGNSLSHSAVKSLCKTLRRPRCLLETLRLAGCGLTAEDCKDLAFGLRANQTLTELDLSFNVLTDAGAKHLCQRLRQPSCKLQRLQLVSCGLTSDCCQDLASVLSASPSLKELDLQQNNLDDVGVRLLCEGLRHPACKLIRLGKPSVMTPTEGLDTGEMSNSTSSLKRQRLGSERAASHVAQANLKLLDVSKIFPIAEIAEESSPEVVPVELLCVPSPASQGDLHTKPLGTDDDFWGPTGPVATEVVDKEKNLYRVHFPVAGSYRWPNTGLCFVMREAVTVEIEFCVWDQFLGEINPQHSWMVAGPLLDIKAEPGAVEAVHLPHFVALQGGHVDTSLFQMAHFKEEGMLLEKPARVELHHIVLENPSFSPLGVLLKMIHNALRFIPVTSVVLLYHRVHPEEVTFHLYLIPSDCSIRKAIDDLEMKFQFVRIHKPPPLTPLYMGCRYTVSGSGSGMLEILPKELELCYRSPGEDQLFSEFYVGHLGSGIRLQVKDKKDETLVWEALVKPGDLMPATTLIPPARIAVPSPLDAPQLLHFVDQYREQLIARVTSVEVVLDKLHGQVLSQEQYERVLAENTRPSQMRKLFSLSQSWDRKCKDGLYQALKETHPHLIMELWEKGSKKGLLPLSS

SEQ ID NO: 76 - protein, 1399 aa, Homo sapiens
MAGGAWGRLACYLEFLKKEELKEFQLLLANKAHSRSSSGETPAQPEKTSGMEVASYLVAQYGEQRAWDLALHTWEQMGLRSLCAQAQEGAGHSPSFPYSPSEPHLGSPSQPTSTAVLMPWIHELPAGCTQGSERRVLRQLPDTSGRRWREISASLLYQALPSSPDHESPSQESPNAPTSTAVLGSWGSPPQPSLAPREQEAPGTQWPLDETSGIYYTEIREREREKSEKGRPPWAAVVGTPPQAHTSLQPHHHPWEPSVRESLCSTWPWKNEDFNQKFTQLLLLQRPHPRSQDPLVKRSWPDYVEENRGHLIEIRDLFGPGLDTQEPRIVILQGAAGIGKSTLARQVKEAWGRGQLYGDRFQHVFYFSCRELAQSKVVSLAELIGKDGTATPAPIRQILSRPERLLFILDGVDEPGWVLQEPSSELCLHWSQPQPADALLGSLLGKTILPEASFLITARTTALQNLIPSLEQARWVEVLGFSESSRKEYFYRYFTDERQAIRAFRLVKSNKELWALCLVPWVSWLACTCLMQQMKRKEKLTLTSKTTTTLCLHYLAQALQAQPLGPQLRDLCSLAAEGIWQKKTLFSPDDLRKHGLDGAIISTFLKMGILQEHPIPLSYSFIHLCFQEFFAAMSYVLEDEKGRGKHSNCIIDLEKTLEAYGIHGLFGASTTRFLLGLLSDEGEREMENIFHCRLSQGRNLMQWVPSLQLLLQPHSLESLHCLYETRNKTFLTQVMAHFEEMGMCVETDMELLVCTFCIKFSRHVKKLQLIEGRQHRSTWSPTMVVLFRWVPVTDAYWQILFSVLKVTRNLKELDLSGNSLSHSAVKSLCKTLRRPRCLLETLRLAGCGLTAEDCKDLAFGLRANQTLTELDLSFNVLTDAGAKHLCQRLRQPSCKLQRLQLVSCGLTSDCCQDLASVLSASPSLKELDLQQNNLDDVGVRLLCEGLRHPACKLIRLGKPSVMTPTEGLDTGEMSNSTSSLKRQRLGSERAASHVAQANLKLLDVSKIFPIAEIAEESSPEVVPVELLCVPSPASQGDLHTKPLGTDDDFWGPTGPVATEVVDKEKNLYRVHFPVAGSYRWPNTGLCFVMREAVTVEIEFCVWDQFLGEINPQHSWMVAGPLLDIKAEPGAVEAVHLPHFVALQGGHVDTSLFQMAHFKEEGMLLEKPARVELHHIVLENPSFSPLGVLLKMIHNALRFIPVTSVVLLYHRVHPEEVTFHLYLIPSDCSIRKELELCYRSPGEDQLFSEFYVGHLGSGIRLQVKDKKDETLVWEALVKPGDLMPATTLIPPARIAVPSPLDAPQLLHFVDQYREQLIARVTSVEVVLDKLHGQVLSQEQYERVLAENTRPSQMRKLFSLSQSWDRKCKDGLYQALKETHPHLIMELWEKGSKKGLLPLSS

SEQ ID NO: 77 - protein, 1375 aa, Homo sapiens
MAGGAWGRLACYLEFLKKEELKEFQLLLANKAHSRSSSGETPAQPEKTSGMEVASYLVAQYGEQRAWDLALHTWEQMGLRSLCAQAQEGAGHSPSFPYSPSEPHLGSPSQPTSTAVLMPWIHELPAGCTQGSERRVLRQLPDTSGRRWREISASLLYQALPSSPDHESPSQESPNAPTSTAVLGSWGSPPQPSLAPREQEAPGTQWPLDETSGIYYTEIREREREKSEKGRPPWAAVVGTPPQAHTSLQPHHHPWEPSVRESLCSTWPWKNEDFNQKFTQLLLLQRPHPRSQDPLVKRSWPDYVEENRGHLIEIRDLFGPGLDTQEPRIVILQGAAGIGKSTLARQVKEAWGRGQLYGDRFQHVFYFSCRELAQSKVVSLAELIGKDGTATPAPIRQILSRPERLLFILDGVDEPGWVLQEPSSELCLHWSQPQPADALLGSLLGKTILPEASFLITARTTALQNLIPSLEQARWVEVLGFSESSRKEYFYRYFTDERQAIRAFRLVKSNKELWALCLVPWVSWLACTCLMQQMKRKEKLTLTSKTTTTLCLHYLAQALQAQPLGPQLRDLCSLAAEGIWQKKTLFSPDDLRKHGLDGAIISTFLKMGILQEHPIPLSYSFIHLCFQEFFAAMSYVLEDEKGRGKHSNCIIDLEKTLEAYGIHGLFGASTTRFLLGLLSDEGEREMENIFHCRLSQGRNLMQWVPSLQLLLQPHSLESLHCLYETRNKTFLTQVMAHFEEMGMCVETDMELLVCTFCIKFSRHVKKLQLIEGRQHRSTWSPTMVVLFRWVPVTDAYWQILFSVLKVTRNLKELDLSGNSLSHSAVKSLCKTLRRPRCLLETLRLAGCGLTAEDCKDLAFGLRANQTLTELDLSFNVLTDAGAKHLCQRLRQPSCKLQRLQLVSCGLTSDCCQDLASVLSASPSLKELDLQQNNLDDVGVRLLCEGLRHPACKLIRLGLDQTTLSDEMRQELRALEQEKPQLLIFSRRKPSVMTPTEGLDTGEMSNSTSSLKRQRLGSERAASHVAQANLKLLDVSKIFPIAEIAGKSHEESSPEVVPVELLCVPSPASQGDLHTKPLGTDDDFWGPTGPVATEVVDKEKNLYRVHFPVAGSYRWPNTGLCFVMREAVTVEIEFCVWDQFLGEINPQHSWMVAGPLLDIKAEPGAVEAVHLPHFVALQGGHVDTSLFQMAHFKEEGMLLEKPARVELHHIVLENPSFSPLGVLLKMIHNALRFIPVTSVVLLYHRVHPEEVTFHLYLIPSDCSIRKAIDDLEMKFQFVRIHKPPPLTPLYMGCRYTVSGSGSGMLEILPKELELCYRSPGEDQLFSEFYVGHLGSGIRLQVKDKKDETLVWEALVKPGRNTSQPWNLRCNRDARRY

SEQ ID NO: 78 - protein, 120 aa, Homo sapiens
MVQPAPPSRSRTVGPSTCRKALWDGSLSFLPFGASLLWFLLWVLWDGAWLWPRGLSRRGAGRGNAATLSLVSRLRRPVSEVSGAVNKGSGLASGLRSHVWKRGASSICVYIIDYAREFSR

SEQ ID NO: 79 - protein, 521 aa, Homo sapiens
MEFGLSWVLLVVFLQGVQCEVQLVDSGGGLVQPGGSLRLSCAASGFIVSDHYVEWVRQAPGKGPEWVGCFRSKAHKSTTEYAASVKGRFTILRDDSKNSVHLQMNSLKTDDTAVYYCVRDLEGAGKYDWYFDIWGRGILVTVSSASTKGPSVFPLAPCSRSTSGGTAALGCLVKDYFPEPVTVSWNSGALTSGVHTFPAVLQSSGLYSLSSVVTVPSSSLGTQTYTCNVNHKPSNTKVDKRVELKTPLGDTTHTCPRCPEPKSCDTPPPCPRCPEPKSCDTPPPCPRCPEPKSCDTPPPCPRCPAPELLGGPSVFLFPPKPKDTLMISRTPEVTCVVVDVSHEDPEVQFKWYVDGVEVHNAKTKPREEQFNSTFRVVSVLTVLHQDWLNGKEYKCKVSNKALPAPIEKTISKTKGQPREPQVYTLPPSREEMTKNQVSLTCLVKGFYPSDIAVEWESSGQPENNYNTTPPMLDSDGSFFLYSKLTVDKSRWQQGNIFSCSVMHEALHNRFTQKSLSLSPGK

SEQ ID NO: 80 - protein, 10 aa, Artificial Sequence
  source: /note=“Description of Artificial Sequence Synthetic peptide”
ACDCRGDCFC

SEQ ID NO: 81 - DNA, 7 bp, Artificial Sequence
  source: /note=“Description of Artificial Sequence Synthetic oligonucleotide”
ctgtctt

(19) United States
(12) Patent Application Publication
(10) Pub. No.: US 2011/0104679 A1
(43) Pub. Date: May 5, 2011

(54) Methods and Compositions for the Diagnosis and Treatment of Angiogenic Disorders

(76) Inventors: Margaret M. DeAngelis, Salt Lake City, UT (US); Fei Ji, Rockville, MD (US); Jurg Ott, West Orange, NJ (US)

(21) Appl. No.: 12/890,406

(22) Filed: Sep. 24, 2010

Related U.S. Application Data
(63) Continuation-in-part of application No. PCT/US2009/040220, filed on Apr. 10, 2009.
(60) Provisional application No. 61/044,393, filed on Apr. 11, 2008.
(60) Provisional application No. 61/085,124, filed on Jul. 31, 2008.

(51) Int. Cl. C12Q 1/68; G01N 33/68

(52) U.S. Cl. 435/6; 436/501

(57) ABSTRACT

The invention provides methods and compositions for determining whether an individual is at risk of developing, or has, one or more angiogenic disorders. The methods detect the presence and/or amount of one or more genes or gene products in a sample, including a RORA, CRIM1, CXCR4, C5orf26, IGHG3, NALP2, PLA2G4A, IGLJ3, SHQ1, UCHL1, TANC1, PKP2, DNAJC6, C6orf105, NALP1, RGS13, CXCL13, RPS6KA2, MMP7, IL1A, ABCA1, VCAN, KIAA0888, ENPP2, and FAM38B gene or gene product. In addition, the invention provides methods for using one or more of these genes or gene products as a target for preventing or delaying the onset of one or more angiogenic disorders or treating a patient with one or more such disorders. The angiogenic disorder can be, for example, an ocular angiogenic disorder, for example, a disorder associated with choroidal neovascularization, for example, age-related macular degeneration.

### RELATED APPLICATIONS

This application is a continuation-in-part of International Patent Application No. PCT/US2009/40220 filed Apr. 10, 2009, which claims the benefit of and priority to U.S. Provisional Patent Application Ser. Nos. 61/044,393, filed Apr. 11, 2008, and 61/085,124, filed Jul. 31, 2008, the entire disclosures of each of which are incorporated by reference herein for all purposes.

### FIELD OF THE INVENTION

The present invention relates generally to methods and compositions for the diagnosis and treatment of one or more angiogenic disorders, for example, an ocular angiogenic disorder, for example, a disorder associated with choroidal neovascularization, for example, age-related macular degeneration. More particularly, the invention relates to genes and gene products that are markers useful in the diagnosis of one or more angiogenic disorders, for example, an ocular angiogenic disorder, for example, a disorder associated with choroidal neovascularization, for example, age-related macular degeneration, and that are targets for the treatment of one or more of such angiogenic disorders.

### BACKGROUND

Angiogenic disorders can cause severe health problems without diagnosis and treatment. For example, there are a variety of chronic ocular angiogenic disorders, which, if untreated, may lead to partial or even complete vision loss. One prominent chronic ocular disorder is age-related macular degeneration, which is the leading cause of blindness amongst elderly Americans affecting a third of patients aged 75 years and older. (Fine et al. (2000) New Engl. J. Med. 342:483-492.) There are two forms of age-related macular degeneration, a dry form and a wet (also known as a neovascular) form.

The dry form involves a gradual degeneration of a specialized tissue beneath the retina, called the retina pigment epithelium, accompanied by the loss of the overlying photoreceptor cells. These changes result in a gradual loss of vision. The wet form is characterized by the growth of new blood vessels beneath the retina which can bleed and leak fluid, resulting in a rapid, severe and irreversible loss of central vision in the majority cases. This loss of central vision adversely affects one's every day life by impairing the ability to read, drive and recognize faces. In some cases, the macular degeneration progresses from the dry form to the wet form, and there are at least 200,000 newly diagnosed cases a year of the wet form. (See Hawkins et al. (1999) Mol. Vision. 5: 26-29.) The wet form accounts for approximately 90% of the severe vision loss associated with age-related macular degeneration.

At this time, current diagnostic methods cannot predict the risk of age-related macular degeneration for an individual. Unfortunately, the degeneration of the retina has already begun by the time age-related macular degeneration is diagnosed in the clinic. Further, most current treatments are limited in their applicability, and are unable to prevent or reverse the loss of vision especially in the case of the wet type, the more severe form of the disease. (Miller et al. (1999) Arch. Ophthalmol. 117(9): 1161-1173.)

Currently, the treatment of the dry form of age-related macular degeneration includes administration of antioxidant vitamins and/or zinc. Treatment of the wet form of age-related macular degeneration, however, has proved to be more difficult. A variety of methods have been approved in the United States of America for treating the wet form of age-related macular degeneration. Two approaches are laser-based therapies, which include laser photocoagulation and photodynamic therapy (“PDT”) using a benzoporphyrin derivative photosensitizer. Two other approaches include the delivery of pharmaceutically active agents, known as Lucentis®, from Genentech, Inc., and Macugen®, from Pfizer, Inc.

During laser photocoagulation, thermal laser light is used to heat and photocoagulate the neovasculature of the choroid. A problem associated with this approach is that the laser light must pass through the photoreceptor cells of the retina in order to photocoagulate the blood vessels in the underlying choroid. As a result, this treatment destroys the photoreceptor cells of the retina creating blind spots with associated vision loss. During photodynamic therapy, a benzoporphyrin derivative photosensitizer is administered to the individual to be treated. Once the photosensitizer accumulates in the choroidal neovasculature, non-thermal light from a laser is applied to the region to be treated, which activates the photosensitizer in that region. The activated photosensitizer generates free radicals that damage the vasculature in the vicinity of the photosensitizer (see, U.S. Pat. Nos. 5,798,349 and 6,225,303). This approach is more selective than laser photocoagulation and is less likely to result in blind spots. Under certain circumstances, this treatment has been found to restore vision in patients afflicted with the disorder (see, U.S. Pat. Nos. 5,756,541 and 5,910,510). Lucentis® is a fragment of a humanized, anti-VEGF (vascular endothelial growth factor) antibody. Macugen® is an RNA molecule capable of binding to and inhibiting VEGF. Lucentis® and Macugen® are injected into the eye, where the anti-VEGF antibody or RNA molecule, respectively, inhibits VEGF, thereby inhibiting the formation of blood vessels.

There is still an ongoing need for methods of identifying individuals at risk of developing one or more angiogenic disorders, for example, an ocular angiogenic disorder, for example, a disorder associated with choroidal neovascularization, for example, age-related macular degeneration, as well as methods of preventing the onset of such disorders, and, once established, the treatment of such disorders.

### SUMMARY OF THE INVENTION

The present invention is based, in part, upon the discovery that twenty-five genes and/or gene products, namely, RAR-related orphan receptor A (“RORA”); cysteine-rich motor neuron 1, also known as cysteine rich transmembrane BMP regulator 1 (choroid like) (“CRIM1”); chemokine (C—X—C motif) receptor 4 (“CXCR4”); chromosome 5 open reading frame 26 (“C5orf26”); immunoglobulin heavy constant gamma 3 (G3m marker) (“IGHG3”); NACHT, leucine rich repeat and PYD containing 2, also known as NLR family, pyrin domain containing 2 or NLRP2 (“NALP2”); phospholipase A2, group IVA (cytosolic, calcium-dependent) (“PLA2G4A”); immunoglobulin lambda joining 3 (“IGLJ3”); regulator of G-protein signaling 13 (“RGS13”); chemokine (C—X—C motif) ligand 13 (B-cell chemoattractant) (“CXCL13”); ribosomal protein S6 kinase, 90 kDa, polypeptide 2 (“RPS6KA2”); matrix metalloproteinase 7 (matrilysin, uterine), also known as matrix metallopeptidase 7 (“MMP7”); Interleukin 1, alpha (“IL1A”); ATP-binding cassette, sub-family A, member 1 (“ABCA1”); Versican (“VCAN”); Small nucleolar RNAs of the box H/ACA family quantitative accumulation protein 1 (“SHQ1”); ubiquitin carboxyl-terminal esterase L1 (ubiquitin thiolesterase) (“UCHL1”); tetratricopeptide repeat, ankyrin repeat and coiled-coil containing 1 (“TANC1”); plakophilin 2 (“PKP2”); DnaJ (Hsp40) homolog, subfamily C, member 6 (“DNAJC6”); KIAA0888, also known as LOC26049 (“KIAA0888”); ectonucleotide pyrophosphatase/phosphodiesterase 2 (autotaxin) (“ENPP2”); family with sequence similarity 38, member B (“FAM38B”); chromosome 6 open reading frame 105 (“C6orf105”); and NLR family, pyrin domain containing 1 or NLRP1 (“NALP1”) are associated with an angiogenic disorder, particularly an ocular angiogenic disorder, particularly a disorder associated with choroidal neovascularization, particularly age-related macular degeneration. As a result, the invention provides methods of determining whether an individual has, or is at risk of developing, one or more angiogenic disorders. The invention also provides targets useful for the treatment of one or more angiogenic disorders.

Herein, one or more angiogenic disorders can include, but is not limited to, one or more ocular angiogenic disorders, for example, (i) ocular disorders associated with choroidal neovascularization, for example, age-related macular degeneration (more specifically, the wet or neovascular form and the dry form of age-related macular degeneration), pathologic myopia, angioid streaks, choroidal ruptures, ocular histoplasmosis syndrome, multifocal choroiditis, idiosyncratic macular degeneration, and idiopathic choroidal neovascularization, (ii) ocular disorders associated with corneal neovascularization, including, for example, infections, burns, certain inflammatory disorders, trauma-related disorders, and immunological disorders, (iii) ocular disorders associated with iris neovascularization, including, for example, diabetes, retinal detachment, tumors, and central retinal vein occlusion, and (iv) ocular disorders associated with retinal neovascularization including, for example, diabetic retinopathy, branch retinal vein occlusion, certain inflammatory disorders, sickle cell retinopathy, and retinopathy of prematurity.

In one aspect, the invention provides a method of determining whether a mammal is at risk of developing, or has, one or more angiogenic disorders, for example, an ocular angiogenic disorder, for example, a disorder associated with choroidal neovascularization, for example, age-related macular degeneration. In particular, the method can be used to determine if a mammal, such as a human, has an ocular angiogenic disorder. The method includes the steps of: (a) measuring the amount of a gene or gene product in a test sample harvested from the mammal; and (b) comparing the amount of the gene or gene product against a control value, wherein an amount of the gene or gene product in the sample greater than the control value is indicative that the mammal is at risk of developing, or has, the ocular angiogenic disorder. The gene or gene product is selected from the group consisting of a CXCL13 gene, a RPS6KA2 gene, a MMP7 gene, an IL1A gene, a KIAA0888 gene, an ENPP2 gene, a CRIM1 gene, a CXCR4 gene, a C5orf26 gene, an IGHG3 gene, an IGLJ3 gene, a SHQ1 gene, a DNAJC6 gene, a C6orf105 gene, a NALP1 gene, a RGS13 gene, a CXCL13 gene product, a RPS6KA2 gene product, a MMP7 gene product, a IL1A gene product, KIAA0888 gene product, an ENPP2 gene product, a CRIM1 gene product, a CXCR4 gene product, a C5orf26 gene product, an IGHG3 gene product, an IGLJ3 gene product, a SHQ1 gene product, a DNAJC6 gene product, a C6orf105 gene product, a NALP1 gene product, and a RGS13 gene product. In certain embodiments, more than one gene and/or gene product is measured and compared against corresponding control values. For example, in certain embodiments, a gene and/or a gene product from two, three, four, five, six, or more of a CXCL13 gene, a RPS6KA2 gene, a MMP7 gene, an IL1A gene, a KIAA0888 gene, an ENPP2 gene, a CRIM1 gene, a CXCR4 gene, a C5orf26 gene, an IGHG3 gene, an IGLJ3 gene, a SHQ1 gene, a DNAJC6 gene, a C6orf 105 gene, a NALP1 gene, a RGS13 gene, a CXCL13 gene product, a RPS6KA2 gene product, a MMP7 gene product, a IL1A gene product, KIAA0888 gene product, an ENPP2 gene product, a CRIM1 gene product, a CXCR4 gene product, a C5orf26 gene product, an IGHG3 gene product, an IGLJ3 gene product, a SHQ1 gene product, a DNAJC6 gene product, a C6orf105 gene product, a NALP1 gene product, and a RGS13 gene product are measured and compared against corresponding control values.

In another aspect, the invention provides a method of determining whether a mammal is at risk of developing, or has, one or more angiogenic disorders, for example, an ocular angiogenic disorder, for example, a disorder associated with choroidal neovascularization, for example, age-related macular degeneration. In particular, the method can be used to determine if a mammal, such as a human, has an ocular angiogenic disorder. The method includes the steps: of (a) measuring the amount of a gene or gene product in a test sample harvested from the mammal; and (b) comparing the amount of the gene or gene product against a control value, wherein an amount of the gene or gene product in the sample less than the control value is indicative that the mammal is at risk of developing, or has, the ocular angiogenic disorder. The gene or gene product is selected from the group consisting of a RORA gene, a NALP2 gene, a PLA2G4A gene, a PKP2 gene, a UCHL1 gene, a TANC1 gene, an ABCA1 gene, a VCAN gene, a FAM38B gene, a RORA gene product, a NALP2 gene product, a PLA2G4A gene product, a PKP2 gene product, a UCHL1 gene product, a TANC1 gene product, an ABCA1 gene product, a VCAN gene product, a and a FAM38B gene product. In certain embodiments, more than one gene or gene product is measured and compared against corresponding control values. For example, in certain embodiments, a gene and/or a gene product from two, three, four, or more of a RORA gene, a NALP2 gene, a PLA2G4A gene, a PKP2 gene, a UCHL1 gene, a TANC1 gene, an ABCA1 gene, a VCAN gene, a FAM38B gene, a RORA gene product, a NALP2 gene product, a PLA2G4A gene product, a PKP2 gene product, a UCHL1 gene product, a TANC1 gene product, an ABCA1 gene product, a VCAN gene product, and a FAM38B gene product are measured and compared against corresponding control values.

The invention also includes a method of determining whether a mammal is at risk of developing, or has, one or more angiogenic disorders, for example, an ocular angiogenic disorder, for example, a disorder associated with choroidal neovascularization, for example, age-related macular degeneration by measuring the amount of one or more markers in a test sample harvested from the mammal. In particular, the method can be used to determine if a mammal, such as a human, is at risk of developing, or has, an ocular angiogenic disorder. The one or more markers are selected from the group consisting of a RORA gene, a CRIM1 gene, a CXCR4 gene, a C5orf26 gene, an IGHG3 gene, a NALP2 gene, a PLA2G4A gene, an IGLJ3 gene, a SHQ1 gene, a UCHL1 gene, a TANC1 gene, a PKP2 gene, a DNAJC6 gene, a C6orf105 gene, a NALP1 gene, a RGS13 gene, a CXCL13 gene, a RPS6KA2 gene, a MMP7 gene, an IL1A gene, an ABCA1 gene, a VCAN gene, a KIAA0888 gene, an ENPP2 gene, a FAM38B gene, a RORA gene product, a CRIM1 gene product, a CXCR4 gene product, a C5orf26 gene product, an IGHG3 gene product, a NALP2 gene product, a PLA2G4A gene product, an IGLJ3 gene product, a SHQ1 gene product, a UCHL1 gene product, a TANC1 gene product, a PKP2 gene product, a DNAJC6 gene product, a C6orf105 gene product, a NALP1 gene product, a RGS13 gene product, a CXCL13 gene product, a RPS6KA2 gene product, a MMP7 gene product, an IL1A gene product, an ABCA1 gene product, a VCAN gene product, a KIAA0888 gene product, an ENPP2 gene product, and a FAM38B gene product. In addition, the amount of the one or more markers in the test sample is compared against one or more corresponding control values. When the measured marker is a CXCL13 gene, a RPS6KA2 gene, a MMP7 gene, an IL1A gene, a KIAA0888 gene, an ENPP2 gene, a CRIM1 gene, a CXCR4 gene, a C5orf26 gene, an IGHG3 gene, an IGLJ3 gene, a SHQ1 gene, a DNAJC6 gene, a C6orf105 gene, a NALP1 gene, a RGS13 gene, a CXCL13 gene product, a RPS6KA2 gene product, a MMP7 gene product, an IL1A gene product, a KIAA0888 gene product, an ENPP2 gene product, a CRIM1 gene product, a CXCR4 gene product, a C5orf26 gene product, an IGHG3 gene product, an IGLJ3 gene product, a SHQ1 gene product, a DNAJC6 gene product, a C6orf105 gene product, a NALP1 gene product, or a RGS13 gene product, an amount of the marker in the sample greater than its corresponding control value is indicative that the mammal is at risk of developing, or has, the ocular angiogenic disorder. When the measured marker is a RORA gene, a NALP2 gene, a PLA2G4A gene, a PKP2 gene, a UCHL1 gene, a TANC1 gene, an ABCA1 gene, a VCAN gene, a FAM38B gene, a RORA gene product, a NALP2 gene product, a PLA2G4A gene product, a PKP2 gene product, a UCHL1 gene product, a TANC1 gene product, an ABCA1 gene product, a VCAN gene product, or a FAM38B gene product, an amount of the marker in the sample less than its corresponding control value is indicative that the mammal is at risk of developing, or has, the ocular angiogenic disorder. In certain embodiments, when two or more measured amounts of markers are different from corresponding control values, it is indicative that the mammal is at risk of developing, or has, the ocular angiogenic disorder. In certain embodiments, when several measured markers are different from corresponding control values, it is indicative that the mammal is at risk of developing, or has, the ocular angiogenic disorder.

The test sample can be any appropriate sample, for example, a tissue or body fluid sample. In one example, the body fluid sample is blood, serum or plasma. In another example, the tissue sample is choroid or retina.

The marker to be determined can be a gene product and a nucleic acid, for example, a RNA molecule, for example, a nucleic acid, for example, a mRNA molecule. Any appropriate method can be used to determine the nucleic acid in the sample. In one example, the nucleic acid is measured, for example, by a hybridization assay. Alternatively, gene product is a protein. The protein can be measured, for example, by a known immunoassay such as a sandwich immunoassay.

In another aspect, the invention provides a method of preventing, slowing or stopping the development of one or more angiogenic disorders, for example, an ocular angiogenic disorder, for example, a disorder associated with choroidal neovascularization, for example, age-related macular degeneration. In particular, the method can be used to prevent, slow or stop the development of an ocular angiogenic disorder. The method includes administering to a mammal, such as a human, suspected of having an ocular angiogenic disorder a therapeutically effective amount of one or more of a CRIM1 antagonist, a CXCR4 antagonist, a C5orf26 antagonist, an IGHG3 antagonist, an IGLJ3 antagonist, a SHQ1 antagonist, a DNAJC6 antagonist, a C6orf105 antagonist, a NALP1 antagonist, a RGS13 antagonist, a CXCL13 antagonist, a RPS6KA2 antagonist, a MMP7 antagonist, an IL1A antagonist, a KIAA0888 antagonist, an ENPP2 antagonist, a RORA agonist, a NALP2 agonist, a PLA2G4A agonist, a PKP2 agonist, a UCHL1 agonist, a TANC1 agonist, an ABCA1 agonist, a VCAN agonist, and a FAM38B agonist to prevent, slow or stop the progression of the disorder. In one example, the ocular angiogenic disorder is age-related macular degeneration. The one or more antagonists and/or agonists can be administered by any known method in the art, for example, the one or more antagonists and/or agonists can be administered orally, parentally, or locally to an eye of the mammal.

In another aspect, the invention provides a kit to determine if a mammal is at risk of developing, or has, one or more angiogenic disorders, for example, an ocular angiogenic disorder, for example, a disorder associated with choroidal neovascularization, for example, age-related macular degeneration. In particular, the kit can be used to determine if a mammal, such as a human, is at risk of developing, or has, an ocular angiogenic disorder. The kit includes (i) an agent for determining the amount of one or more of a RORA gene, a CRIM1 gene, a CXCR4 gene, a C5orf26 gene, an IGHG3 gene, a NALP2 gene, a PLA2G4A gene, an IGLJ3 gene, a SHQ1 gene, a UCHL1 gene, a TANC1 gene, a PKP2 gene, a DNAJC6 gene, a C6orf105 gene, a NALP1 gene, a RGS13 gene, a CXCL13 gene, a RPS6KA2 gene, a MMP7 gene, an IL1A gene, an ABCA1 gene, a VCAN gene, a KIAA0888 gene, an ENPP2 gene, a FAM38B gene, a RORA gene product, a CRIM1 gene product, a CXCR4 gene product, a C5orf26 gene product, an IGHG3 gene product, a NALP2 gene product, a PLA2G4A gene product, an IGLJ3 gene product, a SHQ1 gene product, a UCHL1 gene product, a TANC1 gene product, a PKP2 gene product, a DNAJC6 gene product, a C6orf105 gene product, a NALP1 gene product, a RGS13 gene product, a CXCL13 gene product, a RPS6KA2 gene product, a MMP7 gene product, an IL1A gene product, an ABCA1 gene product, a VCAN gene product, a KIAA0888 gene product, an ENPP2 gene product, and a FAM38B gene product in a test sample, and (ii) instructions on how to determine the amount of the one or more genes or gene products in the sample. The instructions may also describe how to compare the test results against control values to determine whether an individual has, or is at risk of developing, the ocular angiogenic disorder. In one example, the ocular angiogenic disorder is the neovascular form of age-related macular degeneration.

Herein, the angiogenic disorder, such as the ocular angiogenic disorder, can be age-related macular degeneration. Age-related macular degeneration can refer to a wet form of age-related macular degeneration, also referred to as a neovascular form of age-related macular degeneration, and a dry form of age-related macular degeneration.

In another aspect, the invention provides a method for downregulating CRIM1, downregulating CXCR4, downregulating C5orf26, downregulating IGHG3, down-regulating IGLJ3, downregulating RGS13, downregulating SHQ1, downregulating DNAJC6, downregulating C6orf105, downregulating NALP1, downregulating CXCL13, down-regulating RPS6KA2, downregulating MMP7, downregulating IL1A, downregulating KIAA0888, downregulating ENPP2, upregulating RORA, upregulating NALP2, upregulating PLA2G4A, upregulating PKP2, upregulating UCHL1, upregulating TANC1, upregulating ABCA1, upregulating VCAN, or upregulating FAM38B in vascular or ocular tissue. In particular, the method can be used to deliver at least one agent selected from the group consisting of an antagonist of CRIM1, an antagonist of CXCR4, an antagonist of C5orf26, an antagonist of IGHG3, an antagonist of IGLJ3, an antagonist of RGS13, an antagonist of SHQ1, an antagonist of DNAJC6, an antagonist of C6orf105, an antagonist of NALP1, an antagonist of CXCL13, an antagonist of RPS6KA2, an antagonist of MMP7, an antagonist of IL1A, an antagonist of KIAA0888, an antagonist of ENPP2, an agonist of UCHL1, an agonist of TANC1, agonist of RORA, an agonist of NALP2, an agonist of PLA2G4A, an agonist of PKP2, an agonist of ABCA1, an agonist of VCAN, or an agonist of FAM38B to the vascular or ocular tissue in an amount sufficient to downregulate CRIM1, downregulate CXCR4, downregulate C5orf26, downregulate IGHG3, downregulate IGLJ3, downregulate RGS13, downregulate SHQ1, downregulate DNAJC6, downregulate C6orf105, downregulate NALP1, downregulate CXCL13, downregulate RPS6KA2, downregulate MMP7, downregulate IL1A, downregulate KIAA0888, downregulate ENPP2, upregulate RORA, upregulate NALP2, upregulate PLA2G4A, upregulate PKP2, upregulate UCHL1, upregulate TANC1, upregulate ABCA1, upregulate VCAN, upregulate FAM38B, or a combination thereof in the vascular or ocular tissue.

In another aspect, the invention provides a method of assisting in diagnosing or assessing the risk of developing an ocular angiogenic disorder. For example, the method includes communicating a report indicating increased CXCL13, RPS6KA2, MMP7, IL1A, KIAA0888, ENPP2, CRIM1, CXCR4, C5orf26, IGHG3, IGLJ3, RGS13, SHQ1, DNAJC6, C6orf105, or NALP1 gene or gene product relative to a control value or decreased RORA, NALP2, PLA2G4A, PKP2, UCHL1, TANC1, ABCA1, VCAN, or FAM38B gene or gene product relative to a control value. In one embodiment, increased CXCL13, RPS6KA2, MMP7, IL1A, KIAA0888, ENPP2, CRIM1, CXCR4, C5orf26, IGHG3, IGLJ3, RGS13, SHQ1, DNAJC6, C6orf105, or NALP1 gene or gene product or decreased RORA, NALP2, PLA2G4A, PKP2, UCHL1, TANC1, ABCA1, VCAN, or FAM38B gene or gene product is indicative of having, or having an increased risk of developing, an ocular angiogenic disorder.

The foregoing aspects and embodiments of the invention may be more fully understood by reference to the following figures, detailed description and claims.

### BRIEF DESCRIPTION OF THE DRAWINGS

FIGS. 1A and 1B depict the twenty-five genes discovered to be associated with one or more angiogenic disorders, particularly, an ocular angiogenic disorder, particularly, a disorder associated with choroidal neovascularization, particularly, age-related macular degeneration (AMD). FIG. 1A depicts genes that are upregulated in siblings affected with AMD relative to unaffected, control paired siblings. FIG. 1B depicts genes that are downregulated in siblings affected with AMD relative to unaffected, control paired siblings.

FIG. 2A depicts an mRNA sequence (SEQ ID NO: 1) of human CRIM1.

FIG. 2B depicts an amino acid sequence of human CRIM1 (SEQ ID NO: 2).

FIG. 3A depicts the transcript variant 1 mRNA sequence of human CXCR4 (SEQ ID NO: 3).

FIG. 3B depicts the transcript variant 2 mRNA sequence of human CXCR4 (SEQ ID NO: 4).

FIG. 3C depicts the isoform a amino acid sequence of human CXCR4 (SEQ ID NO: 5).

FIG. 3D depicts the isoform b amino acid sequence of human CXCR4 (SEQ ID NO: 6).

FIG. 4A depicts a transcript sequence of human C5orf26 (SEQ ID NO: 7).

FIG. 4B depicts an amino acid sequence of human C5orf26 (SEQ ID NO: 78).

FIG. 5A depicts a genomic nucleotide sequence of human IGHG3 (SEQ ID NO: 8).

FIG. 5B depicts an amino acid sequence of human IGHG3 (SEQ ID NO: 79).

FIG. 6A depicts an mRNA sequence of human NALP2 (SEQ ID NO: 9).

FIG. 6B depicts an amino acid sequence of human NALP2 (SEQ ID NO: 10).

FIG. 7A depicts an mRNA sequence of human PLA2G4A (SEQ ID NO: 11).

FIG. 7B depicts an amino acid sequence of human PLA2G4A (SEQ ID NO: 12).

FIG. 8 depicts a genomic nucleotide sequence of human IGLJ3 (SEQ ID NO: 13).

FIG. 9A depicts the transcript variant 1 mRNA sequence of human RGS13 (SEQ ID NO: 14).

FIG. 9B depicts the transcript variant 2 mRNA sequence of human RGS13 (SEQ ID NO: 15).

FIG. 9C depicts the amino acid sequence corresponding to transcript variant 1 of human RGS13 (SEQ ID NO: 16).

FIG. 9D depicts the amino acid sequence corresponding to transcript variant 2 of human RGS13 (SEQ ID NO: 17).

FIG. 10A depicts an mRNA sequence of human CXCL13 (SEQ ID NO: 18).

FIG. 10B depicts an amino acid sequence of human CXCL13 (SEQ ID NO: 19).

FIG. 11A depicts the transcript variant 1 mRNA sequence of human RPS6KA2 (SEQ ID NO: 20).

FIG. 11B depicts the transcript variant 2 mRNA sequence of human RPS6KA2 (SEQ ID NO: 21).

FIG. 11C depicts the isoform a amino acid sequence of human RPS6KA2 (SEQ ID NO: 22).

FIG. 11D depicts the isoform b amino acid sequence of human RPS6KA2 (SEQ ID NO: 23).

FIG. 12A depicts an mRNA sequence of human MMP7 (SEQ ID NO: 24).

FIG. 12B depicts an amino acid sequence of human MMP7 (SEQ ID NO: 25).

FIG. 13A depicts the transcript variant 1 mRNA sequence of human RORA (SEQ ID NO: 26).

FIG. 13B depicts the transcript variant 2 nucleotide sequence of human RORA (SEQ ID NO: 27).

FIG. 13C depicts the transcript variant 3 nucleotide sequence of human RORA (SEQ ID NO: 28).

FIG. 13D depicts the transcript variant 4 nucleotide sequence of human RORA (SEQ ID NO: 29).

FIG. 13E depicts the isoform a amino acid sequence of human RORA (SEQ ID NO: 30).

FIG. 13F depicts the isoform b amino acid sequence of human RORA (SEQ ID NO: 31).

FIG. 13G depicts the isoform c amino acid sequence of human RORA (SEQ ID NO: 32).

FIG. 13H depicts the isoform d amino acid sequence of human RORA (SEQ ID NO: 33).

FIG. 14A depicts an mRNA sequence of human IL1A (SEQ ID NO: 34).

FIG. 14B depicts an amino acid sequence of human IL1A (SEQ ID NO: 35).

FIG. 15A depicts an mRNA sequence of human ABCA1 (SEQ ID NO: 36).

FIG. 15B depicts an amino acid sequence of human ABCA1 (SEQ ID NO: 37).

FIG. 16A depicts the transcript variant 1 mRNA sequence of human VCAN (SEQ ID NO: 38).

FIG. 16B depicts the isoform 1 amino acid sequence of human VCAN (SEQ ID NO: 39).

FIG. 16C depicts the transcript variant 2 mRNA sequence of human VCAN (SEQ ID NO: 40).

FIG. 16D depicts the isoform 2 amino acid sequence of human VCAN (SEQ ID NO: 41).

FIG. 17A depicts an mRNA sequence of human SHQ1 (SEQ ID NO: 42).

FIG. 17B depicts an amino acid sequence of human SHQ1 (SEQ ID NO: 43).

FIG. 18A depicts an mRNA sequence of human UCHL1 (SEQ ID NO: 44).

FIG. 18B depicts an amino acid sequence of human UCHL1 (SEQ ID NO: 45).

FIG. 19A depicts an mRNA sequence of human TANC1 (SEQ ID NO: 46).

FIG. 19B depicts an amino acid sequence of human TANC1 (SEQ ID NO: 47).

FIG. 20A depicts the transcript variant 2a mRNA sequence of human PKP2 (SEQ ID NO: 48).

FIG. 20B depicts the transcript variant 2b mRNA sequence of human PKP2 (SEQ ID NO: 49).

FIG. 20C depicts the isoform 2a amino acid sequence of human PKP2 (SEQ ID NO: 50).

FIG. 20D depicts the isoform 2b amino acid sequence of human PKP2 (SEQ ID NO: 51).

FIG. 21A depicts an mRNA sequence of human DNAJC6 (SEQ ID NO: 52).

FIG. 21B depicts an amino acid sequence of human DNAJC6 (SEQ ID NO: 53).

FIG. 22A depicts an mRNA sequence of human KIAA0888 (SEQ ID NO: 54).

FIG. 22B depicts an amino acid sequence of human KIAA0888 (SEQ ID NO:55).

FIG. 23A depicts the transcript variant 1 mRNA sequence of human ENPP2 (SEQ ID NO: 56).

FIG. 23B depicts the transcript variant 2 mRNA sequence of human ENPP2 (SEQ ID NO: 57).

FIG. 23C depicts the transcript variant 3 mRNA sequence of human ENPP2 (SEQ ID NO: 58).

FIG. 23D depicts the isoform 1 amino acid sequence of human ENPP2 (SEQ ID NO: 59).

FIG. 23E depicts the isoform 2 amino acid sequence of human ENPP2 (SEQ ID NO: 60).

FIG. 23F depicts the isoform 3 amino acid sequence of human ENPP2 (SEQ ID NO: 61).

FIG. 24A depicts an mRNA sequence of human FAM38B (SEQ ID NO: 62).

FIG. 24B depicts an amino acid sequence of human FAM38B (SEQ ID NO: 63).

FIG. 25A depicts the transcript variant 1 mRNA sequence of human C6orf105 (SEQ ID NO: 64).

FIG. 25B depicts the transcript variant 2 mRNA sequence of human C6orf105 (SEQ ID NO: 65).

FIG. 25C depicts the isoform 1 amino acid sequence of human C6orf105 (SEQ ID NO: 66).

FIG. 25D depicts the isoform 2 amino acid sequence of human C6orf105 (SEQ ID NO: 67).

FIG. 26A depicts the transcript variant 1 mRNA sequence of human NALP1 (SEQ ID NO: 68).

FIG. 26B depicts the transcript variant 2 mRNA sequence of human NALP1 (SEQ ID NO: 69).

FIG. 26C depicts the transcript variant 3 mRNA sequence of human NALP1 (SEQ ID NO: 70).

FIG. 26D depicts the transcript variant 4 mRNA sequence of human NALP1 (SEQ ID NO: 71).

FIG. 26E depicts the transcript variant 5 mRNA sequence of human NALP1 (SEQ ID NO: 72).

FIG. 26F depicts the isoform 1 amino acid sequence of human NALP1 (SEQ ID NO: 73).

FIG. 26G depicts the isoform 2 amino acid sequence of human NALP1 (SEQ ID NO: 74).

FIG. 26H depicts the isoform 3 amino acid sequence of human NALP1 (SEQ ID NO: 75).

FIG. 26I depicts the isoform 4 amino acid sequence of human NALP1 (SEQ ID NO: 76).

FIG. 26J depicts the isoform 5 amino acid sequence of human NALP1 (SEQ ID NO: 77).

### DETAILED DESCRIPTION OF THE INVENTION

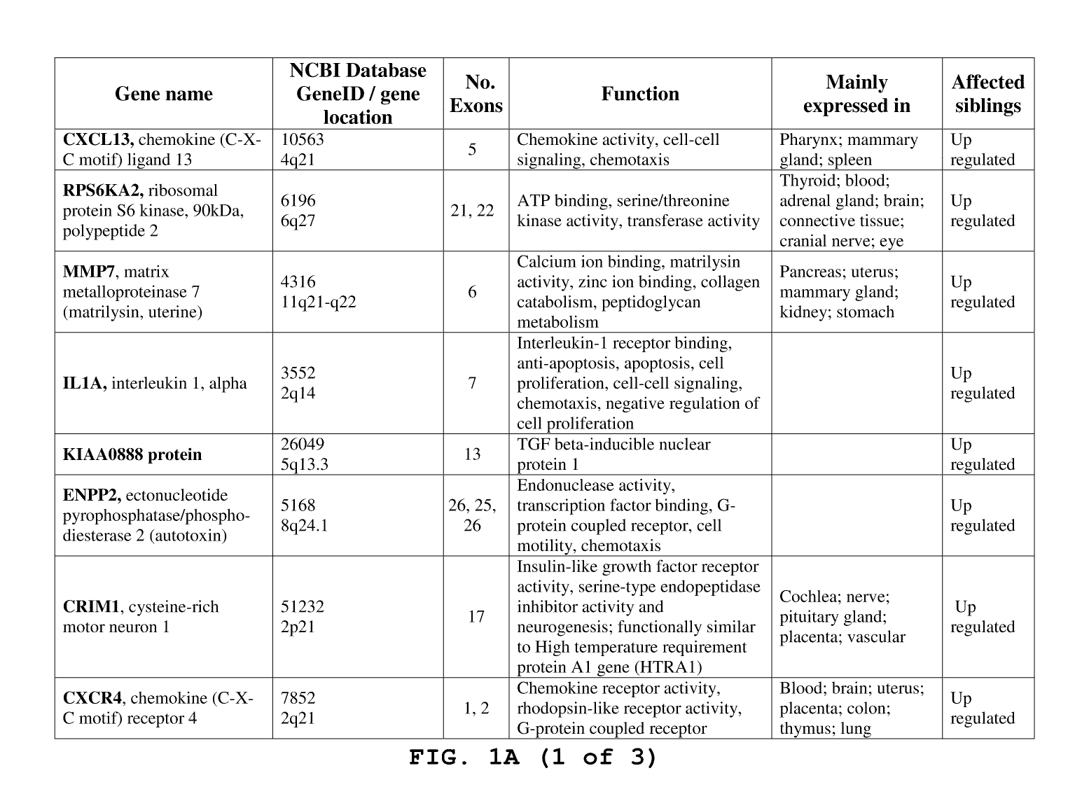

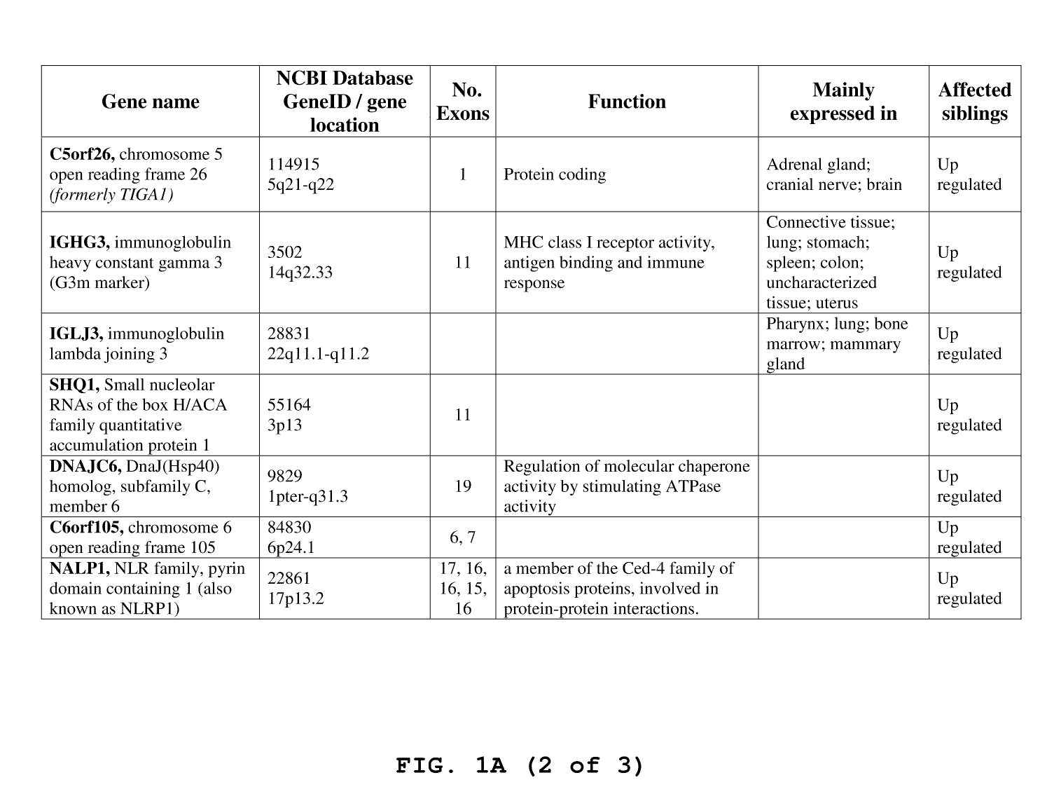

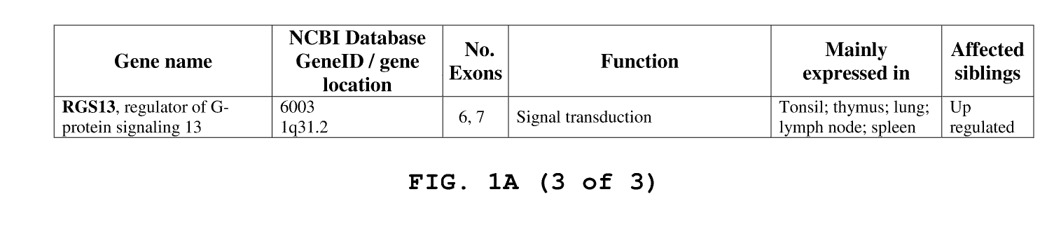

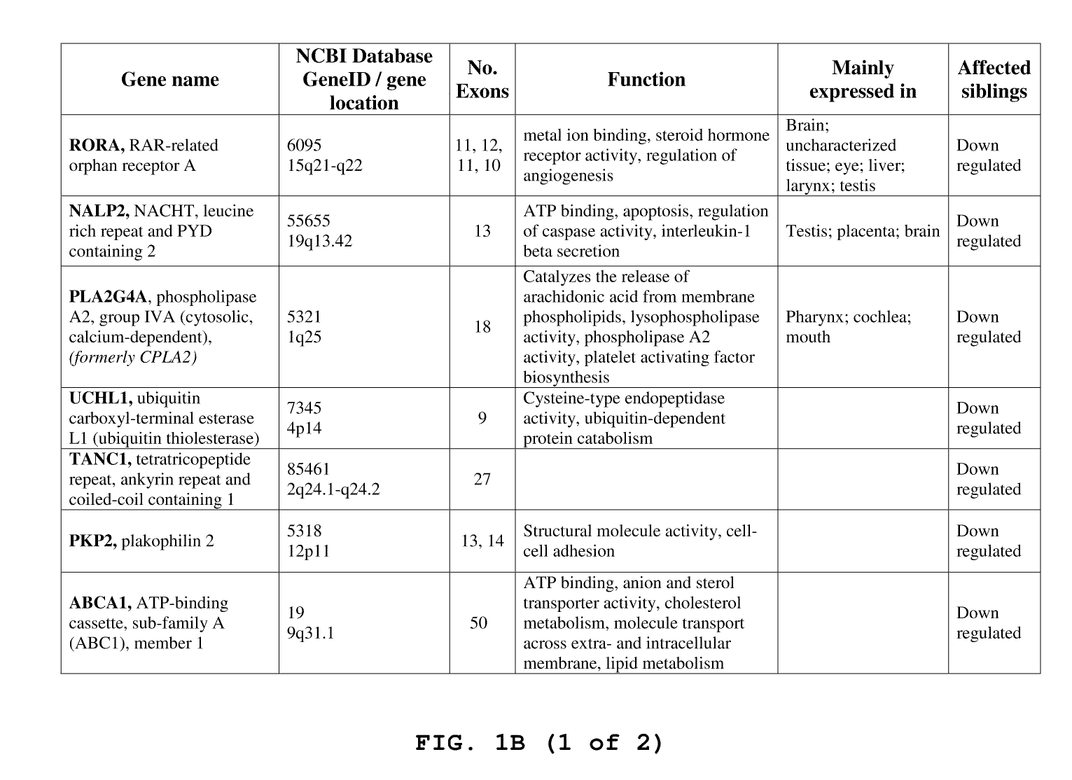

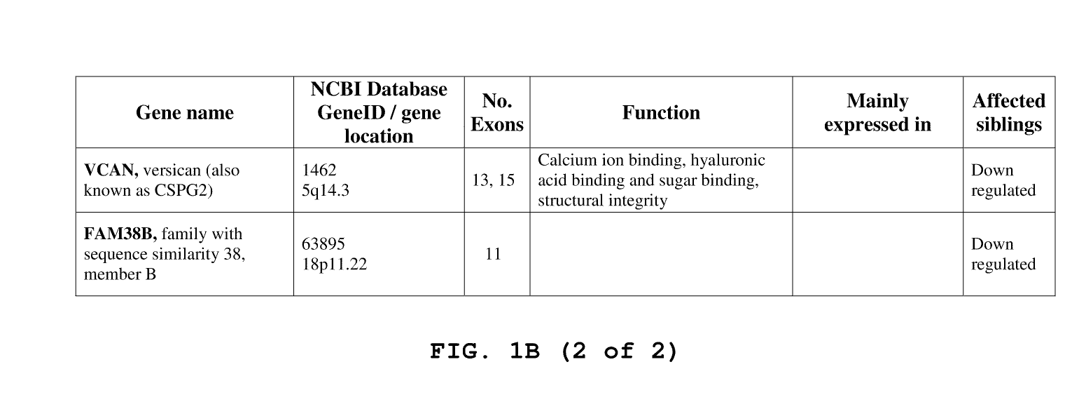

The invention is based, in part, upon the discovery that twenty-five genes and/or their gene products are associated with the development of one or more angiogenic disorders, for example, an ocular angiogenic disorder, for example, a disorder associated with choroidal neovascularization, for example, age-related macular degeneration. The twenty-five genes and/or their gene products include CRIM1, CXCR4, C5orf26, IGHG3, NALP2, PLA2G4A, IGLJ3, SHQ1, UCHL1, TANC1, PKP2, DNAJC6, C6orf105, NALP1, RGS13, CXCL13, RPS6KA2, MMP7, RORA, IL1A, ABCA1, VCAN, KIAA0888, ENPP2, and FAM38B as shown in FIGS. 1A and 1B. It is shown below that CXCL13, RPS6KA2, MMP7, IL1A, KIAA0888, ENPP2, CRIM1, CXCR4, C5orf26, IGHG3, IGLJ3, SHQ1, DNAJC6, C6orf105, NALP1, and RGS13 gene expression increases in those with one or more angiogenic disorders, for example, an ocular angiogenic disorder, for example, a disorder associated with choroidal neovascularization, for example, age-related macular degeneration, relative to controls and that RORA, NALP2, PLA2G4A, PKP2, UCHL1, TANC1, ABCA1, VCAN, and FAM38B expression decreases in those with one or more angiogenic disorders, for example, an ocular angiogenic disorder, for example, a disorder associated with choroidal neovascularization, for example, age-related macular degeneration, relative to controls.

A. Genes and Gene Products Associated with Angiogenic Disorders

A.1. CRIM1

CRIM1 is a transmembrane protein containing cysteine-rich repeats. It is believed to be developmentally regulated and it is implicated in vertebrate CNS development and organogenesis. (Kolle et al. (2000) “CRIM1, a novel gene encoding a cysteine-rich repeat protein, is developmentally regulated and implicated in vertebrate CNS development and organogenesis,” Mech Dev. 90(2):181-93.) As used herein, the term “CRIM1 gene” is understood to mean a nucleic acid sequence that is (i) at least 90%, more preferably at least 95%, and more preferably at least 98% identical to at least 75, at least 150, at least 225, at least 500, or at least 750 nucleotides in length of the known sequence for the CRIM1 gene as reported in the NCBI gene database under gene ID: 51232, gene location accession no. NC_{—}000002.10 (36436901.36631782) (available at the web site, www.ncbi.nlm.nih.gov) or a strand complementary thereto; (ii) the full length sequence of the CRIM1 gene reported in the NCBI gene database under gene ID: 51232, gene location accession no. NC_{—}000002.10 (36436901.36631782); (iii) a naturally occurring allelic variant of one of the foregoing sequences; or (iv) a nucleic acid sequence complementary to one of the foregoing sequences.

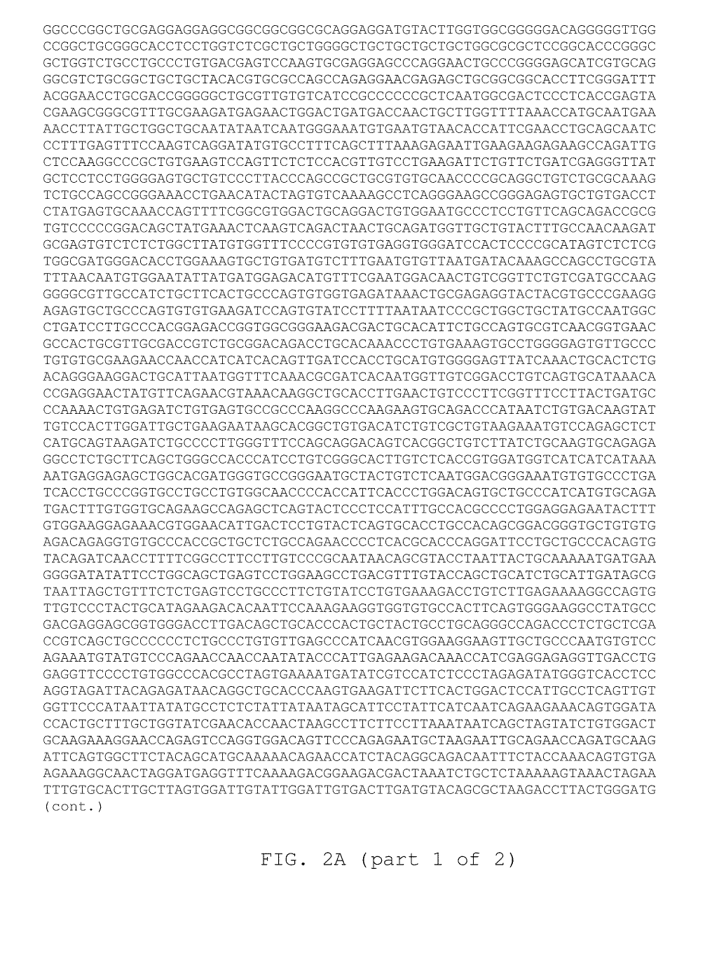

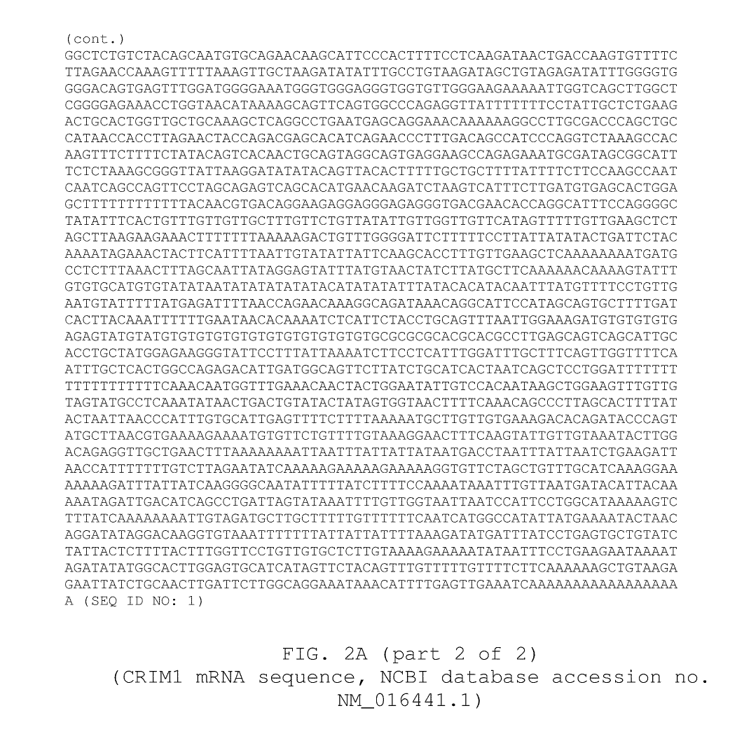

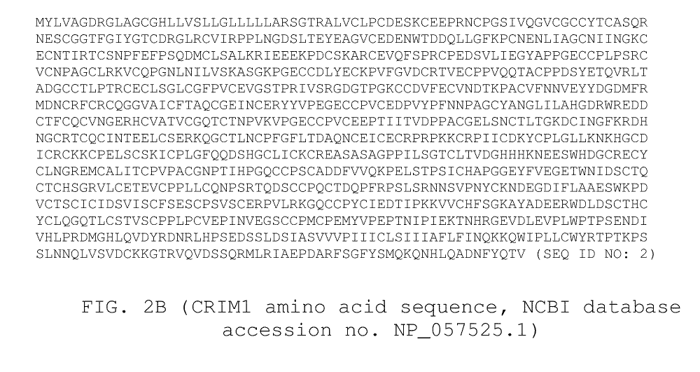

As used herein, a “CRIM1 gene product” is understood to mean (i) a nucleic acid sequence at least 75, at least 150, or at least 225 nucleotides in length that hybridizes under specific hybridization and washing conditions to the CRIM1 gene (either the sense or anti-sense sequence); (ii) a nucleic acid sequence that is at least 90%, more preferably at least 95%, and more preferably at least 98% identical to the mRNA sequence shown in FIG. 2A, or a nucleic acid sequence that hybridizes under specific hybridization and washing conditions to the sequence shown in FIG. 2A; or (iii) a peptide or protein at least 25, at least 50, or at least 75 amino acids in length that is at least 95%, more preferably at least 98%, and more preferably at least 99% identical to the amino acid sequence shown in FIG. 2B.

The nucleic acid encoding the human CRIM1 gene spans about 195 kb in length and comprises seventeen exons and sixteen introns as reported in the NCBI gene database under gene ID: 51232, gene location accession no. NC_{—}000002.10 (36436901.36631782). The CRIM1 protein itself is 1036 amino acids in length as reported in the NCBI protein database for gene ID: 51232, accession no. NP_{—}057525 (available at the web site, www.ncbi.nlm.nih.gov). Polymorphisms have been identified in the coding regions and untranslated regions of the exons, as well as in the introns and in the chromosome outside of the transcript region or regions of the CRIM1 gene. As examples of the polymorphisms in the CRIM1 gene, the NCBI SNP database (available at the web site, www.ncbi.nlm.nih.gov) reports 1374 specific polymorphic sites in the CRIM1 gene under gene ID: 51232. The mRNA sequence and the amino acid sequence of CRIM1 are set forth in FIGS. 2A and 2B, respectively.

Herein, specific hybridization and washing conditions can include high stringency conditions, for example, from at least about 31% v/v to at least about 50% v/v formamide and from at least about 0.01M to at least about 0.15M salt for hybridization, and at least about 0.01M to at least about 0.15M salt for washing conditions. Alternative stringency conditions may be applied where desired, such as medium stringency conditions including, for example, from at least about 16% v/v to at least about 30% v/v formamide and from at least about 0.5M to at least about 0.9M salt for hybridization, and at least about 0.5M to at least about 0.9M salt for washing conditions or, alternatively, low stringency conditions including, for example, from at least about 1% v/v to at least about 15% v/v formamide and from at least about 1M to at least about 2M salt for hybridization, and at least about 1M to at least about 2M salt for washing conditions. Various temperatures can be employed for each condition, for example, all conditions can be carried out at from about 30° to about 50° C., or at about 42° C. Additional examples of stringency conditions for polynucleotide hybridization are provided in Sambrook et al., Molecular Cloning: A Laboratory Manual (Cold Spring Harbor, N.Y.: Cold Spring Harbor Laboratory Press 1989) chapters 9 and 11, and in Ausubel et al., Current Protocols in Molecular Biology (N.Y.: John Wiley & Sons, Inc. 1995) sections 2.10.

In addition, herein, to determine whether a candidate protein or peptide has the requisite percentage similarity or identity to a reference polypeptide or peptide oligomer, the candidate amino acid sequence and the reference amino acid sequence are first aligned using the dynamic programming algorithm described in Smith et al. (1981), J. Mol. Biol., 147:195-7, in combination with the BLOSUM62 substitution matrix described in FIG. 2 of Henikoff et al. (1992), PNAS (USA), 89:10915-9. An appropriate value for the gap insertion penalty is −12, and an appropriate value for the gap extension penalty is −4. Computer programs performing alignments using the algorithm of Smith-Waterman and the BLOSUM62 matrix, such as the GCG program suite (Oxford Molecular Group, Oxford, England), are commercially available and widely used by those skilled in the art.

Once the alignment between the candidate and reference sequence is made, a percent similarity score may be calculated. The individual amino acids of each sequence are compared sequentially according to their similarity to each other. If the value in the BLOSUM62 matrix corresponding to the two aligned amino acids is zero or a negative number, the pairwise similarity score is zero; otherwise the pairwise similarity score is 1.0. The raw similarity score is the sum of the pairwise similarity scores of the aligned amino acids. The raw score is then normalized by dividing it by the number of amino acids in the smaller of the candidate or reference sequences. The normalized raw score is the percent similarity. Alternatively, to calculate a percent identity, the aligned amino acids of each sequence are again compared sequentially. If the amino acids are non-identical, the pairwise identity score is zero; otherwise the pairwise identity score is 1.0. The raw identity score is the sum of the identical aligned amino acids. The raw score is then normalized by dividing it by the number of amino acids in the smaller of the candidate or reference sequences. The normalized raw score is the percent identity. Insertions and deletions are ignored for the purposes of calculating percent similarity and identity. Accordingly, gap penalties are not used in this calculation, although they are used in the initial alignment.

In addition, herein, the percent identity between two nucleotide sequences can be determined, for example, by using the GAP program in the GCG software package (available at the url address gcg.com), using a NWSgapdna.CMP matrix and a gap weight of 40, 50, 60, 70, or 80 and a length weight of 1, 2, 3, 4, 5, or 6. In another embodiment, the percent identity between two nucleotide sequences is determined using the algorithm of E. Meyers and W. Miller (1988) Comput. Appl. Biosci. 4:11-17, which has been incorporated into the ALIGN program (version 2.0), using a PAM120 weight residue table, a gap length penalty of 12 and a gap penalty of 4.

A.2. CXCR4

CXCR4 is a G protein-coupled receptor (GPCR) that has multiple critical functions in normal and pathologic physiology including regulation of the metastatic behavior of mammary carcinoma and activity as a coreceptor for infection by T-tropic strains of human immunodeficiency virus-1. (Trent et al. (2003) “Lipid bilayer simulations of CXCR4 with inverse agonists and weak partial agonists,” J. Biol. Chem. 278(47): 47136-47144.) As used herein, the term “CXCR4 gene” is understood to mean a nucleic acid sequence that is (i) at least 90%, more preferably at least 95%, and more preferably at least 98% identical to at least 75, at least 150, at least 225, at least 500, or at least 750 nucleotides in length of the known sequence for the CXCR4 gene reported in the NCBI gene database under gene ID: 7852, gene location accession no. NC_{—}000002.10 (136588389.136592195, complement) or a strand complementary thereto; (ii) the full length sequence of the CXCR4 gene reported in the NCBI gene database under gene ID: 7852, gene location accession no. NC_{—}000002.10 (136588389 . . . 136592195, complement); (iii) a naturally occurring allelic variant of one of the foregoing sequences; or (iv) a nucleic acid sequence complementary to one of the foregoing sequences.

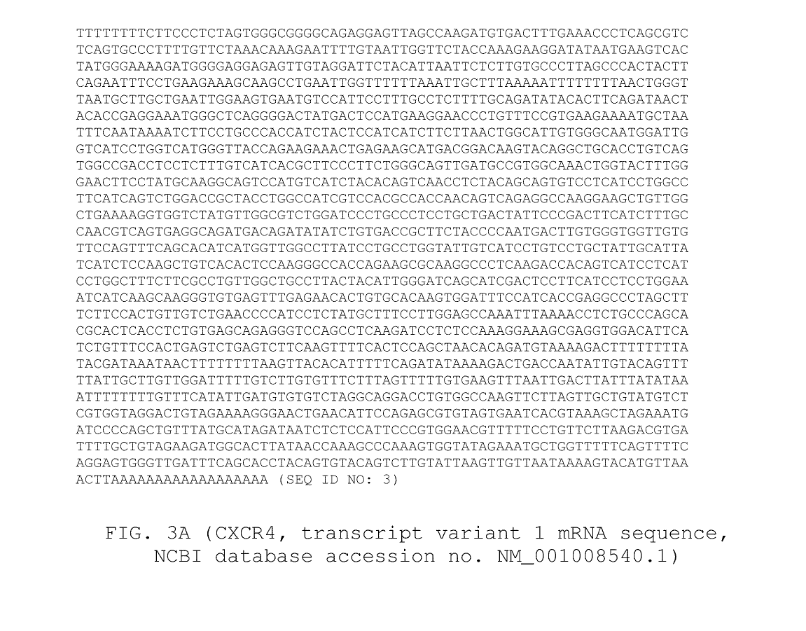

As used herein, a “CXCR4 gene product” is understood to mean (i) a nucleic acid sequence at least 75, at least 150, or at least 225 nucleotides in length that hybridizes under specific hybridization and washing conditions to the CXCR4 gene (either the sense or anti-sense sequence); (ii) a nucleic acid sequence that is at least 90%, more preferably at least 95%, and more preferably at least 98% identical to the mRNA sequence shown in one of FIGS. 3A and 3B, or a nucleic acid sequence that hybridizes under specific hybridization and washing conditions to the sequence shown in one of FIGS. 3A and 3B; or (iii) a peptide or protein at least 25, at least 50, or at least 75 amino acids in length that is at least 95%, more preferably at least 98%, and more preferably at least 99% identical to the amino acid sequence shown in one of FIGS. 3C and 3D.

The nucleic acid encoding the human CXCR4 gene spans approximately 3,807 base pairs in length as reported in the NCBI gene database under gene ID: 7852, gene location accession no. NC_{—}000002.10 (136588389 . . . 136592195, complement). The CXCR4 gene has been reported to generate two splicing transcript variants. Transcript variant 1 comprises one exon as reported in the NCBI nucleotide database under accession no. NM_{—}001008540; the protein encoded by transcript variant 1 is 356 amino acids in length as reported in the NCBI protein database under accession no. NP_{—}001008540. Transcript variant 2 comprises two exons as reported in the NCBI nucleotide database under accession no. NM_{—}003467; the protein encoded by transcript variant 2 is 352 amino acids in length as reported in the NCBI protein database under accession no. NP_{—}003458. Polymorphisms have also been identified in the coding regions and untranslated regions of the exons, as well as in the introns and in the chromosome outside of the transcript region or regions of the CXCR4 gene. As examples of the polymorphisms in the CXCR4 gene, the NCBI SNP database reports 36 specific polymorphic sites for the CXCR4 gene under gene ID: 7852. The mRNA sequences and the amino acid sequences of CXCR4 are set forth in FIGS. 3A-3B and in FIGS. 3C-3D, respectively.

A.3. C5orf26

C5orf26 encodes a small protein that has a transmembrane domain without a signal peptide motif and is believed to be a regulator of ion transport in the mitochondrial transmembrane. (Yabuta et al. (2006) “Isolation and characterization of the TIGA genes, whose transcripts are induced by growth arrest,” Nucleic Acids Res 34(17): 4878-4892.) As used herein, the term “C5orf26 gene” is understood to mean a nucleic acid sequence that is (i) at least 90%, more preferably at least 95%, and more preferably at least 98% identical to at least 75, at least 150, at least 225, or at least 500 nucleotides in length of the known sequence for the C5orf26 gene as reported in the NCBI gene database under gene ID: 114915, gene location accession no. NC_{—}000005.8 (111524125 . . . 111524816) or a strand complementary thereto; (ii) the full length sequence of the C5orf26 gene reported in the NCBI gene database under gene ID: 114915, gene location accession no. NC_{—}000005.8 (111524125.111524816); (iii) a naturally occurring allelic variant of one of the foregoing sequences; or (iv) a nucleic acid sequence complementary to one of the foregoing sequences.

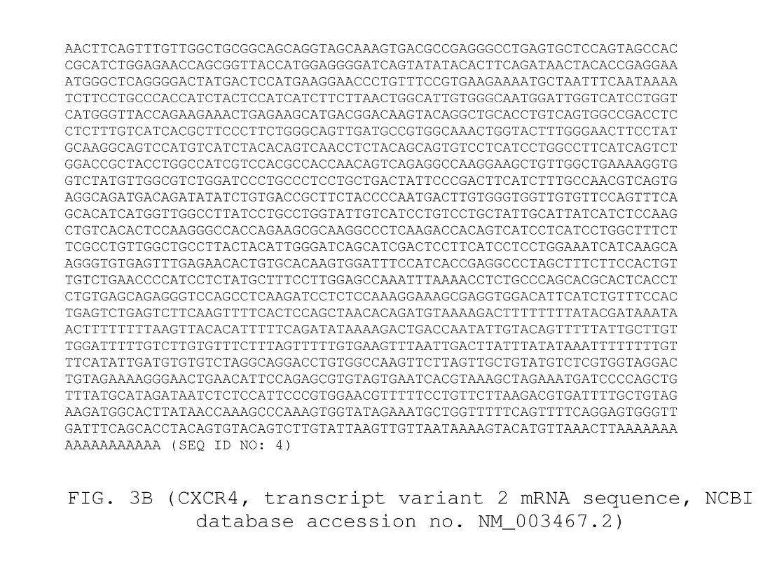

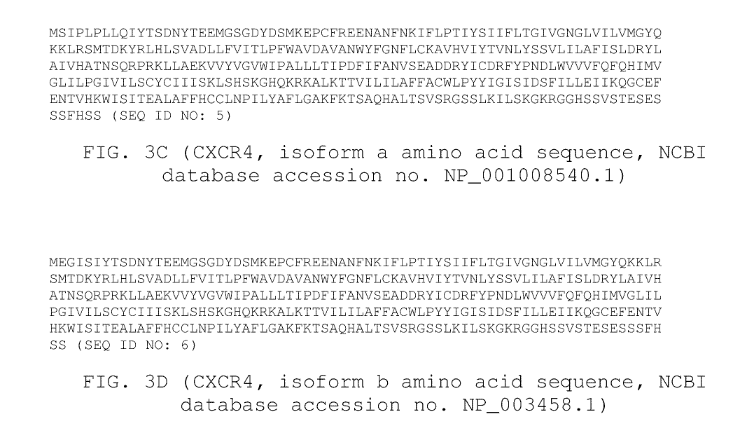

As used herein, a “C5orf26 gene product” is understood to mean (i) a nucleic acid, for example, a sequence at least 75, at least 150, or at least 225 nucleotides in length that hybridizes under specific hybridization and washing conditions to the C5orf26 gene (either the sense or anti-sense sequence); (ii) a nucleic acid sequence that is at least 90%, more preferably at least 95%, and more preferably at least 98% identical to the transcript sequence shown in FIG. 4A, or a nucleic acid sequence that hybridizes under specific hybridization and washing conditions to the sequence shown in FIG. 4A; or (iii) a peptide or protein at least 25, at least 50, or at least 75 amino acids in length that is at least 95%, more preferably at least 98%, and more preferably at least 99% identical to the amino acid sequence shown in FIG. 4B.

The nucleic acid encoding human C5orf26 gene spans approximately 692 base pairs in length as reported in the NCBI gene database for gene ID: 114915 under gene location accession no. NC_{—}000005.8 (111524125 . . . 111524816). Polymorphisms have been identified in the C5orf26 gene. As examples of the polymorphisms in the C5orf26 gene, the NCBI SNP database reports seventeen specific polymorphic sites for the C5orf26 gene under gene ID: 114915 in the corresponding SNP database. The gene transcript and amino acid sequences of C5orf26 are set forth in FIGS. 4A and 4B, respectively.

A.4. IGHG3

IGHG3 is the heavy constant domain of the human immunoglobulin gamma 3 chain. As used herein, the term “IGHG3 gene” is understood to mean a nucleic acid sequence that is (i) at least 90%, more preferably at least 95%, and more preferably at least 98% identical to at least 75, at least 150, at least 225, at least 500, or at least 750 nucleotides in length of the known sequence for the IGHG3 gene as reported in the NCBI gene database under gene ID: 3502, gene location accession no. NC_{—}000014.7 (105303296 . . . 105308787, complement) or a strand complementary thereto (ii) the full length sequence of the IGHG3 gene reported in the NCBI gene database under gene ID: 3502, gene location accession no. NC_{—}000014.7 (105303296 . . . 105308787, complement); (iii) a naturally occurring allelic variant of one of the foregoing sequences; or (iv) a nucleic acid sequence complementary to one of the foregoing sequences.

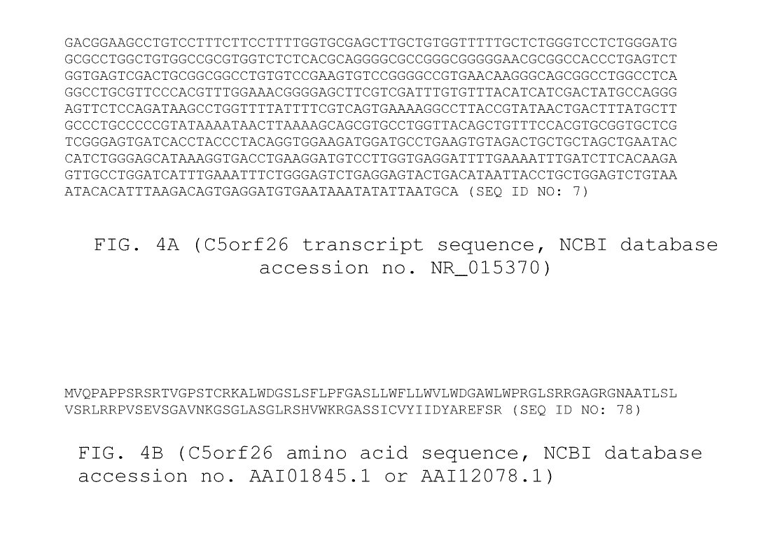

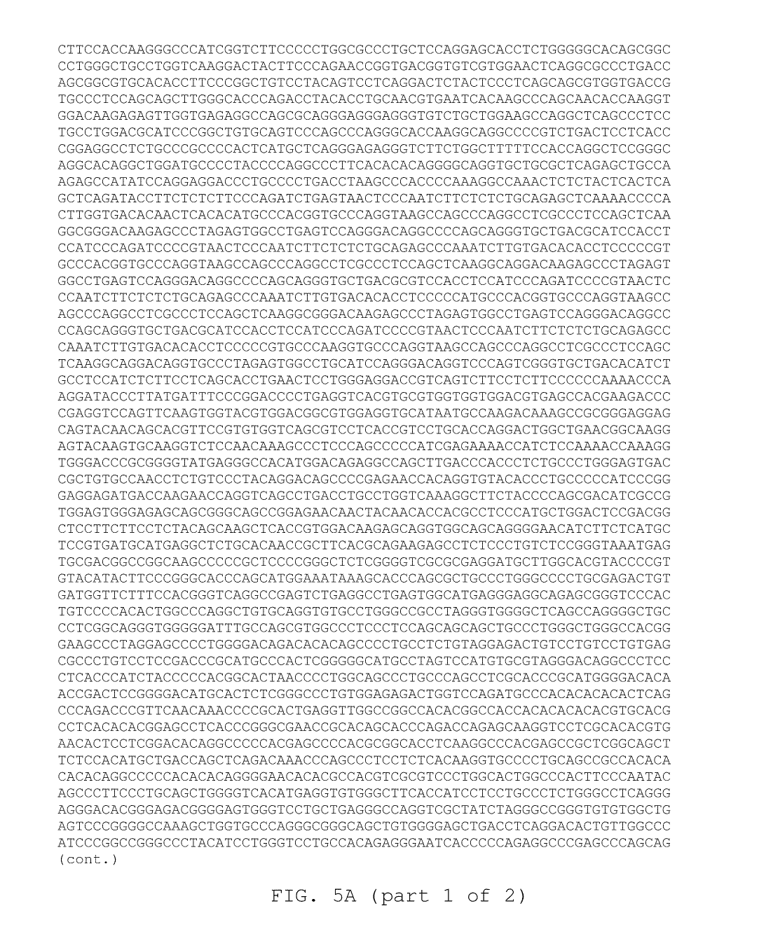

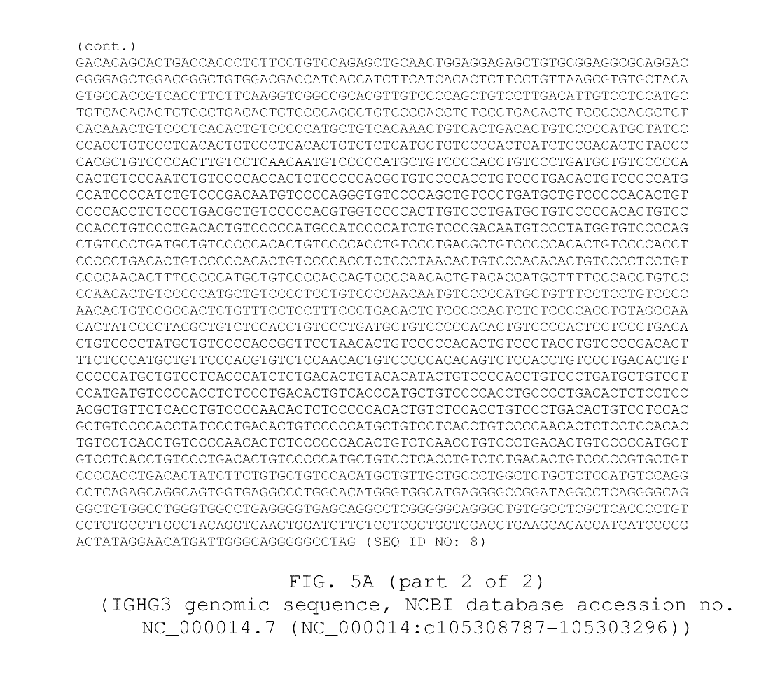

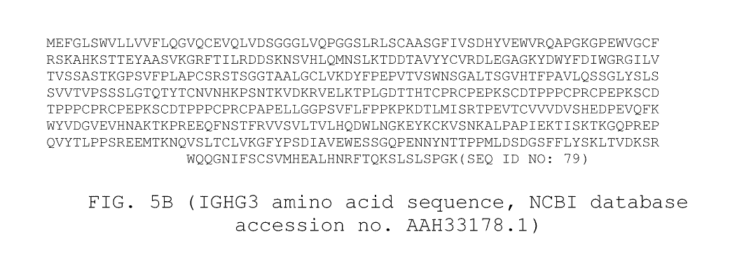

As used herein, a “IGHG3 gene product” is understood to mean (i) a nucleic acid sequence at least 75, at least 150, or at least 225 nucleotides in length that hybridizes under specific hybridization and washing conditions to the IGHG3 gene (either the sense or anti-sense sequence); (ii) a nucleic acid sequence that is at least 90%, more preferably at least 95%, and more preferably at least 98% identical to a transcript of the genomic sequence shown in FIG. 5A, or a nucleic acid sequence that hybridizes under specific hybridization and washing conditions to a transcript of the genomic sequence shown in FIG. 5A; or (iii) a peptide or protein at least 25, at least 50, or at least 75 amino acids in length that is at least 95%, more preferably at least 98%, and more preferably at least 99% identical to the amino acid sequence shown in FIG. 5B.

The nucleic acid encoding human IGHG3 gene spans about 5,492 base pairs in length as reported in the NCBI gene database under gene ID: 3502, gene location accession no. NC_{—}000014.7 (105303296 . . . 105308787, complement). It is understood that the IGHG3 gene may have many transcript variants. For example, it has been suggested that the IGHG3 gene may generate at least six transcript variants (see, e.g., the Ensembl database, available at the web site, http://www.ensembl.org/index.html, under entry ENSG00000211897). At least eleven polymorphisms have been identified in the IGHG3 gene. The genomic nucleotide and amino acid sequences of IGHG3 are set forth in FIGS. 5A and 5B, respectively.

A.5. NALP2

NALP2 is characterized by an N-terminal pyrin domain (PYD) and is believed to be involved in the activation of caspase-1 by Toll-like receptors and in protein complexes that activate proinflammatory caspases. (Tschopp et al. (2003) “NALPs: a novel protein family involved in inflammation,” Nat Rev Mol Cell Biol. 4(2):95-104.) As used herein, the term “NALP2 gene” is understood to mean a nucleic acid sequence that is (i) at least 90%, more preferably at least 95%, and more preferably at least 98% identical to at least 75, at least 150, at least 225, at least 500, or at least 750 nucleotides in length of the known sequence for the NALP2 gene as reported in the NCBI gene database under gene ID: 55655, gene location accession no. NC_{—}000019.8 (60169579 . . . 60204318) or a strand complementary thereto; (ii) the full length sequence of the NALP2 gene as reported in the NCBI gene database under gene ID: 55655, gene location accession no. NC_{—}000019.8 (60169579 . . . 60204318); (iii) a naturally occurring allelic variant of one of the foregoing sequences; or (iv) a nucleic acid sequence complementary to one of the foregoing sequences.

As used herein, a “NALP2 gene product” is understood to mean (i) a nucleic acid, for example, a sequence at least 75, at least 150, or at least 225 nucleotides in length that hybridizes under specific hybridization and washing conditions to the NALP2 gene (either the sense or anti-sense sequence); (ii) a nucleic acid sequence that is at least 90%, more preferably at least 95%, and more preferably at least 98% identical to the mRNA sequence shown in FIG. 6A, or a nucleic acid sequence that hybridizes under specific hybridization and washing conditions to the sequence shown in FIG. 6A; or (iii) a peptide or protein at least 25, at least 50, or at least 75 amino acids in length that is at least 95%, more preferably at least 98%, and more preferably at least 99% identical to the amino acid sequence shown in FIG. 6B.

The nucleic acid encoding human NALP2 gene spans approximately 34,740 base pairs in length and contains thirteen exons and twelve introns as reported in the NCBI gene database under gene ID: 55655, gene location accession no. NC_{—}000019.8 (60169579 . . . 60204318). The NALP2 protein itself is 1,062 amino acids in length as reported in the NCBI protein database under accession no. NP_{—}060322. It is understood that the NALP2 gene may have many transcript variants. For example, it has been suggested that the NALP2 gene may generate at least 10 transcript variants (see, e.g. the ECGENE database, available at the web site, http://genome.ewha.ac.kr/ECgene/, under entry H19C1617). In addition, polymorphisms have also been identified in the coding regions and untranslated regions of the exons, as well as in the introns and in the chromosome outside of the transcript region or regions of the NALP2 gene. As examples of the polymorphisms in the NALP2 gene, the NCBI SNP database reports 486 specific polymorphic sites for the NALP2 gene under gene ID: 55655. The mRNA sequence and the amino acid sequence of NALP2 are set forth in FIGS. 6A and 6B, respectively.

A.6. PLA2G4A

PLA2G4A is understood to be involved in calcium ion binding, lysophospholipase activity, and platelet activating factor biosynthesis. In particular, PLAG4A is involved in catalyzing the cleavage of arachidonic acid from the sn-2 position of phospholipids. (Angelika et al. (1998), “Identification of the Phosphorylation Sites of Cytosolic Phospholipase A2 in Agonist-stimulated Human Platelets and HeLa Cells,” J Biol Chem 273(8): 4449-4458.) As used herein, the term “PLA2G4A gene” is understood to mean a nucleic acid sequence that is (i) at least 90%, more preferably at least 95%, and more preferably at least 98% identical to at least 75, at least 150, at least 225, at least 500, or at least 750 nucleotides in length of the known sequence for the PLA2G4A gene as reported in the NCBI gene database under gene ID: 5321, gene location accession no. NC_{—}000001.9 (185064655 . . . 185224736) or a strand complementary thereto; (ii) the full length sequence of the PLA2G4A gene reported in the NCBI gene database under gene ID: 5321, gene location accession no. NC_{—}000001.9 (185064655 . . . 185224736); (iii) a naturally occurring allelic variant of one of the foregoing sequences; or (iv) a nucleic acid sequence complementary to one of the foregoing sequences.

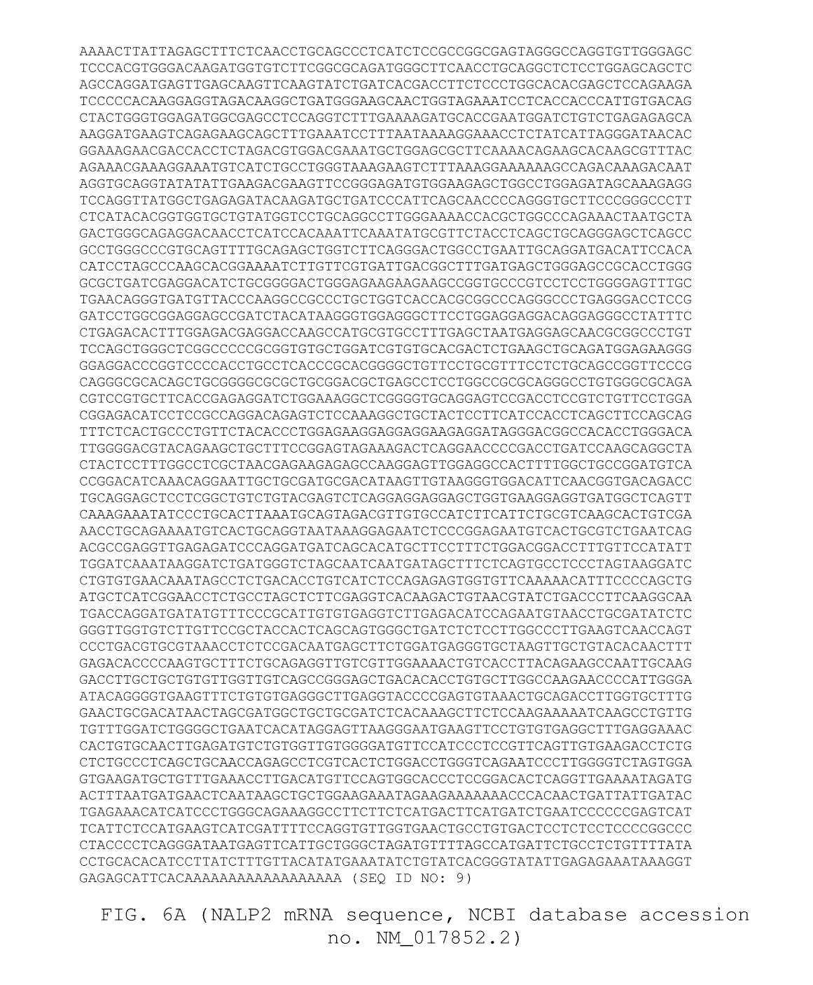

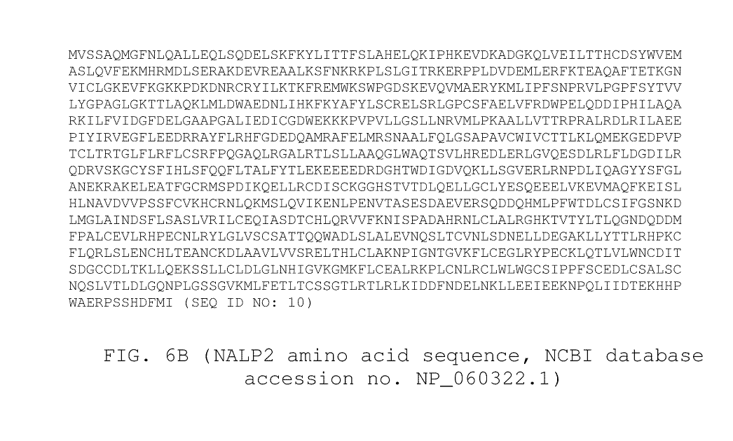

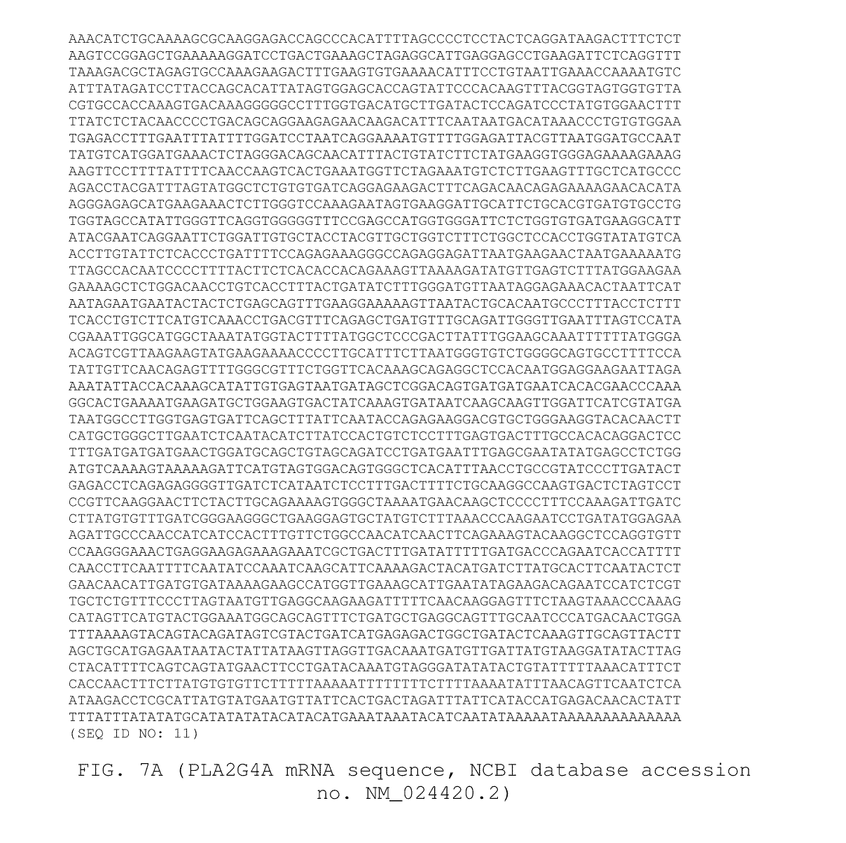

As used herein, a “PLA2G4A gene product” is understood to mean (i) a nucleic acid, for example, a sequence at least 75, at least 150, or at least 225 nucleotides in length that hybridizes under specific hybridization and washing conditions to the PLA2G4A gene (either the sense or anti-sense sequence); (ii) a nucleic acid sequence that is at least 90%, more preferably at least 95%, and more preferably at least 98% identical to the mRNA sequence shown in FIG. 7A, or a nucleic acid sequence that hybridizes under specific hybridization and washing conditions to the sequence shown in FIG. 7A; or (iii) a peptide or protein at least 25, at least 50, or at least 75 amino acids in length that is at least 95%, more preferably at least 98%, and more preferably at least 99% identical to the amino acid sequence shown in FIG. 7B.

The nucleic acid encoding human PLA2G4A gene spans about 160 kb in length and comprises eighteen exons and seventeen introns as reported in the NCBI gene database under gene ID: 5321, gene location accession no. NC_{—}000001.9(185064655 . . . 185224736). The PLA2G4A protein itself is 749 amino acids in length as reported in the NCBI protein database under accession no. NP_{—}077734. Polymorphisms have been identified in the coding regions and untranslated regions of the exons, as well as in the introns and in the chromosome outside of the transcript region or regions of the PLA2G4A gene. As examples of the polymorphisms in the PLA2G4A gene, the NCBI SNP database reports 1417 specific polymorphic sites in the PLA2G4A gene under gene ID: 5321. The mRNA sequence and the amino acid sequence of PLA2G4A are set forth in FIGS. 7A and 7B, respectively.

A.7. IGLJ3

IGLJ3 is a short genomic sequence identified as immunoglobulin lambda joining 3. The nucleic acid encoding human IGLJ3 spans 38 base pairs in length as reported in the NCBI gene database under gene ID: 28831, gene location accession no. NC_{—}000022.9 (21577168 . . . 21577205). As used herein, the term “IGLJ3 gene” is understood to mean a nucleic acid sequence that is (i) at least 90%, more preferably at least 95%, and more preferably at least 98% identical to at least 10, at least 20, or at least 30 nucleotides in length of the known sequence for IGLJ3 as reported in the NCBI gene database under gene ID: 28831, gene location accession no. NC_{—}000022.9 (21577168 . . . 21577205) or a strand complementary thereto; (ii) the full length sequence of the IGLJ3 gene reported in the NCBI gene database under gene ID: 28831, gene location accession no. NC_{—}000022.9 (21577168 . . . 21577205); (iii) a naturally occurring allelic variant of one of the foregoing sequences; or (iv) a nucleic acid sequence complementary to one of the foregoing sequences.

As used herein, an “IGLJ3 gene product” is understood to mean (i) a nucleic acid, for example, a sequence at least 10, at least 20, or at least 30 nucleotides in length that hybridizes under specific hybridization and washing conditions to the IGLJ3 gene (either the sense or anti-sense sequence); (ii) a nucleic acid sequence that is at least 90%, more preferably at least 95%, and more preferably at least 98% identical to a transcript of the genomic sequence shown in FIG. 8, or a nucleic acid sequence that hybridizes under specific hybridization and washing conditions to a transcript of the genomic sequence shown in FIG. 8; or (iii) a peptide at least 6, at least 8, or at least 10 amino acids in length that corresponds to at least a portion of the translated 38 base pair nucleic acid sequence set forth in FIG. 8.

A.8. RGS13

RGS13 is a member of Regulator of G protein-signaling (RGS) proteins that attenuate G protein-mediated pathways by acting as GTPase-activating proteins (GAPs) for G-alpha subunits. It is understood that RGS13 may regulate G protein-mediated processes in the lung and immune system. (Johnson et al. (2002), “Functional characterization of the G protein regulator RGS13,” J. Biol. Chem. 277(19):16768-74.) As used herein, the term “RGS13” is understood to mean a nucleic acid sequence that is (i) at least 90%, more preferably at least 95%, and more preferably at least 98% identical to at least 75, at least 150, at least 225, at least 500, or at least 750 nucleotides in length of the known sequence for the RGS13 gene as reported in the NCBI gene database under gene ID: 6003, gene location accession no. NC_{—}000001.9 (190871905 . . . 190896013) or a strand complementary thereto; (ii) the full length sequence of the RGS13 gene as reported in the NCBI gene database under gene ID: 6003, gene location accession no. NC_{—}000001.9 (190871905 . . . 190896013); (iii) a naturally occurring allelic variant of one of the foregoing sequences; or (iv) a nucleic acid sequence complementary to one of the foregoing sequences.

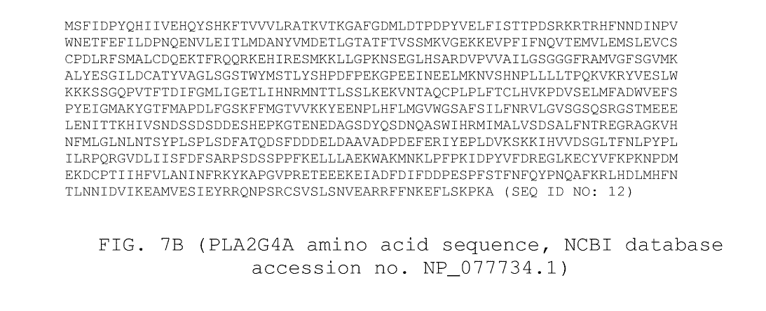

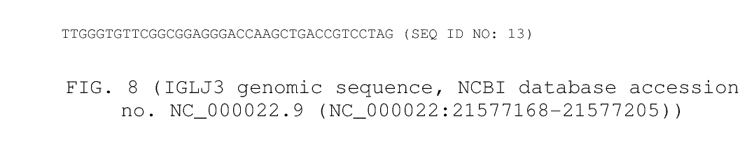

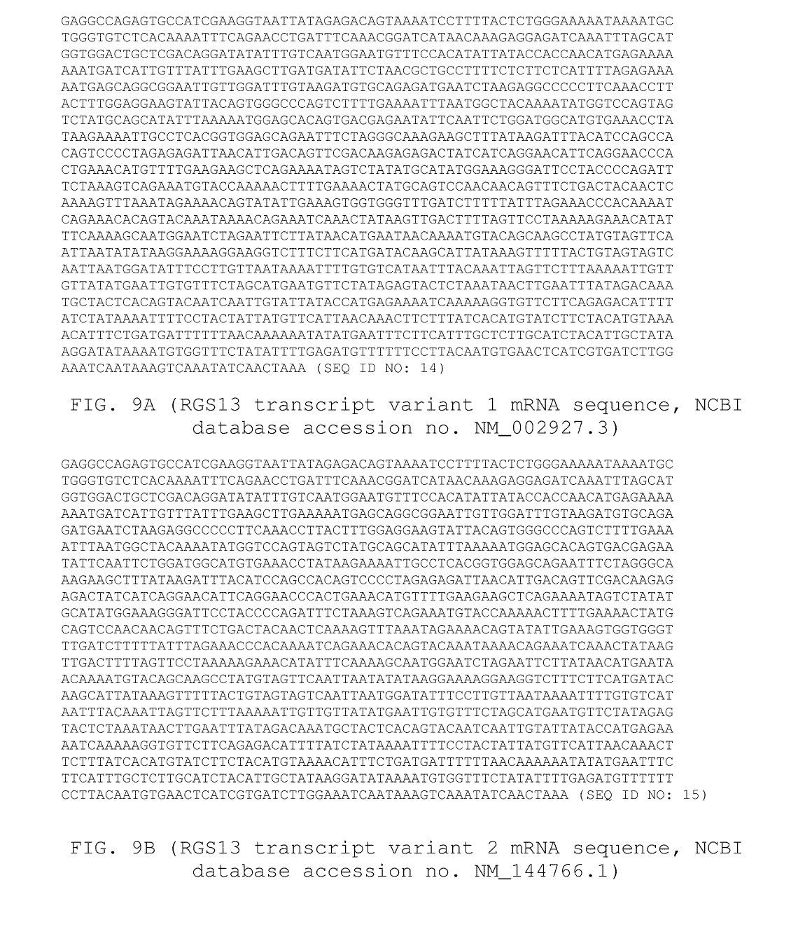

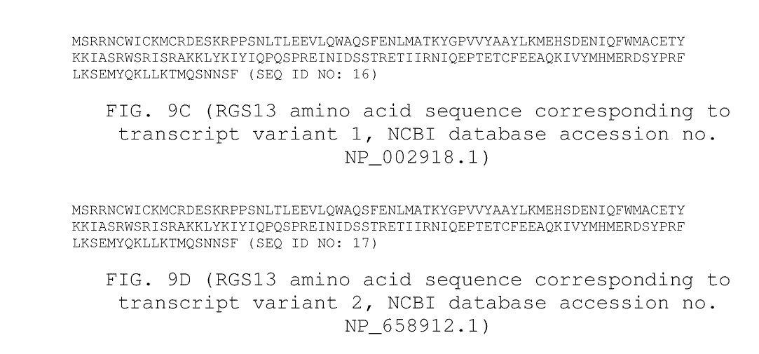

As used herein, a “RGS13 gene product” is understood to mean (i) a nucleic acid, for example, a sequence at least 75, at least 150, or at least 225 nucleotides in length that hybridizes under specific hybridization and washing conditions to the RGS13 gene (either the sense or anti-sense sequence); (ii) a nucleic acid sequence that is at least 90%, more preferably at least 95%, and more preferably at least 98% identical to the mRNA sequence shown in one of FIGS. 9A and 9B, or a nucleic acid sequence that hybridizes under specific hybridization and washing conditions to the sequence shown in one of FIGS. 9A and 9B; or (iii) a peptide or protein at least 25, at least 50, or at least 75 amino acids in length that is at least 95%, more preferably at least 98%, and more preferably at least 99% identical to the amino acid sequence shown in one of FIGS. 9C and 9D.

The nucleic acid encoding human RGS13 gene spans about 24,109 base pairs in length as reported in the NCBI gene database under gene ID: 6003, gene location accession no. NC_{—}000001.9 (190871905 . . . 190896013). The RGS13 gene has been reported to generate two splicing transcript variants. Transcript variant 1 comprises seven exons as reported in the NCBI nucleotide database under accession no. NM_{—}002927; the protein encoded by transcript variant 1 is 159 amino acids in length as reported in the NCBI protein database under accession no. NP_{—}002918. Transcript variant 2 comprises six exons as reported in the NCBI nucleotide database under accession no. NM_{—}144766; the protein encoded by transcript variant 2 is 159 amino acids in length as reported in the NCBI protein database under accession no. NP_{—}658912, and has the same amino acid sequence as the protein encoded by transcript 1. It is understood that the RGS13 gene may have more transcript variants. For example, it has been suggested that the RGS13 gene may generate at least six transcript variants (see the ECGENE database, available at the web site, http://genome.ewha.ac.kr/ECgene/, under entry H1C26175.) In addition, polymorphisms have been identified in the coding regions and untranslated regions of the exons, as well as in the introns and in the chromosome outside of the transcript region or regions of the RGS13 gene. As examples of the polymorphisms in the RGS13 gene, the NCBI SNP database reports 292 specific polymorphic sites in the RGS13 gene for gene ID: 6003. The mRNA sequences and the amino acid sequences of PLA2G4A are set forth in FIGS. 9A-9B and in FIGS. 9C-9D, respectively.

A.9. CXCL13

CXCL13 is a small cytokine belonging to the CXC chemokine family. CXCL13 is selectively chemotactic for B cells and can elicit its effect by interacting with chemokine receptor CXCR5. CXCL13 and its receptor CXCR5 control the organization of B cells within follicles of lymphoid tissues. (Ansel et al. (2002) “CXCL13 is required for B1 cell homing, natural antibody production, and body cavity immunity,” Immunity 16: 67-76.) As used herein, the term “CXCL13 gene” is understood to mean a nucleic acid sequence that is (i) at least 90%, more preferably at least 95%, and more preferably at least 98% identical to at least 75, at least 150, at least 225, at least 500, or at least 750 nucleotides in length of the known sequence for the CXCL13 gene as reported in the NCBI gene database under gene ID: 10563, gene location accession no. NC_{—}000004.10 (78651931 . . . 78752010) or a strand complementary thereto; (ii) the full length sequence of the CXCL13 gene as reported in the NCBI gene database under gene ID: 10563, gene location accession no. NC_{—}000004.10 (78651931 . . . 78752010); (iii) a naturally occurring allelic variant of one of the foregoing sequences; or (iv) a nucleic acid sequence complementary to one of the foregoing sequences.

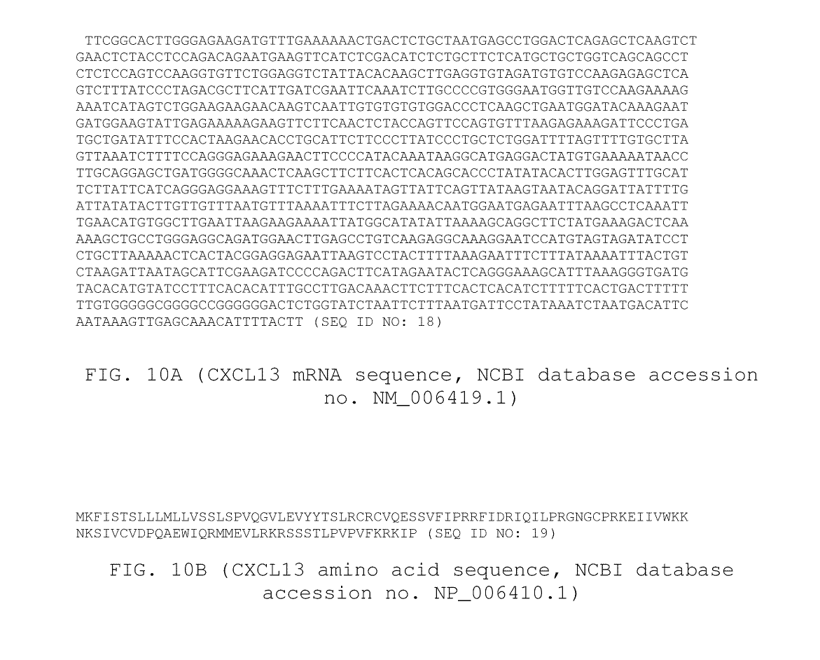

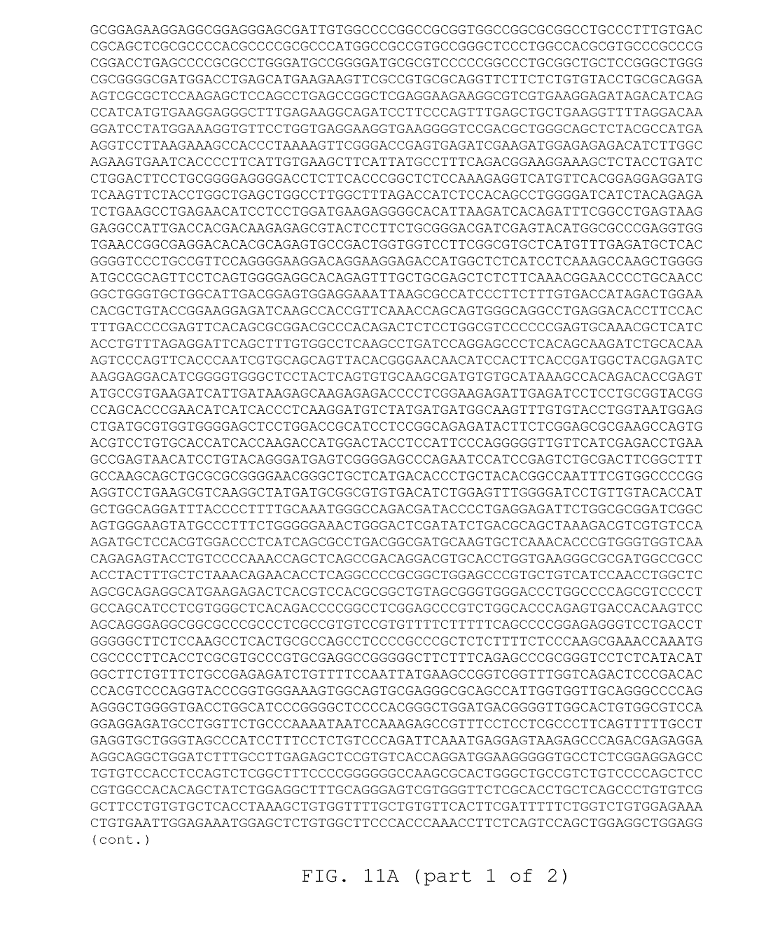

As used herein, a “CXCL13 gene product” is understood to mean (i) a nucleic acid, for example, a sequence at least 75, at least 150, or at least 225 nucleotides in length that hybridizes under specific hybridization and washing conditions to the CXCL13 gene (either the sense or anti-sense sequence); (ii) a nucleic acid sequence that is at least 90%, more preferably at least 95%, and more preferably at least 98% identical to the mRNA sequence shown in FIG. 10A, or a nucleic acid sequence that hybridizes under specific hybridization and washing conditions to the sequence shown in FIG. 10A; or (iii) a peptide or protein at least 25, at least 50, or at least 75 amino acids in length that is at least 95%, more preferably at least 98%, and more preferably at least 99% identical to the amino acid sequence shown in FIG. 10B.

The nucleic acid encoding human CXCL13 gene spans approximately 100 kb in length and comprises five exons and four introns as reported in the NCBI gene database under gene ID: 10563, gene location accession no. NC_{—}000004.10 (78651931 . . . 78752010). The CXCL13 protein itself is 109 amino acids in length as reported in the NCBI protein database under accession no. NP_{—}006410. It is understood that the CXCL13 gene may have transcript variants. For example, it has been suggested that the RGS13 gene may generate at least two transcript variants (see the ECGENE database, available at the web site, http://genome.ewha.ac.kr/ECgene/, under entry H4C7790). In addition, polymorphisms have been identified in untranslated regions of the exons, as well as in the introns and in the chromosome outside of the transcript region or regions of the CXCL13 gene. As examples of the polymorphisms in the CXCL13 gene, the NCBI SNP database reports 555 specific polymorphic sites for the CXCL13 gene under gene ID: 10563. The mRNA sequence and the amino acid sequence of CXCL13 are set forth in FIGS. 10A and 10B, respectively.

A.10. RPS6KA2

RPS6KA2 is a serine-threonine kinase in the mitogen-activated protein kinase pathway and is believed to be a putative tumor suppressor gene. (Bignone et al. (2007), “RPS6KA2, a putative tumour suppressor gene at 6q27 in sporadic epithelial ovarian cancer,” Oncogene 26(5):683-700.) As used herein, the term “RPS6KA2 gene” is understood to mean a nucleic acid sequence that is (i) at least 90%, more preferably at least 95%, and more preferably at least 98% identical to at least 75, at least 150, at least 225, at least 500, or at least 750 nucleotides in length of the known sequence for the RPS6KA2 gene as reported in the NCBI gene database under gene ID: 6196, gene location accession no. NC_{—}000006.10 (166742844 . . . 167195761, complement) or a strand complementary thereto; (ii) the full length sequence of the RPS6KA2 gene as reported in the NCBI gene database under gene ID: 6196, gene location accession no. NC_{—}000006.10 (166742844 . . . 167195761, complement); (iii) a naturally occurring allelic variant of one of the foregoing sequences; or (iv) a nucleic acid sequence complementary to one of the foregoing sequences.

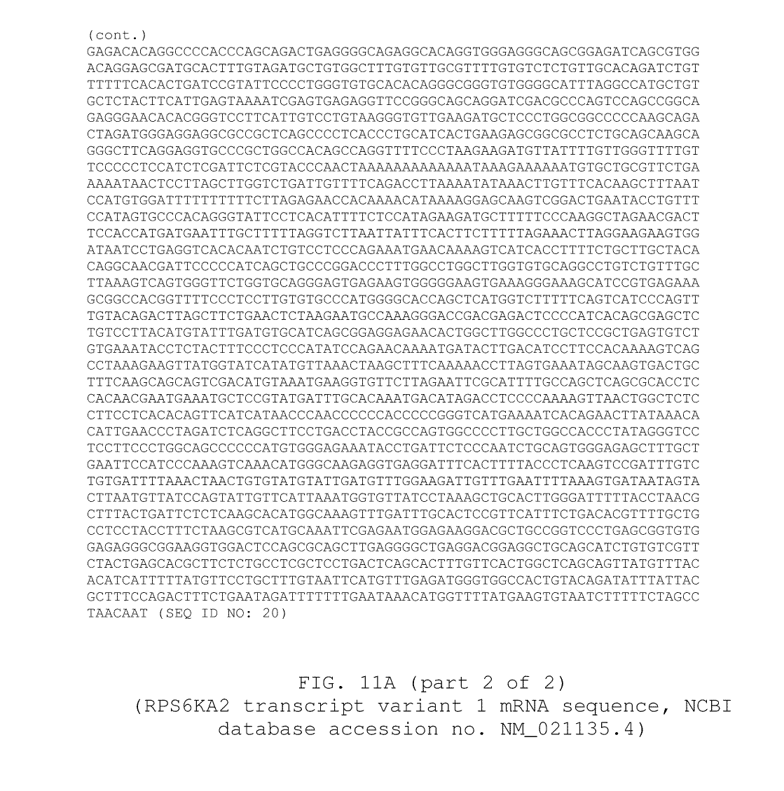

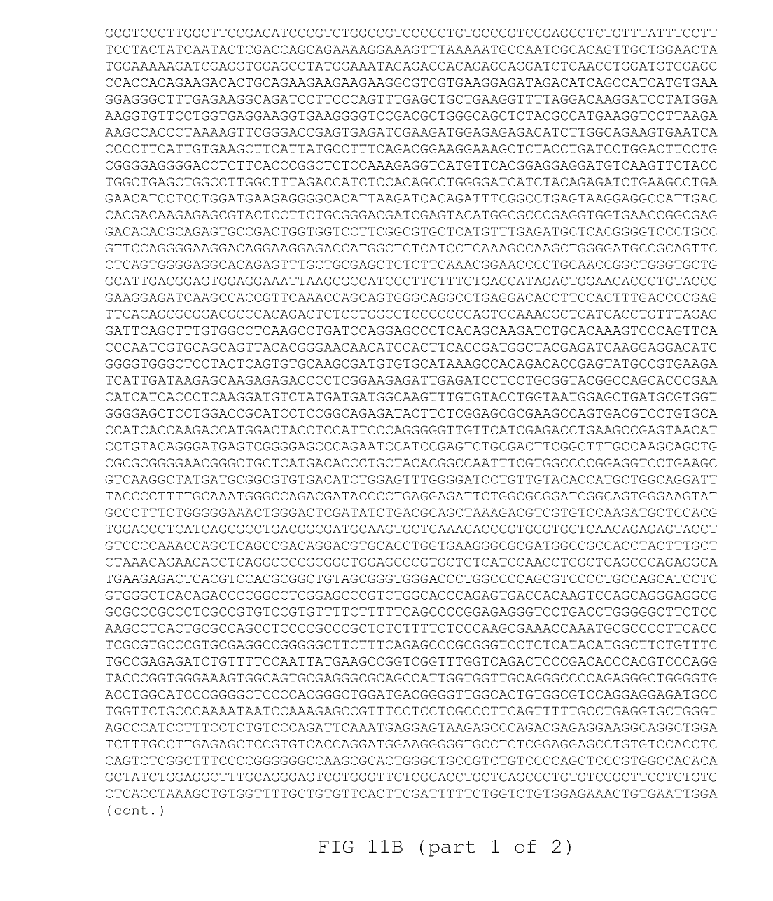

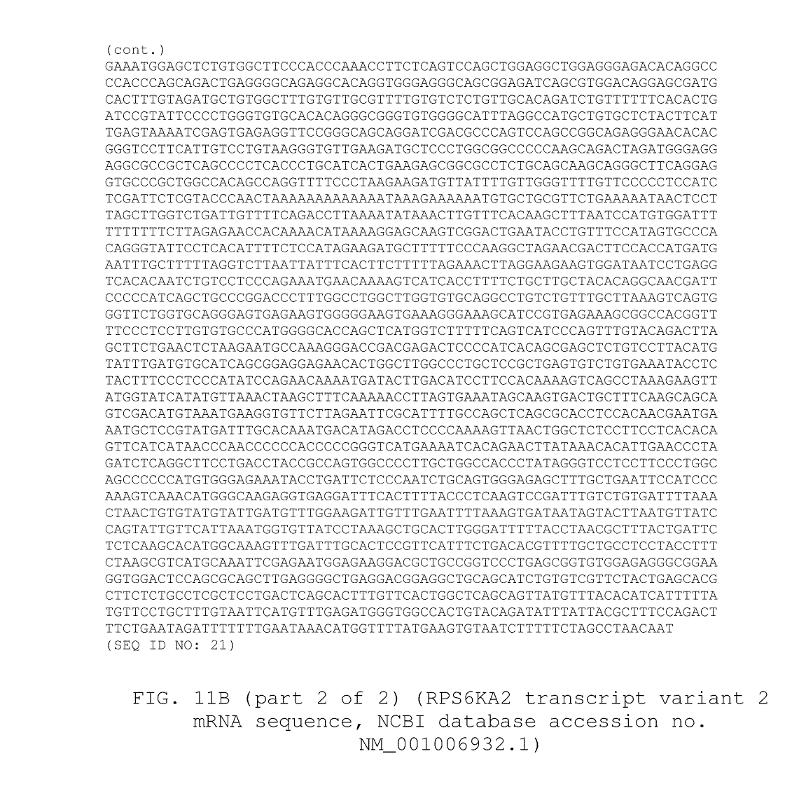

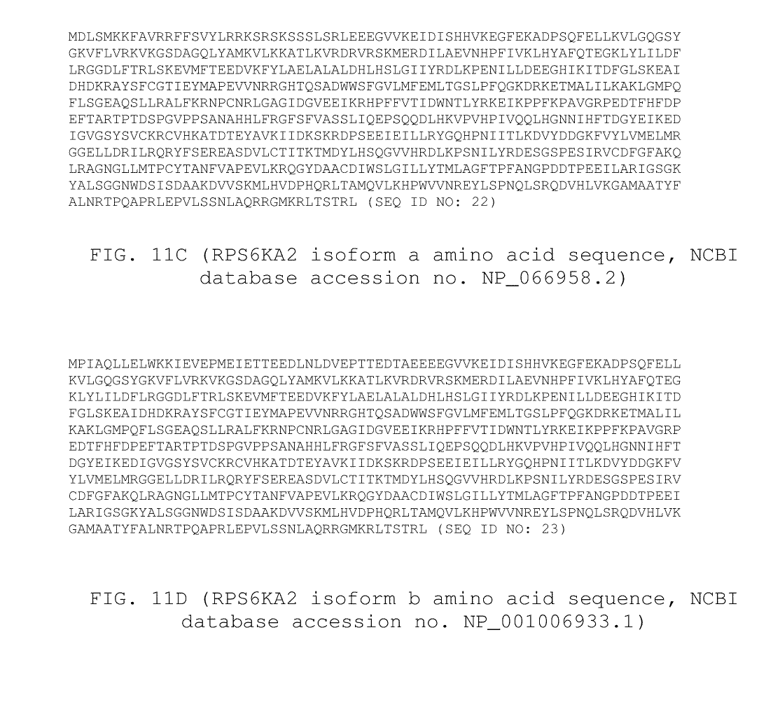

As used herein, a “RPS6KA2 gene product” is understood to mean (i) a nucleic acid, for example, a sequence at least 75, at least 150, or at least 225 nucleotides in length that hybridizes under specific hybridization and washing conditions to the RPS6KA2 gene (either the sense or anti-sense sequence); (ii) a nucleic acid sequence that is at least 90%, more preferably at least 95%, and more preferably at least 98% identical to the mRNA sequence shown in one of FIGS. 11A and 11B, or a nucleic acid sequence that hybridizes under specific hybridization and washing conditions to the sequence shown in one of FIGS. 11A and 11B; or (iii) a peptide or protein at least 25, at least 50, or at least 75 amino acids in length that is at least 95%, more preferably at least 98%, and more preferably at least 99% identical to the amino acid sequence shown in one of FIGS. 11C and 11D.

The nucleic acid encoding human RPS6KA2 gene spans approximately 453 kb in length as reported in the NCBI gene database under gene ID: 6196, gene location accession no. NC_{—}000006.10 (166742844 . . . 167195761, complement). The RPS6KA2 gene has been reported to generate two splicing transcript variants. Transcript variant 1 comprises 21 exons as reported in the NCBI nucleotide database under accession no. NM_{—}021135; the protein encoded by transcript variant 1 is 733 amino acids in length as reported in the NCBI protein database under accession no. NP_{—}066958. Transcript variant 2 comprises 22 exons as reported in the NCBI nucleotide database under accession no. NM_{—}001006932; the protein encoded by transcript variant 2 is 741 amino acids in length as reported in the NCBI protein database under accession no. NP_{—}001006933. It is understood that the RPS6KA2 gene may have more transcript variants. For example, it has been suggested that the RPS6KA2 gene may generate at least thirty-one transcript variants (see the ECGENE database, available at the web site, http://genome.ewha.ac.kr/ECgene/, under entry:H6C19508). In addition, polymorphisms have also been identified in the coding regions and untranslated regions of the exons, as well as in the introns and in the chromosome outside of the transcript region or regions of the RPS6KA2 gene. As examples of the polymorphisms in the RPS6KA2 gene, the NCBI SNP database reports 4,374 specific polymorphic sites for the RPS6KA2 gene under gene ID: 6196. The mRNA sequences and the amino acid sequences of RPS6KA2 are set forth in FIGS. 11A-11B and in FIGS. 11C-11D, respectively.

A.11. MMP7

MMP7 is involved in timely breakdown of extracellular matrix, which is essential for embryonic development, morphogenesis, reproduction, and tissue resorption and remodeling. (Massova et al. (1998) “Matrix metalloproteinases: structures, evolution, and diversification,” FASEB J. 12(12):1075-95.) As used herein, the term “MMP7 gene” is understood to mean a nucleic acid sequence that is (i) at least 90%, more preferably at least 95%, and more preferably at least 98% identical to at least 75, at least 150, at least 225, at least 500, or at least 750 nucleotides in length of the known sequence for the MMP7 gene as reported in the NCBI gene database under gene ID: 4316, gene location accession no. NC_{—}000011.8 (101896449 . . . 101906688, complement) or a strand complementary thereto; (ii) the full length sequence of the MMP7 gene as reported in the NCBI gene database under gene ID: 4316, gene location accession no. NC_{—}000011.8 (101896449 . . . 101906688, complement); (iii) a naturally occurring allelic variant of one of the foregoing sequences; or (iv) a nucleic acid sequence complementary to one of the foregoing sequences.

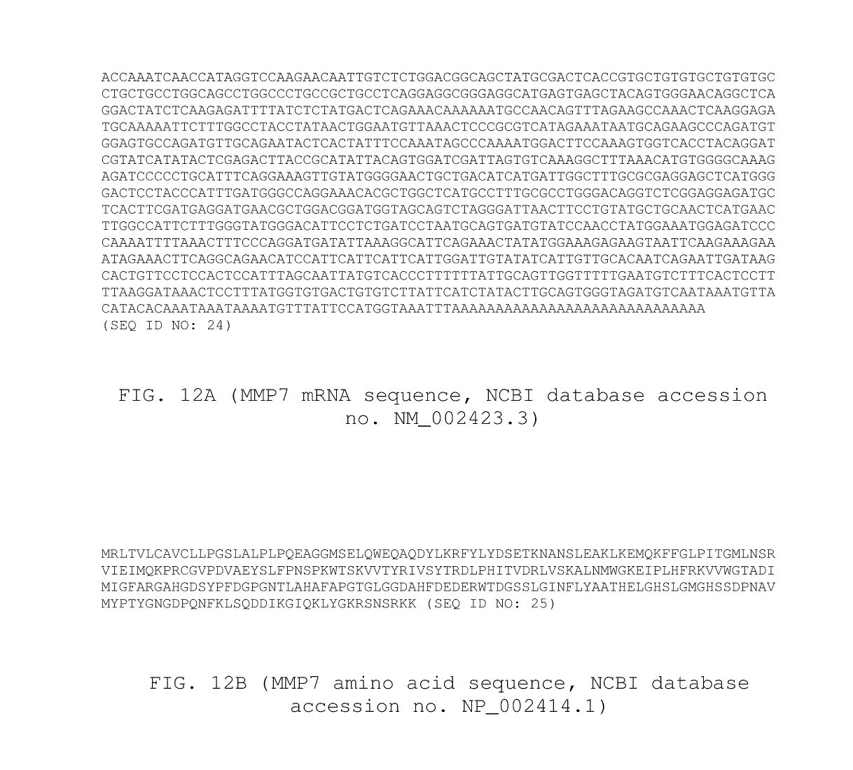

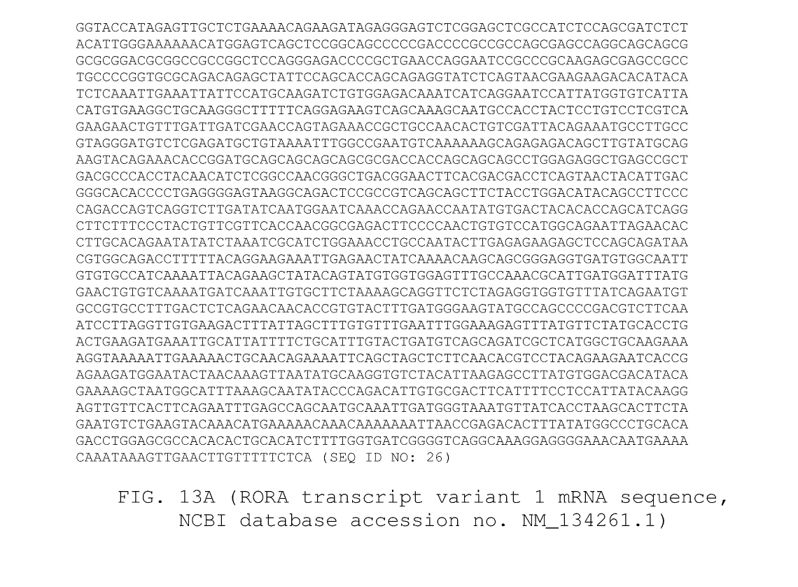

As used herein, a “MMP7 gene product” is understood to mean (i) a nucleic acid, for example, a sequence at least 75, at least 150, or at least 225 nucleotides in length that hybridizes under specific hybridization and washing conditions to the MMP7 gene (either the sense or anti-sense sequence); (ii) a nucleic acid sequence that is at least 90%, more preferably at least 95%, and more preferably at least 98% identical to the mRNA sequence shown in FIG. 12A, or a nucleic acid sequence that hybridizes under specific hybridization and washing conditions to the sequence shown in FIG. 12A; or (iii) a peptide or protein at least 25, at least 50, or at least 75 amino acids in length that is at least 95%, more preferably at least 98%, and more preferably at least 99% identical to the amino acid sequence shown in FIG. 12B.

The nucleic acid encoding human MMP7 gene spans 10,240 base pairs in length and comprises six exons and five introns as reported in the NCBI gene database under gene ID: 4316, gene location accession no. NC_{—}000011.8 (101896449 . . . 101906688, complement), and under accession no. NM_{—}002423. The MMP7 protein itself is 267 amino acids in length as reported in the NCBI protein database under accession no. NP_{—}002414. Polymorphisms have been identified in the coding regions and untranslated regions of the exons, as well as in the introns and in the chromosome outside of the transcript region or regions of the MMP7 gene. As examples of the polymorphisms in the MMP7 gene, the NCBI SNP database reports 177 specific polymorphic sites in the MMP7 gene under gene ID: 4316. The mRNA sequence and the amino acid sequence of MMP7 are set forth in FIGS. 12A and 12B, respectively.

A.12. RORA

RORA is understood to be a nuclear receptor involved in many pathophysiological processes such as cerebellar ataxia, inflammation, atherosclerosis and angiogenesis. (Chauvet et al. (2004) “The gene encoding human retinoic acid-receptor-related orphan receptor a is a target for hypoxia-inducible factor 1,” Biochem J 384(1):79-85.) As used herein, the term “RORA gene” is understood to mean a nucleic acid sequence that is (i) at least 90%, more preferably at least 95%, and more preferably at least 98% identical to at least 75, at least 150, at least 225, at least 500, or at least 750 nucleotides in length of the known sequence for the RORA gene as reported in the NCBI gene database under gene ID: 6095, gene location accession no. NC_{—}000015.8 (58576755 . . . 59308794, complement) or a strand complementary thereto; (ii) the full length sequence of the RORA gene reported in the NCBI gene database under gene ID: 6095, gene location accession no. NC_{—}000015.8 (58576755 . . . 59308794, complement); (iii) a naturally occurring allelic variant of one of the foregoing sequences; or (iv) a nucleic acid sequence complementary to one of the foregoing sequences.

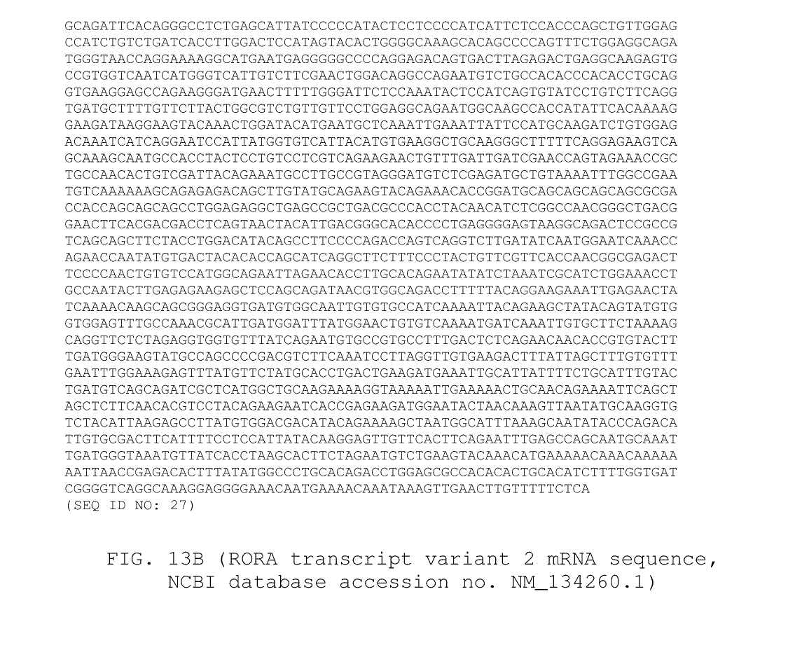

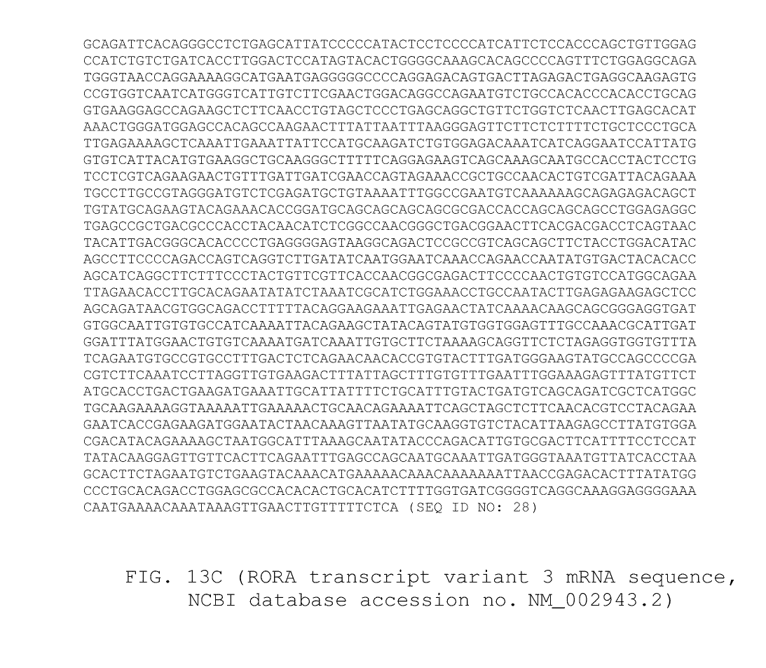

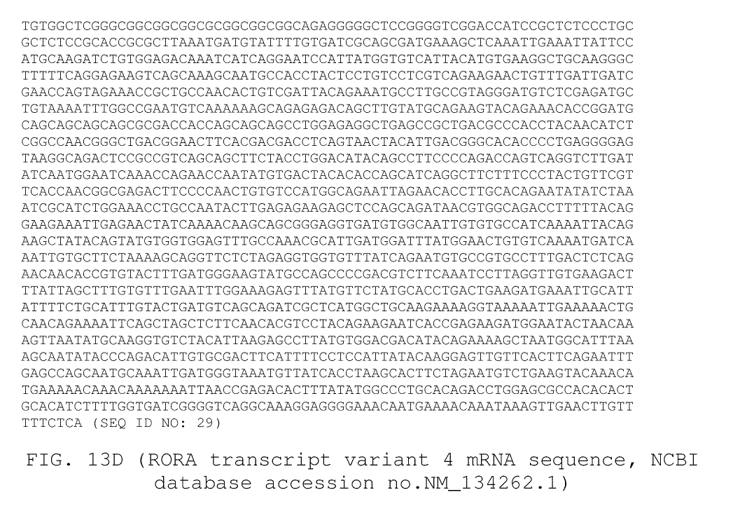

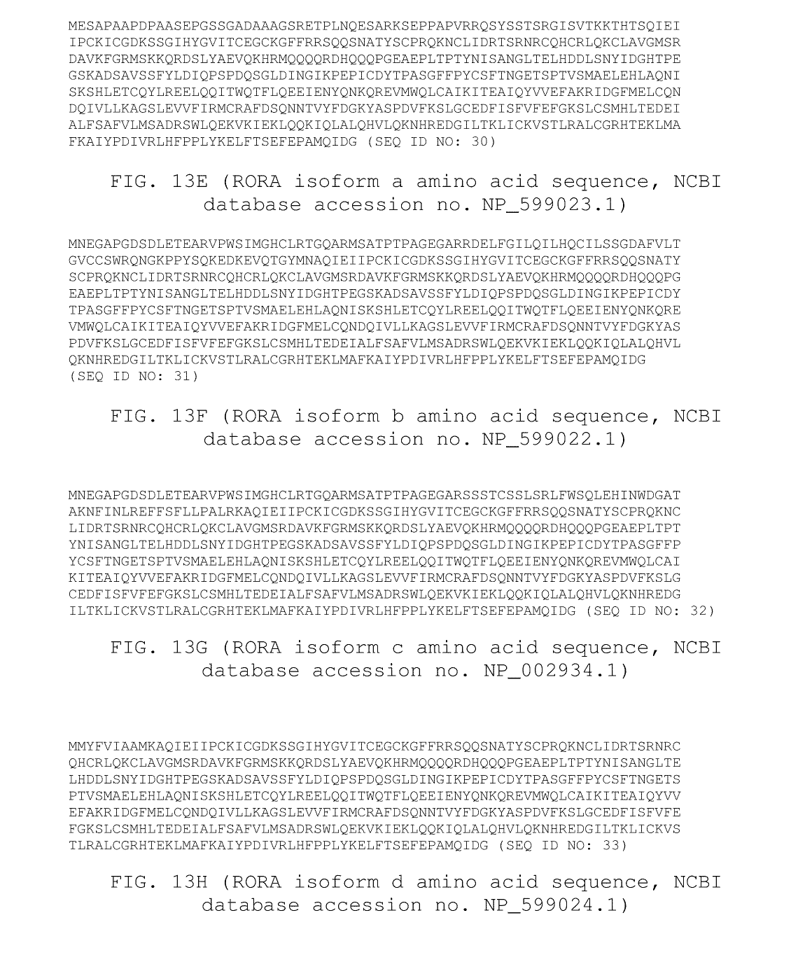

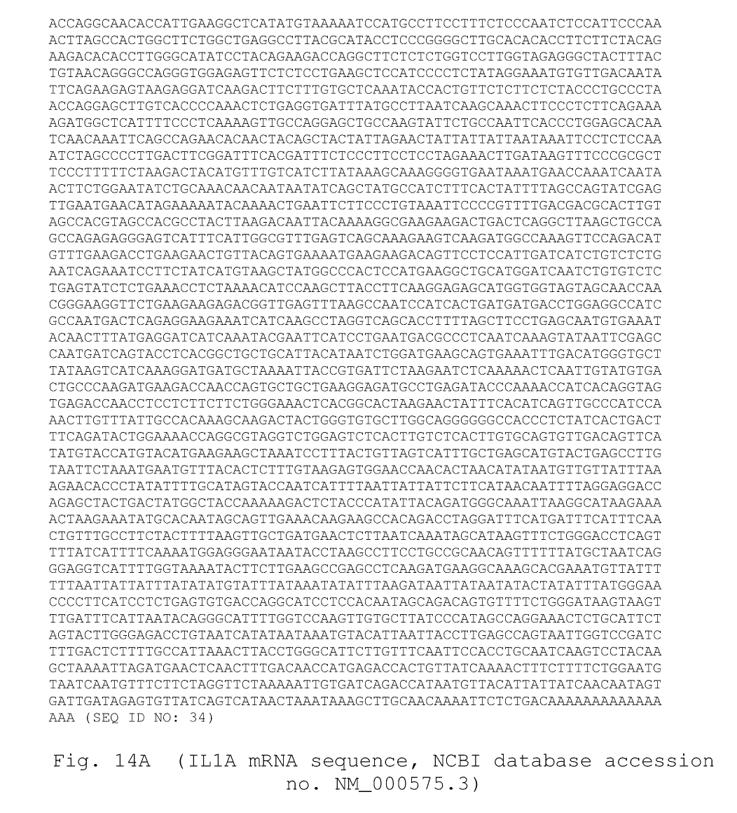

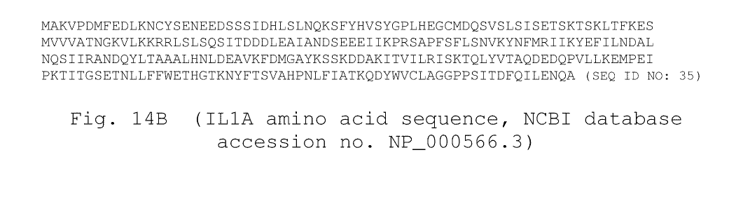

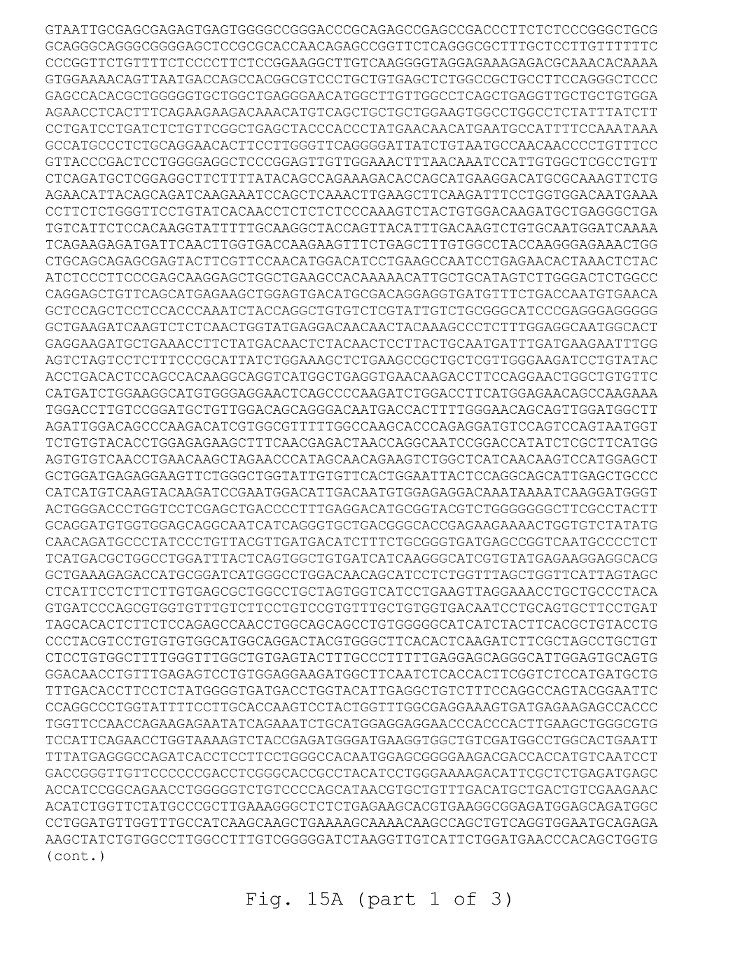

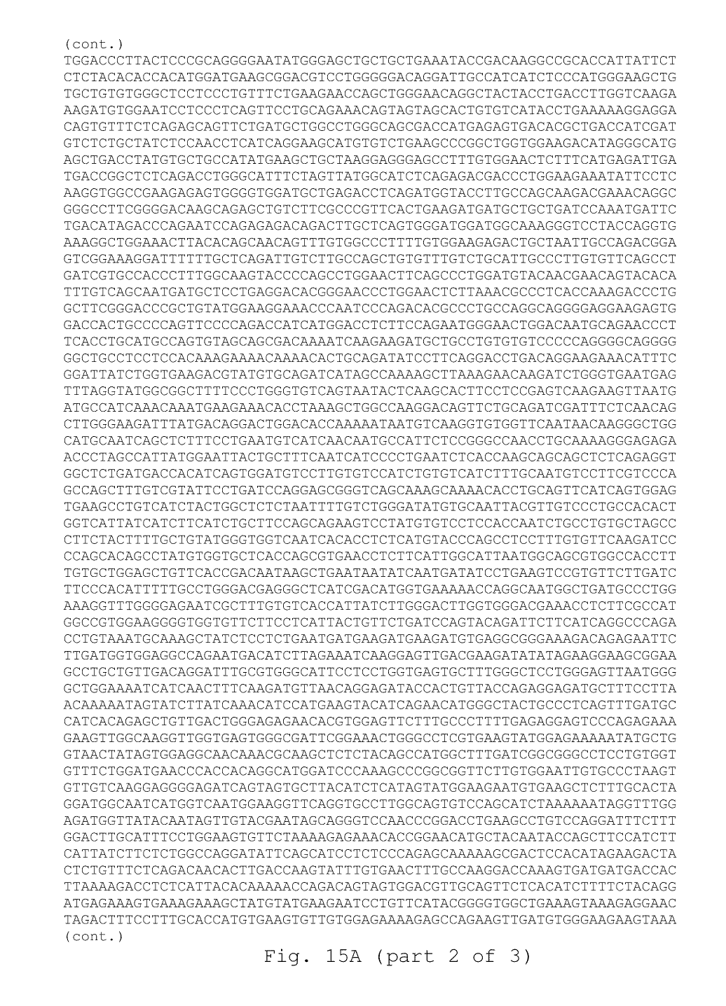

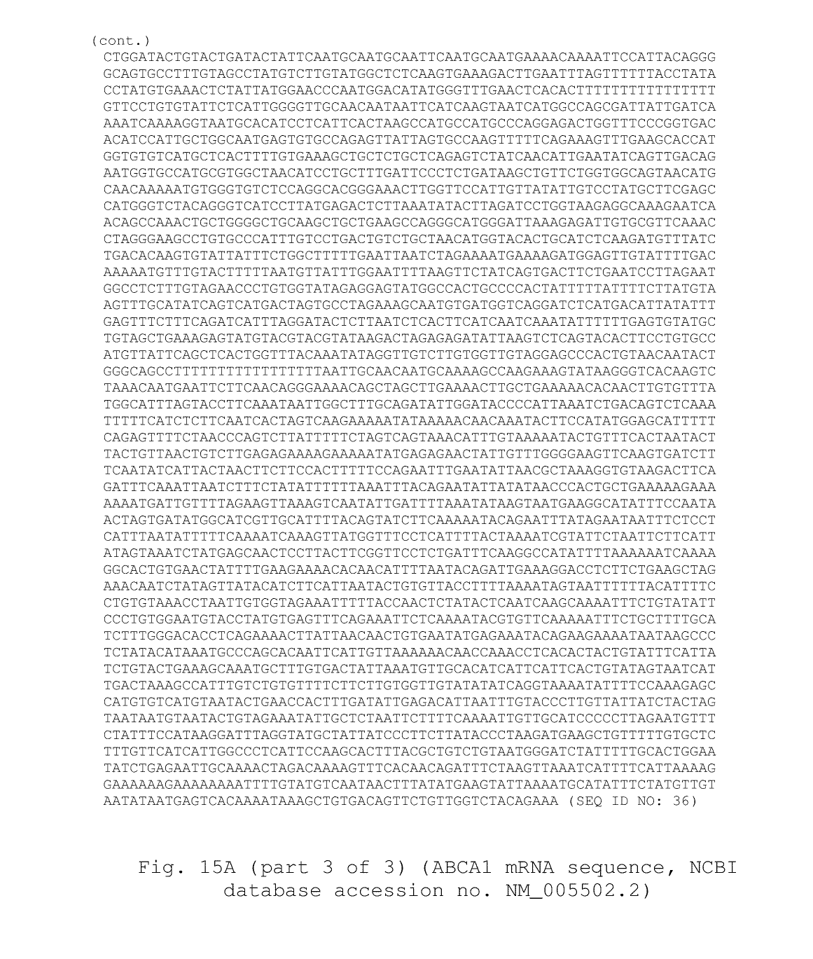

As used herein, a “RORA gene product” is understood to mean (i) a nucleic acid, for example, a sequence at least 75, at least 150, or at least 225 nucleotides in length that hybridizes under specific hybridization and washing conditions to the RORA gene (either the sense or anti-sense sequence); (ii) a nucleic acid sequence that is at least 90%, more preferably at least 95%, and more preferably at least 98% identical to the mRNA sequence shown in one of FIGS. 13A-13D, or a nucleic acid sequence that hybridizes under specific hybridization and washing conditions to the sequence shown in one of FIGS. 13A-13D; or (iii) a peptide or protein at least 25, at least 50, or at least 75 amino acids in length that is at least 95%, more preferably at least 98%, and more preferably at least 99% identical to the amino acid sequence shown in one of FIGS. 13E-13H.

The nucleic acid encoding human RORA gene spans approximately 732 kb in length as reported in the NCBI gene database under gene ID: 6095, gene location accession no. NC_{—}000015.8 (58576755 . . . 59308794, complement). The RORA gene has been reported to generate four splicing transcript variants. The transcript variant 1 comprises eleven exons as reported in the NCBI nucleotide database under accession no. NM_{—}134261; the protein encoded by transcript variant 1 is 523 amino acids in length as reported in the NCBI protein database under accession no. NP_{—}599023. The transcript variant 2 comprises twelve exons as reported in the NCBI nucleotide database under accession no. NM_{—}134260; the protein encoded by transcript variant 2 is 556 amino acids in length as reported in the NCBI protein database under accession no. NP_{—}599022. Transcript variant 3 comprises eleven exons as reported in the NCBI nucleotide database under accession no. NM_{—}002943; the protein encoded by transcript variant 3 is 548 amino acids in length as reported in the NCBI protein database under accession no. NP_{—}002934. Transcript variant 4 comprises ten exons as reported in the NCBI nucleotide database under accession no. NM_{—}134262; the protein encoded by transcript variant 4 is 468 amino acids in length as reported in the NCBI protein database under accession no. NP_{—}599024.

It is understood that the RORA gene may have more transcript variants. For example, it has been suggested that the RORA gene may generate at least fifteen transcript variants (see the ECGENE database, available at the web site, http://genome.ewha.ac.kr/ECgene/, under entry H15C5901). Polymorphisms have also been identified in the coding regions and untranslated regions of the exons, as well as in the introns and in the chromosome outside of the transcript region or regions of the RORA gene. As examples of the polymorphisms in the RORA gene, the NCBI SNP database reports 5,746 specific polymorphic sites for the RORA gene under gene ID: 6095. The mRNA sequences and the amino acid sequences of RORA are set forth in FIGS. 13A-13D and in FIGS. 13E-13G, respectively.

A.13. ILIA

IL1A is a member of the interleukin 1 cytokine family. This cytokine is a pleiotropic cytokine involved in various immune responses, inflammatory processes, and hematopoiesis. (Lord et al. (1991), “Expression of interleukin-1 alpha and beta genes by human blood polymorphonuclear leukocytes.” J. Clin. Invest. 87(4): 1312-1321.) As used herein, the term “IL1A gene” is understood to mean a nucleic acid sequence that is (i) at least 90%, more preferably at least 95%, and more preferably at least 98% identical to at least 75, at least 150, at least 225, at least 500, or at least 750 nucleotides in length of the known sequence for the ILIA gene as reported in the NCBI gene database under gene ID: 3552, gene location accession no. NC_{—}000002.10 (113247963 . . . 113259442, complement) (available at the web site, www.ncbi.nlm.nih.gov) or a strand complementary thereto; (ii) the full length sequence of the ILIA gene reported in the NCBI gene database under gene ID: 3552, gene location accession no. NC_{—}000002.10 (113247963 . . . 113259442, complement); (iii) a naturally occurring allelic variant of one of the foregoing sequences; or (iv) a nucleic acid sequence complementary to one of the foregoing sequences.

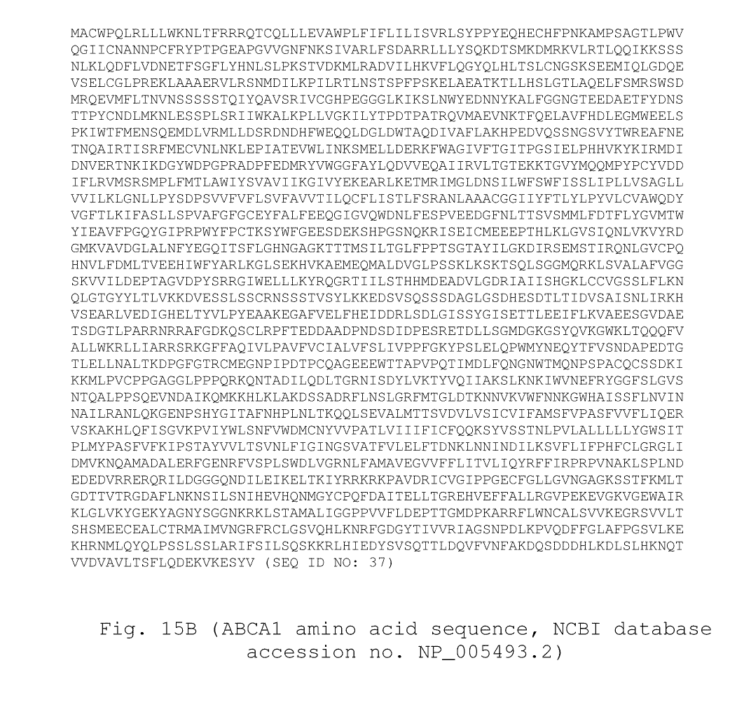

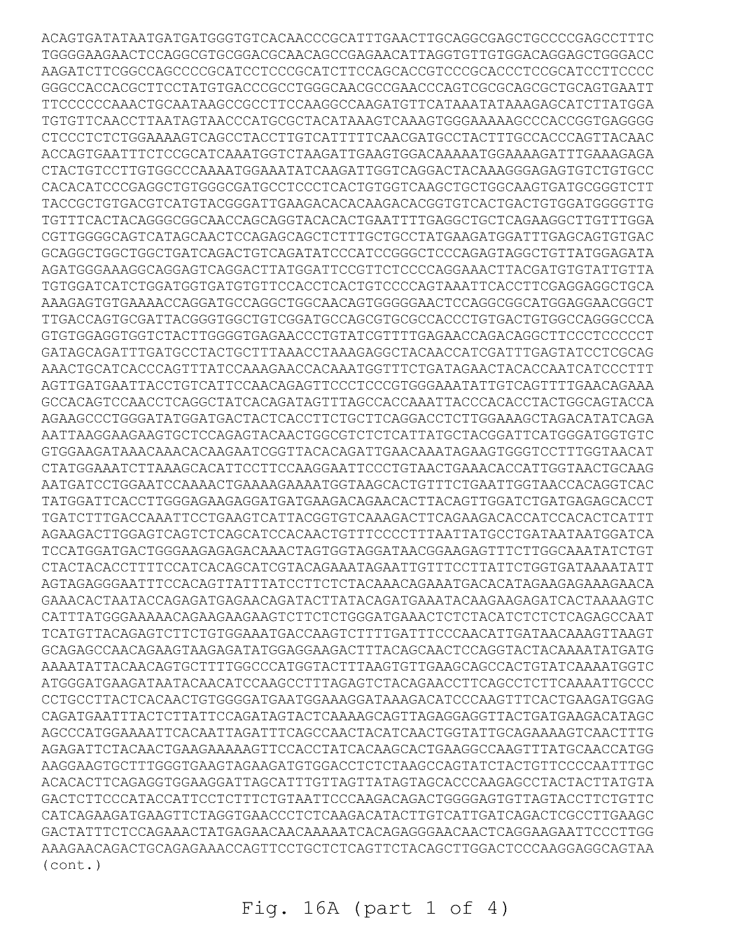

As used herein, a “IL1A gene product” is understood to mean (i) a nucleic acid sequence at least 75, at least 150, or at least 225 nucleotides in length that hybridizes under specific hybridization and washing conditions to the IL1A gene (either the sense or anti-sense sequence); (ii) a nucleic acid sequence that is at least 90%, more preferably at least 95%, and more preferably at least 98% identical to the mRNA sequence shown in FIG. 14A, or a nucleic acid sequence that hybridizes under specific hybridization and washing conditions to the sequence shown in FIG. 14A; or (iii) a peptide or protein at least 25, at least 50, or at least 75 amino acids in length that is at least 95%, more preferably at least 98%, and more preferably at least 99% identical to the amino acid sequence shown in FIG. 14B.

The nucleic acid encoding the human IL1A gene spans about 11 kb in length as reported in the NCBI gene database under gene ID: 3552, gene location accession no. NC_{—}000002.10 (113247963 . . . 113259442, complement). It has been reported that the IL1A gene generates one transcript, which comprises seven exons as reported in the NCBI nucleotide database under gene ID: 3552, accession no. NM_{—}00575.3; the protein encoded by this transcript is 271 amino acids in length as reported in the NCBI protein database for gene ID: 3552, accession no. NP_{—}000566.3 (available at the web site, www.ncbi.nlm.nih.gov). It is also understood that the IL1A gene may have many transcript variants. For example, it has been suggested that the IL1A gene may generate at least two transcript variants (see, e.g., the ECGENE database, available at the web site, http://genome.ewha.ac.kr/ECgene/, under entry H2C14377). Polymorphisms have been identified in the coding regions and untranslated regions of the exons, as well as in the introns and in the chromosome outside of the transcript region or regions of the IL1A gene. As examples of the polymorphisms in the IL1A gene, the NCBI SNP database (available at the web site, www.ncbi.nlm.nih.gov) reports 184 specific polymorphic sites in the IL1A gene under gene ID: 3552. The mRNA sequence and the amino acid sequence of IL1A are set forth in FIGS. 14A and 14B, respectively.

A.14. ABCA1

ABCA1 is a member of the superfamily of ATP-binding cassette (ABC) transporters. With cholesterol as its substrate, this protein functions as a cholesterol efflux pump in the cellular lipid removal pathway. (Denis et al. (2008), “ATP-binding cassette A1-mediated lipidation of apoliproprotein A-I occurs at the plasma membrane and not in the endocytic compartments,” J. Biol. Chem. 283(23): 16178-16186.) As used herein, the term “ABCA1 gene” is understood to mean a nucleic acid sequence that is (i) at least 90%, more preferably at least 95%, and more preferably at least 98% identical to at least 75, at least 150, at least 225, at least 500, or at least 750 nucleotides in length of the known sequence for the ABCA1 gene reported in the NCBI gene database under gene ID: 19, gene location accession no. NC_{—}000009.10 (106583104 . . . 106730257, complement) or a strand complementary thereto; (ii) the full length sequence of the ABCA1 gene reported in the NCBI gene database under gene ID: 19, gene location accession no. NC_{—}000009.10 (106583104 . . . 106730257, complement); (iii) a naturally occurring allelic variant of one of the foregoing sequences; or (iv) a nucleic acid sequence complementary to one of the foregoing sequences.

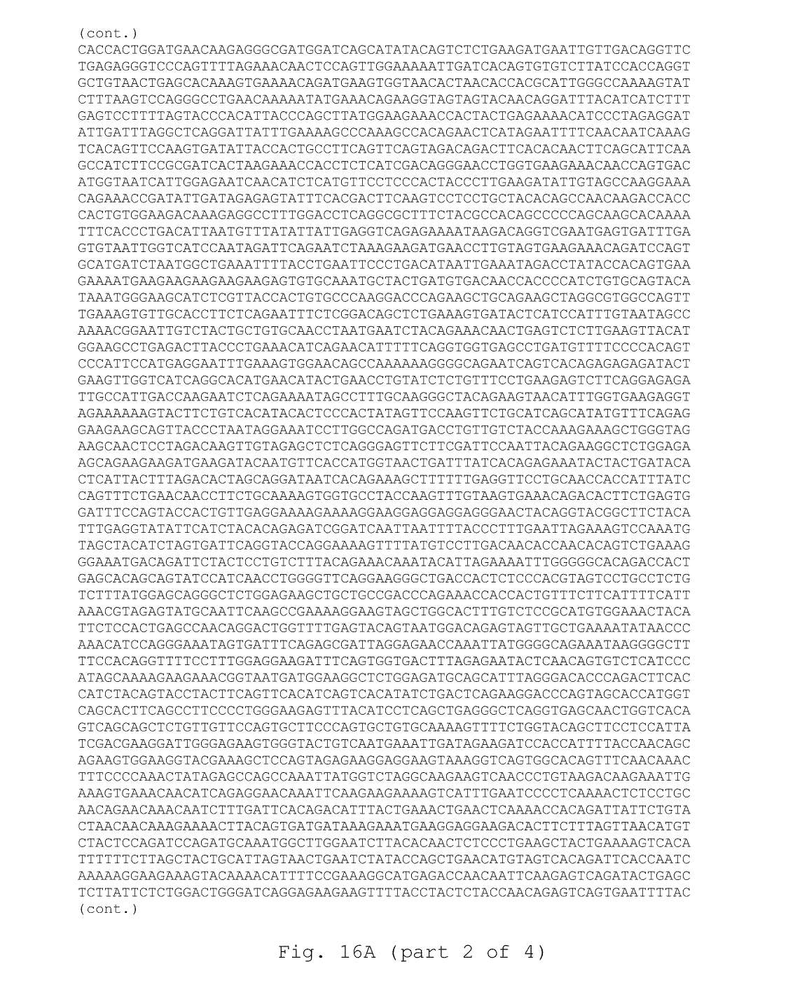

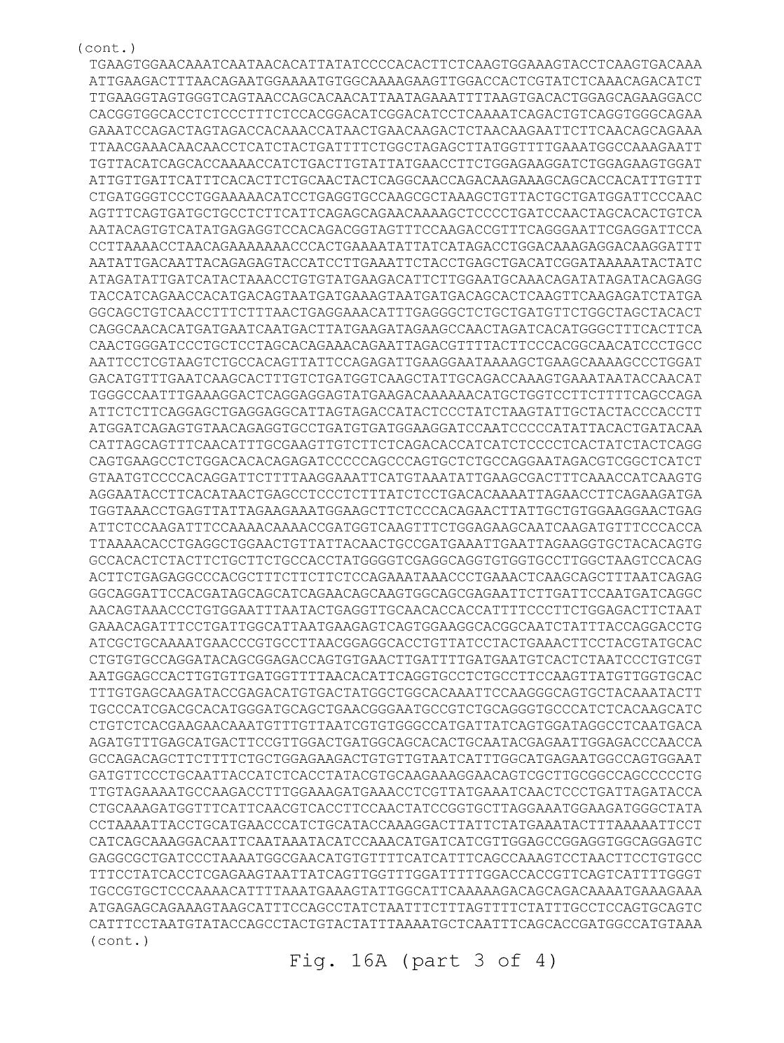

As used herein, an “ABCA1 gene product” is understood to mean (i) a nucleic acid sequence at least 75, at least 150, or at least 225 nucleotides in length that hybridizes under specific hybridization and washing conditions to the ABCA1 gene (either the sense or anti-sense sequence); (ii) a nucleic acid sequence that is at least 90%, more preferably at least 95%, and more preferably at least 98% identical to the mRNA sequence shown in FIG. 15A, or a nucleic acid sequence that hybridizes under specific hybridization and washing conditions to the sequence shown in FIG. 15A; or (iii) a peptide or protein at least 25, at least 50, or at least 75 amino acids in length that is at least 95%, more preferably at least 98%, and more preferably at least 99% identical to the amino acid sequence shown in FIG. 15B.

The nucleic acid encoding the human ABCA1 gene spans approximately 147 kb in length as reported in the NCBI gene database under gene ID: 19, gene location accession no. NC_{—}000009.10 (106583104 . . . 106730257, complement). It has been reported that the ABCA1 gene generates one transcript, which comprises fifty exons as reported in the NCBI nucleotide database under gene ID: 19, accession no. NM_{—}005502.2; the protein encoded by this transcript is 2261 amino acids in length as reported in the NCBI protein database for gene ID: 19, accession no. NP_{—}005493.2 (available at the web site, www.ncbi.nlm.nih.gov). It is also understood that the ABCA1 gene may have many transcript variants. For example, it has been suggested that the ABCA1 gene may generate at least three transcript variants (see, e.g., the Ensembl database, available at the website, http://ensembl.org/index.html, under entry ENSG00000165029). Polymorphisms have been identified in the coding regions and untranslated regions of the exons, as well as in the introns and in the chromosome outside of the transcript region or regions of the ABCA1 gene. As examples of the polymorphisms in the ABCA1 gene, the NCBI SNP database (available at the web site, www.ncbi.nlm.nih.gov) reports 1439 specific polymorphic sites in the ABCA1 gene under gene ID: 19. The mRNA sequence and the amino acid sequence of ABCA1 are set forth in FIGS. 15A and 15B, respectively.

A.15. VCAN

VCAN, a chondroitin sulfate proteoglycan, also known as CSPG2, is one of the main components of the extracellular matrix which provides a loose and hydrated matrix during key events in development and disease. (Rahmani et al. (2006), “Versican: signaling to transcriptional control pathways,” Can. J. Physiol. Pharmacol. 84(1): 77-92.) As used herein, the term “VCAN gene” is understood to mean a nucleic acid sequence that is (i) at least 90%, more preferably at least 95%, and more preferably at least 98% identical to at least 75, at least 150, at least 225, at least 500, or at least 750 nucleotides in length of the known sequence for the VCAN gene reported in the NCBI gene database under gene ID: 1462, gene location accession no. NC_{—}000005.8 (82803339.82912737) or a strand complementary thereto; (ii) the full length sequence of the VCAN gene reported in the NCBI gene database under gene ID: 1462, gene location accession no. NC_{—}000005.8 (82803339 . . . 82912737); (iii) a naturally occurring allelic variant of one of the foregoing sequences; or (iv) a nucleic acid sequence complementary to one of the foregoing sequences.

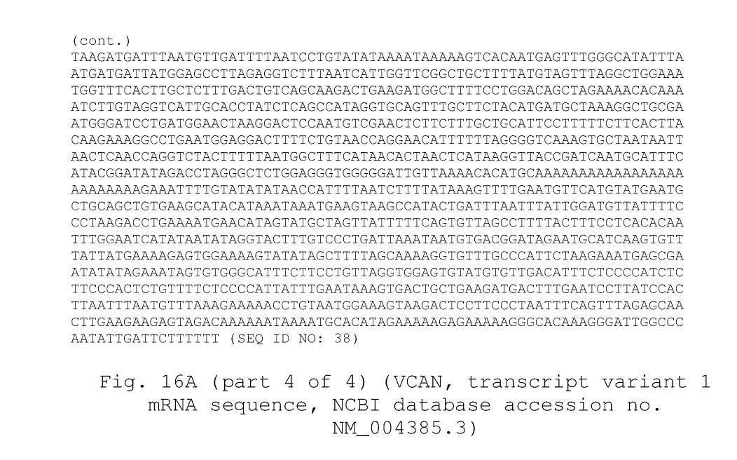

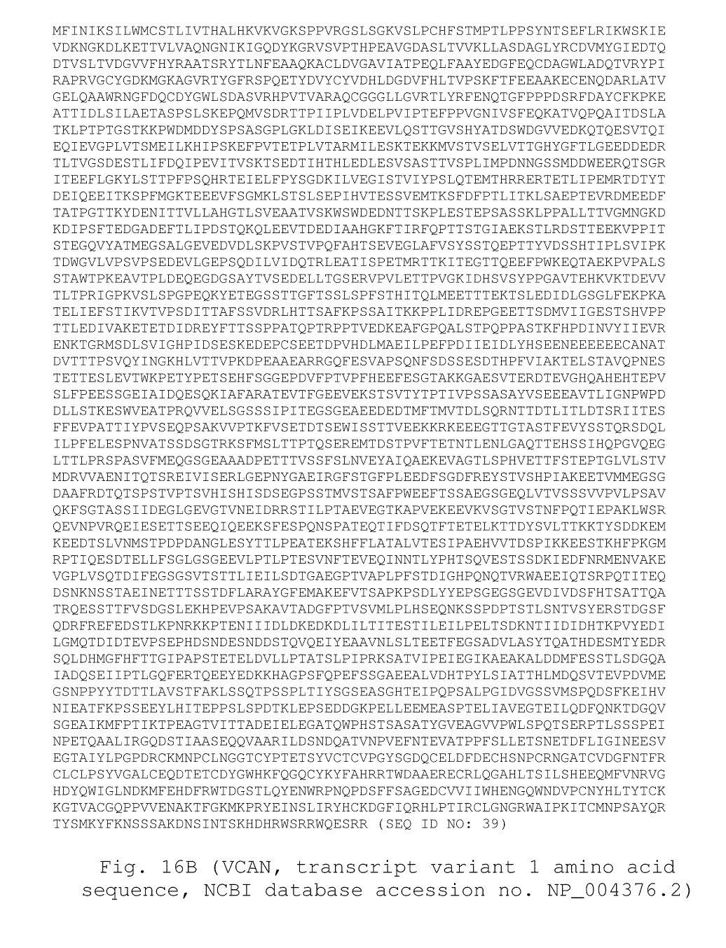

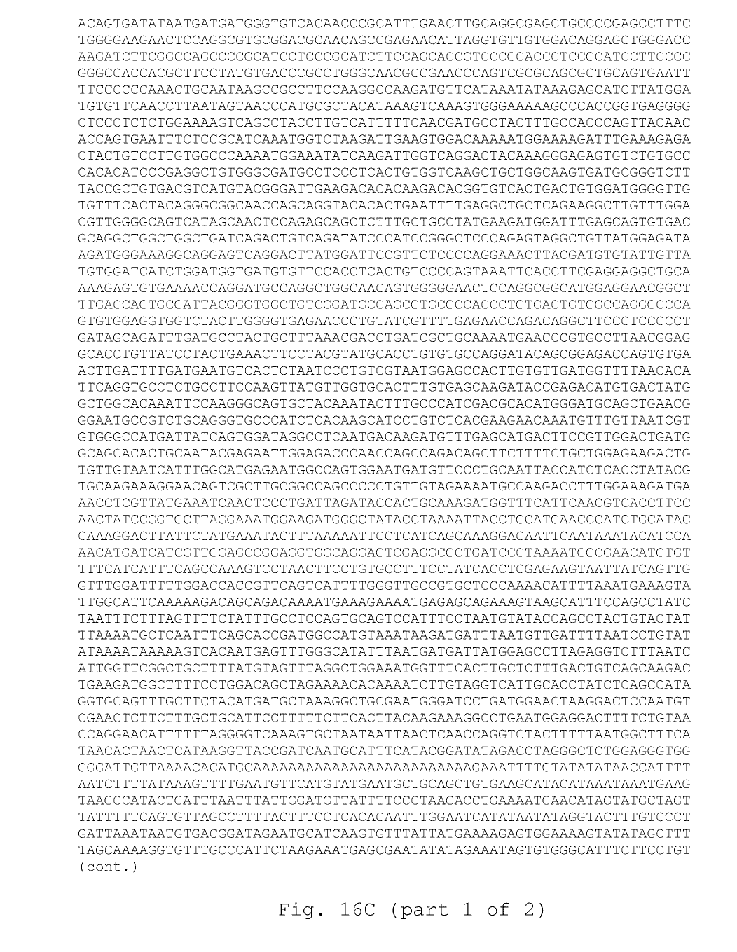

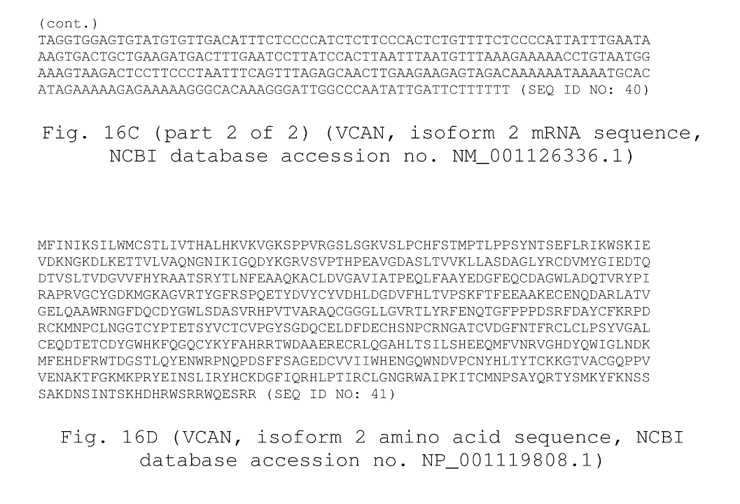

As used herein, a “VCAN gene product” is understood to mean (i) a nucleic acid sequence at least 75, at least 150, or at least 225 nucleotides in length that hybridizes under specific hybridization and washing conditions to the VCAN gene (either the sense or anti-sense sequence); (ii) a nucleic acid sequence that is at least 90%, more preferably at least 95%, and more preferably at least 98% identical to the mRNA sequence shown in one of FIGS. 16A and 16C, or a nucleic acid sequence that hybridizes under specific hybridization and washing conditions to the sequence shown in one of FIGS. 16A and 16C; or (iii) a peptide or protein at least 25, at least 50, or at least 75 amino acids in length that is at least 95%, more preferably at least 98%, and more preferably at least 99% identical to the amino acid sequence shown in one of FIGS. 16B and 16D.

The nucleic acid encoding the human VCAN gene spans approximately 109 kb in length as reported in the NCBI gene database under gene ID: 1462, gene location accession no. NC_{—}000005.8 (82803339 . . . 82912737). It has been reported that the VCAN gene generates two transcript variants. Transcript variant 1 comprises fifteen exons as reported in the NCBI nucleotide database under gene ID: 1462, accession no. NM_{—}004385.3; the protein encoded by this transcript is 3396 amino acids in length as reported in the NCBI protein database for gene ID: 1462, accession no. NP_{—}004376.2 (available at the web site, www.ncbi.nlm.nih.gov). Transcript variant 2 comprises 13 exons as reported in the NCBI nucleotide database under accession no. NM_{—}001126336.1; the protein encoded by this transcript is 655 amino acids in length as reported in the NCBI protein database under accession no. NP_{—}001119808.1. It is understood that the VCAN gene may have more transcript variants. For example, it has been suggested that the VCAN gene may generate at least four transcript variants (see, e.g., the Ensembl database, available at the website, http://ensembl.org/index.html, under entry ENSG00000038427). Polymorphisms have been identified in the coding regions of the exons, as well as in the introns and in the chromosome outside of the transcript region or regions of the VCAN gene. As examples of the polymorphisms in the VCAN gene, the NCBI SNP database (available at the web site, www.ncbi.nlm.nih.gov) reports 841 specific polymorphic sites in the VCAN gene under gene ID: 1462. The mRNA sequences and the amino acid sequences of VCAN are set forth in FIGS. 16A and 16C and FIGS. 16B and 16D, respectively.

A.16. SHQ1

SHQ1 is an essential nuclear protein, required for accumulation of box H/ACA snoRNAs and for rRNA processing. (Yang et al. (2002), “The Shq1p.Naf1p complex is required for box H/ACA small nucleolar ribonucleoprotein particle biogenesis,” J Biol Chem. 277(47):45235-45242). As used herein, the term “SHQ1 gene” is understood to mean a nucleic acid sequence that is (i) at least 90%, more preferably at least 95%, and more preferably at least 98% identical to at least 75, at least 150, at least 225, at least 500, or at least 750 nucleotides in length of the known sequence for the SHQ1 gene as reported in the NCBI gene database under gene ID: 55164, gene location accession no. NC_{—}000003.10 (72881118 . . . 72980288, complement) (available at the web site, www.ncbi.nlm.nih.gov) or a strand complementary thereto; (ii) the full length sequence of the SHQ1 gene reported in the NCBI gene database under gene ID: 55164, gene location accession no. NC_{—}000003.10 (72881118 . . . 72980288, complement); (iii) a naturally occurring allelic variant of one of the foregoing sequences; or (iv) a nucleic acid sequence complementary to one of the foregoing sequences.

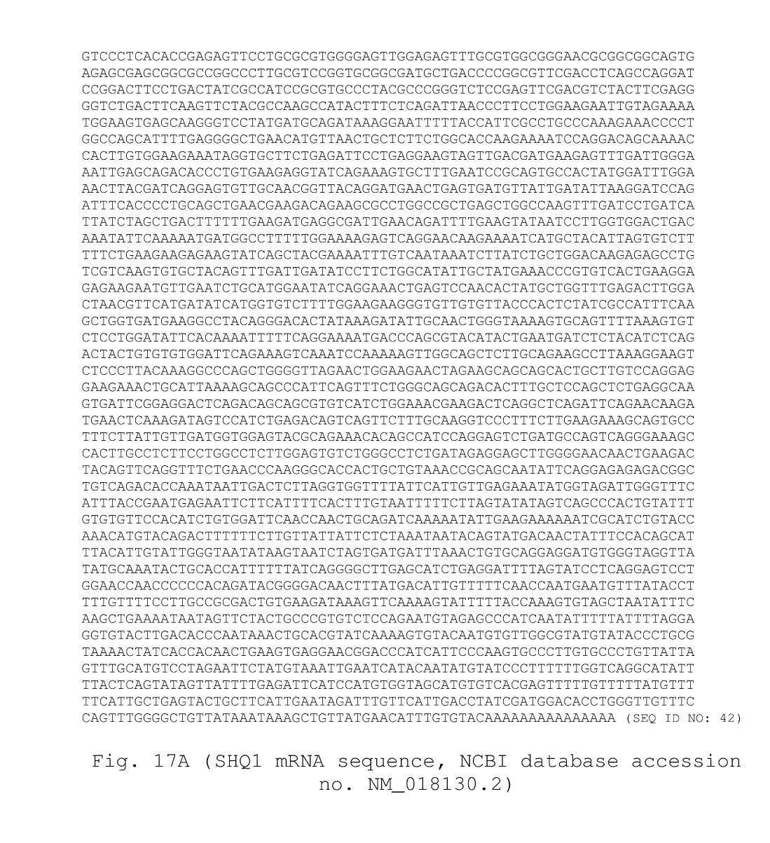

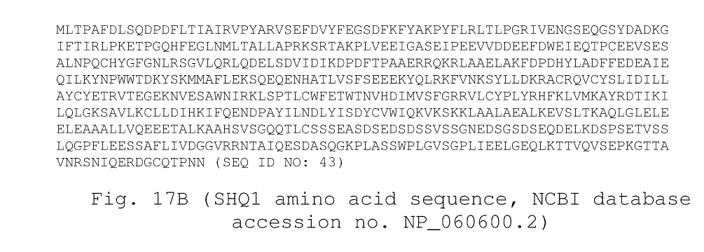

As used herein, a “SHQ1 gene product” is understood to mean (i) a nucleic acid sequence at least 75, at least 150, or at least 225 nucleotides in length that hybridizes under specific hybridization and washing conditions to the SHQ1 gene (either the sense or anti-sense sequence); (ii) a nucleic acid sequence that is at least 90%, more preferably at least 95%, and more preferably at least 98% identical to the mRNA sequence shown in FIG. 17A, or a nucleic acid sequence that hybridizes under specific hybridization and washing conditions to the sequence shown in FIG. 17A; or (iii) a peptide or protein at least 25, at least 50, or at least 75 amino acids in length that is at least 95%, more preferably at least 98%, and more preferably at least 99% identical to the amino acid sequence shown in FIG. 17B.

The nucleic acid encoding the human SHQ1 gene spans about 99 kb in length as reported in the NCBI gene database under gene ID: 55164, gene location accession no. NC_{—}000003.10 (72881118 . . . 72980288, complement). It has been reported that the SHQ1 gene generates one transcript, which comprises eleven exons as reported in the NCBI nucleotide database under gene ID: 55164, accession no. NM_{—}018130.2; the protein encoded by this transcript is 577 amino acids in length as reported in the NCBI protein database for gene ID: 55164, accession no. NP_{—}060600.2 (available at the web site, www.ncbi.nlm.nih.gov). It is also understood that the SHQ1 gene may have many transcript variants. For example, it has been suggested that the SHQ1 gene may generate at least five transcript variants (see, e.g., the ECGENE database, available at the web site, http://genome.ewha.ac.kr/ECgene/, under entry H3C10117). Polymorphisms have been identified in the coding regions and untranslated regions of the exons, as well as in the introns and in the chromosome outside of the transcript region or regions of the SHQ1 gene. As examples of the polymorphisms in the SHQ1 gene, the NCBI SNP database (available at the web site, www.ncbi.nlm.nih.gov) reports 398 specific polymorphic sites in the SHQ1 gene under gene ID: 55164. The mRNA sequence and the amino acid sequence of SHQ1 are set forth in FIGS. 17A and B, respectively.

A.17. UCHL1

UCHL1 is a member of a gene family whose products hydrolyze small C-terminal adducts of ubiquitin to generate the ubiquitin monomer. Expression of UCHL1 is highly specific to neurons and to cells of the diffuse neuroendocrine system and their tumors. It is present in all neurons (Doran et al. (1983), Isolation of PGP 9.5, a new human neurone-specific protein detected by high-resolution two-dimensional electrophoresis. J. Neurochem., 40(6):1542-7.) As used herein, the term “UCHL1 gene” is understood to mean a nucleic acid sequence that is (i) at least 90%, more preferably at least 95%, and more preferably at least 98% identical to at least 75, at least 150, at least 225, at least 500, or at least 750 nucleotides in length of the known sequence for the UCHL1 gene as reported in the NCBI gene database under gene ID: 7345, gene location accession no. NC_{—}000004.10 (40953686 . . . 40965203) (available at the web site, www.ncbi.nlm.nih.gov) or a strand complementary thereto; (ii) the full length sequence of the UCHL1 gene reported in the NCBI gene database under gene ID: 7345, gene location accession no. NC_{—}000004.10 (40953686 . . . 40965203); (iii) a naturally occurring allelic variant of one of the foregoing sequences; or (iv) a nucleic acid sequence complementary to one of the foregoing sequences.

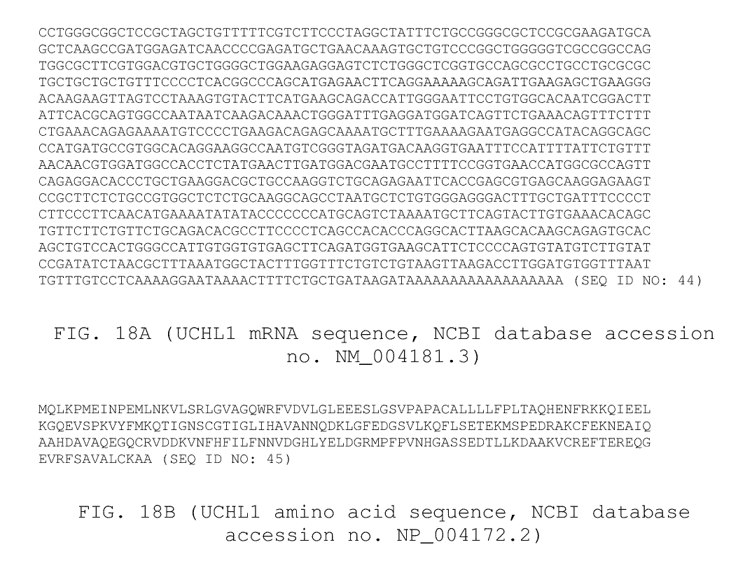

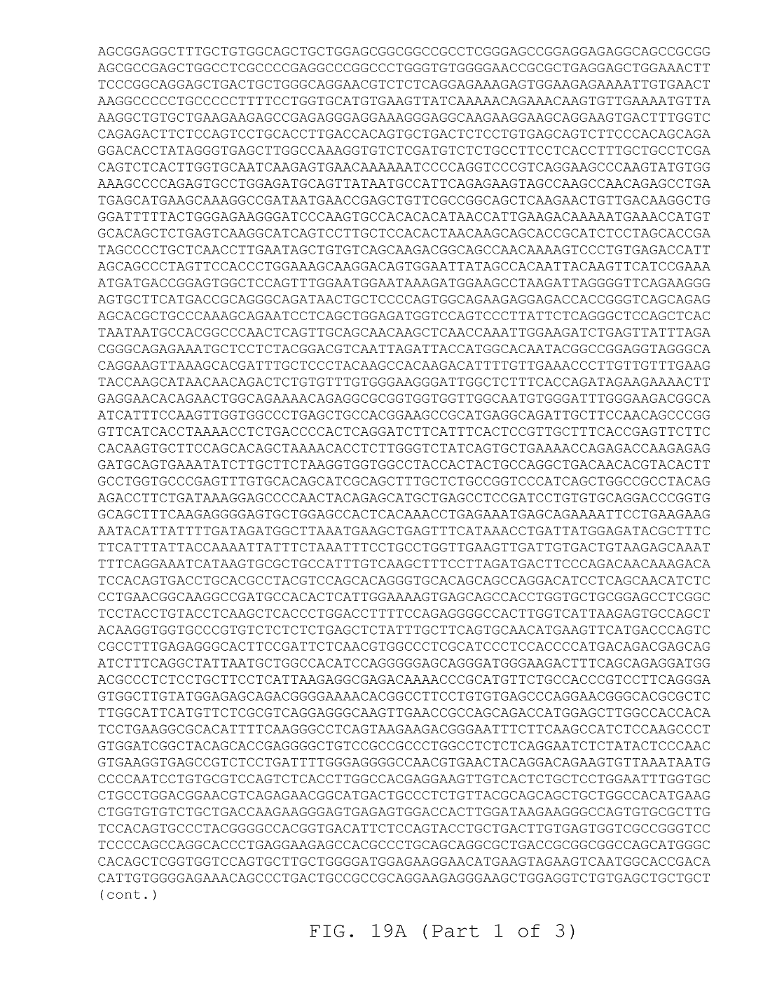

As used herein, a “UCHL1 gene product” is understood to mean (i) a nucleic acid sequence at least 75, at least 150, or at least 225 nucleotides in length that hybridizes under specific hybridization and washing conditions to the UCHL1 gene (either the sense or anti-sense sequence); (ii) a nucleic acid sequence that is at least 90%, more preferably at least 95%, and more preferably at least 98% identical to the mRNA sequence shown in FIG. 18A, or a nucleic acid sequence that hybridizes under specific hybridization and washing conditions to the sequence shown in FIG. 18A; or (iii) a peptide or protein at least 25, at least 50, or at least 75 amino acids in length that is at least 95%, more preferably at least 98%, and more preferably at least 99% identical to the amino acid sequence shown in FIG. 18B.

The nucleic acid encoding the human UCHL1 gene spans about 12 kb in length as reported in the NCBI gene database under gene ID: 7345, gene location accession no. NC_{—}000004.10 (40953686 . . . 40965203). It has been reported that the UCHL1 gene generates one transcript, which comprises nine exons as reported in the NCBI nucleotide database under gene ID: 7345, accession no. NM_{—}004181.3; the protein encoded by this transcript is 223 amino acids in length as reported in the NCBI protein database under gene ID: 7345, accession no. NP_{—}004172.2 (available at the web site, www.ncbi.nlm.nih.gov). It is also understood that the UCHL1 gene may have many transcript variants. For example, it has been suggested that the UCHL1 gene may generate at least fifteen transcript variants (see, e.g., the ECGENE database, available at the web site, http://genome.ewha.ac.kr/ECgene/, under entry H4C4831). Polymorphisms have been identified in the coding regions and untranslated regions of the exons, as well as in the introns and in the chromosome outside of the transcript region or regions of the UCHL1 gene. As examples of the polymorphisms in the UCHL1 gene, the NCBI SNP database (available at the web site, www.ncbi.nlm.nih.gov) reports 80 specific polymorphic sites in the UCHL1 gene under gene ID: 7345. The mRNA sequence and the amino acid sequence of UCHL1 are set forth in FIGS. 18A and 18B, respectively.

A.18. TANC1

TANC1 is a tetratricopeptide repeat protein. It may work as a postsynaptic scaffold component by forming a multiprotein complex with various postsynaptic density proteins (Suzuki et al. (2005), A novel scaffold protein, TANC, possibly a rat homolog of Drosophila rolling pebbles (rols), forms a multiprotein complex with various postsynaptic density proteins, Eur. J. Neurosci., 21(2):339-50.) As used herein, the term “TANC1 gene” is understood to mean a nucleic acid sequence that is (i) at least 90%, more preferably at least 95%, and more preferably at least 98% identical to at least 75, at least 150, at least 225, at least 500, or at least 750 nucleotides in length of the known sequence for the TANC1 gene reported in the NCBI gene database under gene ID: 85461, gene location accession no. NC_{—}000002.10 (159533392 . . . 159797416) or a strand complementary thereto; (ii) the full length sequence of the TANC1 gene reported in the NCBI gene database under gene ID: 85461, gene location accession no. NC_{—}000002.10 (159533392 . . . 159797416); (iii) a naturally occurring allelic variant of one of the foregoing sequences; or (iv) a nucleic acid sequence complementary to one of the foregoing sequences.

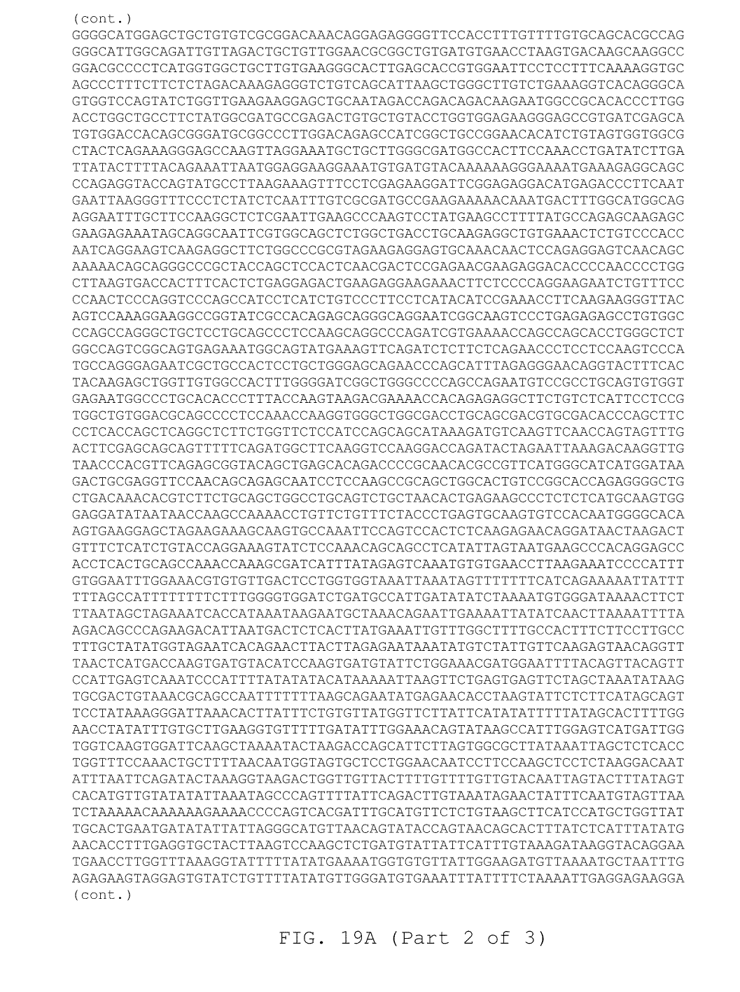

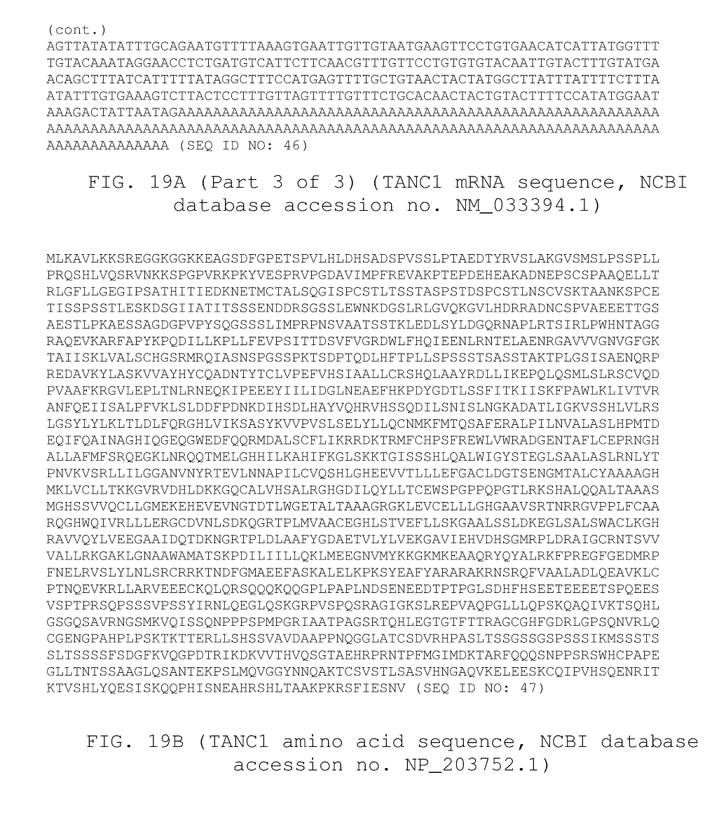

As used herein, a “TANC1 gene product” is understood to mean (i) a nucleic acid sequence at least 75, at least 150, or at least 225 nucleotides in length that hybridizes under specific hybridization and washing conditions to the TANC1 gene (either the sense or anti-sense sequence); (ii) a nucleic acid sequence that is at least 90%, more preferably at least 95%, and more preferably at least 98% identical to the mRNA sequence shown in FIG. 19A, or a nucleic acid sequence that hybridizes under specific hybridization and washing conditions to the sequence shown in FIG. 19A; or (iii) a peptide or protein at least 25, at least 50, or at least 75 amino acids in length that is at least 95%, more preferably at least 98%, and more preferably at least 99% identical to the amino acid sequence shown in FIG. 19B.

The nucleic acid encoding the human TANC1 gene spans about 264 kb in length as reported in the NCBI gene database under gene ID: 85461, gene location accession no. NC_{—}000002.10 (159533392 . . . 159797416). It has been reported that the TANC1 gene generates one transcript, which comprises twenty seven exons as reported in the NCBI nucleotide database under gene ID: 85461, accession no. NM_{—}033394.1; the protein encoded by this transcript is 1861 amino acids in length as reported in the NCBI protein database under gene ID: 85461, accession no. NP_{—}203752.1. It is also understood that the TANC1 gene may have many transcript variants. For example, it has been suggested that the TANC1 gene may generate at least ten transcript variants (see, e.g. the ECGENE database, available at the web site, http://genome.ewha.ac.kr/ECgene/, under entry H2C18651). Polymorphisms have also been identified in the coding regions and untranslated regions of the exons, as well as in the introns and in the chromosome outside of the transcript region or regions of the TANC1 gene. As examples of the polymorphisms in the TANC1 gene, the NCBI SNP database reports 1781 specific polymorphic sites for the TANC1 gene under gene ID: 85461. The mRNA sequence and the amino acid sequence of TANC1 are set forth in FIG. 19A and in FIG. 19B, respectively.

A.19. PKP2

PKP2 encodes a member of the arm-repeat (armadillo) and plakophilin gene families, which contain numerous armadillo repeats, localize to cell desmosomes and nuclei, and participate in linking cadherins to intermediate filaments in the cytoskeleton. PKP2 may regulate the signaling activity of beta-catenin (Mertens et al. (1996), Plakophilins 2a and 2b: constitutive proteins of dual location in the karyoplasm and the desmosomal plaque, J. Cell Biol. 135 (4):1009-25.) As used herein, the term “PKP2 gene” is understood to mean a nucleic acid sequence that is (i) at least 90%, more preferably at least 95%, and more preferably at least 98% identical to at least 75, at least 150, at least 225, at least 500, or at least 750 nucleotides in length of the known sequence for the PKP2 gene as reported in the NCBI gene database under gene ID: 5318, gene location accession no. NC_{—}000012.10 (32834947 . . . 32941047, complement) or a strand complementary thereto; (ii) the full length sequence of the PKP2 gene reported in the NCBI gene database under gene ID: 5318, gene location accession no. NC_{—}000012.10 (32834947 . . . 32941047, complement); (iii) a naturally occurring allelic variant of one of the foregoing sequences; or (iv) a nucleic acid sequence complementary to one of the foregoing sequences.

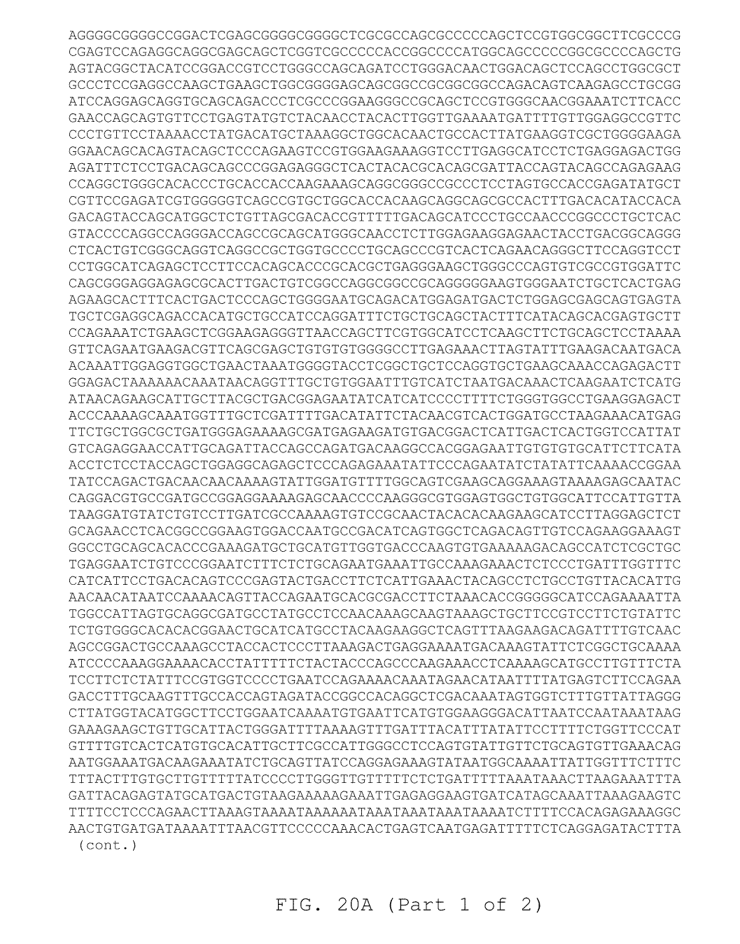

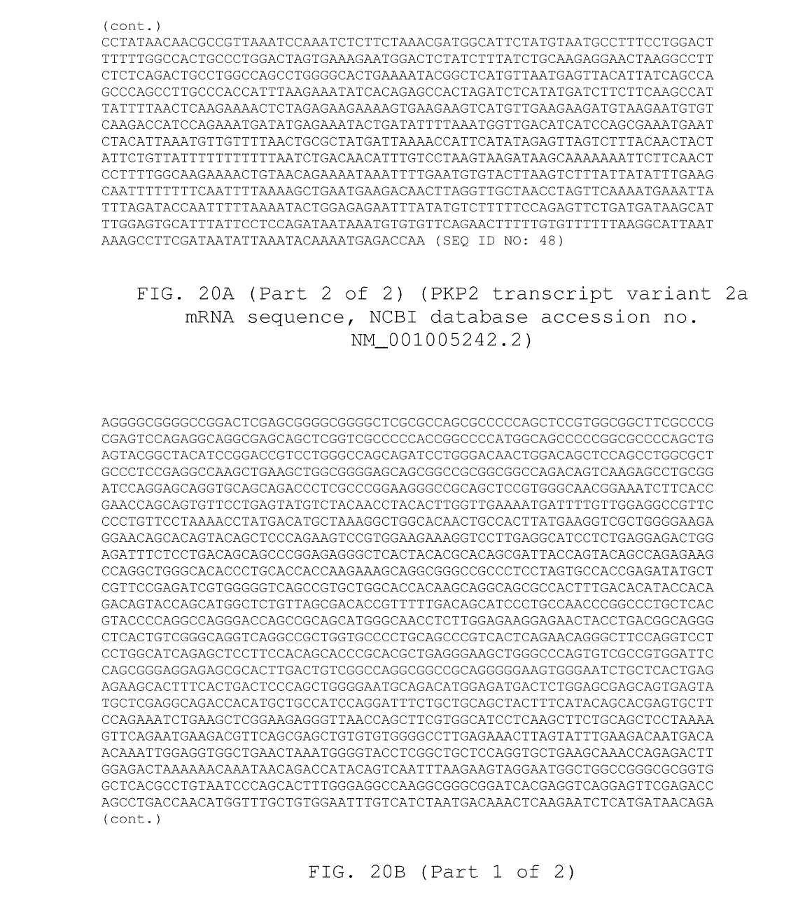

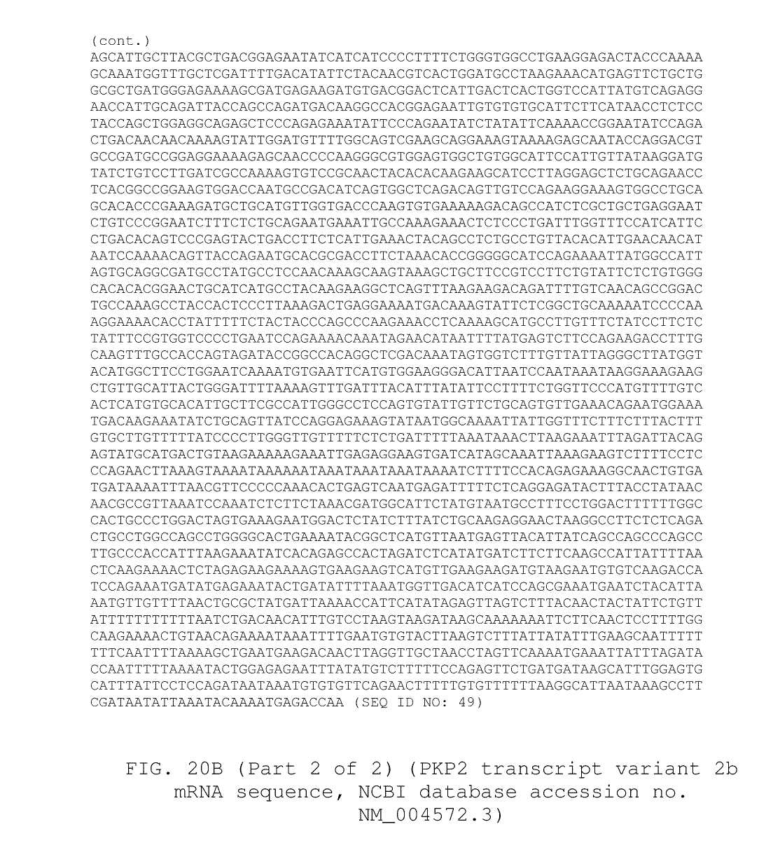

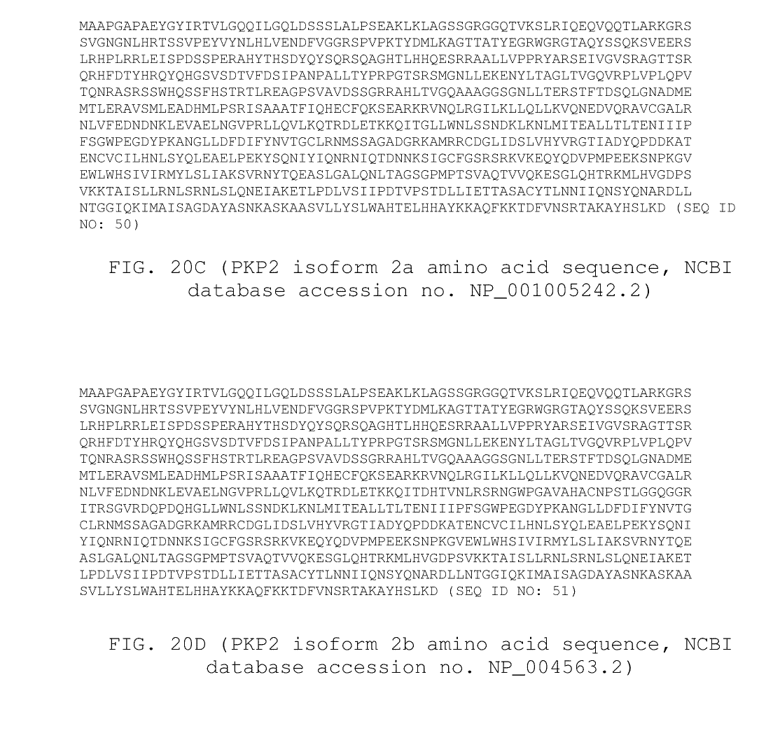

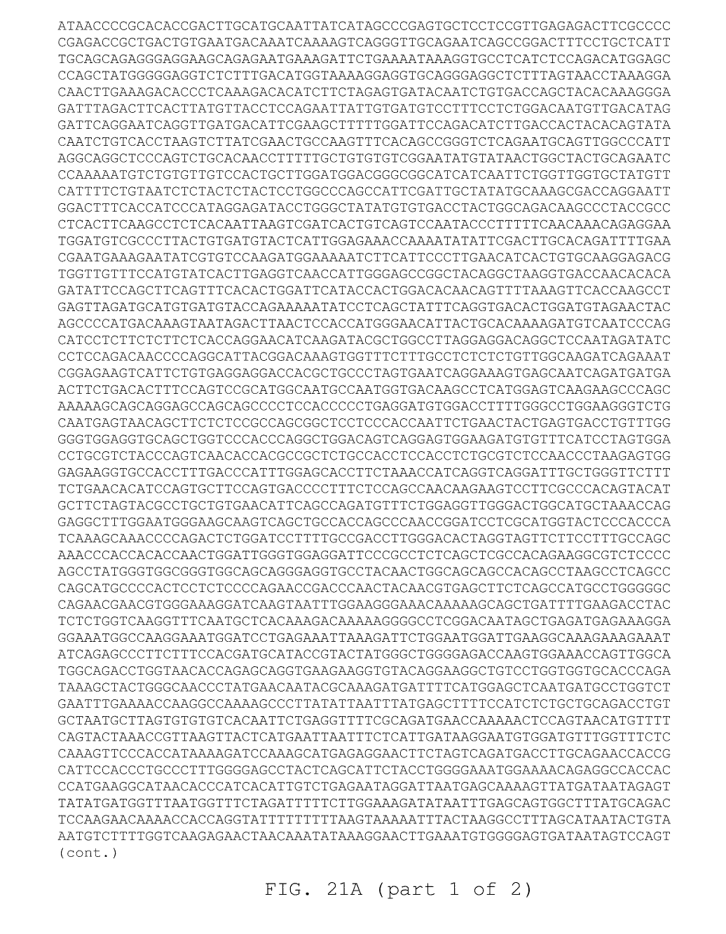

As used herein, a “PKP2 gene product” is understood to mean (i) a nucleic acid sequence at least 75, at least 150, or at least 225 nucleotides in length that hybridizes under specific hybridization and washing conditions to the PKP2 gene (either the sense or anti-sense sequence); (ii) a nucleic acid sequence that is at least 90%, more preferably at least 95%, and more preferably at least 98% identical to the transcript sequence shown in one of FIGS. 20A and 20B, or a nucleic acid sequence that hybridizes under specific hybridization and washing conditions to the sequence shown in one of FIGS. 20A and 20B; or (iii) a peptide or protein at least 25, at least 50, or at least 75 amino acids in length that is at least 95%, more preferably at least 98%, and more preferably at least 99% identical to the amino acid sequence shown in one of FIGS. 20C-D.

The nucleic acid encoding human PKP2 gene spans about 106 kb in length as reported in the NCBI gene database for gene ID: 5318, location accession no. NC_{—}000012.10 (32834947 . . . 32941047, complement). It has been reported that the PKP2 gene generates two splicing transcript variants: isoform 2a and isoform 2b. The transcript for isoform 2a comprises thirteen exons as reported in the NCBI nucleotide database under gene ID: 5318, accession no. NM_{—}001005242.2; the protein encoded by this transcript variant is 837 amino acids in length as reported in the NCBI protein database under gene ID:5318, accession no. NP_{—}001005242.2. The transcript for isoform 2b comprises fourteen exons as reported in the NCBI nucleotide database under gene ID: 5318, accession no. NM_{—}004572.3; the protein encoded by this transcript variant is 881 amino acids in length as reported in the NCBI protein database under gene ID: 5318, accession no. NP_{—}004563.2. It is also understood that the PKP2 gene may have more transcript variants. For example, it has been suggested that the PKP2 gene may generate at least four transcript variants (see, e.g., the ECGENE database, available at the web site, http://genome.ewha.ac.kr/ECgene/, under entry H12C5161). Polymorphisms have been identified in the coding regions and untranslated regions of the exons, as well as in the introns and in the chromosome outside of the transcript region or regions of the PKP2 gene. As examples of the polymorphisms in the PKP2 gene, the NCBI SNP database reports 657 specific polymorphic sites for the PKP2 gene under gene ID: 5318 in the corresponding SNP database. The mRNA sequences and amino acid sequences of PKP2 are set forth in FIGS. 20A-20B and 20C-20D, respectively.

A.20. DNAJC6

DNAJC6 belongs to the evolutionarily conserved DNAJ/HSP40 family of proteins, which regulate molecular chaperone activity by stimulating ATPase activity (Ohtsuka et al. (2000), Mammalian HSP40/DNAJ homologs: cloning of novel cDNAs and a proposal for their classification and nomenclature, Cell Stress Chaperones, 5(2):98-112.) As used herein, the term “DNAJC6 gene” is understood to mean a nucleic acid sequence that is (i) at least 90%, more preferably at least 95%, and more preferably at least 98% identical to at least 75, at least 150, at least 225, at least 500, or at least 750 nucleotides in length of the known sequence for the DNAJC6 gene as reported in the NCBI gene database under gene ID: 9829, gene location accession no. NC_{—}000001.9 (65503018 . . . 65654140) or a strand complementary thereto (ii) the full length sequence of the DNAJC6 gene reported in the NCBI gene database under gene ID: 9829, gene location accession no. NC_{—}000001.9 (65503018 . . . 65654140); (iii) a naturally occurring allelic variant of one of the foregoing sequences; or (iv) a nucleic acid sequence complementary to one of the foregoing sequences.

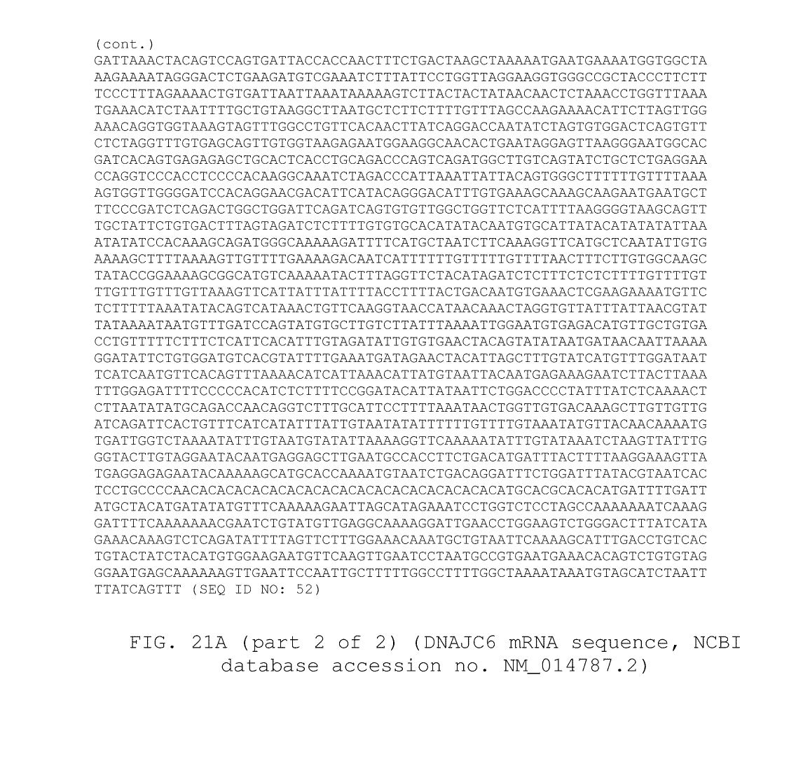

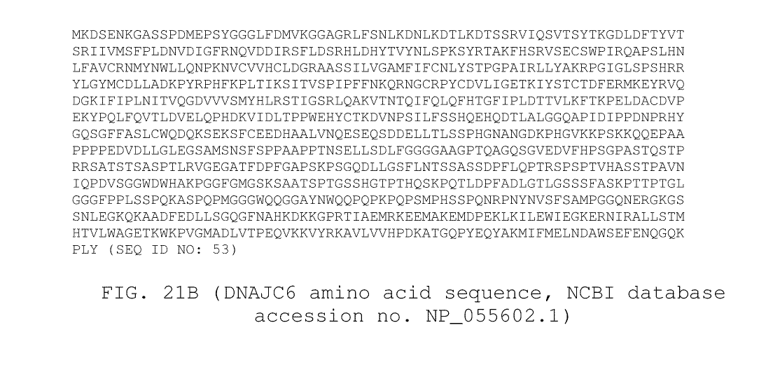

As used herein, a “DNACJ6 gene product” is understood to mean (i) a nucleic acid sequence at least 75, at least 150, or at least 225 nucleotides in length that hybridizes under specific hybridization and washing conditions to the DNACJ6 gene (either the sense or anti-sense sequence); (ii) a nucleic acid sequence that is at least 90%, more preferably at least 95%, and more preferably at least 98% identical to a transcript of the genomic sequence shown in FIG. 21A, or a nucleic acid sequence that hybridizes under specific hybridization and washing conditions to a transcript of the genomic sequence shown in FIG. 21A; or (iii) a peptide or protein at least 25, at least 50, or at least 75 amino acids in length that is at least 95%, more preferably at least 98%, and more preferably at least 99% identical to the amino acid sequence shown in FIG. 21B.

The nucleic acid encoding human DNAJC6 spans about 151 kb in length as reported in the NCBI gene database for gene ID: 9829, location accession no. NC_{—}000001.9 (65503018 . . . 65654140). It has been reported that the DNAJC6 gene generates one transcript, which comprises nineteen exons as reported in the NCBI nucleotide database under gene ID: 9829, accession no. NM_{—}014787.2; the protein encoded by this transcript is 913 amino acids in length as reported in the NCBI protein database under gene ID: 9829, accession no. NP_{—}055602.1. It is also understood that the DNAJC6 gene may have many transcript variants. For example, it has been suggested that the DNAJC6 gene may generate at least two transcript variants (see, e.g. the ECGENE database, available at the web site, http://genome.ewha.ac.kr/ECgene/, under entry H1C11947). Polymorphisms have been identified in the coding regions and untranslated regions of the exons, as well as in the introns and in the chromosome outside of the transcript region or regions of the DNAJC6 gene. As examples of the polymorphisms in the DNAJC6 gene, the NCBI SNP database reports 1111 specific polymorphic sites for the DNAJC6 gene under gene ID: 9829 in the corresponding SNP database. The mRNA sequence and amino acid sequence of DNAJC6 are set forth in FIGS. 21A and 21B, respectively.

A.21. KIAA0888

As used herein, the term “KIAA0888 gene” is understood to mean a nucleic acid sequence that is (i) at least 90%, more preferably at least 95%, and more preferably at least 98% identical to at least 75, at least 150, at least 225, at least 500, or at least 750 nucleotides in length of the known sequence for the KIA0888 gene as reported in the NCBI gene database under gene ID: 26049, gene location accession no. NC_{—}000005.8 (74109155 . . . 74198371, complement) or a strand complementary thereto; (ii) the full length sequence of the KIAA0888 gene as reported in the NCBI gene database under gene ID: 26049, gene location accession no. NC_{—}000005.8 (74109155 . . . 74198371, complement); (iii) a naturally occurring allelic variant of one of the foregoing sequences; or (iv) a nucleic acid sequence complementary to one of the foregoing sequences.

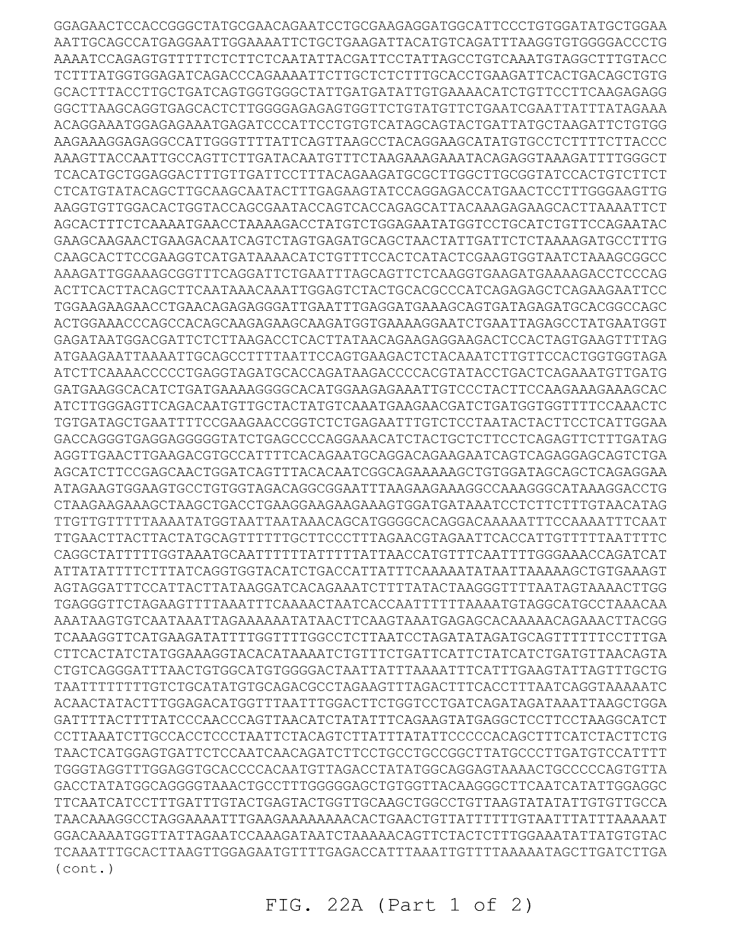

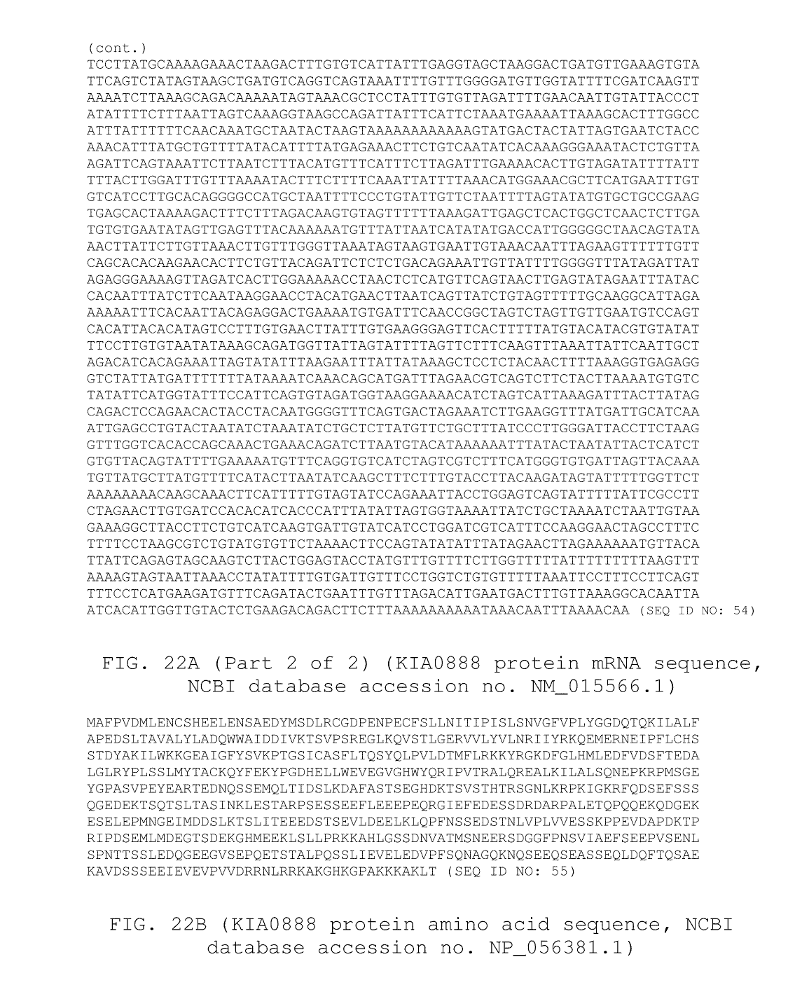

As used herein, a “KIAA0888 gene product” is understood to mean (i) a nucleic acid, sequence at least 75, at least 150, or at least 225 nucleotides in length that hybridizes under specific hybridization and washing conditions to the KIAA0888 gene (either the sense or anti-sense sequence); (ii) a nucleic acid sequence that is at least 90%, more preferably at least 95%, and more preferably at least 98% identical to the mRNA sequence shown in FIG. 22A, or a nucleic acid sequence that hybridizes under specific hybridization and washing conditions to the sequence shown in FIG. 22A; or (iii) a peptide or protein at least 25, at least 50, or at least 75 amino acids in length that is at least 95%, more preferably at least 98%, and more preferably at least 99% identical to the amino acid sequence shown in FIG. 22B.

The nucleic acid encoding human KIAA0888 spans about 89 kb in length as reported in the NCBI gene database for gene ID: 26049, location accession no. NC_{—}000005.8 (74109155 . . . 74198371, complement). It has been reported that the KIAA0888 gene generates one transcript, which comprises thirteen exons as reported in the NCBI nucleotide database under gene ID: 26049, accession no. NM_{—}015566.1; the protein encoded by this transcript is 670 amino acids in length as reported in the NCBI protein database under gene ID: 26049, accession no. NP_{—}056381.1. It is understood that the KIAA0888 gene may have many transcript variants. For example, it has been suggested that the KIAA0888 protein gene may generate at least two transcript variants (see, e.g., the Ensembl database, available at the web site, http://http://www.ensembl.org/, under entry ENSG00000198780). Polymorphisms have been identified in the KIAA0888 gene. As examples of the polymorphisms in the KIAA0888 gene, the NCBI SNP database reports 423 specific polymorphic sites for the KIAA0888 gene under gene ID: 26049 in the corresponding SNP database. The mRNA sequence and amino acid sequence of KIAA0888 are set forth in FIGS. 22A and 22B, respectively.

A.22. ENPP2

ENPP2 functions as both a phosphodiesterase, which cleaves phosphodiester bonds at the 5′ end of oligonucleotides, and as a phospholipase, which catalyzes production of lysophosphatidic acid (LPA) in extracelluar fluids. It has been suggested that ENPP2 may stimulate the motility of tumor cells and has angiogenic properties. (Umezu-Goto et al. (2002), Autotaxin has lysophospholipase D activity leading to tumor cell growth and motility by lysophosphatidic acid production, J. Cell Biol., 158(2):227-33.) As used herein, the term “ENPP2 gene” is understood to mean a nucleic acid sequence that is (i) at least 90%, more preferably at least 95%, and more preferably at least 98% identical to at least 75, at least 150, at least 225, at least 500, or at least 750 nucleotides in length of the known sequence for the ENPP2 gene as reported in the NCBI gene database under gene ID: 5168, gene location accession no. NC_{—}000008.9 (120638500 . . . 120720287, complement) or a strand complementary thereto; (ii) the full length sequence of the ENPP2 gene reported in the NCBI gene database under gene ID: 5168, gene location accession no. NC_{—}000008.9 (120638500 . . . 120720287, complement); (iii) a naturally occurring allelic variant of one of the foregoing sequences; or (iv) a nucleic acid sequence complementary to one of the foregoing sequences.

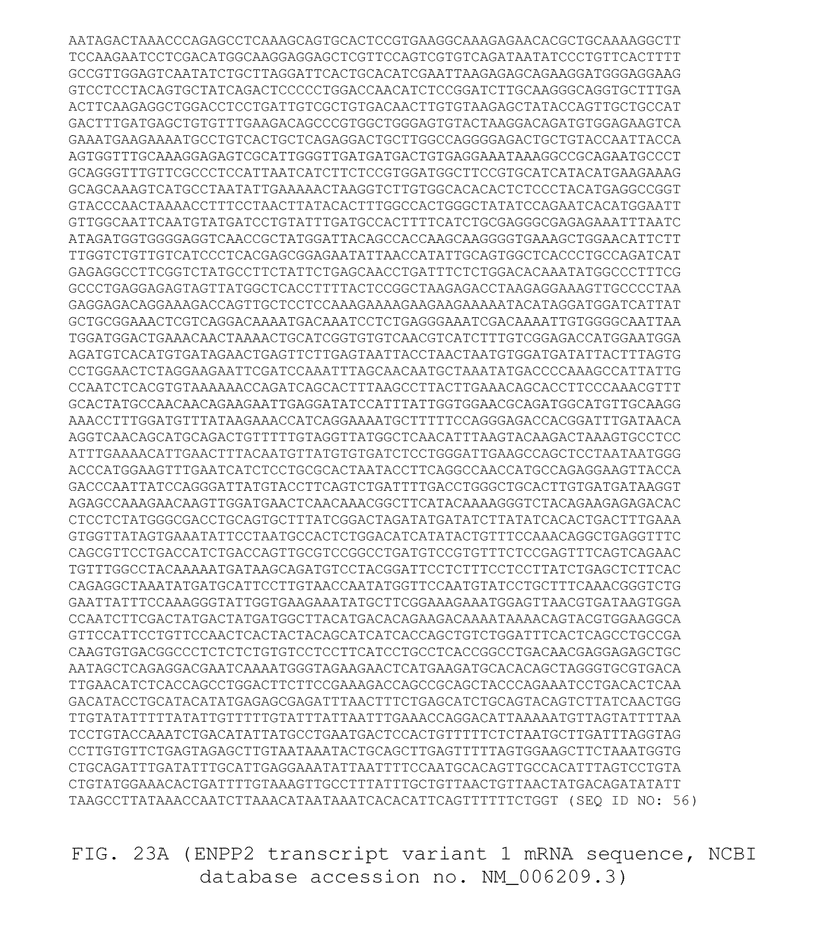

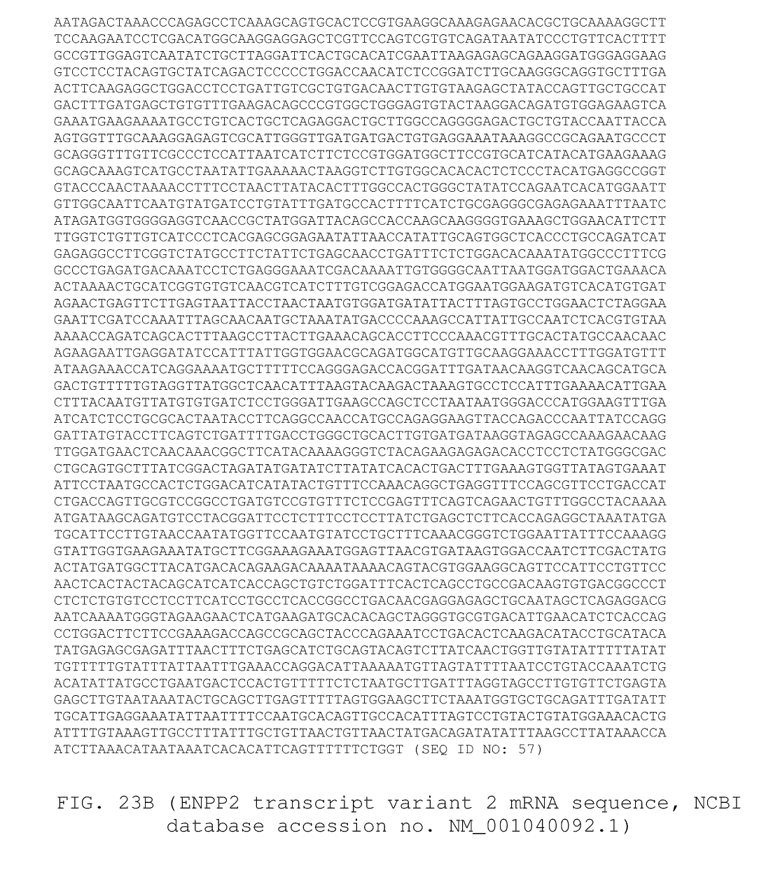

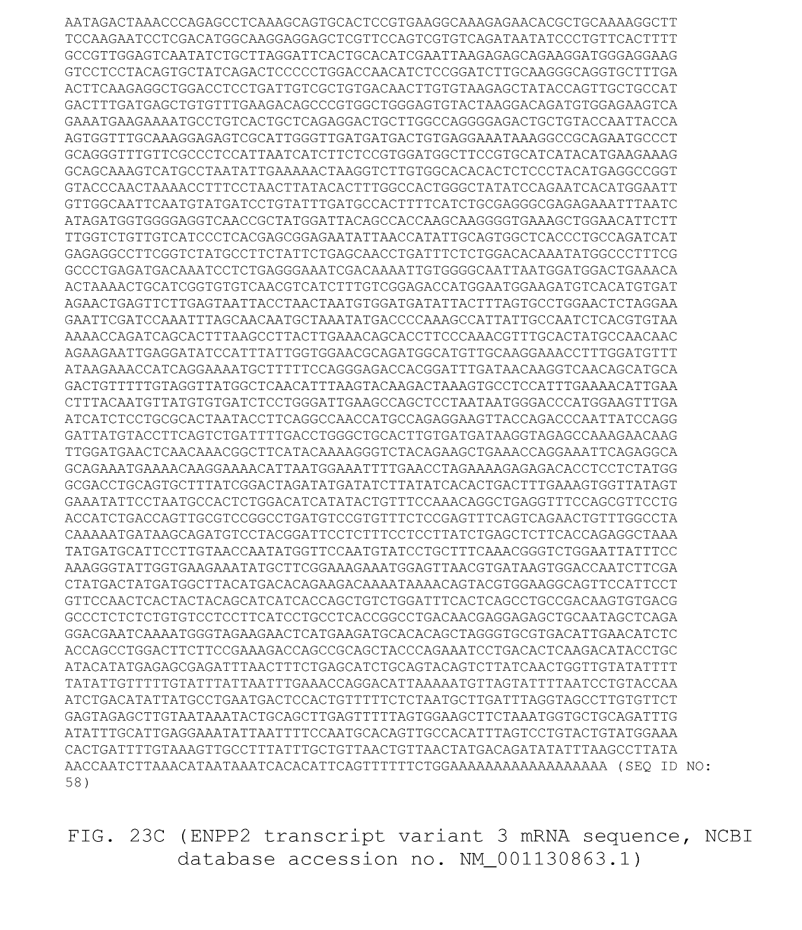

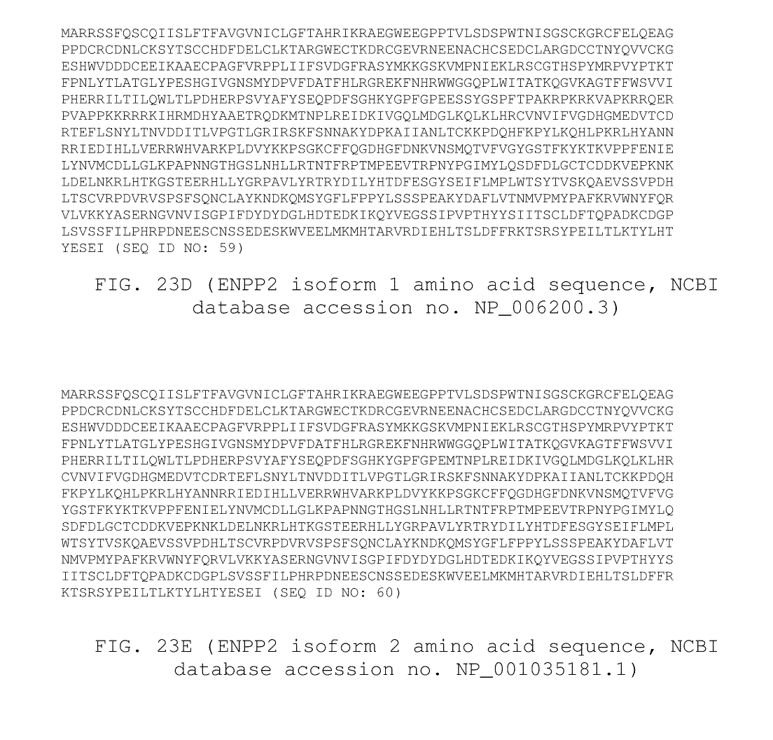

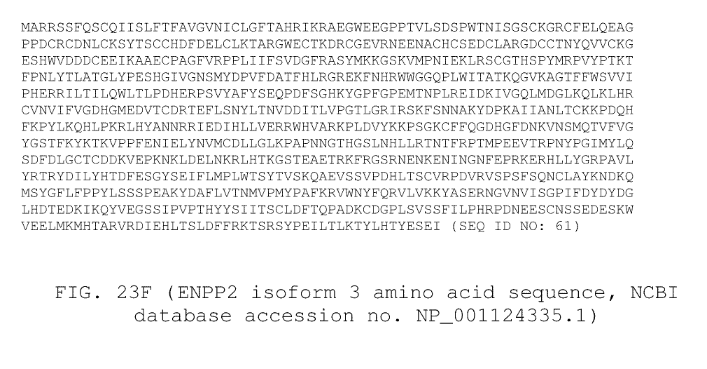

As used herein, a “ENPP2 gene product” is understood to mean (i) a nucleic acid sequence at least 75, at least 150, or at least 225 nucleotides in length that hybridizes under specific hybridization and washing conditions to the ENPP2 gene (either the sense or anti-sense sequence); (ii) a nucleic acid sequence that is at least 90%, more preferably at least 95%, and more preferably at least 98% identical to the mRNA sequence shown in one of FIGS. 23A and 23B, or a nucleic acid sequence that hybridizes under specific hybridization and washing conditions to the sequence shown in one of FIGS. 23A and 23B; or (iii) a peptide or protein at least 25, at least 50, or at least 75 amino acids in length that is at least 95%, more preferably at least 98%, and more preferably at least 99% identical to the amino acid sequence shown in one of FIGS. 23C and 23D.

The nucleic acid encoding human ENPP2 spans about 82 kb in length as reported in the NCBI gene database for gene ID: 5168, location accession no. NC_{—}000008.9 (120638500 . . . 120720287, complement). It has been reported that the ENPP2 gene generates three transcripts: isoform 1, isoform 2, and isoform 3. The transcript of isoform 1 comprises twenty-six exons as reported in the NCBI nucleotide database under gene ID: 5168, accession no. NM_{—}006209.3; the protein encoded by this transcript variant is 915 amino acids in length as reported in the NCBI protein database under gene ID: 5168, accession no. NP_{—}006200.3. The transcript of isoform 2 comprises twenty-five exons as reported in the NCBI nucleotide database under gene ID: 5168, accession no. NM_{—}001040092.1; the protein encoded by this transcript variant is 863 amino acids in length as reported in the NCBI protein database under gene ID: 5168, accession no. NP_{—}001035181.1. The transcript of isoform 3 comprises twenty-six exons as reported in the NCBI nucleotide database under gene ID: 5168, accession no. NM_{—}001130863.1; the protein encoded by this transcript variant is 888 amino acids in length as reported in the NCBI protein database under gene ID: 5168, accession no. NP_{—}001124335.1. It is also understood that the ENPP2 gene may have more transcript variants. For example, it has been suggested that the ENPP2 gene may generate at least five transcript variants (see, e.g. the ECGENE database, available at the web site, http://genome.ewha.ac.kr/ECgene/, under entry H8C12384). Polymorphisms have been identified in the coding regions and untranslated regions of the exons, as well as in the introns and in the chromosome outside of the transcript region or regions of the ENPP2 gene. As examples of the polymorphisms in the ENPP2 gene, the NCBI SNP database reports 495 specific polymorphic sites for the ENPP2 gene under gene ID: 5168 in the corresponding SNP database. The mRNA sequences and amino acid sequences of ENPP2 are set forth in FIGS. 23A-23B and 23C-23D, respectively.

A.23. FAM38B

As used herein, the term “FAM38B gene” is understood to mean a nucleic acid sequence that is (i) at least 90%, more preferably at least 95%, and more preferably at least 98% identical to at least 75, at least 150, at least 225, at least 500, or at least 750 nucleotides in length of the known sequence for the FAM38B gene as reported in the NCBI gene database under gene ID: 63895, gene location accession no. NC_{—}000018.8 (10660850 . . . 10687814, complement) or a strand complementary thereto; (ii) the full length sequence of the FAM38B gene as reported in the NCBI gene database gene ID: 63895, gene location accession no. NC_{—}000018.8 (10660850 . . . 10687814, complement); (iii) a naturally occurring allelic variant of one of the foregoing sequences; or (iv) a nucleic acid sequence complementary to one of the foregoing sequences.

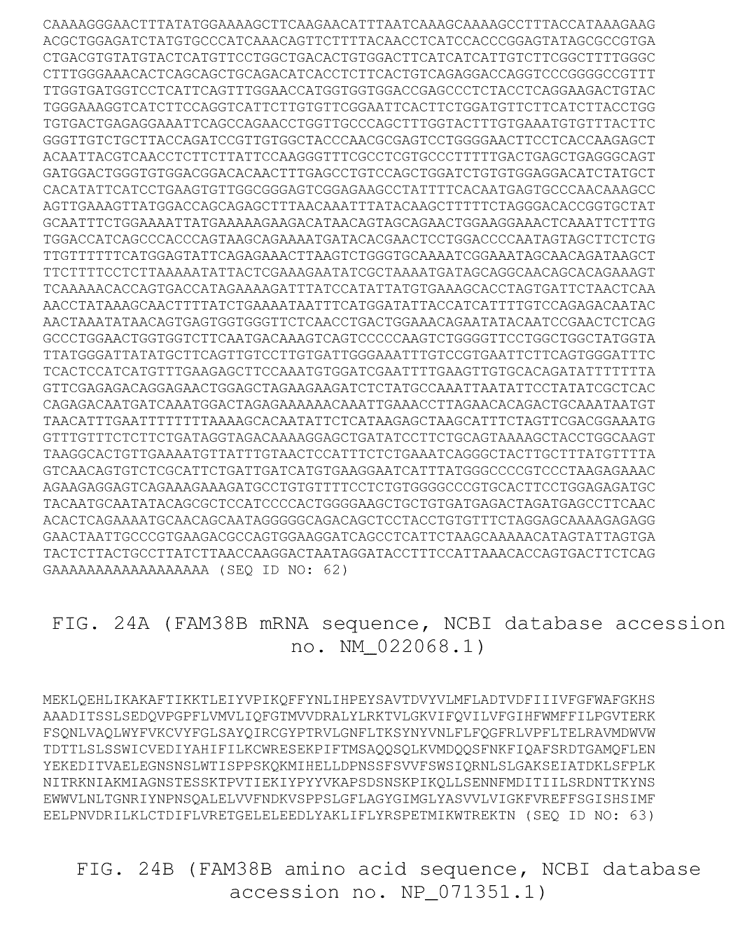

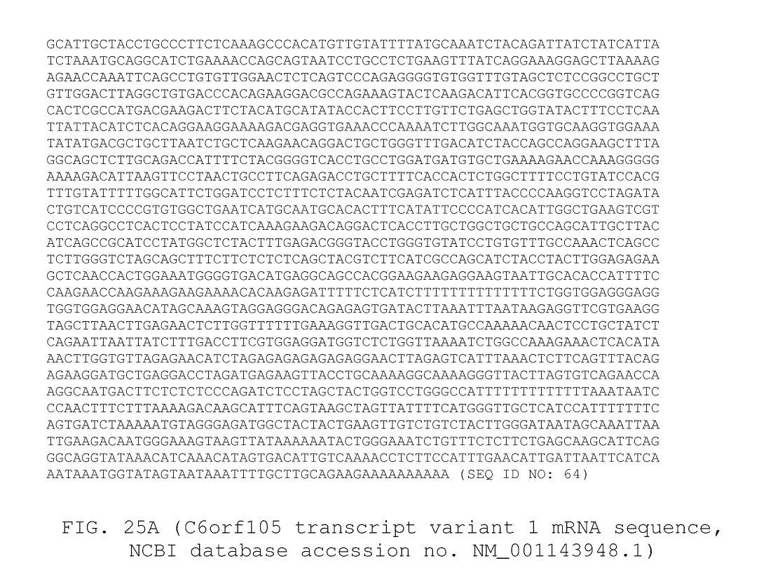

As used herein, a “FAM38B gene product” is understood to mean (i) a nucleic acid sequence at least 75, at least 150, or at least 225 nucleotides in length that hybridizes under specific hybridization and washing conditions to the FAM38B gene (either the sense or anti-sense sequence); (ii) a nucleic acid sequence that is at least 90%, more preferably at least 95%, and more preferably at least 98% identical to the mRNA sequence shown in FIG. 24A, or a nucleic acid sequence that hybridizes under specific hybridization and washing conditions to the sequence shown in FIG. 24A; or (iii) a peptide or protein at least 25, at least 50, or at least 75 amino acids in length that is at least 95%, more preferably at least 98%, and more preferably at least 99% identical to the amino acid sequence shown in FIG. 24B.

The nucleic acid encoding human FAM38B spans about 27 kb in length as reported in the NCBI gene database for gene ID: 63895, location accession no. NC_{—}000018.8 (10660850 . . . 10687814, complement). It has been reported that the FAM38B gene generates one transcript, which comprises eleven exons as reported in the NCBI nucleotide database under gene ID: 63895, accession no. NM_{—}022068.1; the protein encoded by this transcript is 544 amino acids in length as reported in the NCBI protein database under gene ID: 63895, accession no. NP_{—}071351.1. It is also understood that the FAM38B gene may have many transcript variants. For example, it has been suggested that the FAM38B gene may generate at least six transcript variants (see, e.g., the ECGENE database, available at the web site, http://genome.ewha.ac.kr/ECgene/, under entry H18C1357). Polymorphisms have been identified in the coding regions of the exons, as well as in the introns and in the chromosome outside of the transcript region or regions of the FAM38B gene. As examples of the polymorphisms in the FAM38B gene, the NCBI SNP database reports 361 specific polymorphic sites for the FAM38B gene under gene ID: 63895 in the corresponding SNP database. The mRNA sequence and amino acid sequence of FAM38B are set forth in FIGS. 24A and 24B, respectively.

A.24. C6orf105

As used herein, the term “C6orf105 gene” is understood to mean a nucleic acid sequence that is (i) at least 90%, more preferably at least 95%, and more preferably at least 98% identical to at least 75, at least 150, at least 225, at least 500, or at least 750 nucleotides in length of the known sequence for the C6orf105 gene as reported in the NCBI gene database under gene ID: 84830, gene location accession no. NC_{—}000006.10 (11821895 . . . 11887052, complement) or a strand complementary thereto; (ii) the full length sequence of the C6orf105 gene as reported in the NCBI gene database gene ID: 84830, gene location accession no. NC_{—}000006.10 (11821895 . . , 11887052, complement); (iii) a naturally occurring allelic variant of one of the foregoing sequences; or (iv) a nucleic acid sequence complementary to one of the foregoing sequences.

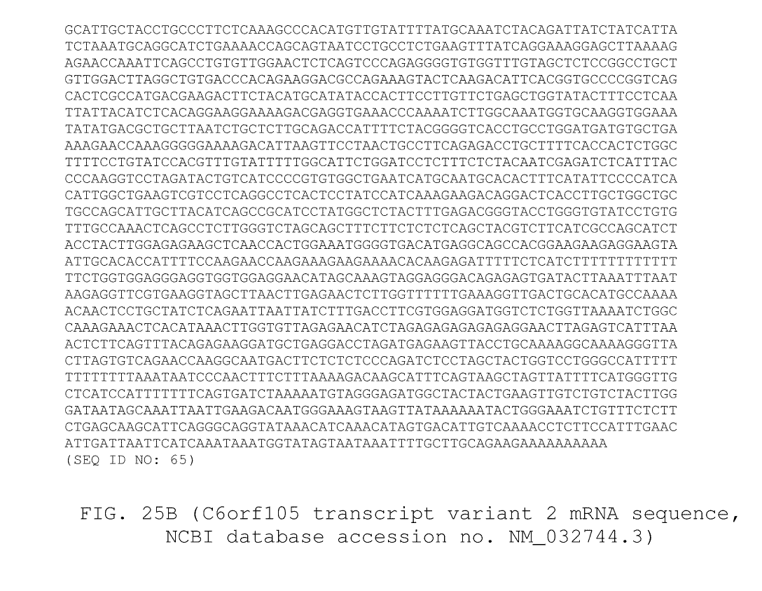

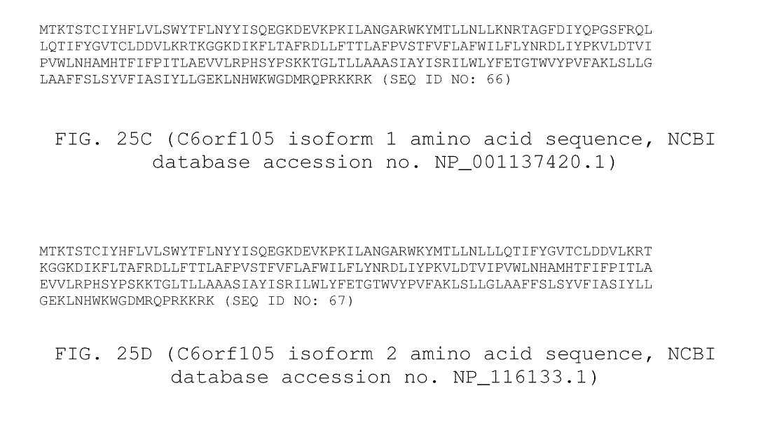

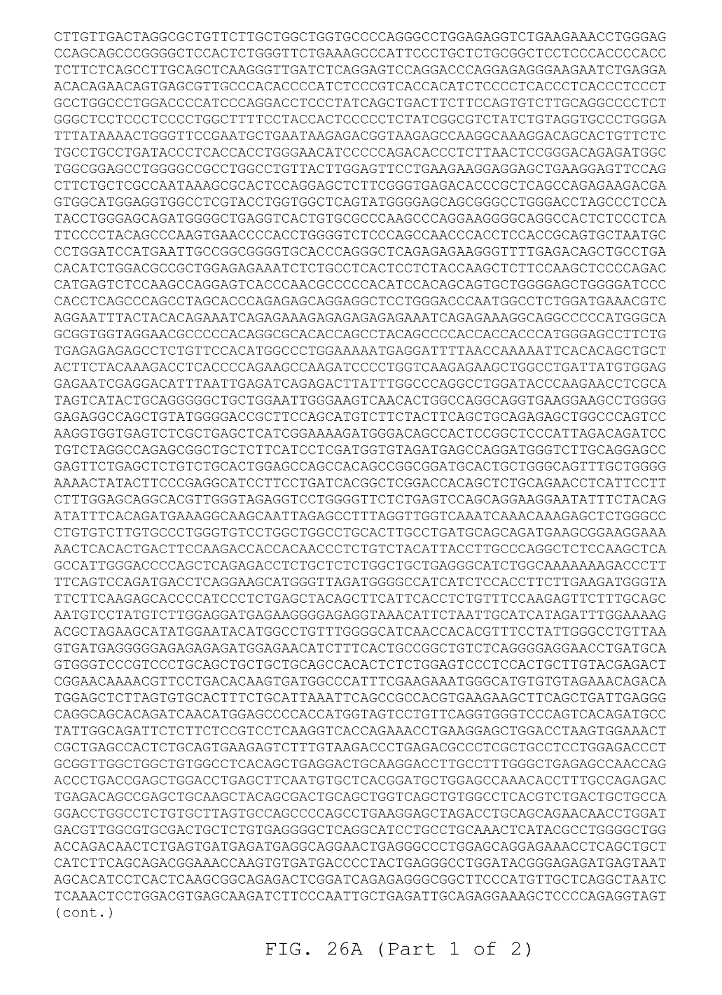

As used herein, a “C6orf105 gene product” is understood to mean (i) a nucleic acid, for example, a sequence at least 75, at least 150, or at least 225 nucleotides in length that hybridizes under specific hybridization and washing conditions to the C6orf105 gene (either the sense or anti-sense sequence); (ii) a nucleic acid sequence that is at least 90%, more preferably at least 95%, and more preferably at least 98% identical to the mRNA sequence shown in FIG. 25A, or a nucleic acid sequence that hybridizes under specific hybridization and washing conditions to the sequence shown in FIG. 25A; or (iii) a peptide or protein at least 25, at least 50, or at least 75 amino acids in length that is at least 95%, more preferably at least 98%, and more preferably at least 99% identical to the amino acid sequence shown in FIG. 25B.

The nucleic acid encoding human C6orf105 spans about 65 kb in length as reported in the NCBI gene database for gene ID: 84830, gene location accession no. NC_{—}000006.10 (11821895 . . . 11887052, complement). It has been reported that the C6orf105 gene generates two transcripts: isoform 1 and isoform 2. The transcript of isoform 1 comprises seven exons as reported in the NCBI nucleotide database under gene ID: 84830, accession no. NM_{—}001143948.1; the protein encoded by this transcript variant is 248 amino acids in length as reported in the NCBI protein database under gene ID: 84830, accession no. NP_{—}001137420.1. The transcript of isoform 2 comprises six exons as reported by the NCBI nucleotide database under gene ID: 84830, accession no. NM_{—}032744.3; the protein encoded by this transcript variant is 230 amino acids in length as reported in the NCBI protein database under gene ID: 84830, accession no. NP_{—}116133.1. It is also understood that the C6orf105 gene may have more transcript variants. For example, it has been suggested that the C6orf105 gene may generate at least six transcript variants (see, e.g., the ECGENE database, available at the web site, http://genome.ewha.ac.kr/ECgene/, under entry H6C1816). Polymorphisms have been identified in the coding regions and untranslated regions of the exons, as well as in the introns and in the chromosome outside of the transcript region or regions of the C6orf105 gene. As examples of the polymorphisms in the C6orf105 gene, the NCBI SNP database reports 646 specific polymorphic sites for the C6orf105 gene under gene ID: 84830 in the corresponding SNP database. The mRNA sequence and amino acid sequence of C6orf 105 are set forth in FIGS. 25A and 25B, respectively.

A.25. NALP1

NALP1 is characterized by an N-terminal pyrin domain and has been known to be involved in the activation of caspase-1 by Toll-like receptors and in protein complexes that activate proinflammatory caspases (Tschopp J et al. (2003), NALPs: a novel protein family involved in inflammation, Nat Rev Mol Cell Biol. 4(2):95-104.) As used herein, the term “NALP1 gene” is understood to mean a nucleic acid sequence that is (i) at least 90%, more preferably at least 95%, and more preferably at least 98% identical to at least 75, at least 150, at least 225, at least 500, or at least 750 nucleotides in length of the known sequence for the NALP1 gene as reported in the NCBI gene database under gene ID: 22861, gene location accession no. NC_{—}0000017.9 (5345443 . . . 5428556, complement) or a strand complementary thereto; (ii) the full length sequence of the NALP1 gene as reported in the NCBI gene database gene ID: 22861, gene location accession no. NC_{—}0000017.9 (5345443 . . . 5428556, complement); (iii) a naturally occurring allelic variant of one of the foregoing sequences; or (iv) a nucleic acid sequence complementary to one of the foregoing sequences.

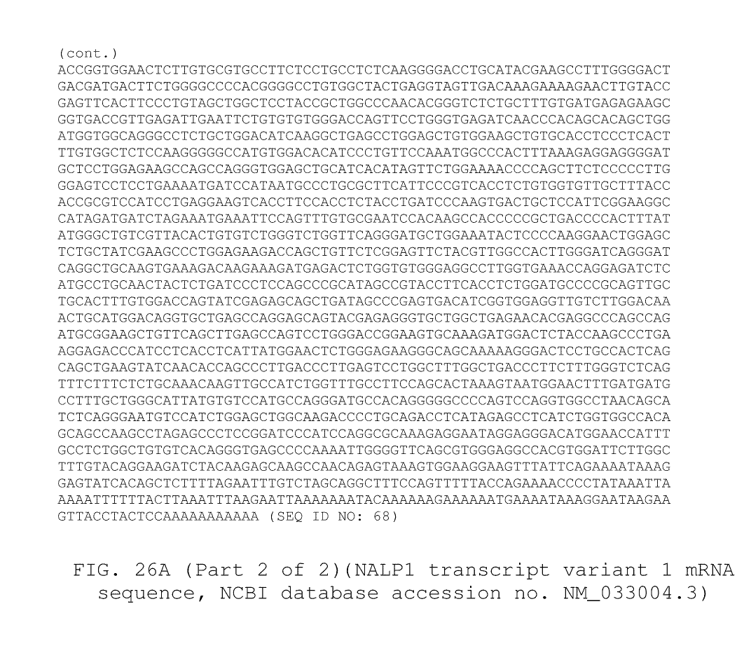

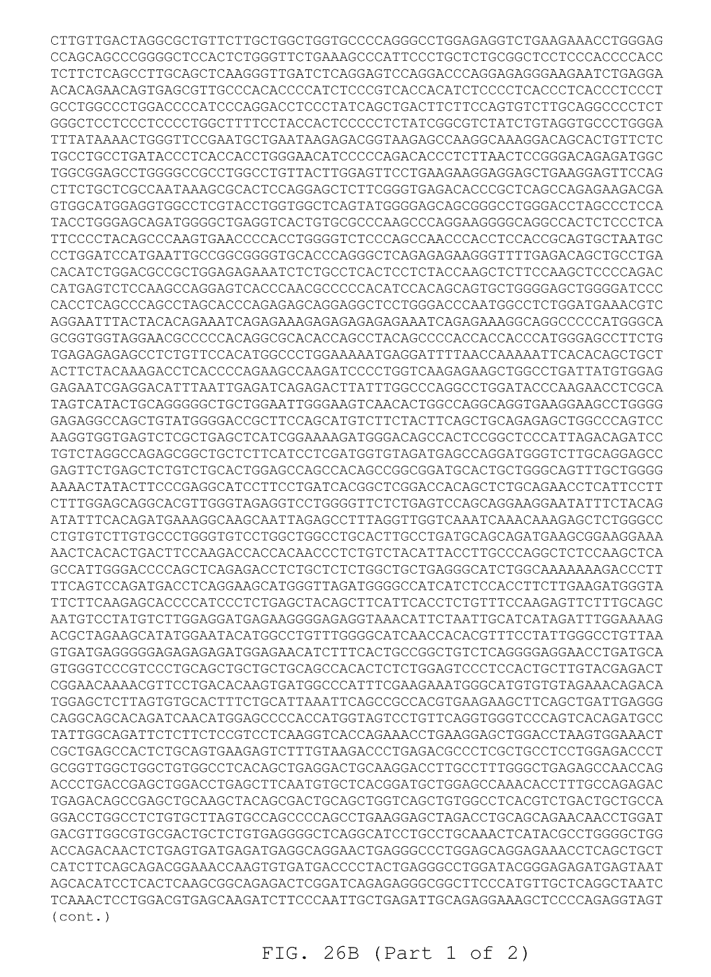

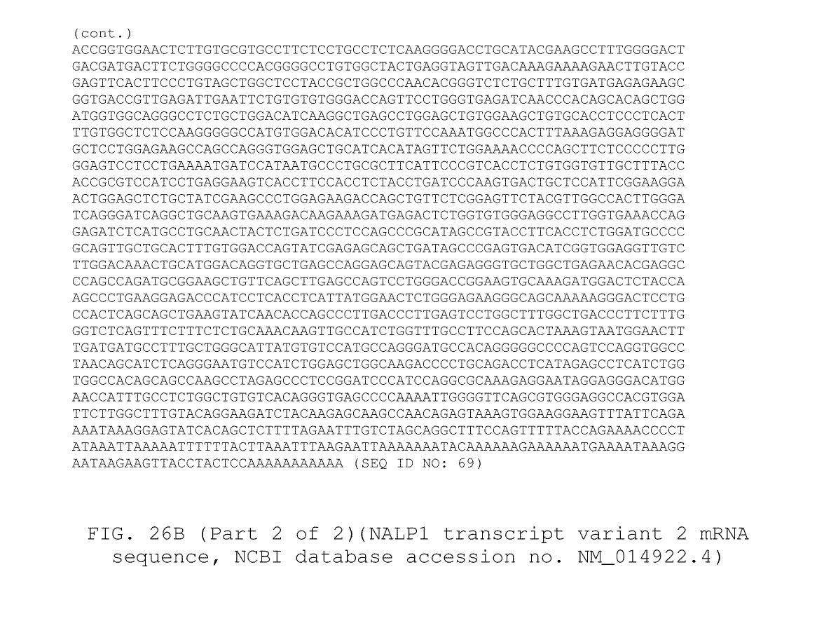

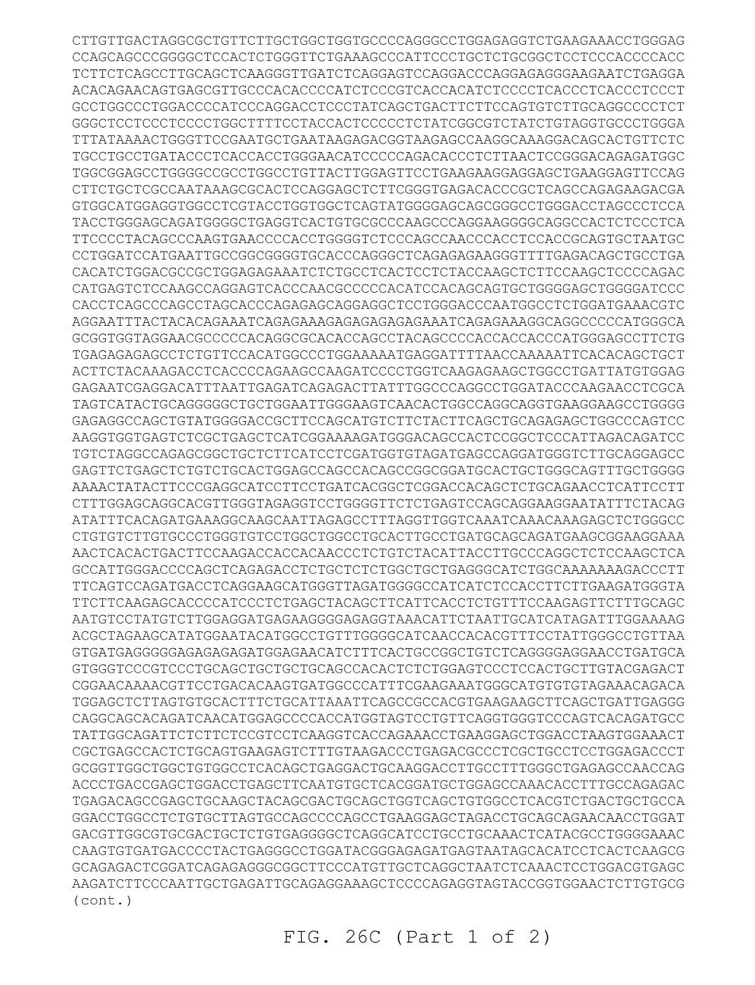

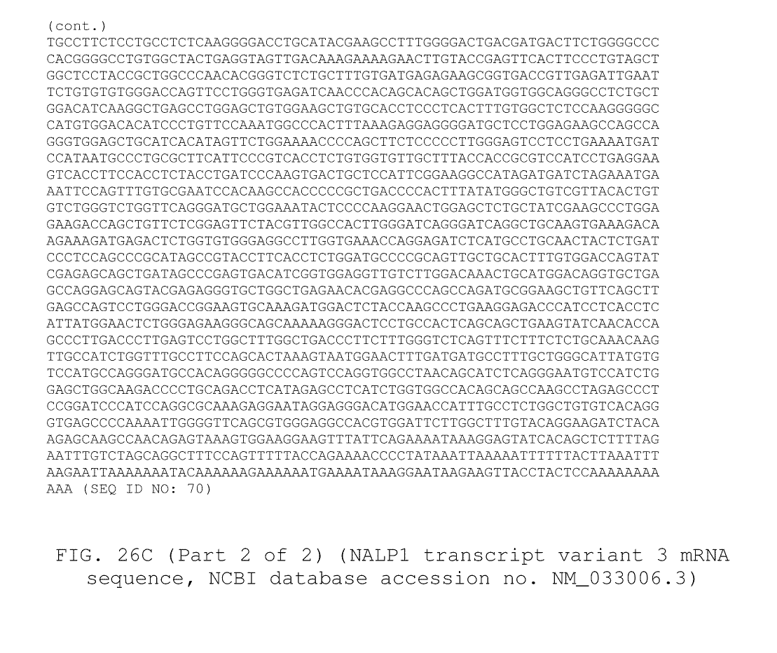

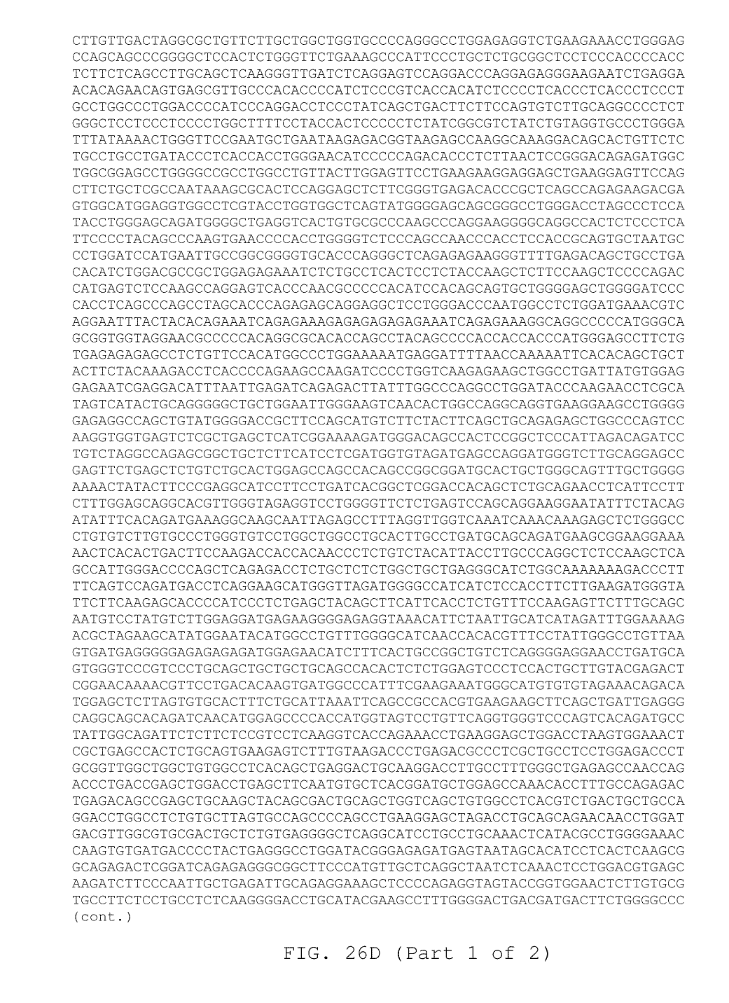

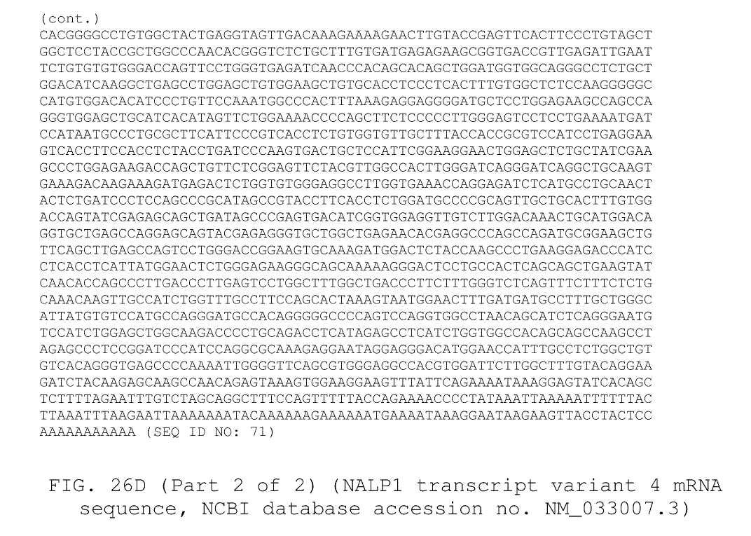

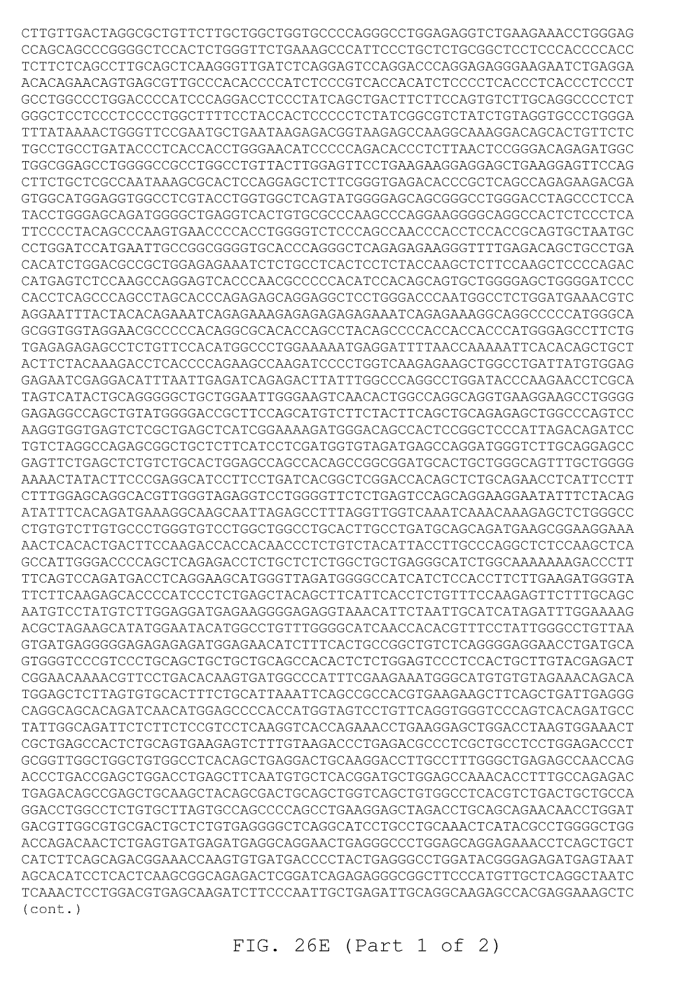

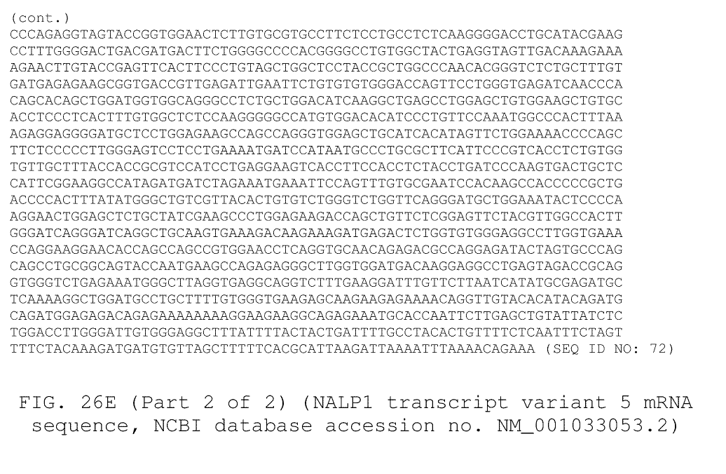

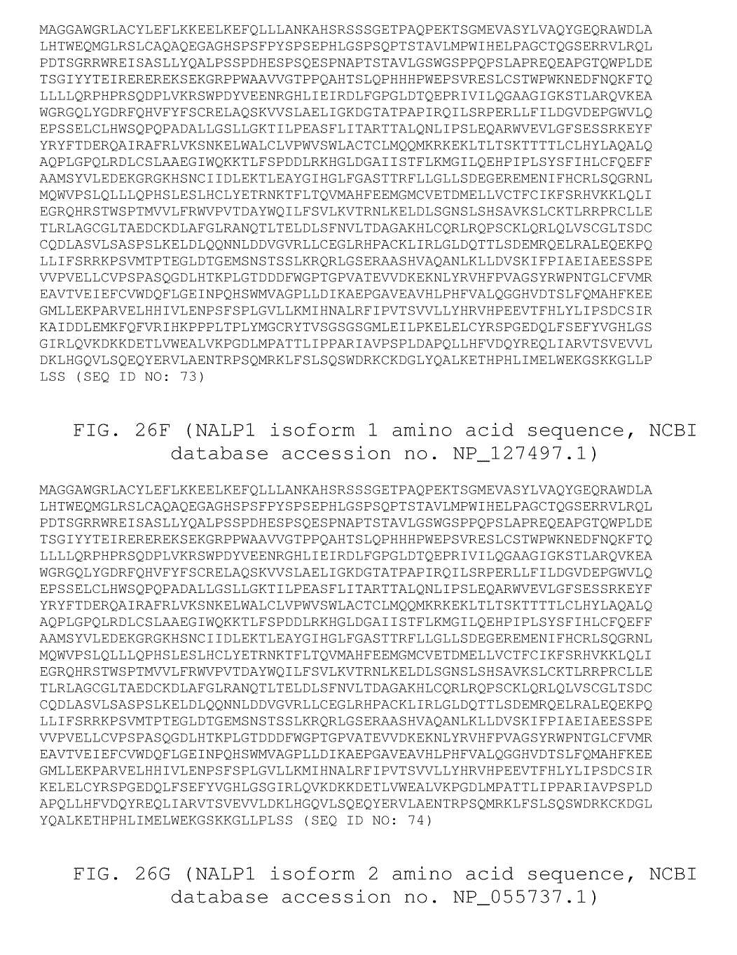

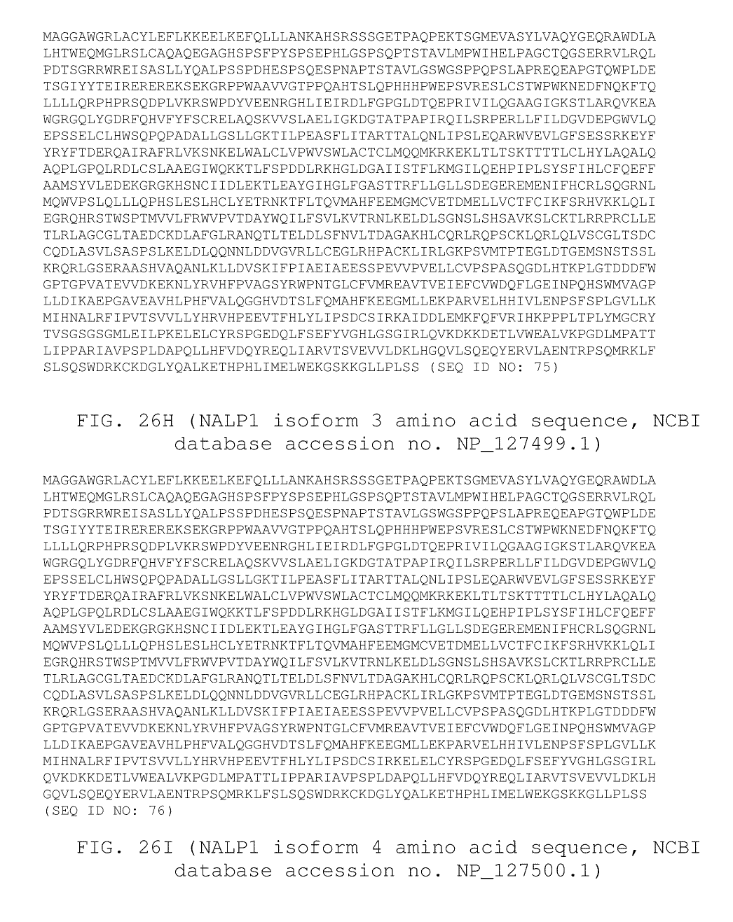

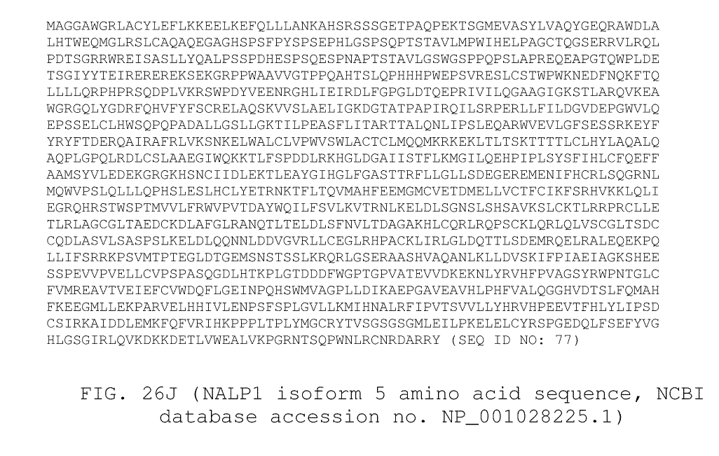

As used herein, a “NALP1 gene product” is understood to mean (i) a nucleic acid, for example, a sequence at least 75, at least 150, or at least 225 nucleotides in length that hybridizes under specific hybridization and washing conditions to the NALP1 gene (either the sense or anti-sense sequence); (ii) a nucleic acid sequence that is at least 90%, more preferably at least 95%, and more preferably at least 98% identical to the mRNA sequence shown in one of FIGS. 26A-26E, or a nucleic acid sequence that hybridizes under specific hybridization and washing conditions to the sequence shown in one of FIGS. 26A-26E; or (iii) a peptide or protein at least 25, at least 50, or at least 75 amino acids in length that is at least 95%, more preferably at least 98%, and more preferably at least 99% identical to the amino acid sequence shown in one of FIGS. 26F-26J.

The nucleic acid encoding human NALP1 spans about 83 kb in length as reported in the NCBI gene database for gene ID: 22861, gene location accession no. NC_{—}0000017.9 (5345443 . . . 5428556, complement). It has been reported that the NALP1 gene generates five transcripts: isoforms 1-5. The transcript of isoform 1 comprises seventeen exons as reported in the NCBI nucleotide database under gene ID: 22861, accession no. NM_{—}033004.3; the protein encoded by this transcript variant is 1473 amino acids in length as reported in the NCBI protein database under gene ID: 22861, accession no. NP_{—}127497.1. The transcript of isoform 2 comprises sixteen exons as reported in the NCBI nucleotide database under gene ID: 22861, accession no. NM_{—}014922.4; the protein encoded by this transcript variant is 1429 amino acids in length as reported in the NCBI protein database under gene ID: 22861, accession no. NP_{—}055737.1. The transcript of isoform 3 comprises sixteen exons as reported in the NCBI nucleotide database under gene ID: 22861, accession no. NM_{—}033006.3; the protein encoded by this transcript variant is 1443 amino acids in length as reported in the NCBI protein database under gene ID: 22861, accession no. NP_{—}127499.1. The transcript of isoform 4 comprises fifteen exons as reported in the NCBI nucleotide database under gene ID: 22861, accession no. NM_{—}033007.3; the protein encoded by this transcript variant is 1399 amino acids in length as reported in the NCBI protein database under gene ID: 22861, accession no. NP_{—}127500.1. The transcript for isoform 5 comprises sixteen exons as reported in the NCBI nucleotide database under gene ID: 22861, accession no. NM_{—}001033053.2; the protein encoded by this transcript variant is 1375 amino acids in length as reported in the NCBI protein database under gene ID: 22861, accession no. NP_{—}001028225.1. It is also understood that the NALP1 gene may have more transcript variants. For example, it has been suggested that the NALP1 gene may generate at least twenty-two transcript variants (see, e.g., the ECGENE database, available at the web site, http://genome.ewha.ac.kr/ECgene/, under entry H17C1503). Polymorphisms have been identified in the coding regions and untranslated regions of the exons, as well as in the introns and in the chromosome outside of the transcript region or regions of the NALP1 gene. As examples of the polymorphisms in the NALP1 gene, the NCBI SNP database reports 727 specific polymorphic sites for the NALP1 gene under gene ID: 22861 in the corresponding SNP database. The mRNA sequences and amino acid sequences of NALP1 are set forth in FIGS. 26A-26E and 26F-26J, respectively.

A.26. Networks

The RORA, CRIM1, CXCR4, C5orf26, IGHG3, NALP2, PLA2G4A, IGLJ3, SHQ1, UCHL1, TANC1, PKP2, DNAJC6, C6orf105, NALP1, RGS13, CXCL13, RPS6KA2, MMP7, IL1A, ABCA1, VCAN, KIAA0888, ENPP2, and/or FAM38B genes and/or gene products may function together, and/or with other genes and/or gene products, in biological pathways. Using data relating to the expression changes of the genes of interest, namely RORA, CRIM1, CXCR4, C5orf26, IGHG3, NALP2, PLA2G4A, IGLJ3, SHQ1, UCHL1, TANC1, PKP2, DNAJC6, C6orf105, NALP1, RGS13, CXCL13, RPS6KA2, MMP7, IL1A, ABCA1, VCAN, KIAA0888, ENPP2, and FAM38B, as inputs, Ingenuity Pathway Analysis (IPA) software (available from Ingenuity® Systems, Redwood City, Calif.) was used to predict biological networks. IPA software uses information about interactions among genes and gene products from publications and biological databases to make the predictions. The IPA software generates a group of networks in which the genes of interest are most likely to be involved. In addition, the IPA software determines additional genes known to interact with the genes of interest. Interactions may be positive or negative, or direct or indirect. The results of the IPA analysis for RORA, CRIM1, CXCR4, C5orf26, IGHG3, NALP2, PLA2G4A, IGLJ3, SHQ1, UCHL1, TANC1, PKP2, DNAJC6, C6orf105, NALP1, RGS13, CXCL13, RPS6KA2, MMP7, IL1A, ABCA1, VCAN, KIAA0888, ENPP2, and FAM38B are shown in Table 1.

As indicated in Table 1, six networks, which include the molecules shown, were predicted. A score was given to each network, with a higher score corresponding to a more significant interaction. The number of focus molecules involved in each network (i.e., the genes of interest that are present in a particular network) is indicated, as well as the biological functions with which each network may be involved. Bolded names are focus molecules (and are selected from the genes of interest) and unbolded names are also associated with the biological network.

TABLE 1

| | | | Focus | |
|---|---|---|---|---|
| Network | Molecules in Network | Score | Molecules | Functions |
| 1 | ABCA1, cholesterol sulfate, CXCL13, | 33 | 12 | Tissue |
| | CXCR4, DEFB104A, DEFB4 (includes | | | Morphology, |
| | EG: 56519), DOK5, ERK, FCGR1B, | | | Dermatological |
| | FCGR1C, IGHG3, IL1, IL1/IL6/TNF, | | | Diseases and |
| | IL1A, IL1F5, IL1F6, IL1F7, IL1F8, | | | Conditions, Organ |
| | IL1F9, IL1F10, LDL, Mapk, MMP7, | | | Morphology |
| | NFkB (complex), NALP2, P38 MAPK, | | | |
| | PELI2, PLA2G4A, RGS13, RORA, | | | |
| | RPS6KA2, S100A3, Tgf beta, TRIB1, | | | |
| | VCAN | | | |
| 2 | ALDH1A1, COL4A1, CRIM1, DSP, | 8 | 4 | Protein Synthesis, |
| | EEF1D, EIF3C, EIF4A1, EIF5A, | | | Drug Metabolism, |
| | ELAVL2, ENPP2, IGFBP7, KRT5, | | | Lipid Metabolism |
| | MYCN, NMI, PKP2, retinoic acid, | | | |
| | RPL3, RPL4, RPL6, RPL11, RPL29, | | | |
| | RPL23A (includes EG: 6147), RPS3, | | | |
| | RPS16, RPS19, RPS20, RPS4X, | | | |
| | SLC38A2, TPI1, UCHL1, USP3, | | | |
| | ZBTB17, ZEB2, ZFAND5, ZNF217 | | | |
| 3 | APOA1, FAM169A | 3 | 1 | Antigen |
| | | | | Presentation, |
| | | | | Carbohydrate |
| | | | | Metabolism, |
| | | | | Cardiovascular |
| | | | | Disease |
| 4 | MIRN93 (includes EG: 407050), TANC1 | 3 | 1 | Cancer, |
| | | | | Reproductive |
| | | | | System Disease |
| 5 | DNAJC, DNAJC6, | 2 | 1 | |
| | Hsp22/Hsp40/Hsp90, MIRN128-1 | | | |
| | (includes EG: 406915), MIRN128-2 | | | |
| | (includes EG: 406916) | | | |
| 6 | FAM38B, MIRN34C (includes | 2 | 1 | Cancer, |
| | EG: 407042), MIRN98 (includes | | | Gastrointestinal |
| | EG: 407054), MIRNLET7A1, | | | Disease, Hepatic |
| | MIRNLET7A2, MIRNLET7A3, | | | System Disease |
| | MIRNLET7B (includes EG: 406884), | | | |
| | MIRNLET7C, MIRNLET7F1 (includes | | | |
| | EG: 406888), MIRNLET7F2 (includes | | | |
| | EG: 406889), MIRNLET7G (includes | | | |
| | EG: 406890) | | | |

A.27. Functions

Further analysis of the biological functions in which more than one of RORA, CRIM1, CXCR4, C5orf26, IGHG3, NALP2, PLA2G4A, IGLJ3, SHQ1, UCHL1, TANC1, PKP2, DNAJC6, C6orf105, NALP1, RGS13, CXCL13, RPS6KA2, MMP7, IL1A, ABCA1, VCAN, KIAA0888, ENPP2, and/or FAM38B genes and/or gene products play a role also was examined using the IPA software. As indicated in Table 2, one or more of RORA, CRIM1, CXCR4, C5orf26, IGHG3, NALP2, PLA2G4A, IGLJ3, SHQ1, UCHL1, TANC1, PKP2, DNAJC6, C6orf105, NALP1, RGS13, CXCL13, RPS6KA2, MMP7, IL1A, ABCA1, VCAN, KIAA0888, ENPP2, and FAM38B genes and/or gene products share similar biological functions. Each row of Table 2 shows a group of genes or gene products that are associated with a particular biological function. The P-value indicates the likelihood that the association between the genes and the biological function indicated is due to random chance. A lower P-value indicates a greater likelihood that the association between the genes and the biological function is significant.

TABLE 2

| Biological Function | P-value | Molecules |
|---|---|---|
| Genetic Disorder | 4.29 × 10⁻⁶-3.59 × 10⁻² | IL1A, MMP7, PKP2, CXCR4, VCAN, |
| | | ABCA1, UCHL1, PLA2G4A, IGHG3, |
| | | CXCL13, RORA, ENPP2, RGS13, NALP2, |
| | | CRIM1 |
| Tissue Development | 4.52 × 10⁻⁶-3.61 × 10⁻² | PLA2G4A, IL1A, PKP2, CXCL13, CXCR4, |
| | | ENPP2, VCAN |
| Cellular Function and | 9.04 × 10⁻⁶-1.76 × 10⁻² | IL1A, CXCL13, CXCR4, ABCA1 |
| Maintenance | | |
| Cellular Movement | 9.04 × 10⁻⁶-3.98 × 10⁻² | PLA2G4A, IL1A, MMP7, CXCL13, CXCR4, |
| | | ENPP2, VCAN |
| Hematological System | 9.04 × 10⁻⁶-3.86 × 10⁻² | PLA2G4A, IL1A, CXCL13, RORA, CXCR4, |
| Development and | | ABCA1 |
| Function | | |
| Humoral Immune | 9.04 × 10⁻⁶-3.86 × 10⁻² | PLA2G4A, IL1A, MMP7, IGHG3, CXCL13, |
| Response | | RORA, CXCR4 |
| Lipid Metabolism | 1.32 × 10⁻⁵-3.98 × 10⁻² | PLA2G4A, MMP7, IL1A, RORA, ENPP2, |
| | | ABCA1 |
| Molecular Transport | 1.32 × 10⁻⁵-3.98 × 10⁻² | PLA2G4A, MMP7, IL1A, CXCL13, RORA, |
| | | CXCR4, ENPP2, ABCA1 |
| Small Molecule | 1.32 × 10⁻⁵-3.98 × 10⁻² | PLA2G4A, IL1A, MMP7, RORA, ENPP2, |
| Biochemistry | | RGS13, VCAN, ABCA1 |
| Carbohydrate Metabolism | 5.4 × 10⁻⁵-3.36 × 10⁻² | PLA2G4A, MMP7, IL1A, ENPP2, ABCA1 |
| Respiratory System | 5.4 × 10⁻⁵-3.79 × 10⁻³ | PLA2G4A, IL1A, ABCA1 |
| Development and | | |
| Function | | |
| Tissue Morphology | 5.4 × 10⁻⁵-3.86 × 10⁻² | PLA2G4A, MMP7, IL1A, CXCL13, CXCR4, |
| | | ABCA1 |
| Hematological Disease | 7.53 × 10⁻⁵-3.86 × 10⁻² | PLA2G4A, MMP7, IL1A, PKP2, CXCL13, |
| | | CXCR4, RORA, ABCA1 |
| Skeletal and Muscular | 1.17 × 10⁻⁴-3 × 10⁻² | PLA2G4A, IL1A, CXCL13, CXCR4, |
| Disorders | | RPS6KA2 |
| Immunological Disease | 1.25 × 10⁻⁴-3.12 × 10⁻² | PLA2G4A, IL1A, CXCL13, RORA, CXCR4, |
| | | RGS13, NALP2, ABCA1 |
| Reproductive System | 1.42 × 10⁻⁴-3 × 10⁻² | UCHL1, PLA2G4A, IL1A, MMP7, CXCL13, |
| Disease | | CXCR4, CRIM1, VCAN |
| Cancer | 2.83 × 10⁻⁴-3.67 × 10⁻² | PLA2G4A, MMP7, IL1A, IGHG3, CXCL13, |
| | | CXCR4, ENPP2, CRIM1, VCAN |
| Cell-To-Cell Signaling | 2.83 × 10⁻⁴-3.98 × 10⁻² | UCHL1, IL1A, MMP7, CXCL13, PKP2, |
| and Interaction | | CXCR4, VCAN, ABCA1 |
| Cellular Growth and | 3.56 × 10⁻⁴-3 × 10⁻² | UCHL1, PLA2G4A, MMP7, IL1A, CXCR4, |
| Proliferation | | ENPP2, VCAN |
| Cardiovascular Disease | 4.76 × 10⁻⁴-3.49 × 10⁻² | PLA2G4A, MMP7, IL1A, PKP2, CXCR4, |
| | | ABCA1 |
| Metabolic Disease | 4.82 × 10⁻⁴-1.13 × 10⁻² | IL1A, RORA, ABCA1 |
| Cell Death | 6.87 × 10⁻⁴-3 × 10⁻² | PLA2G4A, MMP7, IL1A, CXCR4, |
| | | RPS6KA2, VCAN |
| Connective Tissue | 6.87 × 10⁻⁴-3 × 10⁻² | PLA2G4A, MMP7, IL1A, CXCL13, CXCR4, |
| Disorders | | ENPP2, RPS6KA2 |
| Inflammatory Disease | 9.27 × 10⁻⁴-3 × 10⁻² | PLA2G4A, MMP7, IL1A, CXCL13, CXCR4, |
| | | ABCA1 |
| Cardiovascular System | 9.79 × 10⁻⁴-3.98 × 10⁻² | PLA2G4A, IL1A, CXCL13, PKP2, CXCR4, |
| Development and | | ENPP2, VCAN |
| Function | | |
| Cell Morphology | 9.79 × 10⁻⁴-3.86 × 10⁻² | PLA2G4A, IL1A, CXCR4 |
| Cellular Development | 9.79 × 10⁻⁴-3.86 × 10⁻² | IL1A, RORA, CXCR4, RPS6KA2, VCAN |
| Dermatological Diseases | 9.99 × 10⁻⁴-3 × 10⁻² | IL1A, CXCL13, CXCR4, RGS13 |
| and Conditions | | |
| Skeletal and Muscular | 1.03 × 10⁻³-3.98 × 10⁻² | PLA2G4A, MMP7, IL1A, PKP2, CXCR4, |
| System Development and | | ENPP2, RGS13 |
| Function | | |
| Tumor Morphology | 1.03 × 10⁻³-3 × 10⁻² | IL1A, MMP7, CXCR4, ENPP2 |
| Drug Metabolism | 1.14 × 10⁻³-3.86 × 10⁻² | PLA2G4A, IL1A, ABCA1 |
| Gastrointestinal Disease | 1.14 × 10⁻³-2.02 × 10⁻² | PLA2G4A, IL1A, MMP7, IGHG3 |
| Cell-mediated Immune | 1.2 × 10⁻³-2.5 × 10⁻² | PLA2G4A, IL1A, MMP7, IGHG3, CXCL13, |
| Response | | RORA, CXCR4 |
| Hematopoiesis | 1.2 × 10⁻³-3 × 10⁻² | IL1A, MMP7, CXCL13, RORA, CXCR4 |
| Lymphoid Tissue | 1.2 × 10⁻³-3 × 10⁻² | IL1A, CXCL13, RORA, CXCR4 |
| Structure and | | |
| Development | | |
| Organismal Injury and | 1.2 × 10⁻³-3.86 × 10⁻² | PLA2G4A, MMP7, IL1A, PKP2, CXCR4, |
| Abnormalities | | ABCA1 |
| Nervous System | 1.26 × 10⁻³-2.87 × 10⁻² | UCHL1, IL1A, CXCR4, RORA |
| Development and | | |
| Function | | |
| Organ Development | 1.26 × 10⁻³-2.66 × 10⁻² | PLA2G4A, CXCL13, PKP2, RORA, CXCR4, |
| | | VCAN, ABCA1 |
| Cellular Assembly and | 1.27 × 10⁻³-3.86 × 10⁻² | UCHL1, PLA2G4A, IGHG3, CXCR4, |
| Organization | | ENPP2, VCAN, ABCA1 |
| Cellular Compromise | 1.27 × 10⁻³-3.12 × 10⁻² | CXCR4, RGS13, ABCA1 |
| Connective Tissue | 1.27 × 10⁻³-3.98 × 10⁻² | PLA2G4A, IL1A, CXCL13, ENPP2, VCAN |
| Development and | | |
| Function | | |
| Embryonic Development | 1.27 × 10⁻³-3.12 × 10⁻² | CXCR4, ENPP2, RPS6KA2, ABCA1 |
| Endocrine System | 1.27 × 10⁻³-1.51 × 10⁻² | IL1A, CXCR4 |
| Development and | | |
| Function | | |
| Endocrine System | 1.27 × 10⁻³-8.83 × 10⁻³ | MMP7, IL1A, CXCR4 |
| Disorders | | |
| Gene Expression | 1.27 × 10⁻³-4.04 × 10⁻² | PLA2G4A, IL1A, RORA |
| Hair and Skin | 1.27 × 10⁻³-3.12 × 10⁻² | IL1A, RORA, ABCA1 |
| Development and | | |
| Function | | |
| Immune Cell Trafficking | 1.27 × 10⁻³-2.26 × 10⁻² | PLA2G4A, MMP7, IL1A, CXCL13, CXCR4 |
| Inflammatory Response | 1.27 × 10⁻³-3.73 × 10⁻² | PLA2G4A, MMP7, IL1A, IGHG3, CXCL13, |
| | | CXCR4, ABCA1 |
| Ophthalmic Disease | 1.27 × 10⁻³-1.27 × 10⁻³ | VCAN |
| Organ Morphology | 1.27 × 10⁻³-1.89 × 10⁻² | PLA2G4A, IL1A, CXCL13, PKP2, RORA, |
| | | ABCA1 |
| Reproductive System | 1.27 × 10⁻³-2.75 × 10⁻² | PLA2G4A, CXCR4, ABCA1 |
| Development and | | |
| Function | | |
| Vitamin and Mineral | 1.27 × 10⁻³-1.83 × 10⁻² | CXCL13, CXCR4, ABCA1 |
| Metabolism | | |
| Respiratory Disease | 2 × 10⁻³-3.86 × 10⁻² | PLA2G4A, MMP7, ABCA1 |
| Cell Signaling | 2.23 × 10⁻³-3.98 × 10⁻² | IL1A, CXCL13, CXCR4, RORA, RGS13, |
| | | RPS6KA2, ABCA1 |
| Amino Acid Metabolism | 2.53 × 10⁻³-2.5 × 10⁻² | IL1A, VCAN |
| Cell Cycle | 2.53 × 10⁻³-5.06 × 10⁻³ | IL1A, RPS6KA2 |
| Developmental Disorder | 2.53 × 10⁻³-1.26 × 10⁻² | PLA2G4A, MMP7 |
| Infection Mechanism | 2.53 × 10⁻³-3 × 10⁻² | CXCR4 |
| Infectious Disease | 2.53 × 10⁻³-2.11 × 10⁻² | IL1A, CXCR4, CRIM1 |
| Neurological Disease | 2.53 × 10⁻³-1.26 × 10⁻² | UCHL1, PLA2G4A, IL1A, RORA, CXCR4, |
| | | ENPP2, CRIM1, VCAN, ABCA1 |
| Organismal Development | 2.53 × 10⁻³-4.1 × 10⁻² | PLA2G4A, IL1A |
| Renal and Urological | 2.53 × 10⁻³-3.79 × 10⁻³ | IL1A, ABCA1 |
| Disease | | |
| Antigen Presentation | 2.97 × 10⁻³-3.12 × 10⁻² | PLA2G4A, IL1A, MMP7, IGHG3, CXCL13, |
| | | CXCR4, ABCA1 |
| Hypersensitivity Response | 3.79 × 10⁻³-8.83 × 10⁻³ | IL1A |
| Nucleic Acid Metabolism | 5.06 × 10⁻³-3.98 × 10⁻² | RORA, RGS13, ABCA1 |
| Hepatic System | 6.32 × 10⁻³-6.32 × 10⁻³ | IL1A |
| Development and | | |
| Function | | |
| Hepatic System Disease | 7.57 × 10⁻³-1.26 × 10⁻² | IL1A, MMP7 |
| Organismal Functions | 7.57 × 10⁻³-7.57 × 10⁻³ | IL1A |
| Behavior | 1.01 × 10⁻²-3.61 × 10⁻² | UCHL1 |
| Protein Synthesis | 1.01 × 10⁻²-1.88 × 10⁻² | ABCA1 |
| Post-Translational | 1.38 × 10⁻²-3.61 × 10⁻² | UCHL1, MMP7, RPS6KA2, ABCA1 |
| Modification | | |
| RNA Damage and Repair | 2.13 × 10⁻²-2.13 × 10⁻² | IL1A |
| RNA Post-Transcriptional | 2.13 × 10⁻²-2.13 × 10⁻² | IL1A |
| Modification | | |

Accordingly, the invention provides methods for determining whether an individual has or is at risk of developing one or more angiogenic disorders, for example, an ocular angiogenic disorder, for example, a disorder associated with choroidal neovascularization, for example, age-related macular degeneration. As described below, a variety of methods may be used to detect the presence and/or amount of one or more of the RORA, CRIM1, CXCR4, C5orf26, IGHG3, NALP2, PLA2G4A, IGLJ3, SHQ1, UCHL1, TANC1, PKP2, DNAJC6, C6orf105, NALP1, RGS13, CXCL13, RPS6KA2, MMP7, IL1A, ABCA1, VCAN, KIAA0888, ENPP2, and FAM38B genes and/or gene products in a sample. A gene product is a molecule that results from the transcription and/or translation of a gene, for example, one or more of the RORA, CRIM1, CXCR4, C5orf26, IGHG3, NALP2, PLA2G4A, IGLJ3, SHQ1, UCHL1, TANC1, PKP2, DNAJC6, C6orf105, NALP1, RGS13, CXCL13, RPS6KA2, MMP7, IL1A, ABCA1, VCAN, KIAA0888, ENPP2, and FAM38B genes. The gene product can include without limitation, for example, (i) a nucleic acid, for example, an RNA, for example, a messenger RNA (mRNA) and (ii) a protein. The RORA, CRIM1, CXCR4, C5orf26, IGHG3, NALP2, PLA2G4A, IGLJ3, SHQ1, UCHL1, TANC1, PKP2, DNAJC6, C6orf105, NALP1, RGS13, CXCL13, RPS6KA2, MMP7, IL1A, ABCA1, VCAN, KIAA0888, ENPP2, and FAM38B genes and gene products also include, for example, polymorphic variants, promoter regions, introns, exons, and untranslated regions of the genes and/or gene products, and/or fragments thereof.

### B. Prognosis and Diagnosis of Angiogenic Disorders

As discussed, the invention provides a method of determining whether a mammal is at risk of developing, or has, one or more angiogenic disorders, for example, an ocular angiogenic disorder, for example, a disorder associated with choroidal neovascularization, for example, age-related macular degeneration. In particular, the method can be used to determine if a mammal, such as, a human, is at risk of developing or has an ocular angiogenic disorder, such as age-related macular degeneration. The method includes the steps of: (a) measuring the amount of a gene or gene product in a test sample harvested from the mammal; and (b) comparing the amount of the gene or gene product against a control value, wherein an amount of the gene or gene product in the sample greater than the control value is indicative that the mammal is at risk of developing, or has, the ocular angiogenic disorder (e.g. the neovascular form of age-related macular degeneration). The gene or gene product is selected from the group consisting of CXCL13, RPS6KA2, MMP7, IL1A, KIAA0888, ENPP2, CRIM1, CXCR4, C5orf26, IGHG3, IGLJ3, SHQ1, DNAJC6, C6orf105, NALP1, and RGS13. In certain embodiments, one or more markers are measured and compared against corresponding control values. For example, in certain embodiments, the markers are selected from and include two, three, four, five, six, and more of a CXCL13 gene, a RPS6KA2 gene, a MMP7 gene, an IL1A gene, a KIAA0888 gene, an ENPP2 gene, a CRIM1 gene, a CXCR4 gene, a C5orf26 gene, an IGHG3 gene, an IGLJ3 gene, a SHQ1 gene, a DNAJC6 gene, a C6orf105 gene, a NALP1 gene, a RGS13 gene, a CXCL13 gene product, a RPS6KA2 gene product, a MMP7 gene product, an IL1A gene product, a KIAA0888 gene product, an ENPP2 gene product, a CRIM1 gene product, a CXCR4 gene product, a C5orf26 gene product, an IGHG3 gene product, an IGLJ3 gene product, a SHQ1 gene product, a DNAJC6 gene product, a C6orf105 gene product, a NALP1 gene product, and a RGS13 gene product, and the markers are measured and compared against corresponding control values. For example, but without limitation, groups of one or more markers to be measured can be selected according to those grouped in a particular network, as shown in Table 1, or according to those grouped by a particular biological function, as shown in Table 2. Moreover, any of the molecules shown in Table 1 can be used in combination as groups of markers. It should be understood that any one or more of the upregulated markers can be combined with any one or more downregulated marker, as well.

The corresponding control values can be the median amount of the CXCL13, RPS6KA2, MMP7, IL1A, KIAA0888, ENPP2, CRIM1, CXCR4, C5orf26, IGHG3, IGLJ3, SHQ1, DNAJC6, C6orf105, NALP1, and RGS13 genes and/or gene products present in samples of similar origin as the test sample harvested from individuals without the angiogenic condition, for example, without the ocular angiogenic condition, such as age-related macular degeneration. When the diagnostic method is for predicting whether an individual with the dry form of age-related macular degeneration is at risk of developing the wet form of age-related macular degeneration, the control value can be the median amount of the CXCL13, RPS6KA2, MMP7, IL1A, KIAA0888, ENPP2, CRIM1, CXCR4, C5orf26, IGHG3, IGLJ3, SHQ1, DNAJC6, C6orf105, NALP1, and RGS13 genes and/or gene products present in samples of similar origin as the test sample harvested from individuals diagnosed as having the dry form of age-related macular degeneration.

In addition, the invention provides a method of determining whether a mammal is at risk of developing, or has, one or more angiogenic disorders, for example, an ocular angiogenic disorder, for example, a disorder associated with choroidal neovascularization, for example, age-related macular degeneration. In particular, the method can be used to determine if a mammal, such as, a human, is at risk of developing an ocular angiogenic disorder, such as age-related macular degeneration. The method includes the steps: of (a) measuring the amount of a gene or gene product in a test sample harvested from the mammal; and (b) comparing the amount of the gene or gene product against a control value, wherein an amount of the gene or gene product in the sample less than the control value is indicative that the mammal is at risk of developing, or has, the ocular angiogenic disorder (e.g. age-related macular degeneration). The gene or gene product is selected from the group consisting of RORA, NALP2, PLA2G4A, PKP2, UCHL1, TANC1, ABCA1, VCAN, and FAM38B. In certain embodiments, one or more markers are measured and compared against corresponding control values. For example, in certain embodiments, the markers are selected from and include two, three, four, five, six, and more of a RORA gene, a NALP2 gene, a PLA2G4A gene, a PKP2 gene, an UCHL1 gene, a TANC1 gene, an ABCA1 gene, a VCAN gene, a FAM38B gene, a RORA gene product, a NALP2 gene product, a PLA2G4A gene product, a PKP2 gene product, an UCHL1 gene product, a TANC1 gene product, an ABCA1 gene product, a VCAN gene product, and a FAM38B gene product, and the markers are measured and compared against corresponding control values. For example, but without limitation, groups of one or more markers to be measured can be selected according to those grouped in a particular network, as shown in Table 1, or according to those grouped by a particular biological function, as shown in Table 2. Moreover, any of the molecules shown in Table 1 can be used in combination as groups of markers. It should be understood that any one or more of the upregulated markers can be combined with any one or more downregulated markers, as well.

The corresponding control values can be the median amounts of the RORA, NALP2, PLA2G4A, PKP2, UCHL1, TANC1, ABCA1, VCAN, and FAM38B genes or gene products present in samples of similar origin as the test sample harvested from individuals without the angiogenic condition, for example, without the ocular angiogenic condition, such as age-related macular degeneration, that is under investigation. When the diagnostic method is for predicting whether an individual with the dry form of age-related macular degeneration is at risk of developing the wet form of age-related macular degeneration, the control value can be the median amount of the RORA, NALP2, PLA2G4A, PKP2, UCHL1, TANC1, ABCA1, VCAN, and FAM38B genes or gene products present in samples of similar origin as the test sample harvested from individuals diagnosed as having the dry form of age-related macular degeneration.

The test sample can be any appropriate sample, for example, a tissue or body fluid sample. The body fluid sample, for example, can be selected from blood, serum, plasma, lacrimal fluid, vitreous, aqueous, and synovial fluid. The tissue sample, for example, can be selected from the group consisting of conjunctiva, cornea, sclera, uvea, retina, choroid, neovascular tissue, and optic nerve. The tissue sample can also include a plurality of cells, for example, 10-1000 cells, harvested from one of the foregoing tissues.

As discussed, the present invention includes diagnostic assays for determining the presence and/or amount of one or more of RORA, CRIM1, CXCR4, C5orf26, IGHG3, NALP2, PLA2G4A, IGLJ3, SHQ1, UCHL1, TANC1, PKP2, DNAJC6, C6orf105, NALP1, RGS13, CXCL13, RPS6KA2, MMP1, IL1A, ABCA1, VCAN, KIAA0888, ENPP2, and/or FAM38B genes and gene products (including, for example, polymorphic variants, promoter regions, introns, exons, and untranslated regions of the genes and/or gene products, and/or fragments thereof) in a test sample.

B.1. Protein Detection

The presence and/or amount of a marker protein, for example, the RORA, CRIM1, CXCR4, C5orf26, IGHG3, NALP2, PLA2G4A, IGLJ3, SHQ1, UCHL1, TANC1, PKP2, DNAJC6, C6orf105, NALP1, RGS13, CXCL13, RPS6KA2, MMP1, IL1A, ABCA1, VCAN, KIAA0888, ENPP2, and/or FAM38B protein, in a sample may be detected, for example, by combining the sample with a binding moiety capable of binding specifically to the marker protein. The binding moiety may comprise, for example, a member of a ligand-receptor pair, i.e., a pair of molecules capable of specific binding interactions. The binding moiety may comprise, for example, a member of a specific binding pair, such as antibody-antigen, enzyme-substrate, nucleic acid-nucleic acid, protein-nucleic acid, protein-protein or other specific binding pairs known in the art. Binding proteins may be designed which have enhanced affinity for the marker protein. Optionally, the binding moiety may be linked with a detectable label, such as an enzymatic, fluorescent, radioactive, phosphorescent or colored particle label. The labeled complex may be detected, e.g., visually or with the aid of a machine, for example, a spectrophotometer or other detector.

The marker proteins also may be detected using one- and two-dimensional gel electrophoresis techniques available in the art, such as those disclosed, for example, in Sambrook and Maniatis et al. eds. (1989) Molecular Cloning: A Laboratory Manual, Cold Spring Harbor Press. In one-dimensional gel electrophoresis, the proteins are usually separated according to their molecular weight. In two-dimensional gel electrophoresis, the proteins are first separated in a pH gradient gel according to their isoelectric point. The resulting gel then is placed on a second polyacrylamide gel, and the proteins separated according to molecular weight (see, for example, O'Farrell (1975) J. Biol. Chem. 250: 4007-4021).

The resulting gel pattern may then be compared with a standard gel pattern derived from a control sample (harvested, for example, from an individual without the angiogenic disorder, for example, without the ocular disorder, such as age-related macular degeneration, that is under study or from an individual with the dry form of age-related macular degeneration, as the case may be) and run under the same or similar conditions. The standard may be stored or obtained in an electronic database of electrophoresis patterns. The presence of a greater amount of a CXCL13, RPS6KA2, MMP7, IL1A, KIAA0888, ENPP2, CRIM1, CXCR4, C5orf26, IGHG3, IGLJ3, SHQ1, DNAJC6, C6orf105, NALP1, or RGS13 protein or a decreased amount of a RORA, NALP2, PLA2G4A, PKP2, UCHL1, TANC1, ABCA1, VCAN, or FAM38B protein in the two-dimensional gel of the test sample compared to a control provides an indication that the individual has, or is at risk of developing, the disorder under study. The detection of two or more proteins in the two-dimensional gel electrophoresis pattern further enhances the accuracy of the assay. For example, assaying for an increased amount of one, two, three, four, five, six, or more of the CXCL13, RPS6KA2, MMP7, IL1A, KIAA0888, ENPP2, CRIM1, CXCR4, C5orf26, IGHG3, IGLJ3, SHQ1, DNAJC6, C6orf105, NALP1, and RGS13 proteins and/or a decreased amount of one, two, three, four, or more of the RORA, NALP2, PLA2G4A, PKP2, UCHL1, TANC1, ABCA1, VCAN, and FAM38B proteins provides a stronger indication that the individual has or is at risk of developing the disorder under study.

Furthermore, a RORA, CRIM1, CXCR4, C5orf26, IGHG3, NALP2, PLA2G4A, IGLJ3, SHQ1, UCHL1, TANC1, PKP2, DNAJC6, C6orf105, NALP1, RGS13, CXCL13, RPS6KA2, MMP7, IL1A, ABCA1, VCAN, KIAA0888, ENPP2, and/or FAM38B protein in a sample may be detected using any of a wide range of immunoassay techniques available in the art such as enzyme linked immunosorbent assays (ELISAs), Western blots, immunoprecipitations and immunofluorescence. For example, the skilled artisan may take advantage of the sandwich immunoassay format to detect if an individual has or is at risk of developing one or more angiogenic disorders, for example, an ocular angiogenic disorder, for example, a disorder associated with choroidal neovascularization, for example, age-related macular degeneration. Alternatively, the skilled artisan may use conventional immuno-histochemical procedures for detecting the presence of RORA, CRIM1, CXCR4, C5orf26, IGHG3, NALP2, PLA2G4A, IGLJ3, SHQ1, UCHL1, TANC1, PKP2, DNAJC6, C6orf105, NALP1, RGS13, CXCL13, RPS6KA2, MMP7, IL1A, ABCA1, VCAN, KIAA0888, ENPP2, and/or FAM38B in a tissue sample, for example, using one or more labeled binding proteins, for example, a labeled antibody.

In a sandwich immunoassay, two antibodies capable of binding the marker protein are used, e.g., one immobilized onto a solid support, and one free in solution and labeled with detectable chemical compound. Examples of chemical labels that may be used for the second antibody include radioisotopes, fluorescent compounds, and enzymes or other molecules which generate colored or electrochemically active products when exposed to a reactant or enzyme substrate. When a sample containing the marker protein is placed in this system, the marker protein binds to both the immobilized antibody and the labeled antibody, to form a “sandwich” immune complex on the support's surface. The complexed marker protein is detected by washing away non-bound sample components and excess labeled antibody, and measuring the amount of labeled antibody complexed to protein on the support's surface.

Both the sandwich immunoassay and the tissue immunohistochemical procedure are highly specific and very sensitive, provided that labels with good limits of detection are used. A detailed review of immunological assay design, theory and protocols can be found in numerous texts in the art, including Butt, ed. (1984) Practical Immunology, Marcel Dekker, New York and Harlow et al., eds. (1988) Antibodies, A Laboratory Approach, Cold Spring Harbor Laboratory.

In general, immunoassay design considerations include preparation of antibodies (e.g., monoclonal or polyclonal antibodies) having sufficiently high binding specificity for the marker or target protein to form a complex that can be distinguished reliably from products of nonspecific interactions. As used herein, the term “antibody” is understood to mean intact an antibody (for example, polyclonal or monoclonal antibody); an antigen binding fragment thereof, for example, an Fab, Fab′ and (Fab′)₂ fragment; and a biosynthetic antibody binding site, for example, an sFv, as described in U.S. Pat. Nos. 5,091,513; and 5,132,405; and 4,704,692. A binding moiety, for example, an antibody, is understood to bind specifically to the target, for example, the RORA, CRIM1, CXCR4, C5orf26, IGHG3, NALP2, PLA2G4A, IGLJ3, SHQ1, UCHL1, TANC1, PKP2, DNAJC6, C6orf105, NALP1, RGS13, CXCL13, RPS6KA2, MMP7, ILIA, ABCA1, VCAN, KIAA0888, ENPP2, or FAM38B protein, when the binding moiety has a binding affinity for the target greater than about 10⁵ M⁻¹, more preferably greater than about 10⁷ M⁻¹.

Antibodies against the RORA, CRIM1, CXCR4, C5orf26, IGHG3, NALP2, PLA2G4A, IGLJ3, SHQ1, UCHL1, TANC1, PKP2, DNAJC6, C6orf105, NALP1, RGS13, CXCL13, RPS6KA2, MMP7, ILIA, ABCA1, VCAN, KIAA0888, ENPP2, and FAM38B proteins which are useful in assays for detecting if an individual has or is at risk of developing age-related macular degeneration may be generated using standard immunological procedures well known and described in the art. (See, e.g., Butt, N. R., ed. (1984) Practical Immunology, Marcel Dekker, New York). Briefly, an isolated RORA, CRIM1, CXCR4, C5orf26, IGHG3, NALP2, PLA2G4A, IGLJ3, SHQ1, UCHL1, TANC1, PKP2, DNAJC6, C6orf105, NALP1, RGS13, CXCL13, RPS6KA2, MMP7, IL1A, ABCA1, VCAN, KIAA0888, ENPP2, or FAM38B protein or fragment thereof is used to raise antibodies in a xenogeneic host, such as a mouse, goat or other suitable mammal.

The RORA, CRIM1, CXCR4, C5orf26, IGHG3, NALP2, PLA2G4A, IGLJ3, SHQ1, UCHL1, TANC1, PKP2, DNAJC6, C6orf105, NALP1, RGS13, CXCL13, RPS6KA2, MMP7, IL1A, ABCA1, VCAN, KIAA0888, ENPP2, or FAM38B protein or fragment thereof is combined with a suitable adjuvant capable of enhancing antibody production in the host, and injected into the host, for example, by intraperitoneal administration. Any adjuvant suitable for stimulating the host's immune response may be used. A commonly used adjuvant is Freund's complete adjuvant (an emulsion comprising killed and dried microbial cells). Where multiple antigen injections are desired, the subsequent injections may comprise the antigen in combination with an incomplete adjuvant (for example, a cell-free emulsion).

Polyclonal antibodies may be isolated from the antibody-producing host by extracting serum containing antibodies to the protein of interest. Monoclonal antibodies may be produced by isolating host cells that produce the desired antibody, fusing these cells with myeloma cells using standard procedures known in the immunology art, and screening for hybrid cells (hybridomas) that react specifically with the target protein and have the desired binding affinity.

Antibody binding domains also may be produced biosynthetically and the amino acid sequence of the binding domain manipulated to enhance binding affinity with a preferred epitope on the target protein. Specific antibody methodologies are well understood and described in the literature. A more detailed description of their preparation can be found, for example, in Butt, N. R., ed. (1984) Practical Immunology, Marcel Dekker, New York.

B.2. Nucleic Acid Detection

The presence and/or amount of a RORA, CRIM1, CXCR4, C5orf26, IGHG3, NALP2, PLA2G4A, IGLJ3, SHQ1, UCHL1, TANC1, PKP2, DNAJC6, C6orf105, NALP1, RGS13, CXCL13, RPS6KA2, MMP7, IL1A, ABCA1, VCAN, KIAA0888, ENPP2, or FAM38B nucleic acid molecule (including, for example, polymorphic variants, promoter regions, introns, exons, and untranslated regions of the genes and/or gene products, and/or fragments thereof), for example, a mRNA, encoding a RORA, CRIM1, CXCR4, C5orf26, IGHG3, NALP2, PLA2G4A, IGLJ3, SHQ1, UCHL1, TANC1, PKP2, DNAJC6, C6orf105, NALP1, RGS13, CXCL13, RPS6KA2, MMP7, IL1A, ABCA1, VCAN, KIAA0888, ENPP2, or FAM38B protein may be determined using a labeled binding moiety capable of specifically binding the RORA, CRIM1, CXCR4, C5orf26, IGHG3, NALP2, PLA2G4A, IGLJ3, SHQ1, UCHL1, TANC1, PKP2, DNAJC6, C6orf105, NALP1, RGS13, CXCL13, RPS6KA2, MMP7, IL1A, ABCA1, VCAN, KIAA0888, ENPP2, or FAM38B nucleic acid, respectively. The binding moiety may comprise, for example, a protein, a nucleic acid or a peptide nucleic acid. Additionally, a target nucleic acid, such as an mRNA encoding RORA, CRIM1, CXCR4, C5orf26, IGHG3, NALP2, PLA2G4A, IGLJ3, SHQ1, UCHL1, TANC1, PKP2, DNAJC6, C6orf105, NALP1, RGS13, CXCL13, RPS6KA2, MMP7, IL1A, ABCA1, VCAN, KIAA0888, ENPP2, or FAM38B protein, may be determined by conducting, for example, a Northern blot analysis using labeled oligonucleotides, e.g., nucleic acid fragments, complementary to and capable of hybridizing specifically with at least a portion of a target nucleic acid.

More specifically, gene probes comprising complementary RNA or DNA to the target nucleotide sequences or mRNA sequences encoding the RORA, CRIM1, CXCR4, C5orf26, IGHG3, NALP2, PLA2G4A, IGLJ3, SHQ1, UCHL1, TANC1, PKP2, DNAJC6, C6orf105, NALP1, RGS13, CXCL13, RPS6KA2, MMP7, IL1A, ABCA1, VCAN, KIAA0888, ENPP2, or FAM38B proteins may be produced using established recombinant techniques or oligonucleotide synthesis. The probes hybridize with complementary nucleic acid sequences presented in the test sample, and can provide exquisite specificity. A short, well-defined probe, coding for a single unique sequence is most precise and preferred. Larger probes are generally less specific. While an oligonucleotide of any length may hybridize to an mRNA transcript, oligonucleotides typically within the range of 8-100 nucleotides, preferably within the range of 15-50 nucleotides, are envisioned to be useful in standard hybridization assays. Choices of probe length and sequence allow one to choose the degree of specificity desired. Hybridization is carried out at from 50° to 65° C. in a high salt buffer solution, formamide or other agents to set the degree of complementarity required. Furthermore, the state of the art is such that probes can be manufactured to recognize essentially any DNA or RNA sequence. For additional particulars, see, for example, Berger et al. (1987) “Guide to Molecular Techniques,” Methods of Enzymol 152.

A wide variety of different labels coupled to the probes may be employed in the protein and nucleic acid assays described herein. The labeled reagents may be provided in solution or coupled to an insoluble support, depending on the design of the assay. The various conjugates may be joined covalently or noncovalently, directly or indirectly. When bonded covalently, the particular linkage group will depend upon the nature of the two moieties to be bonded. A large number of linking groups and methods for linking are taught in the literature. Broadly, the labels may be divided into the following categories: chromogens; catalyzed reactions; chemiluminescence; radioactive labels; and colloidal-sized colored particles. The chromogens include compounds which absorb light in a distinctive range so that a color may be observed, or emit light when irradiated with light of a particular wavelength or wavelength range, e.g., fluorescence. Both enzymatic and nonenzymatic catalysts may be employed. In choosing an enzyme, there will be many considerations including the stability of the enzyme, whether it is normally present in samples of the type for which the assay is designed, the nature of the substrate, and the effect if any of conjugation on the enzyme's properties. Potentially useful enzyme labels include oxiodoreductases, transferases, hydrolases, lyases, isomerases, ligases, or synthetases. Interrelated enzyme systems may also be used. A chemiluminescent label involves a compound that becomes electronically excited by a chemical reaction and may then emit light that serves as a detectable signal or donates energy to a fluorescent acceptor. Radioactive labels include various radioisotopes found in common use such as the unstable forms of hydrogen, iodine, phosphorus or the like. Colloidal-sized colored particles involve material such as colloidal gold that, in aggregate, form a visually detectable distinctive spot corresponding to the site of a substance to be detected. Additional information on labeling technology is disclosed, for example, in U.S. Pat. No. 4,366,241.

A common method of in vitro labeling of nucleotide probes involves nick translation wherein the unlabeled DNA probe is nicked with an endonuclease to produce free 3′ hydroxyl termini within either strand of the double-stranded fragment. Simultaneously, an exonuclease removes the nucleotide residue from the 5′ phosphoryl side of the nick. The sequence of replacement nucleotides is determined by the sequence of the opposite strand of the duplex. Thus, if labeled nucleotides are supplied, DNA polymerase will fill in the nick with the labeled nucleotides. For smaller probes, known methods involving 3′ end labeling may be used. Furthermore, there are currently commercially available methods of labeling DNA with fluorescent molecules, catalysts, enzymes, or chemiluminescent materials. Biotin labeling kits are commercially available. This type of system permits the probe to be coupled to avidin which in turn is labeled with, for example, a fluorescent molecule, enzyme, antibody, etc. For further disclosure regarding probe construction and technology, see, for example, Sambrook et al. (1982) Molecular Cloning, A Laboratory Manual Cold Spring Harbor, N.Y.

The oligonucleotide selected for hybridizing to the target nucleic acid, whether synthesized chemically or by recombinant DNA methodologies, is isolated and purified using standard techniques and then preferably labeled (e.g., with ³⁵S or ³²P) using standard labeling protocols. A sample containing the target nucleic acid then is run on an electrophoresis gel, the dispersed nucleic acids transferred to a nitrocellulose filter and the labeled oligonucleotide exposed to the filter under stringent hybridization and washing conditions. Specific hybridization and washing conditions include hybridization in, for example, 50% formamide, 5×SSPE, 2×Denhardt's solution, 0.1% SDS at 42° C., as described in Sambrook et al. (1989) supra, followed by washing in, for example, 2×SSPE, 0.1% SDS at 68° C., and/or 0.1×SSPE, 0.1% SDS at 68° C. Other useful procedures known in the art include solution hybridization, and dot and slot RNA hybridization. Optionally, the amount of the target nucleic acid present in a sample is then quantitated by measuring the radioactivity of hybridized fragments, using standard procedures known in the art.

In addition, it is anticipated that using a combination of appropriate oligonucleotide primers, i.e., more than one primer, the skilled artisan may determine the level of expression of a target gene by standard polymerase chain reaction (PCR) procedures, for example, by quantitative PCR. Conventional PCR based assays are discussed, for example, in Innes et al. (1990) PCR Protocols; A guide to methods and Applications, Academic Press and Innes et al. (1995) PCR Strategies, Academic Press, San Diego, Calif. Alternatively, the level of gene expression of the RORA, CRIM1, CXCR4, C5orf26, IGHG3, NALP2, PLA2G4A, IGLJ3, SHQ1, UCHL1, TANC1, PKP2, DNAJC6, C6orf105, NALP1, RGS13, CXCL13, RPS6KA2, MMP7, IL1A, ABCA1, VCAN, KIAA0888, ENPP2, and/or FAM38B genes in the test sample and a control sample can be quantified by Northern blot analysis as known in the art.

B.3. Considerations for Detection of Single Nucleotide Polymorphisms

In certain aspects, the invention provides methods of determining a subject's, for example, a mammal subject's, such as a human subject's, risk of developing one or more angiogenic disorders, for example, an ocular angiogenic disorder, for example, a disorder associated with choroidal neovascularization, for example, age-related macular degeneration by determining whether the subject has a variant at one or more polymorphic sites of one or more of the RORA, CRIM1, CXCR4, C5orf26, IGHG3, NALP2, PLA2G4A, IGLJ3, SHQ1, UCHL1, TANC1, PKP2, DNAJC6, C6orf105, NALP1, RGS13, CXCL13, RPS6KA2, MMP7, IL1A, ABCA1, VCAN, KIAA0888, ENPP2, and/or FAM38B genes. If the subject has at least one protective variant, the subject is less likely to develop one or more angiogenic disorders, for example, an ocular angiogenic disorder, for example, a disorder associated with choroidal neovascularization, for example, age-related macular degeneration than a person without the protective variant, and if the subject has at least one risk variant, the subject is more likely to develop one or more angiogenic disorders, for example, an ocular angiogenic disorder, for example, a disorder associated with choroidal neovascularization, for example, age-related macular degeneration than a person without the risk variant.

For example, in certain embodiments, the invention provides methods of determining a subject's, for example, a mammal subject's, such as a human subject's, risk of developing one or more angiogenic disorders, for example, an ocular angiogenic disorder, for example, a disorder associated with choroidal neovascularization, for example, age-related macular degeneration including determining whether the subject has a protective variant at one or more polymorphic sites of one or more of the RORA, CRIM1, CXCR4, C5orf26, IGHG3, NALP2, PLA2G4A, IGLJ3, SHQ1, UCHL1, TANC1, PKP2, DNAJC6, C6orf105, NALP1, RGS13, CXCL13, RPS6KA2, MMP7, IL1A, ABCA1, VCAN, KIAA0888, ENPP2, and/or FAM38B genes. If the subject has at least one protective variant, the subject is less likely to develop one or more angiogenic disorders, for example, an ocular angiogenic disorder, for example, a disorder associated with choroidal neovascularization, for example, age-related macular degeneration, than a subject without the protective variant.

In certain embodiments, the invention provides methods of determining a subject's, for example, a mammal subject's, such as a human subject's, risk of developing one or more angiogenic disorders, for example, an ocular angiogenic disorder, for example, a disorder associated with choroidal neovascularization, for example, age-related macular degeneration, including determining whether the subject has a risk variant at one or more polymorphic sites of one or more of the RORA, CRIM1, CXCR4, C5orf26, IGHG3, NALP2, PLA2G4A, IGLJ3, SHQ1, UCHL1, TANC1, PKP2, DNAJC6, C6orf105, NALP1, RGS13, CXCL13, RPS6KA2, MMP7, IL1A, ABCA1, VCAN, KIAA0888, ENPP2, and/or FAM38B genes. If the subject has at least one risk variant, the subject is more likely to develop one or more angiogenic disorders, for example, an ocular angiogenic disorder, for example, a disorder associated with choroidal neovascularization, for example, age-related macular degeneration, than a person without the risk variant. Various polymorphic sites for each of the RORA, CRIM1, CXCR4, C5orf26, IGHG3, NALP2, PLA2G4A, IGLJ3, SHQ1, UCHL1, TANC1, PKP2, DNAJC6, C6orf105, NALP1, RGS13, CXCL13, RPS6KA2, MMP7, IL1A, ABCA1, VCAN, KIAA0888, ENPP2, and FAM38B genes are identified above and known in the art as described in the NCBI SNP database, available at the web site, www.ncbi.nlm.nih.gov. Furthermore, it is understood that the determination of whether a subject is at risk of developing the angiogenic disorder can be accomplished by determining the presence of one or more SNPs associated with the foregoing genes or a proxy SNP that is in linkage disequilibrium with (i.e., is expressly associated with) the SNP.

The presence of a protective and/or risk variant can be determined by standard nucleic acid detection assays including, for example, conventional SNP detection assays, which may include, for example, amplification-based assays, probe hybridization assays, restriction fragment length polymorphism assays, and/or direct nucleic acid sequencing. Exemplary protocols for preparing and analyzing samples of interest are discussed in the following paragraphs.

Polymorphisms can be detected in target nucleic acid samples from an individual under investigation. In general, genomic DNA can be analyzed, which can be selected from any biological sample that contains genomic DNA or RNA. For example, genomic DNA can be obtained from peripheral blood leukocytes using standard approaches (QIAamp DNA Blood Maxi kit, Qiagen, Valencia, Calif.). Nucleic acids can be harvested from other samples, for example, cells in saliva, cheek scrapings, skin or tissue biopsies, amniotic fluid. Methods for purifying nucleic acids from biological samples suitable for use in diagnostic or other assays are known in the art.

The identity of bases present at the polymorphic sites of the RORA, CRIM1, CXCR4, C5orf26, IGHG3, NALP2, PLA2G4A, IGLJ3, SHQ1, UCHL1, TANC1, PKP2, DNAJC6, C6orf105, NALP1, RGS13, CXCL13, RPS6KA2, MMP7, IL1A, ABCA1, VCAN, KIAA0888, ENPP2, and FAM38B genes, can be determined in an individual using any of several methods known in the art. The polymorphisms can be detected by direct sequencing, amplification-based assays, probe hybridization-based assays, restriction fragment length polymorphism assays, denaturing gradient gel electrophoresis, single-strand conformation polymorphism analyses, and denaturing high performance liquid chromatography. Other methods to detect nucleic acid polymorphisms include the use of: Molecular Beacons (see, e.g., Piatek et al. (1998) Nat Biotechnol 16:359-63; Tyagi and Kramer (1996) Nat Biotechnol 14:303-308; and Tyagi et al. (1998) Nat Biotechnol 16:49-53), the Invader assay (see, e.g., Neri et al. (2000) Adv Nucl Acid Protein Analysis 3826: 117-125 and U.S. Pat. No. 6,706,471), and the Scorpion assay (see, e.g., Thelwell et al. (2000) Nucl Acids Res 28:3752-3761; and Solinas et al. (2001) Nucl Acids Res 29:20).

The design and use of allele-specific probes for analyzing polymorphisms are described, for example, in EP 235,726, and WO 89/11548. Briefly, allele-specific probes are designed to hybridize to a segment of target DNA from one individual but not to the corresponding segment from another individual, if the two segments represent different polymorphic forms. Hybridization conditions are chosen that are sufficiently stringent so that a given probe essentially hybridizes to only one of two alleles. Typically, allele-specific probes are designed to hybridize to a segment of target DNA such that the polymorphic site aligns with a central position of the probe.

The design and use of allele-specific primers for analyzing polymorphisms are described, for example, in WO 93/22456. Briefly, allele-specific primers are designed to hybridize to a site on target DNA overlapping a polymorphism and to prime DNA amplification according to standard PCR protocols only when the primer exhibits perfect complementarity to the particular allelic form. A single-base mismatch prevents DNA amplification and no detectable PCR product is formed. The method works particularly well when the polymorphic site is at the extreme 3′-end of the primer, because this position is most destabilizing to elongation from the primer.

The primers, once selected, can be used in standard PCR protocols in conjunction with another common primer that hybridizes to the upstream non-coding strand of the RORA, CRIM1, CXCR4, C5orf26, IGHG3, NALP2, PLA2G4A, IGLJ3, SHQ1, UCHL1, TANC1, PKP2, DNAJC6, C6orf105, NALP1, RGS13, CXCL13, RPS6KA2, MMP7, IL1A, ABCA1, VCAN, KIAA0888, ENPP2, and/or FAM38B genes at a specified location upstream from the polymorphisms. The common primers are chosen such that the resulting PCR products can vary from about 100 to about 300 bases in length, or about 150 to about 250 bases in length, although smaller (about 50 to about 100 bases in length) or larger (about 300 to about 500 bases in length) PCR products are possible. The length of the primers can vary from about 10 to 30 bases in length, or about 15 to 25 bases in length.

In addition, individuals with the protective or risk variant can also be identified by restriction fragment length polymorphism (RFLP) assays. It is understood that the presence of a particular SNP substitution can result in the creation of a site of cleavage for a restriction enzyme. In contrast to the common allele, which would not be recognized by the restriction enzyme, the variant can be detected by genotyping the individual by RFLP analysis.

Many of the methods for detecting polymorphisms involve amplifying DNA or RNA from target samples (e.g., amplifying segments of the RORA, CRIM1, CXCR4, C5orf26, IGHG3, NALP2, PLA2G4A, IGLJ3, SHQ1, UCHL1, TANC1, PKP2, DNAJC6, C6orf105, NALP1, RGS13, CXCL13, RPS6KA2, MMP7, IL1A, ABCA1, VCAN, KIAA0888, ENPP2, and/or FAM38B genes) using specific primers, or amplifying segments and analyzing the amplified gene segments. This can be accomplished by standard polymerase chain reaction (PCR & RT-PCR) protocols or other methods known in the art. Amplification products generated using PCR can be analyzed by the use of denaturing gradient gel electrophoresis. Different alleles can be identified based on sequence-dependent melting properties and electrophoretic migration in solution. See Erlich, ed. (1992) PCR Technology, Principles and Applications for DNA Amplification, Chapter 7, W.H. Freeman and Co, New York.

SNP detection can also be accomplished by direct PCR amplification, for example, via Allele-Specific PCR (AS-PCR) which is the selective PCR amplification of one of the alleles to detect SNPs. Selective amplification is usually achieved by designing a primer such that the primer will match/mismatch one of the alleles at the 3′-end of the primer. The amplifying may result in the generation RORA, CRIM1, CXCR4, C5orf26, IGHG3, NALP2, PLA2G4A, IGLJ3, SHQ1, UCHL1, TANC1, PKP2, DNAJC6, C6orf105, NALP1, RGS13, CXCL13, RPS6KA2, MMP7, IL1A, ABCA1, VCAN, KIAA0888, ENPP2, and/or FAM38B gene allele-specific oligonucleotides, which span any of the SNPs. The gene-specific primer sequences and allele-specific oligonucleotides may be derived from the coding (exons) or non-coding (promoter, 5′ untranslated, introns or 3′ untranslated) regions of the corresponding gene.

Direct sequencing analysis of polymorphisms can be accomplished using DNA sequencing procedures known in the art. (See, e.g., Sambrook et al. (1989) Molecular Cloning, A Laboratory Manual (2nd Ed., CSHP, New York) and Zyskind et al. (1988) Recombinant DNA Laboratory Manual (Acad. Press).)

A wide variety of other methods are known in the art for detecting polymorphisms in a biological sample. (See, e.g., U.S. Pat. No. 6,632,606; Shi (2002) Am. J. Pharmacogenomics 2:197-205; Kwok et al. (2003) Curr. Issues Biol. 5:43-60.) Detection of the single nucleotide polymorphic form, alone and/or in combination with each other and/or in combination with additional gene polymorphisms, may increase the probability of an accurate diagnosis. In certain embodiments, the diagnostic method includes determining the presence or absence of one or more variants from one or more genes selected from RORA, CRIM1, CXCR4, C5orf26, IGHG3, NALP2, PLA2G4A, IGLJ3, SHQ1, UCHL1, TANC1, PKP2, DNAJC6, C6orf105, NALP1, RGS13, CXCL13, RPS6KA2, MMP7, IL1A, ABCA1, VCAN, KIAA0888, ENPP2, and FAM38B. This diagnostic method optionally can be combined with analysis of polymorphisms in other genes known to be associated with AMD, with detection of protein markers of AMD (see, e.g., U.S. Patent Application Publication Nos. US2003/0017501 and US2002/0102581 and International Application Publication Nos. WO0184149 and WO0106262), with assessment of other risk factors of AMD (such as family history), with ophthalmological examination, and/or with other assays and procedures.

Screening also can involve detecting a haplotype which includes two or more SNPs. Such SNPs include those described herein and/or additional gene polymorphisms and/or other genes known to be associated with AMD and/or other risk factors. For the detection of two or more SNPs, one can determine if the risk variant is present or absent (for risk variant SNPs) and/or if the common allele is present or absent (for protective variant SNPs) in order to diagnose a subject for being at increased risk of developing AMD. Conversely, for the two or more SNPs, one can determine if the common allele is present or absent (for risk variant SNPs) and/or the protective variant is present or absent (for protective variant SNPs) in order to diagnose a subject for being at reduced risk of developing AMD.

B.4. Diagnostic and Prognostic Kits

The isolated RORA, CRIM1, CXCR4, C5orf26, IGHG3, NALP2, PLA2G4A, IGLJ3, SHQ1, UCHL1, TANC1, PKP2, DNAJC6, C6orf105, NALP1, RGS13, CXCL13, RPS6KA2, MMP7, IL1A, ABCA1, VCAN, KIAA0888, ENPP2, and/or FAM38B genes and/or gene products also may be useful in the development of diagnostic kits and assays to monitor the level of the gene or gene product in a tissue or fluid sample. The kit may include antibodies or other specific binding proteins which bind specifically with one or more of the RORA, CRIM1, CXCR4, C5orf26, IGHG3, NALP2, PLA2G4A, IGLJ3, SHQ1, UCHL1, TANC1, PKP2, DNAJC6, C6orf105, NALP1, RGS13, CXCL13, RPS6KA2, MMP7, IL1A, ABCA1, VCAN, KIAA0888, ENPP2, and/or FAM38B gene products and which permit the presence and/or concentration of the one or more RORA, CRIM1, CXCR4, C5orf26, IGHG3, NALP2, PLA2G4A, IGLJ3, SHQ1, UCHL1, TANC1, PKP2, DNAJC6, C6orf105, NALP1, RGS13, CXCL13, RPS6KA2, MMP7, IL1A, ABCA1, VCAN, KIAA0888, ENPP2, and/or FAM38B gene products to be quantitated in a tissue or fluid sample. Also, the kit may include one or more oligonucleotide probes and/or oligonucleotide primers which hybridize specifically to a gene or mRNA encoding one or more of RORA, CRIM1, CXCR4, C5orf26, IGHG3, NALP2, PLA2G4A, IGLJ3, SHQ1, UCHL1, TANC1, PKP2, DNAJC6, C6orf105, NALP1, RGS13, CXCL13, RPS6KA2, MMP7, IL1A, ABCA1, VCAN, KIAA0888, ENPP2, and/or FAM38B.

The assays described herein can be used to determine if an individual is at risk of developing, or has, one or more angiogenic disorders, for example, an ocular angiogenic disorder, for example, a disorder associated with choroidal neovascularization, for example, age-related macular degeneration. If the individual is identified to be at risk of developing the disorder, the individual may be treated prophylactically to slow down or stop the development of the disorder (e.g. age-related macular degeneration). For example, if a person is identified as being at risk of developing the wet form of age-related macular degeneration, the individual can be treated by using known methods in the art. Alternatively, the individual can be treated with a CXCL13, RPS6KA2, MMP7, IL1A, KIAA0888, ENPP2, CRIM1, CXCR4, C5orf26, IGHG3, IGLJ3, SHQ1, DNAJC6, C6orf105, NALP1, and/or RGS13 antagonist and/or a RORA, NALP2, PLA2G4A, PKP2, UCHL1, TANC1, ABCA1, VCAN, and/or FAM38B agonist as described below. Alternatively, if the individual is identified as having the wet form of age-related macular degeneration, the individual can be treated by any method known in the art, for example, via laser photocoagulation or via photodynamic therapy using the benzoporphyrin derivative mono acid (BPD-MA) photosensitizer (available from QLT, Inc., Vancouver, Canada), optionally in combination with the methods described herein.

Assays can be prepared in any format known in the art. For example, the above-identified proteins, nucleic acids, and or molecules used for analysis and/or detection can be presented in solution or attached to a surface, for example, a bead surface, a chip surface or the surface on the inside of an analytical chromatographic column. Detection can be performed by any method known in the art, for example, optical detection and/or fluorescence detection.

B.5. Analysis Systems and Reports

In a further aspect, the invention provides a system for analyzing one or more biomarkers selected from the group of RORA, CRIM1, CXCR4, C5orf26, IGHG3, NALP2, PLA2G4A, IGLJ3, SHQ1, UCHL1, TANC1, PKP2, DNAJC6, C6orf105, NALP1, RGS13, CXCL13, RPS6KA2, MMP7, IL1A, ABCA1, VCAN, KIAA0888, ENPP2, and/or FAM38B genes and/or gene products comprising: reagents to detect in a sample from the patient the presence, absence, and/or amount of one or more of the RORA, CRIM1, CXCR4, C5orf26, IGHG3, NALP2, PLA2G4A, IGLJ3, SHQ1, UCHL1, TANC1, PKP2, DNAJC6, C6orf105, NALP1, RGS13, CXCL13, RPS6KA2, MMP7, IL1A, ABCA1, VCAN, KIAA0888, ENPP2, and/or FAM38B genes and/or gene products; hardware to perform detection of the biomarkers; and a processor to execute stored instruction sequences (for example, software) that analyze the detected information (e.g., to identify and/or calculate a level of one or more genes or gene products), to determine if the patient is at risk of developing, or has, an ocular angiogenic disorder, and/or to determine if the patient is responsive to a treatment. The reagents to detect one or more of the RORA, CRIM1, CXCR4, C5orf26, IGHG3, NALP2, PLA2G4A, IGLJ3, SHQ1, UCHL1, TANC1, PKP2, DNAJC6, C6orf105, NALP1, RGS13, CXCL13, RPS6KA2, MMP7, IL1A, ABCA1, VCAN, KIAA0888, ENPP2, and/or FAM38B genes and/or gene products may be, for example, any of those described herein, including antibodies, polynucleotides, and other molecules that bind one or more of the RORA, CRIM1, CXCR4, C5orf26, IGHG3, NALP2, PLA2G4A, IGLJ3, SHQ1, UCHL1, TANC1, PKP2, DNAJC6, C6orf105, NALP1, RGS13, CXCL13, RPS6KA2, MMP7, IL1A, ABCA1, VCAN, KIAA0888, ENPP2, and/or FAM38B genes and/or gene products. The hardware is preferably a machine or computer to perform the detection step, and the processor may be by, for example, part of a computer or machine specifically configured to perform the analysis described herein.

Suitable software and processors are well known in the art and are commercially available. The program may be embodied in software and stored on a tangible medium such as CD-ROM, a floppy disk, a hard drive, a DVD, or a memory associated with the processor, but persons of ordinary skill in the art will readily appreciate that the entire program or parts thereof could alternatively be executed by a device other than a processor, and/or embodied in firmware and/or dedicated hardware in a well known manner.

After detecting (including detecting the presence, absence and/or amount) one or more of the RORA, CRIM1, CXCR4, C5orf26, IGHG3, NALP2, PLA2G4A, IGLJ3, SHQ1, UCHL1, TANC1, PKP2, DNAJC6, C6orf105, NALP1, RGS13, CXCL13, RPS6KA2, MMP7, IL1A, ABCA1, VCAN, KIAA0888, ENPP2, and/or FAM38B genes and/or gene products, and producing the assay results, findings, diagnoses, predictions and/or treatment, they are typically recorded and/or communicated to, for example, medical professionals and/or patients. In certain embodiments, the assay results, findings, diagnoses, predictions and/or treatment recommendations are communicated to the patient, directly, or to the patient's treating physician, as soon as possible after the assay and analysis is completed. The assay results, findings, diagnoses, predictions and/or treatment recommendations may be communicated to medical professionals and/or patients by any means of communication, such as a written report (e.g., on paper), an auditory report, or an electronic record.

Communication may be facilitated by use electronic forms of communication and/or by use of a computer, such as in case of email or telephone communications. In certain embodiments, the communication containing assay results, findings, diagnoses, predictions and/or treatment recommendations may be generated and delivered automatically to the subject using a combination of computer hardware and software which will be familiar to artisans skilled in telecommunications. One example of a healthcare-oriented communications system is described in U.S. Pat. No. 6,283,761; however, the present invention is not limited to methods which utilize this particular communications system. In certain embodiments of the methods of the invention, all or some of the method steps, including the assaying of samples, diagnosing/prognosing of diseases, and communicating of assay results, findings, diagnoses, predictions and/or treatment recommendations, may be carried out in diverse (e.g., foreign) jurisdictions. For example, in some embodiments the assays are performed, or the assay results analyzed, in a country or jurisdiction which differs from the country or jurisdiction to which the assay results, findings, diagnoses, predictions and/or treatment recommendations are communicated.

To facilitate diagnosis, the presence, absence, and/or level of one or more of the RORA, CRIM1, CXCR4, C5orf26, IGHG3, NALP2, PLA2G4A, IGLJ3, SHQ1, UCHL1, TANC1, PKP2, DNAJC6, C6orf105, NALP1, RGS13, CXCL13, RPS6KA2, MMP1, IL1A, ABCA1, VCAN, KIAA0888, ENPP2, and/or FAM38B genes and/or gene products can be displayed on a display device or contained electronically or in a machine-readable medium, such as but not limited to, analog tapes like those readable by a VCR, CD-ROM, DVD-ROM, USB flash media, among others. Such machine-readable media can also contain additional test results, such as, without limitation, measurements of clinical parameters and traditional laboratory risk factors. Alternatively or additionally, the machine-readable media can also comprise subject information such as medical history and any relevant family history.

The methods of this invention, when practiced for commercial diagnostic purposes, generally produce a report or summary of the presence, absence, and/or levels (e.g., normalized levels) of one or more of the biomarkers described herein. The methods of this invention also can produce a report comprising one or more predictions and/or diagnoses concerning a patient, for example whether the patient is at risk of developing, or has, an ocular angiogenic disorder.

The methods and reports of this invention can further include storing the report in a database. Alternatively, the method can further create a record in a database for the subject and populate the record with data. Reports can include a paper report, an auditory report, or an electronic record. It is contemplated that the report is provided to a physician and/or the patient. The receiving of the report can further include establishing a network connection to a server computer that includes the data and report and requesting the data and report from the server computer. The methods provided by the present invention may also be automated in whole or in part.

In another aspect, the invention provides an article of manufacture having a computer-readable medium with computer-readable instructions embodied thereon for performing the methods and implementing the systems described herein. In particular, the stored instruction sequences of the present invention may be embedded on a computer-readable medium, such as, but not limited to, a floppy disk, a hard disk, an optical disk, a magnetic tape, a PROM, an EPROM, CD-ROM, or DVD-ROM or downloaded from a server. The stored instruction sequences may be embedded on the computer-readable medium in any number of computer-readable instructions, or languages such as, for example, FORTRAN, PASCAL, C, C++, Java, C#, Tcl, BASIC and assembly language. Further, the computer-readable instructions may, for example, be written in a script, macro, or functionally embedded in commercially available software (such as, e.g., EXCEL or VISUAL BASIC).

### C. Therapies for Preventing the Onset of or Slowing the Development of Angiogenic Disorders

Once an individual has been identified as being at risk of developing one or more angiogenic disorders, for example, an ocular angiogenic disorder, for example, a disorder associated with choroidal neovascularization, for example, age-related macular degeneration, the individual may be monitored on a regular basis using standard methodologies for the onset of the disorder. This approach may facilitate early intervention and treatment of the disorder, which otherwise may progress until substantial irreversible vision loss has occurred. Similarly, the individual may be treated prophylactically, for example, with a sufficient amount of a one or more of a CRIM1 antagonist, a CXCR4 antagonist, a C5orf26 antagonist, an IGHG3 antagonist, an IGLJ3 antagonist, a SHQ1 antagonist, a DNAJC6 antagonist, a C6orf105 antagonist, a NALP1 antagonist, a RGS13 antagonist, a CXCL13 antagonist, a RPS6KA2 antagonist, a MMP7 antagonist, an IL1A antagonist, KIAA0888 antagonist, an ENPP2 antagonist, a RORA agonist, a NALP2 agonist, a PLA2G4A agonist, a PKP2 agonist, a UCHL1 agonist, a TANC1 agonist, an ABCA1 agonist, a VCAN agonist, a and/or a FAM38B agonist to prevent or slow down the onset of the disorder.

The term “treatment agent” is understood to mean any molecule, for example, a protein, peptide, nucleic acid (ribonucleic acid (RNA) or deoxyribonucleic acid (DNA)), peptidyl nucleic acid, or small molecule (organic compound or inorganic compound). Treatment agents can be antagonists that, either directly or indirectly, decrease the transcription of a gene, the translation of the gene into a protein, or the activity of the protein or the biological regulatory system (upstream and downstream) in which it resides (i.e., downregulate the transcription, translation, or activity of the target of interest). Antagonists can be used against the sixteen upregulated genes or their expression or transcription products, namely against the CXCL13, RPS6KA2, MMP7, IL1A, KIAA0888, ENPP2, CRIM1, CXCR4, C5orf26, IGHG3, IGLJ3, SHQ1, DNAJC6, C6orf105, NALP1, and/or RGS13 genes or the corresponding proteins translated therefrom or the RNA transcribed therefrom.

Alternatively, treatment agents can be agonists that, either directly or indirectly, increase the transcription of the gene, the translation of the gene into a protein, or the activity of the protein or the biological regulatory system (upstream and downstream) in which it resides as well as can include providing an exogenous form of the protein, including the protein itself, those proteins or peptides that are at least 85%, 90%, or 95% identical to the full length, wild type sequence of the protein, and those proteins and peptides that have at least 25%, more preferably at least 50%, more preferably at least 75%, and more preferably at least 90% activity of the full length, wild type protein (i.e., upregulate the transcription, translation, activity, or amount of the target of interest). Agonists can be used to target the nine downregulated genes or their expression products, namely the RORA, NALP2, PLA2G4A, PKP2, UCHL1, TANC1, ABCA1, VCAN, and/or FAM38B genes or proteins translated therefrom.

In the invention, an effective amount of treatment agent is used in a subject for a therapeutic purpose. Accordingly, an “effective amount” of a treatment agent is an amount of an agent sufficient to prevent, slow and/or stop the development of one or more angiogenic disorders, for example, an ocular angiogenic disorder, for example, a disorder associated with choroidal neovascularization, for example, age-related macular degeneration.

C.1. Exemplary Treatment Agents—Proteins

Antibodies (e.g., monoclonal or polyclonal antibodies) having sufficiently high binding specificity for a target protein can be used as a treatment agent. For example, anti-CRIM 1, anti-CXCR4, anti-05orf26, anti-IGHG3, anti-CXCL13, anti-RPS6KA2, anti-MMP7, anti-IL1A, anti-KIAA0888, anti-ENPP2, anti-IGLJ3, anti-SHQ1, anti-DNAJC6, anti-C6orf105, anti-NALP1, and/or anti-RGS13 antibodies, can be used as antagonists. As noted above, the term “antibody” is understood to mean an intact antibody (for example, a monoclonal or polyclonal antibody); an antigen binding fragment thereof, for example, an Fv, Fab, Fab′ or (Fab′)₂ fragment; or a biosynthetic antibody binding site, for example, an sFv, as described in U.S. Pat. Nos. 5,091,513; 5,132,405; 5,258,498; and 5,482,858; and 4,704,692. A binding moiety, for example, an antibody, is understood to bind specifically to the target, for example, CXCL13, RPS6KA2, MMP7, IL1A, KIAA0888, ENPP2, CRIM1, CXCR4, C5orf26, IGHG3, IGLJ3, SHQ1, DNAJC6, C6orf105, NALP1, or RGS13, when the binding moiety has a binding affinity for the target greater than about 10⁵M⁻¹, more preferably greater than about 10⁷ M⁻¹. Those antibodies that act with agonistic activity also can be used, for example, when RORA, NALP2, PLA2G4A, PKP2, UCHL1, TANC1, ABCA1, VCAN, and/or FAM38B are targets.

The aforementioned antibodies may be generated using standard immunological procedures well known and described in the art. (See, e.g., Butt, N. R., ed., Practical Immunology, Marcel Dekker, NY, 1984.) Briefly, isolated RORA, CRIM1, CXCR4, C5orf26, IGHG3, NALP2, PLA2G4A, IGLJ3, SHQ1, UCHL1, TANC1, PKP2, DNAJC6, C6orf105, NALP1, RGS13, CXCL13, RPS6KA2, MMP7, IL1A, ABCA1, VCAN, KIAA0888, ENPP2, or FAM38B is used to raise antibodies in a xenogeneic host, such as a mouse, goat or other suitable mammal. Specifically, the target protein (e.g., RORA, CRIM1, CXCR4, C5orf26, IGHG3, NALP2, PLA2G4A, IGLJ3, SHQ1, UCHL1, TANC1, PKP2, DNAJC6, C6orf105, NALP1, RGS13, CXCL13, RPS6KA2, MMP7, IL1A, ABCA1, VCAN, KIAA0888, ENPP2, or FAM38B, respectively) is combined with a suitable adjuvant capable of enhancing antibody production in the host, and injected into the host, for example, by intraperitoneal administration. Any adjuvant suitable for stimulating the host's immune response may be used. A commonly used adjuvant is Freund's complete adjuvant (an emulsion comprising killed and dried microbial cells). Where multiple antigen injections are desired, the subsequent injections may comprise the antigen in combination with an incomplete adjuvant (for example, a cell-free emulsion).

Polyclonal antibodies may be isolated from the antibody-producing host by extracting serum containing antibodies to the protein of interest. Monoclonal antibodies may be produced by isolating host cells that produce the desired antibody, fusing these cells with myeloma cells using standard procedures known in the immunology art, and screening for hybrid cells (hybridomas) that react specifically with the target protein and have the desired binding affinity.

Antibody binding domains also may be produced biosynthetically and the amino acid sequence of the binding domain manipulated to enhance binding affinity with a preferred epitope on the target protein. Specific antibody methodologies are well understood and described in the literature. A more detailed description of their preparation can be found, for example, in Butt, N. R., ed., Practical Immunology, Marcel Dekker, NY, 1984.

Other proteins and peptides also can be used as treatment agents, such as antagonists of CXCL13, RPS6KA2, MMP1, IL1A, KIAA0888, ENPP2, CRIM1, CXCR4, C5orf26, IGHG3, IGLJ3, SHQ1, DNAJC6, C6orf105, NALP1, or RGS13, or agonists of RORA, NALP2, PLA2G4A, PKP2, UCHL1, TANC1, ABCA1, VCAN, or FAM38B. In the case of agonists of any of RORA, NALP2, PLA2G4A, PKP2, UCHL1, TANC1, ABCA1, VCAN, or FAM38B, the agonist can be the protein itself, can be a protein or peptide that is at least 85%, 90%, or 95% identical to the full length, wild type sequence of the protein or can be a protein or peptide that has at least 25%, more preferably at least 50%, more preferably at least 75%, and more preferably at least 90% activity of full length, wild type protein. Proteins and peptides of the invention can be produced in various ways using approaches known in the art. For example, DNA molecules encoding the protein or peptide of interest are chemically synthesized, using a commercial synthesizer and known sequence information. Such synthetic DNA molecules can be ligated to other appropriate nucleotide sequences, including, e.g., expression control sequences, to produce conventional gene expression constructs encoding the desired proteins and peptides. Production of defined gene constructs is within routine skill in the art.

The nucleic acids encoding the desired proteins and peptides can be introduced (ligated) into expression vectors, which can be introduced into a host cell via standard transfection or transformation techniques known in the art. Exemplary host cells include, for example, E. coli cells, Chinese hamster ovary (CHO) cells, HeLa cells, baby hamster kidney (BHK) cells, monkey kidney cells (COS), human hepatocellular carcinoma cells (e.g., Hep G2), and myeloma cells that do not otherwise produce immunoglobulin protein. Transfected host cells can be grown under conditions that permit the host cells to express the genes of interest, for example, the genes that encode the proteins or peptides of interest. The resulting expression products can be harvested using techniques known in the art.

The particular expression and purification conditions will vary depending upon what expression system is employed. For example, if the gene is to be expressed in E. coli, it is first cloned into an expression vector. This is accomplished by positioning the engineered gene downstream from a suitable bacterial promoter, e.g., Trp or Tac, and a signal sequence, e.g., a sequence encoding fragment B of protein A (FB). The resulting expressed fusion protein typically accumulates in refractile or inclusion bodies in the cytoplasm of the cells, and may be harvested after disruption of the cells by French press or sonication. The refractile bodies then are solubilized, and the expressed proteins refolded and cleaved by the methods already established for many other recombinant proteins.

If the engineered gene is to be expressed in eukaryotic host cells, for example, myeloma cells or CHO cells, it is first inserted into an expression vector containing a suitable eukaryotic promoter, a secretion signal, and various introns. The gene construct can be transfected into myeloma cells or CHO cells using established transfection protocols. Such transfected cells can express the proteins or peptides of interest, which may be attached to a protein domain having another function.

Protein treatment agents, such as antibodies and exogenous proteins, are known in the art. For example, CRIM1 antagonists include, but are not limited to, polyclonal antibodies against human CRIM1 (available from Novus Biologicals, Inc., Littleton, Colo., Cat. No. H00051232-A01) and anti-human CRIM1 monoclonal antibodies (available from Novus Biologicals, Inc., Cat. No. H00051232-M01). CXCR4 antagonists include, but are not limited to, polyclonal antibodies against human CXCR4 (available from Novus Biologicals, Cat. No. NB 100-74396) and anti-CXCR4 monoclonal antibodies (available from Sigma, St. Louis, Mo., Cat. No. C6598). C5orf26 antagonists include, but are not limited to, polyclonal antibodies against human C5orf26 and anti-C5orf26 monoclonal antibodies. IGHG3 antagonists include, but are not limited to, polyclonal antibodies against human IGHG3 and anti-IGHG3 monoclonal antibodies (available from Abcam, Inc., Cambridge, Mass., Cat. No. ab1928). IGLJ3 antagonists include, but are not limited to, polyclonal antibodies against human IGLJ3 and anti-IGLJ3 monoclonal antibodies. SHQ1 antagonists include, but are not limited to, polyclonal antibodies against human SHQ1 and anti-SHQ1 monoclonal antibodies. DNAJC6 antagonists include, but are not limited to, polyclonal antibodies against human DNAJC6 and anti-DNAJC6 monoclonal antibodies. C6orf105 antagonists include, but are not limited to, polyclonal antibodies against human C6orf105 and anti-C6orf105 monoclonal antibodies. NALP1 antagonists include, but are not limited to, polyclonal antibodies against human NALP1 (available from Santa Cruz Biotechnology, Inc., Santa Cruz, Calif., Cat. No. sc-34688) and anti NALP1 monoclonal antibodies (available from Genway Biotech, Inc., San Diego, Calif., Cat. No. 20-272-191255). RGS13 antagonists include, but are not limited to, polyclonal antibodies against human RGS13 (available from Santa Cruz Biotechnology, Inc., Santa Cruz, Calif., Cat. No.sc-48279) and anti-RGS13 monoclonal antibodies (available from Abnova, Walnut, Calif., Cat. No. H00006003-M06).

ABCA1 agonists include, but are not limited to, the ABCA1 protein, active peptides and fragments thereof, and stimulators of ABCA1 expression. VCAN agonists include, but are not limited to, the VCAN protein, active peptides and fragments thereof, and stimulators of VCAN expression. FAM38B agonists include, but are not limited to, the FAM38B protein, active peptides and fragments thereof, and stimulators of FAM38B expression.

C.2. Exemplary Treatment Agents—Nucleic Acids

To the extent that the treatment agent is a nucleic acid or peptidyl nucleic acid, such compounds may be synthesized by any of the known chemical oligonucleotide and peptidyl nucleic acid synthesis methodologies known in the art (see, for example, PCT/EP92/20702 and PCT/US94/013523) and used in antisense therapy. Anti-sense oligonucleotide and peptidyl nucleic acid sequences, usually 10 to 100 and more preferably 15 to 50 units in length, are capable of hybridizing to a gene and/or mRNA transcript and, therefore, may be used to inhibit transcription and/or translation of a target protein. CXCL13, RPS6KA2, MMP7, IL1A, KIAA0888, ENPP2, CRIM1, CXCR4, C5orf26, IGHG3, IGLJ3, SHQ1, DNAJC6, C6orf105, NALP1, or RGS13 gene expression therefore can be inhibited by using nucleotide sequences complementary to a regulatory region of any of these genes (e.g., the promoter and/or a enhancer) to form triple helical structures that prevent transcription of any of these gene in target cells. See generally, Helene (1991) Anticancer Drug Des. 6(6): 569-84, Helene et al. (1992) Ann. N.Y. Acad. Sci. 660: 27-36; and Maher (1992) Bioessays 14(12): 807-15. Anti-sense sequences that act with agonistic activity also may be used as a treatment agent such as, for example, agonists for RORA, NALP2, PLA2G4A, PKP2, UCHL1, TANC1,ABCA1, VCAN, and/or FAM38B.

The antisense sequences may be modified at a base moiety, sugar moiety or phosphate backbone to improve, e.g., the stability, hybridization, or solubility of the molecule. For example, in the case of nucleotide sequences, phosphodiester linkages may be replaced by thioester linkages making the resulting molecules more resistant to nuclease degradation. Alternatively, the deoxyribose phosphate backbone of the nucleic acid molecules can be modified to generate peptide nucleic acids (see Hyrup et al. (1996) Bioorg. Med. Chem. 4(1): 5-23). Peptidyl nucleic acids have been shown to hybridize specifically to DNA and RNA under conditions of low ionic strength. Furthermore, it is appreciated that the peptidyl nucleic acid sequences, unlike regular nucleic acid sequences, are not susceptible to nuclease degradation and, therefore, are likely to have greater longevity in vivo. Furthermore, it has been found that peptidyl nucleic acid sequences bind complementary single stranded DNA and RNA strands more strongly than corresponding DNA sequences (PCT/EP92/20702). Similarly, oligoribonucleotide sequences generally are more susceptible to enzymatic attack by ribonucleases than are deoxyribonucleotide sequences, such that oligodeoxyribonucleotides are likely to have greater longevity than oligoribonucleotides for in vivo use.

Additionally, RNAi can serve as a treatment agent. To the extent RNAi is used, double stranded RNA (dsRNA) having one strand identical (or substantially identical) to the target mRNA sequence (e.g. CXCL13, RPS6KA2, MMP7, IL1A, KIAA0888, ENPP2, CRIM1, CXCR4, C5orf26, IGHG3, IGLJ3, SHQ1, DNAJC6, C6orf105, NALP1, or RGS13 mRNA) is introduced to a cell. The dsRNA is cleaved into small interfering RNAs (siRNAs) in the cell, and the siRNAs interact with the RNA induced silencing complex to degrade the target mRNA, ultimately destroying production of a desired gene product (e.g. CXCL13, RPS6KA2, MMP7, IL1A, KIAA0888, ENPP2, CRIM1, CXCR4, C5orf26, IGHG3, IGLJ3, SHQ1, DNAJC6, C6orf105, NALP1, or RGS13 protein, respectively). Alternatively, the siRNA can be introduced directly. RNAi can be used as an antagonist against any of CXCL13, RPS6KA2, MMP7, IL1A, KIAA0888, ENPP2, CRIM1, CXCR4, C5orf26, IGHG3, IGLJ3, SHQ1, DNAJC6, C6orf105, NALP1, or RGS13. RNAi that acts with agonistic activity may also be used as an agonist for any of RORA, NALP2, PLA2G4A, PKP2, UCHL1, TANC1, ABCA1, VCAN, and/or FAM38B in a therapy.

Furthermore, an aptamer to inhibit CXCL13, RPS6KA2, MMP7, IL1A, KIAA0888, ENPP2, CRIM1, CXCR4, C5orf26, IGHG3, IGLJ3, SHQ1, DNAJC6, C6orf105, NALP1, or RGS13 or agonize RORA, NALP2, PLA2G4A, PKP2, UCHL1, TANC1, ABCA1, VCAN, or FAM38B can be used as a treatment agent. Methods for identifying suitable aptamers, for example, via systemic evolution of ligands by exponential enrichment (SELEX), are known in the art and are described, for example, in Ruckman et al. (1998) J. Biol. Chem., 273: 20556-67 and Costantino et al. (1998) J. Pharm. Sci. 87: 1412-20. Additionally, gene therapy can be used, for example to inhibit CXCL13, RPS6KA2, MMP7, IL1A, KIAA0888, ENPP2, CRIM1, CXCR4, C5orf26, IGHG3, IGLJ3, SHQ1, DNAJC6, C6orf105, NALP1, or RGS13 or agonize RORA, NALP2, PLA2G4A, PKP2, UCHL1, TANC1, ABCA1, VCAN, or FAM38B. For example, genes encoding a protein of interest, such as RORA, NALP2, PLA2G4A, PKP2, UCHL1, TANC1, ABCA1, VCAN, or FAM38B, are introduced to target cells by electroporation, either in vitro or in vivo.

Nucleic acid treatment agents, such as siRNAs, are available in the art. For example, siRNAs that target CRIM1 and can be used as CRIM1 antagonists are available from Sigma, St. Louis, Mo. (Cat. No. SASI_Hs01_{—}00096104—SASI_Hs01_{—}00096113). siRNAs that target CXCR4 and can be used as CXCR4 antagonists are available from Sigma, St. Louis, Mo. (Cat. No. SASI_Hs01_{—}00219072—SASI_Hs01_{—}00219081, and Cat. No. SASI_Hs01_{—}00084884-SASI_Hs01_{—}00084893). siRNAs that target C5orf26 and can be used as C5orf26 antagonists are available from Sigma, St. Louis, Mo. (Cat. No. SASI_Hs01_{—}00075304-SASI_Hs01_{—}00075313). siRNAs that target IGHG3 and IGLJ3 can be used as antagonists. siRNAs that target SHQ1 and can be used as antagonists are available from Invitrogen Corp., Carlsbad, Calif. (Cat. No. HSS124015—HSS124017). siRNAs that target DNAJC6 and can be used as antagonists are available from Santa Cruz Biotechnology, Inc. (Cat. No. sc-88612). siRNAs that target C6orf105 and can be used as antagonists are available from Santa Cruz Biotechnology, Inc. (Cat. No. sc-95244). siRNAs that target NALP1 and can be used as antagonists are available from Santa Cruz Biotechnology, Inc. (Cat. No. sc-45479). siRNAs that target RGS13 and can be used as antagonists are available from Sigma, St. Louis, Mo. (Cat. No. SASI_Hs01_{—}00225334-SASI_Hs01_{—}00225343).

C.3. Exemplary Treatment Agents—Small Molecules

To the extent that a treatment agent includes a small molecule that either antagonizes the CXCL13, RPS6KA2, MMP7, IL1A, KIAA0888, ENPP2, CRIM1, CXCR4, C5orf26, IGHG3, G113, SHQ1, DNAJC6, C6orf105, NALP1, or RGS13 gene, or its expression product, or agonizes the RORA, NALP2, PLA2G4A, PKP2, UCHL1, TANC1, ABCA1, VCAN, or FAM38B gene, or its expression product, such compounds may be synthesized by any of the known chemical synthesis methodologies known in the art. Many small molecule treatment agents are already known. For example, stimulators of ABCA1 expression, such as RXR and LXR agonists (e.g., retinoic acid and oxysterols, including 22(R)-hydroxycholesterol and 24-hydroxycholesterol) (see Fukumoto et al. (2002) J. Biol. Chem., 277(5):48508-13), and stimulators of VCAN expression, such as forskolin and phorbol 12 myristate 13-acetate (see Russel et al. (2003) Endocrinology, 144(3):1020-31), can be used as an agonist.

C.4. Combination Therapies

Any one or more of the treatment agents described herein may be combined with any other one or more of the treatment agents described herein. For example, one or more antagonists of CXCL13, RPS6KA2, MMP7, ILIA, KIAA0888, ENPP2, CRIM1, CXCR4, C5orf26, IGHG3, IGLJ3, SHQ1, DNAJC6, C6orf105, NALP1, and RGS13, and/or one or more agonists of RORA, NALP2, PLA2G4A, PKP2, UCHL1, TANC1, ABCA1, VCAN, or FAM38B can be combined.

Furthermore, and without limitation, groups of one or more antagonists of CXCL13, RPS6KA2, MMP7, IL1A, KIAA0888, ENPP2, CRIM1, CXCR4, C5orf26, IGHG3, IGLJ3, SHQ1, DNAJC6, C6orf105, NALP1, and RGS13, and/or groups of one or more agonists of RORA, NALP2, PLA2G4A, PKP2, UCHL1, TANC1, ABCA1, VCAN, or FAM38B can be selected and combined according to those grouped in a particular network, as shown in Table 1, or according to those grouped by a particular biological function, as shown in Table 2. Moreover, treatment agents that target any one or more of the genes or gene products shown in Table 1, or treatment agents that target a network as a whole, can be combined with one another and/or with any other one or more of the treatment agents described herein.

Any one or more of the treatment agents described herein also may be combined with one or more additional AMD treatment modalities. The treatment agent(s) may be administered in any order as well as before, during, or after one or more additional treatment modalities. Additional treatment modalities may include, for example, any one or more of photodynamic therapy (PDT); administration of an anti-angiogenic factor, for example, angiostatin, endostatin or pigment epithelium-derived growth factor; administration of a neuroprotective agent, for example, an apoptosis inhibitor, such as a caspase inhibitor, for example, one or more of a caspase 3 inhibitor, a caspase 7 inhibitor, and a caspase 9 inhibitor; and any combination thereof.

Combination treatments that include PDT have been described, for example, in U.S. Patent Publication No. US-2005-0129684-A1. Generally, PDT requires administration of a photosensitizer to a mammal in need of such treatment. The photosensitizer is administered in an amount sufficient to permit an effective amount (i.e., an amount sufficient to facilitate PDT) of the photosensitizer to localize in the unwanted choroidal neovasculature (CNV).

Following administration of the photosensitizer, the CNV then is irradiated with laser light under conditions such that the light is absorbed by the photosensitizer. The photosensitizer, when activated by the light, generates singlet oxygen and free radicals, for example, reactive oxygen species, that damage surrounding tissue. For example, PDT-induced damage of endothelial cells results in platelet adhesion and degranulation, leading to stasis and aggregation of blood cells and vascular occlusion.

Optionally, the PDT method can also include: (i) administering an anti-angiogenic factor, for example, angiostatin, endostatin or pigment epithelium-derived growth factor, to the mammal prior to, concurrent with or after administration of the photosensitizer, (ii) administering a neuroprotective agent, for example, an apoptosis inhibitor, such as a caspase inhibitor, for example, one or more of a caspase 3 inhibitor, a caspase 7 inhibitor, and a caspase 9 inhibitor prior to, concurrent with, or after administration of the photosensitizer, (iii) administering a therapeutically effective amount of one or more of an antagonist of CXCL13, RPS6KA2, MMP7, IL1A, KIAA0888, ENPP2, CRIM1, CXCR4, C5orf26, IGHG3, IGLJ3, SHQ1, DNAJC6, C6orf105, NALP1, and RGS13, and/or an agonist of RORA, NALP2, PLA2G4A, PKP2, UCHL1, TANC1, ABCA1, VCAN, or FAM38B prior to, concurrent with, or after administration of the photosensitizer, or (iv) a combination of any of the foregoing.

It is contemplated that a variety of photosensitizers useful in PDT may be useful in the practice of the invention and include, for example, amino acid derivatives, azo dyes, xanthene derivatives, chlorins, tetrapyrrole derivatives, phthalocyanines, and assorted other photosensitizers. Amino acid derivatives include, for example, 5-aminolevulinic acid (Berg et al. (1997) Photochem. Photobiol 65: 403-409; El-Far et al. (1985) Cell. Biochem. Function 3, 115-119). Azo dyes, include, for example, Sudan I, Sudan II, Sudan III, Sudan IV, Sudan Black, Disperse Orange, Disperse Red, Oil Red O, Trypan Blue, Congo Red, β-carotene (Mosky et al. (1984) Exp. Res. 155, 389-396). Xanthene derivatives, include, for example, rose bengal. Chlorins include, for example, lysyl chlorin p6 (Berg et al. (1997) supra) and etiobenzochlorin (Berg et al. (1997) supra), 5, 10, 15, 20-tetra (m-hydroxyphenyl) chlorin (M-THPC), N-aspartyl chlorin e6 (Dougherty et al. (1998) J. Natl. Cancer Inst. 90: 889-905), and bacteriochlorin (Korbelik et al. (1992) J. Photochem. Photobiol. 12: 107-119).

Tetrapyrrole derivatives include, for example, lutetium texaphrin (Lu-Tex, PCI-0123) (Dougherty et al. (1998) supra, Young et al. (1996) Photochem. Photobiol. 63: 892-897); benzoporphyrin derivative (BPD) (U.S. Pat. Nos. 5,171,749, 5,214,036, 5,283,255, and 5,798,349, Jori et al. (1990) Lasers Med. Sci. 5, 115-120), benzoporphyrin derivative mono acid (BPD-MA) (U.S. Pat. Nos. 5,171,749, 5,214,036, 5,283,255, and 5,798,349, Berg et al. (1997) supra, Dougherty et al. (1998) supra), hematoporphyrin (Hp) (Jori et al. (1990) supra), hematoporphyrin derivatives (HpD) (Berg et al. (1997) supra, West et al. (1990) In. J. Radiat. Biol. 58: 145-156), porfimer sodium or Photofrin (PHP) (Berg et al. (1997) supra), Photofrin II (PII) (He et al. (1994) Photochem. Photobiol. 59: 468-473), protoporphyrin IX (PpIX) (Dougherty et al. (1998) supra, He et al. (1994) supra), meso-tetra (4-carboxyphenyl) porphine (TCPP) (Musser et al. (1982) Res. Commun. Chem. Pathol. Pharmacol. 2, 251-259), meso-tetra (4-sulfonatophenyl) porphine (TSPP) (Musser et al. (1982) supra), uroporphyrin I (UROP-I) (El-Far et al. (1985) Cell. Biochem. Function 3, 115-119), uroporphyrin III (UROP-III) (El-Far et al. (1985) supra), tin ethyl etiopurpurin (SnET2), (Dougherty et al. (1998) supra 90: 889-905) and 13, 17-bis[1-carboxypropionyl]carbamoylethyl-8-etheny-2-hydroxy-3-hydroxyiminoethylidene-2,7,12,18-tetranethyl 6 porphyrin sodium (ATX-S10(Na)) Mori et al. (2000) Jpn. J. Cancer Res. 91:753-759, Obana et al. (2000) Arch. Ophthalmol. 118:650-658, Obana et al. (1999) Lasers Surg. Med. 24:209-222).

Phthalocyanines include, for example, chloroaluminum phthalocyanine (AlPcCl) (Rerko et al. (1992) Photochem. Photobiol. 55, 75-80), aluminum phthalocyanine with 2-4 sulfonate groups (AlPcS₂₋₄) (Berg et al. (1997) supra, Glassberg et al. (1991) Lasers Surg. Med. 11, 432-439), chloro-aluminum sulfonated phthalocyanine (CASPc) (Roberts et al. (1991) J. Natl. Cancer Inst. 83, 18-32), phthalocyanine (PC) (Jori et al. (1990) supra), silicon phthalocyanine (Pc4) (He et al. (1998) Photochem. Photobiol. 67: 720-728, Jori et al. (1990) supra), magnesium phthalocyanine (Mg²⁺-PC) (Jori et al. (1990) supra), zinc phthalocyanine (ZnPC) (Berg et al. (1997) supra). Other photosensitizers include, for example, thionin, toluidine blue, neutral red and azure c.

The photosensitizer preferably is formulated into a delivery system that delivers high concentrations of the photosensitizer to the CNV. Such formulations may include, for example, the combination of a photosensitizer with a carrier that delivers higher concentrations of the photosensitizer to CNV and/or coupling the photosensitizer to a specific binding ligand that binds preferentially to a specific cell surface component of the CNV.

In one preferred embodiment, the photosensitizer can be combined with a lipid based carrier. For example, liposomal formulations have been found to be particularly effective at delivering the photosensitizer, green porphyrin, and more particularly BPD-MA to the low-density lipoprotein component of plasma, which in turn acts as a carrier to deliver the photosensitizer more effectively to the CNV. Increased numbers of LDL receptors have been shown to be associated with CNV, and by increasing the partitioning of the photosensitizer into the lipoprotein phase of the blood, it may be delivered more efficiently to the CNV. Certain photosensitizers, for example, green porphyrins, and in particular BPD-MA, interact strongly with lipoproteins. LDL itself can be used as a carrier, but LDL is considerably more expensive and less practical than a liposomal formulation. LDL, or preferably liposomes, are thus preferred carriers for the green porphyrins since green porphyrins strongly interact with lipoproteins and are easily packaged in liposomes. Compositions of green porphyrins formulated as lipocomplexes, including liposomes, are described, for example, in U.S. Pat. Nos. 5,214,036, 5,707,608 and 5,798,349. Liposomal formulations of green porphyrin can be obtained from QLT, Inc., Vancouver, Canada. It is contemplated that certain other photosensitizers may likewise be formulated with lipid carriers, for example, liposomes or LDL, to deliver the photosensitizer to CNV.

Furthermore, the photosensitizer can be coupled to a specific binding ligand that binds preferentially to a cell surface component of the CNV, for example, neovascular endothelial homing motif. It appears that a variety of cell surface ligands are expressed at higher levels in new blood vessels relative to other cells or tissues.

Endothelial cells in new blood vessels express several proteins that are absent or barely detectable in established blood vessels (Folkman (1995) Nature Medicine 1:27-31), and include integrins (Brooks et al. (1994) SCIENCE 264: 569-571; Friedlander et al. (1995) Science 270: 1500-1502) and receptors for certain angiogenic factors like vascular endothelial growth factor (VEGF). In vivo selection of phage peptide libraries have also identified peptides expressed by the vasculature that are organ-specific, implying that many tissues have vascular “addresses” (Pasqualini et al. (1996) Nature 380: 364-366). It is contemplated that a suitable targeting moiety can direct a photosensitizer to the CNV endothelium thereby increasing the efficacy and lowering the toxicity of PDT.

Several targeting molecules may be used to target photosensitizers to the neovascular endothelium. For example, α-v integrins, in particular α-v β3 and α-v β5, appear to be expressed in ocular neovascular tissue, in both clinical specimens and experimental models (Corjay et al. (1997) Invest. Ophthalmol. Vis. Sci. 38, 5965; Friedlander et al. (1995) supra). Accordingly, molecules that preferentially bind α-v integrins can be used to target the photosensitizer to CNV. For example, cyclic peptide antagonists of these integrins have been used to inhibit neovascularization in experimental models (Friedlander et al. (1996) Proc. Natl. Acad. Sci. USA 93:9764-9769). A peptide motif having an amino acid sequence, in an N to C-terminal direction, ACDCRGDCFC (SEQ ID NO: 80)—also know as RGD-4C—has been identified that selectively binds to human α-v integrins and accumulates in tumor neovasculature more effectively than other angiogenesis targeting peptides (Arap et al. (1998) Nature 279:377-380; Ellerby et al. (1999) Nature Medicine 5: 1032-1038). Angiostatin may also be used as a targeting molecule for the photosensitizer. Studies have shown, for example, that angiostatin binds specifically to ATP synthase disposed on the surface of human endothelial cells (Moser et al. (1999) Proc. Natl. Acad. Sci. USA 96:2811-2816).

Another potential targeting molecule is an antibody that binds the vascular endothelial growth factor receptor (VEGF-2R). Clinical and experimental evidence strongly supports a role for VEGF in ocular neovascularization, particularly ischemia-associated neovascularization (Adamis et al. (1996) Arch. Ophthalmol. 114:66-71; Tolentino et al. (1996) Arch. Ophthalmol. 114:964-970; Tolentino et al. (1996) Ophthalmol. 103:1820-1828). Antibodies that bind the VEGF receptor (VEGFR-2 also known as KDR) may also bind preferentially to neovascular endothelium. A useful targeting molecule includes the recombinant humanized anti-VEGF monoclonal antibody fragment available from Genentech, Vacaville, Calif.

The targeting molecule may be synthesized using methodologies known and used in the art. For example, proteins and peptides may be synthesized using conventional synthetic peptide chemistries or expressed as recombinant proteins or peptides in a recombinant expression system (see, for example, Sambrook et al. eds, Molecular Cloning, Cold Spring Harbor Laboratories). Similarly, antibodies may be prepared and purified using conventional methodologies, for example, as described in Butt, W. R. ed. (1984) Practical Immunology, Marcel Deckker, New York and Harlow et al., eds. (1988) Antibodies, A Laboratory Approach, Cold Spring Harbor Press. Once created, the targeting agent may be coupled to the photosensitizer using standard coupling chemistries, using, for example, conventional cross linking reagents, for example, heterobifunctional cross linking reagents available, for example, from Pierce, Rockford, Ill.

C.5. Treatment Agent Administration and Dosing

The type and amount of treatment agent(s) to be administered will depend upon various factors including, for example, the age, weight, gender, and health of the individual to be treated, as well as the type and/or severity of the particular disorder to be treated. It is contemplated, however, that optimal treatment agents, modes of administration and dosages may be determined empirically. Protein, peptide or nucleic acid based treatment agents can be administered at doses ranging, for example, from about 0.001 to about 500 mg/kg, from about 0.01 to about 250 mg/kg, and from about 0.1 to about 100 mg/kg. In certain embodiments, an effective amount of dosage of treatment agent will be in the range of from about 1.0 mg/kg to about 50 mg/kg of body weight/day. Small molecule treatment agents may be administered at doses ranging, for example, from 1-1500 mg/m², for example, about 3, 30, 60, 90, 180, 300, 600, 900, 1200 or 1500 mg/m². Pharmaceutical compositions as disclosed herein are not limited to any particular pH. In certain embodiments, pH of a composition ranges from about 3 to about 7, about 3 to about 6, or about 4 to about 6, for example about 5. If adjustment of pH is needed, it can be achieved by the addition of an appropriate acid, such as hydrochloric acid, or base, such as for example, sodium hydroxide.

C.5.a Formulation Considerations

The treatment agent may be formulated with a pharmaceutically acceptable carrier or vehicle to enhance biocompatibility and/or delivery, for example, so that administration of the treatment agent does not otherwise adversely affect the recipient's electrolyte and/or volume balance. Accordingly, formulations of the invention, both for veterinary and for human medical use, include one or more antagonists of CXCL13, RPS6KA2, MMP1, IL1A, KIAA0888, ENPP2, CRIM1, CXCR4, C5orf26, IGHG3, IGLJ3, SHQ1, DNAJC6, C6orf105, NALP1, or RGS13, and/or one or more agonists of RORA, NALP2, PLA2G4A, PKP2, UCHL1, TANC1, ABCA1, VCAN, or FAM38B in association with one or more pharmaceutically acceptable carriers and/or excipients.

Pharmaceutically acceptable carriers, in this regard, are intended to include any and all solvents, dispersion media, coatings, isotonic and absorption delaying agents, and the like, that are compatible with pharmaceutical administration. A pharmaceutically acceptable carrier should be acceptable in the sense of being compatible with the other ingredients of the formulations and not deleterious to the recipient. Similarly, the term “excipient” herein means any substance, not itself a treatment agent, used as a carrier or vehicle for delivery of a treatment agent to a subject or added to a formulation to improve its handling or storage properties or to permit or facilitate formation of a unit dose formulation of the composition. The use of such media and agents for formulating pharmaceutically active compositions is known in the art. Supplementary active compounds (identified or designed according to the invention and/or known in the art) also can be incorporated into the formulations. The formulations may conveniently be presented in dosage unit form and may be prepared by any of the methods well known in the art of pharmacy/microbiology. In general, some formulations are prepared by bringing the treatment agent(s) into association with a liquid carrier or a finely divided solid carrier or both, and then, if necessary, shaping the product into the desired formulation.

Illustrative excipients include antioxidants, surfactants, adhesives, agents to adjust the pH and osmolarity, preservatives, antioxidants, thickening agents, sweetening agents, flavoring agents, taste masking agents, colorants, buffering agents, and penetration enhancers. Generally speaking, a given excipient, if present, will be present in an amount of about 0.001% to about 20% (w/v), about 0.01% (w/v) to about 10% (w/v), about 0.02% (w/v) to about 5% (w/v), or about 0.3% (w/v) to about 2.5% (w/v). Illustrative antioxidants for use in the present invention include, but are not limited to, butylated hydroxytoluene, butylated hydroxyanisole, potassium metabisulfite, and the like. One or more antioxidants, if desired, are typically present in a formulation in an amount of about 0.01% (w/v) to about 2.5% (w/v), for example about 0.01% (w/v), about 0.05% (w/v), about 0.1% (w/v), about 0.5% (w/v), about 1% (w/v), about 1.5% (w/v), about 1.75% (w/v), about 2% (w/v), about 2.25% (w/v), or about 2.5% (w/v).

In certain embodiments, formulations optionally include a preservative. Ideally, the optional preservative will be present in quantities sufficient to preserve the formulation, but in quantities low enough that they do not cause irritation of the area of application of the treatment agent, such as the eye. Exemplary preservatives include, but are not limited to, benzalkonium chloride, methyl, ethyl, propyl or butylparaben, benzyl alcohol, phenylethyl alcohol, benzethonium, or combination thereof. Typically, the optional preservative is present in an amount of about 0.01% (w/v) to about 0.5% (w/v) or about 0.01% (w/v) to about 2.5% (w/v). In other embodiments, formulations are preservative-free. As used herein, the term “preservative-free” includes formulations that do not contain a detectable amount of a preservative.

In certain embodiments, formulations optionally include a buffering agent. The buffering agent, if present, ideally is present in an amount that does not irritate the area of application of the treatment agent, such as the eye. Buffering agents include agents that reduce pH changes. Illustrative classes of buffering agents include a salt of a Group IA metal including, for example, a bicarbonate salt of a Group IA metal, a carbonate salt of a Group IA metal, an alkaline earth metal buffering agent, an aluminum buffering agent, a calcium buffering agent, a sodium buffering agent, or a magnesium buffering agent. Other illustrative classes of buffering agents include alkali (sodium and potassium) or alkaline earth (calcium and magnesium) carbonates, phosphates, bicarbonates, citrates, borates, acetates, phthalates, tartrates, succinates and the like, such as sodium or potassium phosphate, citrate, borate, acetate, bicarbonate and carbonate. Additional exemplary buffering agents include aluminum, magnesium hydroxide, aluminum glycinate, calcium acetate, calcium bicarbonate, calcium borate, calcium carbonate, calcium citrate, calcium gluconate, calcium glycerophosphate, calcium hydroxide, calcium lactate, calcium phthalate, calcium phosphate, calcium succinate, calcium tartrate, dibasic sodium phosphate, dipotassium hydrogen phosphate, dipotassium phosphate, disodium hydrogen phosphate, disodium succinate, dry aluminum hydroxide gel, magnesium acetate, magnesium aluminate, magnesium borate, magnesium bicarbonate, magnesium carbonate, magnesium citrate, magnesium gluconate, magnesium hydroxide, magnesium lactate, magnesium metasilicate aluminate, magnesium oxide, magnesium phthalate, magnesium phosphate, magnesium silicate, magnesium succinate, magnesium tartrate, potassium acetate, potassium carbonate, potassium bicarbonate, potassium borate, potassium citrate, potassium metaphosphate, potassium phthalate, potassium phosphate, potassium polyphosphate, potassium pyrophosphate, potassium succinate, potassium tartrate, sodium acetate, sodium bicarbonate, sodium borate, sodium carbonate, sodium citrate, sodium gluconate, sodium hydrogen phosphate, sodium hydroxide, sodium lactate, sodium phthalate, sodium phosphate, sodium polyphosphate, sodium pyrophosphate, sodium sesquicarbonate, sodium succinate, sodium tartrate, sodium tripolyphosphate, synthetic hydrotalcite, tetrapotassium pyrophosphate, tetrasodium pyrophosphate, tripotassium phosphate, trisodium phosphate, and trometarnol. (Based in part upon the list provided in The Merck Index, Merck & Co. Rahway, N.J. (2001)). Furthermore, combinations or mixtures of any two or more of the above mentioned buffering agents can be used in a formulation. One or more buffering agents, if desired, typically are present in formulations in an amount of about 0.01% (w/v) to about 5% (w/v) or about 0.01% (w/v) to about 3% (w/v).

In various embodiments, formulations optionally comprise one or more surfactants. Optional surfactants are typically present in a formulation of the invention in an amount of about 0.1 mg/mL to about 10 mg/mL, about 0.5 mg/mL to about 5 mg/mL or about 1 mg/mL.

In various embodiments, formulations may include one or more agents that increase viscosity. Illustrative agents that increase viscosity include, but are not limited to, methylcellulose, carboxymethylcellulose sodium, ethylcellulose, carrageenan, carbopol, and/or combinations thereof. Typically, one or more viscosity increasing agents, if desired, are present in compositions of the invention in an amount of about 0.1% (w/v) to about 10% (w/v), or about 0.1% (w/v) to about 5% (w/v).

In various embodiments, formulations (e.g. for oral administration) may include one or more sweeteners and/or flavoring agents. Suitable sweeteners and/or flavoring agents include any agent that sweetens or provides flavor to the formulation. The sweetener or flavoring agent will help mask any bitter or bad taste. Optional sweetening agents and/or flavoring agents are typically present in a composition of the invention in an amount of about 0.1 mg/mL to about 10 mg/mL, about 0.5 mg/mL to about 5 mg/ml or about 1 mg/mL. Illustrative sweeteners or flavoring agents include, without limitation, acacia syrup, anethole, anise oil, aromatic elixir, benzaldehyde, benzaldehyde elixir, cyclodextrins, caraway, caraway oil, cardamom oil, cardamom seed, cardamom spirit compound, cardamom tincture compound, cherry juice, cherry syrup, cinnamon, cinnamon oil, cinnamon water, citric acid, citric acid syrup, clove oil, cocoa, cocoa syrup, coriander oil, dextrose, eriodictyon, eriodictyon fluidextract, eriodictyon syrup, aromatic, ethylacetate, ethyl vanillin, fennel oil, ginger, ginger fluidextract, ginger oleoresin, dextrose, glucose, sugar, maltodextrin, glycerin, glycyrrhiza, glycyrrhiza elixir, glycyrrhiza extract, glycyrrhiza extract pure, glycyrrhiza fluidextract, glycyrrhiza syrup, honey, isoalcoholic elixir, lavender oil, lemon oil, lemon tincture, mannitol, methyl salicylate, nutmeg oil, orange bitter, elixir, orange bitter, oil, orange flower oil, orange flower water, orange oil, orange peel, bitter, orange peel sweet, tincture, orange spirit compound, orange syrup, peppermint, peppermint oil, peppermint spirit, peppermint water, phenylethyl alcohol, raspberry juice, raspberry syrup, rosemary oil, rose oil, rose water, stronger, saccharin, saccharin calcium, saccharin sodium, sarsaparilla syrup, sarsaparilla compound, sorbitol solution, spearmint, spearmint oil, sucrose, sucralose, syrup, thyme oil, tolu balsam, tolu balsam syrup, vanilla, vanilla tincture, vanillin, wild cherry syrup, or combinations thereof. Illustrative taste masking agents include, but are not limited to, cyclodextrins, cyclodextrins emulsions, cyclodextrins particles, cyclodextrins complexes, or combinations thereof.

The foregoing excipients can have multiple roles as is known in the art. For example, some flavoring agents can serve as sweeteners as well as a flavoring agent. Therefore, the above-identified classifications of excipients is understood as non-limiting.

C.5.b Administration Considerations

Treatment agents of the of the invention should be formulated to be compatible with their intended routes of administration. Generally, administration can be local or systemic. Exemplary routes of administration include, for example, topical (e.g. to the eye, skin, or mucosa), intraorbital, periorbital, sub-tenons, intravitreal, transscleral, transdermal, oral, parenteral (e.g., intravenous, intralymphatic, intraspinal, subcutaneous or intramuscular), nasal, otic, intraperitoneal, intracranial, intracerebroventricular, intracerebral, intravaginal, intrauterine intramuscular, intradermal, and rectal administration, as well as via inhalation.

Formulations suitable for topical administration of the treatment agents are optionally formulated as ointments, creams, suspensions, lotions, powders, solutions, pastes, gels, sprays, aerosols or oils. In alternative embodiments, topical formulations can include patches or dressings such as a bandage or adhesive plasters impregnated with active ingredient(s), and optionally one or more excipients or diluents. In some embodiments, the topical formulations include compound(s) that enhance absorption or penetration of the active agent(s) through the skin or other affected areas. Exemplary penetration enhancers include dimethylsulfoxide (DMSO) and related analogues.

Formulations suitable for oral or parenteral administration may be in the form of discrete units such as capsules, gelatin capsules, sachets, tablets, troches, or lozenges, each containing a predetermined amount of the antibiotic; a powder or granular composition; a solution or a suspension in an aqueous liquid or non-aqueous liquid; or an oil-in-water emulsion or a water-in-oil emulsion. Formulations suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor ELTM (BASF, Parsippany, N.J.) or phosphate buffered saline (PBS). Sterile injectable solutions can be prepared by incorporating the active compound in the required amount in an appropriate solvent with one or a combination of ingredients enumerated above, as required, followed by filter sterilization.

Formulations suitable for intra-articular administration may be in the form of a sterile aqueous preparation of the drug which may be in microcrystalline form, for example, in the form of an aqueous microcrystalline suspension. Liposomal formulations or biodegradable polymer systems may also be used to present the drug for both intra-articular and ophthalmic administration. Formulations suitable for topical administration, including eye treatment, include liquid or semi-liquid preparations such as liniments, lotions, gels, applicants, oil-in-water or water-in-oil emulsions such as creams, ointments or pastes; or solutions or suspensions such as drops. Formulations for topical administration to the skin surface can be prepared by dispersing the drug with a dermatologically acceptable carrier such as a lotion, cream, ointment or soap. For inhalation treatments, inhalation of powder (self-propelling or spray formulations) dispensed with a spray can, a nebulizer, or an atomizer can be used. Such formulations can be in the form of a fine powder for pulmonary administration from a powder inhalation device or self-propelling powder-dispensing formulations.

Formulations suitable for administration of treatment agents may include aqueous and non-aqueous sterile injection solutions which may contain anti-oxidants, buffers, bacteriostats and solutes which render the formulation isotonic with the blood of the intended recipient; and aqueous and non-aqueous sterile suspensions which may include suspending agents and thickening agents. The formulations may be presented in unit-dose or multi-dose containers, for example, sealed ampules and vials, and may be stored in a freeze-dried (lyophilized) condition requiring only the addition of the sterile liquid carrier, for example, water for injections, immediately prior to use. The formulations may also be presented in continuous release vehicles. Extemporaneous injection solutions and suspensions may be prepared from sterile powders, granules and tablets of the kind previously described. The excipient formulations may conveniently be prepared by conventional pharmaceutical techniques. Such techniques include the step of bringing into association the active ingredient and the pharmaceutical carrier(s) or excipient(s). In general, the formulations are prepared by uniformly and intimately bringing into association the active ingredient with liquid carriers or finely divided solid carriers or both, and then, if necessary, shaping the product.

C.5.c Considerations for Ocular Delivery

In therapeutic use for treating an ocular disorder, one or more treatment agents can be administered orally, parenterally and/or topically to provide a therapeutically effective amount in the individual, for example, an amount of the active ingredient, for example, in the blood and/or tissue (e.g. ocular or vascular tissue), sufficient to prevent the onset and/or development of the ocular disorder (e.g. age-related macular degeneration).

It is contemplated that one or more treatment agents (e.g. selective antagonists of CXCL13, RPS6KA2, MMP7, IL1A, KIAA0888, ENPP2, CRIM1, CXCR4, C5orf26, IGHG3, IGLJ3, SHQ1, DNAJC6, C6orf105, NALP1, and/or RGS13, and/or selective agonists of RORA, NALP2, PLA2G4A, PKP2, UCHL1, TANC1, ABCA1, VCAN, and/or FAM38B) may be formulated for delivery to the eye (e.g. to the macula). Local modes of administration include, for example, intraocular, intraorbital, subconjuctival, intravitreal, subretinal or transcleral routes. Local routes of administration can be preferable over systemic routes because significantly smaller amounts of the selective treatment agent(s) can exert an effect when administered locally (for example, intravitreally) versus when administered systemically (for example, intravenously). Furthermore, the local modes of administration can reduce or eliminate the incidence of potentially toxic side effects that may occur when amounts of one or more treatment agents (e.g., an amount of a selective antagonist(s) and/or agonist(s) sufficient to reduce or enhance (for example, by 20%, 30%, 40%, 50%, 60%, 70%, 80%, 90%, or 95%) the biological activity or expression of the corresponding protein and/or gene) are administered systemically.

Administration may be provided as a periodic bolus (e.g. intravitreally) or as continuous infusion from an internal reservoir (for example, from an implant disposed at an intra- or extra-ocular location (see, U.S. Pat. Nos. 5,443,505 and 5,766,242)) or from an external reservoir (for example, from an intravenous bag). The treatment agent(s) may be administered locally, for example, by continuous release from a sustained release drug delivery device immobilized to an inner wall of the eye or via targeted transscleral controlled release into the choroid (see, for example, PCT/US00/00207, PCT/US02/14279, Ambati et al. (2000) Invest. Ophthalmol. Vis. Sci. 41:1181-1185, and Ambati et al. (2000) Invest. Ophthalmol. Vis. Sci. 41:1186-1191). A variety of devices suitable for administering selective antagonist(s) and/or agonist(s) locally to the inside of the eye are known in the art. See, for example, U.S. Pat. Nos. 6,251,090, 6,299,895, 6,416,777, 6,413,540, and 6,375,972, and PCT/US00/28187.

Further, it is contemplated that the one or more treatment agents (e.g. selective antagonists of CXCL13, RPS6KA2, MMP1, IL1A, KIAA0888, ENPP2, CRIM1, CXCR4, C5orf26, IGHG3, IGLJ3, SHQ1, DNAJC6, C6orf105, NALP1, and/or RGS13, and/or selective agonists of RORA, NALP2, PLA2G4A, PKP2, UCHL1, TANC1, ABCA1, VCAN, and/or FAM38B) may be formulated so as to permit release of the treatment agent(s) over a prolonged period of time. A release system can include a matrix of a biodegradable material or a material which releases the incorporated treatment agent(s) by diffusion. The treatment agent(s) can be homogeneously or heterogeneously distributed within the release system. A variety of release systems may be useful in the practice of the invention; however, the choice of the appropriate system will depend upon the rate of release required by a particular drug regime. Both non-degradable and degradable release systems can be used. Suitable release systems include polymers and polymeric matrices, non-polymeric matrices, or inorganic and organic excipients and diluents such as, but not limited to, calcium carbonate and sugar (for example, trehalose). Release systems may be natural or synthetic. However, synthetic release systems are preferred because generally they are more reliable, more reproducible and produce more defined release profiles. The release system material can be selected so that treatment agent(s) having different molecular weights are released by diffusion through or degradation of the material.

Representative synthetic, biodegradable polymers include, for example: polyamides such as poly(amino acids) and poly(peptides); polyesters such as poly(lactic acid), poly(glycolic acid), poly(lactic-co-glycolic acid), and poly(caprolactone); poly(anhydrides); polyorthoesters; polycarbonates; and chemical derivatives thereof (substitutions, additions of chemical groups, for example, alkyl, alkylene, hydroxylations, oxidations, and other modifications routinely made by those skilled in the art), copolymers and mixtures thereof. Representative synthetic, non-degradable polymers include, for example: polyethers such as poly(ethylene oxide), poly(ethylene glycol), and poly(tetramethylene oxide); vinyl polymers-polyacrylates and polymethacrylates such as methyl, ethyl, other alkyl, hydroxyethyl methacrylate, acrylic and methacrylic acids, and others such as poly(vinyl alcohol), poly(vinyl pyrolidone), and poly(vinyl acetate); poly(urethanes); cellulose and its derivatives such as alkyl, hydroxyalkyl, ethers, esters, nitrocellulose, and various cellulose acetates; polysiloxanes; and any chemical derivatives thereof (substitutions, additions of chemical groups, for example, alkyl, alkylene, hydroxylations, oxidations, and other modifications routinely made by those skilled in the art), copolymers and mixtures thereof.

One of the primary vehicles currently being developed for the delivery of ocular treatment agents is the poly(lactide-co-glycolide) microsphere for intraocular injection. The microspheres are composed of a polymer of lactic acid and glycolic acid, which are structured to form hollow spheres. These spheres can be approximately 15-30 μm in diameter and can be loaded with a variety of compounds varying in size from simple molecules to high molecular weight proteins such as antibodies. The biocompatibility of these microspheres is well established (see, Sintzel et al. (1996) Eur. J. Pharm. Biopharm. 42: 358-372), and microspheres have been used to deliver a wide variety of pharmacological agents in numerous biological systems. After injection, poly(lactide-co-glycolide) microspheres are hydrolyzed by the surrounding tissues, which cause the release of the contents of the microspheres (Zhu et al. (2000) Nat. Biotech. 18: 52-57). As will be appreciated, the in vivo half-life of a microsphere can be adjusted depending on the specific needs of the system.

By way of example, protein-, peptide- or nucleic acid-based selective antagonists of CXCL13, RPS6KA2, MMP7, IL1A, KIAA0888, ENPP2, CRIM1, CXCR4, C5orf26, IGHG3, IGLJ3, SHQ1, DNAJC6, C6orf105, NALP1, and/or RGS13, and/or selective agonists of RORA, NALP2, PLA2G4A, PKP2, UCHL1, TANC1, ABCA1, VCAN, and/or FAM38B can be administered at doses ranging, for example, from about 0.001 to about 500 mg/kg, optionally from about 0.01 to about 250 mg/kg, and optionally from about 0.1 to about 100 mg/kg. In certain embodiments, nucleic acid-based selective antagonists of CXCL13, RPS6KA2, MMP7, IL1A, KIAA0888, ENPP2, CRIM1, CXCR4, C5orf26, IGHG3, IGLJ3, SHQ1, DNAJC6, C6orf105, NALP1, and/or RGS13, and/or selective agonists of RORA, NALP2, PLA2G4A, PKP2, UCHL1, TANC1, ABCA1, VCAN, and/or FAM38B may be administered at doses ranging from about 1 to about 20 mg/kg daily. Furthermore, antibodies that are selective antagonists of CXCL13, RPS6KA2, MMP7, IL1A, KIAA0888, ENPP2, CRIM1, CXCR4, C5orf26, IGHG3, IGLJ3, SHQ1, DNAJC6, C6orf105, NALP1, and/or RGS13, and/or antibodies and active exogenous proteins or peptides that are selective agonists of RORA, NALP2, PLA2G4A, PKP2, UCHL1, TANC1, ABCA1, VCAN, and/or FAM38B may be administered intravenously at doses ranging from about 0.1 to about 5 mg/kg once every two to four weeks. With regard to intravitreal administration, the selective antagonists of CXCL13, RPS6KA2, MMP7, IL1A, KIAA0888, ENPP2, CRIM1, CXCR4, C5orf26, IGHG3, IGLJ3, SHQ1, DNAJC6, C6orf105, NALP1 and/or RGS13, and/or selective agonists of RORA, NALP2, PLA2G4A, PKP2, UCHL1, TANC1, ABCA1, VCAN, and/or FAM38B, for example, antibodies, proteins, or peptides may be administered periodically as boluses in dosages ranging from about 100 μg to about 5 mg/eye, and optionally from about 10 μg to about 2 mg/eye. With regard to transcleral administration, the selective antagonists of CXCL13, RPS6KA2, MMP7, IL1A, KIAA0888, ENPP2, CRIM1, CXCR4, C5orf26, IGHG3, IGLJ3, SHQ1, DNAJC6, C6orf105, NALP1, and/or RGS13, and/or selective agonists of RORA, NALP2, PLA2G4A, PKP2, UCHL1, TANC1, ABCA1, VCAN, and/or FAM38B may be administered periodically as boluses in dosages ranging from about 0.1 μg to about 1 mg/eye, and optionally from about 0.5 μg to about 0.5 mg/eye.

C.5.d Considerations for Photodynamic Therapy

Photosensitizers as described herein may be administered in any of a wide variety of ways, for example, orally, parenterally, or rectally. Parenteral administration, such as intravenous, intramuscular, or subcutaneous, is preferred. Intravenous injection is preferred. The dose of photosensitizer can vary widely depending on the tissue to be treated; the physical delivery system in which it is carried, such as in the form of liposomes; or whether it is coupled to a target-specific ligand, such as an antibody or an immunologically active fragment.

It should be noted that the various parameters used for effective, selective photodynamic therapy in the invention are interrelated. Therefore, the dose should also be adjusted with respect to other parameters, for example, fluence, irradiance, duration of the light used in PDT, and time interval between administration of the dose and the therapeutic irradiation. All of these parameters should be adjusted to produce significant damage to CNV without significant damage to the surrounding tissue.

Typically, the dose of photosensitizer used is within the range of from about 0.1 to about 20 mg/kg, preferably from about 0.15 to about 5.0 mg/kg, and even more preferably from about 0.25 to about 2.0 mg/kg. Furthermore, as the dosage of photosensitizer is reduced, for example, from about 2 to about 1 mg/kg in the case of green porphyrin or BPD-MA, the fluence required to close CNV may increase, for example, from about 50 to about 100 Joules/cm². Similar trends may be observed with the other photosensitizers discussed herein.

After the photosensitizer has been administered, the CNV is irradiated at a wavelength typically around the maximum absorbance of the photosensitizer, usually in the range from about 550 nm to about 750 nm. A wavelength in this range is especially preferred for enhanced penetration into bodily tissues. Preferred wavelengths used for certain photosensitizers include, for example, about 690 nm for benzoporphyrin derivative mono acid, about 630 nm for hematoporphyrin derivative, about 675 nm for chloro-aluminum sulfonated phthalocyanine, about 660 nm for tin ethyl etiopurpurin, about 730 nm for lutetium texaphyrin, about 670 nm for ATX-S10(NA), about 665 nm for N-aspartyl chlorin e6, and about 650 nm for 5, 10, 15, 20-tetra (m-hydroxyphenyl) chlorin.

As a result of being irradiated, the photosensitizer in its triplet state is thought to interact with oxygen and other compounds to form reactive intermediates, such as singlet oxygen and reactive oxygen species, which can disrupt cellular structures. Possible cellular targets include the cell membrane, mitochondria, lysosomal membranes, and the nucleus. Evidence from tumor and neovascular models indicates that occlusion of the vasculature is a major mechanism of photodynamic therapy, which occurs by damage to the endothelial cells, with subsequent platelet adhesion, degranulation, and thrombus formation.

The fluence during the irradiating treatment can vary widely, depending on the type of photosensitizer used, the type of tissue, the depth of target tissue, and the amount of overlying fluid or blood. Fluences preferably vary from about 10 to about 400 Joules/cm² and more preferably vary from about 50 to about 200 Joules/cm². The irradiance varies typically from about 50 mW/cm² to about 1800 mW/cm², more preferably from about 100 mW/cm² to about 900 mW/cm², and most preferably in the range from about 150 mW/cm² to about 600 mW/cm². It is contemplated that for many practical applications, the irradiance will be within the range of about 300 mW/cm² to about 900 mW/cm². However, the use of higher irradiances may be selected as effective and having the advantage of shortening treatment times.

The time of light irradiation after administration of the photosensitizer may be important as one way of maximizing the selectivity of the treatment, thus minimizing damage to structures other than the target tissues. The optimum time following photosensitizer administration until light treatment can vary widely depending on the mode of administration, the form of administration such as in the form of liposomes or as a complex with LDL, and the type of target tissue. For example, benzoporphyrin derivative typically becomes present within the target neovasculature within one minute post administration and persists for about fifty minutes, lutetium texaphyrin typically becomes present within the target neovasculature within one minute post administration and persists for about twenty minutes, N-aspartyl chlorin e6 typically becomes present within the target neovasculature within one minute post administration and persists for about twenty minutes, and rose bengal typically becomes present in the target vasculature within one minute post administration and persists for about ten minutes.

Effective vascular closure generally occurs at times in the range of about one minute to about three hours following administration of the photosensitizer. However, as with green porphyrins, it is undesirable to perform the PDT within the first five minutes following administration to prevent undue damage to retinal vessels still containing relatively high concentrations of photosensitizer.

The efficacy of PDT may be monitored using conventional methodologies, for example, via fundus photography or angiography. Closure can usually be observed angiographically by hypofluorescence in the treated areas in the early angiographic frames. During the later angiographic frames, a corona of hyperfluorescence may begin to appear which then fills the treated area, possibly representing leakage from the adjacent choriocapillaris through damaged retinal pigment epithelium in the treated area. Large retinal vessels in the treated area typically perfuse following photodynamic therapy.

The present invention includes the use of one or more selective antagonists of CXCL13, RPS6KA2, MMP7, IL1A, KIAA0888, ENPP2, CRIM1, CXCR4, C5orf26, IGHG3, IGLJ3, SHQ1, DNAJC6, C6orf105, NALP1, and/or RGS13, and/or one or more selective agonists of RORA, NALP2, PLA2G4A, PKP2, UCHL1, TANC1, ABCA1, VCAN, and/or FAM38B in the preparation of a medicament for treating one or more angiogenic disorders, for example, an ocular angiogenic disorder, for example, a disorder associated with choridal neovascularization, for example, age-related macular degeneration. The selective antagonist(s) and/or agonist(s) may be provided in a kit which optionally may comprise a package insert with instructions for how to treat the patient with, or at risk of developing, one or more angiogenic disorders, for example, an ocular angiogenic disorder, for example, a disorder associated with choridal neovascularization, for example, age-related macular degeneration. For each administration, the selective antagonist(s) and/or agonist(s) may be provided in unit-dosage or multiple-dosage form. It is understood that the initial dosage administered may be increased beyond the above upper level in order to rapidly achieve the desired blood-level or tissue level, or the initial dosage may be smaller than the optimum and the daily dosage may be progressively increased during the course of treatment depending on the particular situation. If desired, the daily dose may also be divided into multiple doses for administration, for example, two to four times per day.

In light of the foregoing general discussion, the specific examples presented below are illustrative only and are not intended to limit the scope of the invention. Other generic and specific configurations will be apparent to those persons skilled in the art.

### EXAMPLES

### Example 1

### Genome-wide Scan Using Highly Heterozygous Microsatellite Markers

In this experiment, specific genome loci having a correlation to the presence of an angiogenic disorder, namely age-related macular degeneration (AMD), are identified by comparing extremely discordant sibpairs. To analyze the extremely discordant pairs, loci were searched where, on average, the discordant pairs shared fewer than one allele at a convincing level of statistical significance.

Regions of the genome reported to harbor AMD susceptibility genes for both early or advanced forms of AMD were targeted. (DeAngelis et al. (2008) “Genetics of Age-Related Macular Degeneration” in Albert D M, Miller J W. Principles and practice of ophthalmology. Philadelphia, Pa.: Saunders, In Press.) These regions included 1q23-q41; 2p12-p25; 3p13-p25; 3q26-q12; 4q32-q13; 5p13-p14; 5q34-q12; 6q24-6q15; 9p13-9p24; 9q33-9q31; 10q26-10q23; 12q24-q23; 14q32-q13; 15q26-15q11; 16p12-p13; 17q25-17q25; 19q13; and 22q13-12. (Klein et al. (1998) “Age-related macular degeneration. Clinical features in a large family and linkage to chromosome 1q,” Arch Ophthalmol 116:1082-1088; Weeks et al. (2001) “Age-Related Maculopathy: An Expanded Genome-wide Scan with Evidence of Susceptibility Loci Within the 1q31 and 17q25 Regions,” Am J Ophthalmol 132(5): 682-692; Weeks et al. (2004) “Age-related maculopathy: a genomewide scan with continued evidence of susceptibility loci within the 1q31, 10q26, and 17q25 regions,” Am J Hum Genet. 75:174-189; Seddon et al. (2003) “A genomewide scan for age-related macular degeneration provides evidence for linkage to several chromosomal regions,” Am J Hum Genet. 73:780-790; Majewski et al. (2003) “Age-Related Macular Degeneration—a Genome Scan in Extended Families,” Am J. Hum. Genet. 73: 540-550; Abecasis et al. (2004) “Age-Related Macular Degeneration: A High-Resolution Genome Scan for Susceptibility Loci in a Population Enriched for Late-Stage Disease,” Am J. Hum. Genet. 74: 482-494; Jun et al. (2005) “Genome-wide analyses demonstrate novel loci that predispose to drusen formation,” Invest Ophthalmol V is Sci 46:3081-3088; Schick (2003) “A whole-genome screen of a quantitative trait of age-related maculopathy in sibships from the Beaver Dam Eye Study,” Am J Hum Genet. 72:1412-1424; Iyengar et al. (2004) “Dissection of genomewide-scan data in extended families reveals a major locus and oligogenic susceptibility for age-related macular degeneration,” Am J Hum Genet. 74: 20-39; Fisher et al. (2005) “Meta-analysis of genome scans of age-related macular degeneration,” Hum Mol Genet. 14:2257-2264; Klein et al. (2005) “Complement factor H polymorphism in age-related macular degeneration,” Science 308: 385-389; Schmidt et al. (2004) “Ordered subset linkage analysis supports a susceptibility locus for age-related macular degeneration on chromosome 16p12,” BMC Genet: 5:18; Weeks et al. (2000) “A full genome scan for age-related maculopathy,” Hum Mol Genet. 9:1329-1349; Kenealy et al. (2004) “Linkage analysis for age-related macular degeneration supports a gene on chromosome 10q26,” Mol Vis 10: 57-61.)

One approach to examine genetic factors is to study siblings that are discordant for a quantitative trait, as they tend to not share alleles at genetic loci that govern that trait. In this study, siblings with extremely discordant indicia for the onset of AMD were subjected to genetic analysis. The analysis for the genome wide survey included 147 highly polymorphic markers tightly linked to these genomic locations obtained from the Map-O-Mat database (available at the web site, http://compgen.rutgers.edu/mapomat) and the Marshfield maps database (available at the web site, www.ncbi.nlm.nih.gov). All markers were fluorescently labeled with either HEX or FAM on the 5′ end of the reverse primer, and an additional sequence of CTGTCTT (SEQ ID NO: 81) was added to the 5′ of the forward primer.

Polymerase chain reaction was used to amplify genomic DNA fragments from 20 ng of leukocyte DNA from 134 extremely discordant sibpairs (268 subjects). Data was then analyzed using GENEMAPPER v3.7 software (Applied Biosystems, Foster City, Calif.), which interrogates the quality of the size standard and makes the appropriate genotype calls based on size. For quality control purposes, all genotypes were then evaluated manually as well. Using the statistical methods (Risch et al. (1995) “Extreme discordant sib pairs for mapping quantitative trait loci in humans,” Science 268:1584-1589) for calculating the expected IBS scores, it was found that 11 of these regions were more significantly associated with neovascular AMD risk than the 1q32 region harboring the CFH susceptibility gene (p=10⁻²). The regions that showed at least a statistical significance of p=10⁻³ were 2p11-2p25; 3q26-q12; 5q34-q12; 4q32-q13; 9q33-9q31; 10q26-10q23; 12q24-q23; 14q32-q13; 15q26-15q11; 19q13; and 22q13-12. The 4q32-q13 (p=10⁻⁵²) and 22q13-12 (p=10⁻⁴³) were more strongly associated with risk of neovascular AMD than the 10q26 region (p=10¹⁶).) For example, Table 3 shows the results of linkage analysis of 8 microsatellite markers tightly linked to the 10q26 region. (DeAngelis et al. (2007) “Novel Alleles In HTRA1 Both Reduce And Increase Risk Of Neovascular Age-Related Macular Degeneration Independent Of Cfh And Smoking,” Ophthalmology E-pub. Dec. 26, 2007.)

TABLE 3
Exemplary microsatellite markers identified in association with AMD

| D10S1213 | obs | exp | D10S1656 | obs | exp | D10S1723 | obs | exp | D10S587 | obs | exp |
|---|---|---|---|---|---|---|---|---|---|---|---|
| # of 0's = | 21 | 22.2 | # of 0's = | 51 | 18.3 | # of 0's = | 21 | 26.0 | # of 0's = | 20 | 20.6 |
| # of 1's = | 67 | 63.1 | # of 1's = | 55 | 60.9 | # of 1's = | 71 | 65.6 | # of 1's = | 60 | 62.7 |
| # of 2's = | 42 | 44.7 | # of 2's = | 24 | 50.8 | # of 2's = | 41 | 41.4 | # of 2's = | 51 | 47.7 |
| total = | 130 | 130 | total = | 130 | 130 | total = | 133 | 133 | total = | 131 | 131 |
| # of na = | 4 | | # of na = | 4 | | # of na = | 1 | | # of na = | 3 | |
| h = | 0.827 | | h = | 0.75 | | h = | 0.884 | | h = | 0.793 | |
| Chi-sq = | 0.6 | | Chi-sq = | 76.3 | | Chi-sq = | 1.6 | | Chi-sq = | 0.3 | |
| Dof = | 2 | | Dof = | 2 | | Dof = | 2 | | Dof = | 2 | |
| p-value = | 0.7377 | | p-value = | 2.7E-17 | | p-value = | 0.4575 | | p-value = | 0.8695 | |
| adjusted | 1 | | adjusted | 4.3E-16 | | adjusted | 1 | | adjusted | 1 | |
| p = | | | p = | | | p = | | | p = | | |
| D10S1690 | obs | exp | D10S1230 | obs | exp | D10S1483 | obs | exp | D10S1222 | obs | exp |
| # of 0's = | 4 | 7.5 | # of 0's = | 10 | 11.8 | # of 0's = | 10 | 16.5 | # of 0's = | 7 | 13.5 |
| # of 1's = | 37 | 31.2 | # of 1's = | 44 | 40.1 | # of 1's = | 48 | 46.6 | # of 1's = | 59 | 46.8 |
| # of 2's = | 30 | 32.3 | # of 2's = | 32 | 34.1 | # of 2's = | 38 | 32.9 | # of 2's = | 35 | 40.7 |
| total = | 71 | 71 | total = | 86 | 86 | total = | 96 | 96 | total = | 101 | 101 |
| # of na = | 63 | | # of na = | 48 | | # of na = | 38 | | # of na = | 33 | |
| h = | 0.65 | | h = | 0.74 | | h = | 0.83 | | h = | 0.73 | |
| Chi-sq = | 2.9 | | Chi-sq = | 0.8 | | Chi-sq = | 3.4 | | Chi-sq = | 7.1 | |
| Dof = | 2 | | Dof = | 2 | | Dof = | 2 | | Dof = | 2 | |
| p-value = | 0.2344 | | p-value = | 0.6767 | | p-value = | 0.1800 | | p-value = | 0.0291 | |
| adjusted | 1 | | adjusted | 1 | | adjusted | 1 | | adjusted | 0.4661 | |
| p = | | | p = | | | p = | | | p = | | |

# = number; na = non-applicable; h = heterozygosity; Chi-sq = Chi-squared statistic; obs = observed; exp = expected).
^{#} Indicates the number of alleles (0, 1 or 2) shared between the sibling pair.

Identity-by-state (IBS) scores were calculated from the number of alleles (0, 1 or 2) shared between each pair, the index and the discordant sibling, for each of the 8 markers. Using heterozygosities for each marker obtained from the Map-O-Mat database (available at the web site, http://compgen.rutgers.edu/mapomat/) the expected IBS (null hypothesis of no linkage) was calculated and then compared with the observed IBS values. A goodness of fit test was applied to assess the significance of the difference between the observed and expected distribution. Bonferroni Correction was applied to the P values of the association tests on microsatellite markers and AMD risk.

Taken together, the preliminary linkage results underscored the need to evaluate other candidate genes and their interactions with CFH and LOC387715/HTRA1. Accordingly, approximately 90 genes within 2 mb on either side of the statistically significant highly heterozygous markers in the regions listed above and approximately 45 within 1 mb on either side of the significant marker were culled from the ENSEMBL/NCBI databases (available at the web site, http://www.ensembl.org/Homo_sapiens/). Complementary to the genome wide survey, data from RNA microarray experiments were generated as described in Example 2 and candidate genes that overlapped from both types of analyses were identified.

### Example 2

### Identification of Genes Related to Ocular Angiogenic Disorders

For this study, total RNA isolates from transformed lymphocyte cell lines derived from eighteen individuals (nine extremely discordant sibpairs, i.e., nine subjects affected with an angiogenic disorder, namely AMD, and nine matched sibling controls without the disorder) were quantitatively prepared using RNAEASY kits (Qiagen, Valencia, Calif.). RNA quality was determined by analysis using agarose gel or an Agilent 2100 bioanalyzer instrument (Santa Clara, Calif.). RNA was amplified, labeled, and hybridized to human Affymetrix U133A 2.0 PLUS microarrays (Santa Clara, Calif.) containing analytical elements corresponding to approximately 30,000+ genes. The nine discordant sibpairs were analyzed with gene expression microarrays.

Principal component analysis (PCA) showed substantial differences between these nine affected and unaffected siblings, therefore the microarray data was analyzed under a paired two-sample design. This design was comprised of one factor; the AMD affection status and two levels; affected siblings and unaffected siblings. A statistical tool referred to as robust multi-chip analysis, or RMA for short, was employed. The specific procedure entailed the following:

1. Probe-specific correction of the probes using a model based on observed intensity being the sum of signal and (background) noise (Irizarry et al. (2003) “Summaries of Affymetrix GeneChip probe level data,” Nucleic Acids 31:e15; Irizarry et al. (2003) “Exploration, normalization, and summaries of high density oligonucleotide array probe level data,” Biostatistics 4:249-264.);

2. Normalization of corrected PM probes using quantile normalization (Bolstad et al. (2003) “A comparison of normalization methods for high density oligonucleotide array data based on variance and bias,” Bioinformatics 19:185-193.); and

3. Calculation of expression measures using median polish.

Additional normalization was then applied to the summarized data. The local pooled error (LPE) test was then applied to search for differentially expressed genes. The LPE approach is similar to the Significance Analysis of Microarrays (SAM) method and the B-statistic. (Tusher et al. (2001) “Significance analysis of microarrays applied to the ionizing radiation response,” Proc Natl Acad Sci USA 98: 5116-5121; Lonnstedt et al. (2001) “Replicated Microarray Data. Statistical Sinica,” Accepted (available at the web site, http://www.stat.berkeley.edu/users/terry/zarray/Html/papersindex.html).)

To account for the multiple testing issue inherent with analysis of data from microarray experiments, Bonferroni correction was used to control for the family wise error rate equal to 0.05. Using RMA, 90 genes were found to have a statistically significant difference in expression levels in affected patients when compared to their unaffected siblings (p<0.05). These results were further confirmed using a second summarizing method, which is a variation of the RMA called GCRMA. (Wu et al. (2004) “Stochastic Models Inspired by Hybridization Theory for Short Oligonucleotide Arrays,” Proceedings of RECOMB.) With this method, 71 genes were found to be statistically significant (p<0.05). Analysis was completed using S+arrayanalyzer 2.0 from Insightful Corporation (Seattle, Wash.). There were 45 overlapping genes which were found significant by both methods. Genes identified by either method, RMA or GCRMA that were statistically significant and had at least a two-fold change between 9 extremely discordant sib-pairs were then determined to create a short list of candidate genes for further study. From the statistical analysis coupled with the linkage analysis (as described above), as well as certain other studies, ten genes that are also located in regions harboring AMD susceptibility genes were identified. These genes, depicted in Table 4, function in immunity/inflammation processes, apoptosis, cell membrane integrity and structure and transcriptional regulation. Information on genes was derived from freely available public databases such as Ensembl/NCBI, available at the web site, www.ensembl.org/Homo_sapiens/geneview.

TABLE 4
Genes identified in association with an angiogenic disorder, namely AMD

| | | | Gene size |
|---|---|---|---|
| Gene name | Location | Function | (bp) |
| RGS13, regulator of G- | 1q31.2 | signal transduction | 27358 |
| protein signaling 13 | | | |
| CRIM1, cysteine-rich motor | 2p21 | cysteine-type endopeptidase activity, | 195209 |
| neuron 1 | | insulin-like growth factor binding, | |
| | | serine-type endopeptidase inhibitor | |
| | | regulation of cell growth | |
| CXCR4, chemokine (C—X—C | 2q21 | chemokine receptor activity, rhodopsin- | 1070 |
| motif) receptor 4 | | like receptor activity, G-protein coupled | |
| | | receptor | |
| CXCL13, chemokine (C—X—C | 4q21 | chemokine activity, cell-cell signaling, | 6008 |
| motif) ligand 13 (B-cell | | chemotaxis | |
| chemoattractant) | | | |
| C5orf26, chromosome 5 open | 5q21-q22 | Protein coding | 1781 |
| reading frame 26 (formerly | | | |
| TIGA1) | | | |
| RPS6KA2, ribosomal protein | 6q27 | ATP binding, serine/threonine kinase | 452947 |
| S6 kinase, 90 kDa, | | activity, transferase activity | |
| polypeptide 2 | | | |
| MMP7, matrix | 11q21-q22 | calcium ion binding, matrilysin activity, | 10238 |
| metalloproteinase 7 | | zinc ion binding, collagen catabolism, | |
| (matrilysin, uterine) | | peptidoglycan metabolism | |
| IGHG3, immunoglobulin | 14q32.33 | MHC class I receptor activity, antigen | 552224 |
| heavy constant gamma 3 | | binding and processing | |
| (G3m marker) | | | |
| RORA, RAR-related orphan | 15q21-q22 | metal ion binding, steroid hormone | 731954 |
| receptor A | | receptor activity, regulation of | |
| | | angiogenesis | |
| NALP2, NACHT, leucine | 19q13.42 | ATP binding, apoptosis, regulation of | 35848 |
| rich repeat and PYD | | caspase activity, interleukin-1 beta | |
| containing 2 | | secretion | |

In addition to the genes identified in Table 4, fifteen additional genes, PLA2G4A, IGLJ3, SHQ1, UCHL1, TANC1, PKP2, DNAJC6, C6orf105, NALP1, IL1A, ABCA1, VCAN, KIAA0888, ENPP2, and FAM38B, also were identified in connection with the angiogenic disorder, namely AMD. Further analysis was conducted to determine whether the twenty-five identified genes were upregulated or downregulated in affected siblings relative to the unaffected control siblings. Information about each of these twenty-five genes associated with the angiogenic disorder (i.e. AMD), including whether each is upregulated or downregulated in affected siblings, is shown in FIGS. 1A and 1B. This information identifies twenty-five genes as targets for determining a subject's risk of having, or for detecting that the individual has the one or more angiogenic disorders, for example, an ocular angiogenic disorder, for example, a disorder associated with choroidal neovascularization, for example, age-related macular degeneration. Accordingly, if the subject has increased levels of one or more of the CXCL13, RPS6KA2, MMP7, IL1A, KIAA0888, ENPP2, CRIM1, CXCR4, C5orf26, IGHG3, IGLJ3, SHQ1, DNAJC6, C6orf105, NALP1, and/or RGS13 genes or gene products and/or the subject has decreased levels of one or more of the RORA, NALP2, PLA2G4A, PKP2, UCHL1, TANC1, ABCA1, VCAN, and/or FAM38B genes or gene products relative to one or more corresponding control values, the subject is at risk of developing, or has, the angiogenic disorder (i.e. AMD). Additionally, this data identifies therapeutic targets to prevent, slow, or stop development of one or more angiogenic disorders, for example, an ocular angiogenic disorder, for example, a disorder associated with choridal neovascularization, for example, AMD, namely, antagonists (e.g. antibodies) for CXCL13, RPS6KA2, MMP7, IL1A, KIAA0888, ENPP2, CRIM1, CXCR4, C5orf26, IGHG3, IGLJ3, SHQ1, DNAJC6, C6orf105, NALP1, and/or RGS13 and agonists (e.g. exogenous proteins or peptides) for RORA, NALP2, PLA2G4A, PKP2, UCHL1, TANC1, ABCA1, VCAN, and/or FAM38B. Such antagonists and agonists can be used to prevent, slow, or stop development of one or more angiogenic disorders, for example, an ocular angiogenic disorder, for example, a disorder associated with choridal neovascularization, for example, age-related macular degeneration.

ENPP2, IL1A, IGHG3, CXCL13, and CXCR4 can be classified as having a role in immunity/inflammation. ABCA1 and PLA2G4A can be classified as having a role in lipid metabolism. NALP2 and IL1A can be classified as having a role oxidative stress. PKP2, MMP7, VCAN, and ENPP2 can be classified as having a role in maintaining structural integrity. ABCA1 is a regulator of lipid transport, and mutations in this gene may result, indirectly, in atherosclerosis. The Copenhagen Heart Study reported that heterozygous mutations in ABCA1 were associated with abnormally low HDL levels.

The block structure of ABCA1 was determined to estimate whether linkage disequilibrium (LD) between pairs of SNPs in the candidate loci could reduce the number of SNPs for genotyping. This was done by exploring the genotype from HapMap among Caucasians for the large ABCA1 candidate locus (approximately 150 kb). Of the 120 SNPs genotyped by HapMap, 100 were informative with frequency greater than 0.8%. These 100 SNPs gave rise to inferred haplotypes with frequency greater than 1% in ten regions of very high LD or blocks by the haploview algorithm, requiring a subset of 30 tagSNPs for complete specification. An additional 28 SNPs were not assigned to a haplotype. Alternatively, most of the variation can be captured in 49 “LD-tag” SNPs through LD relationships according to the “Tagger” algorithm. The fractions of SNPs required by either approach (58% or 49%) are larger than estimated in a recent study (approximately 30%) designed to capture genetic variation with frequency greater than 10%, but the difference can likely be explained by the intent of the current study to capture genetic variation with a smaller minimum frequency, about 5%. Nevertheless, the reduction in genotyping is substantial, and going forward 0.5 can be used as the fraction of candidate SNPs that need to be genotyped at each locus as a result of LD.

### Example 3

### Use of Selective Agonists and/or Antagonists for Treating Angiogenic Disorders

It is contemplated that a variety of antagonists for one or more of CXCL13, RPS6KA2, MMP7, IL1A, KIAA0888, ENPP2, CRIM1, CXCR4, C5orf26, IGHG3, IGLJ3, SHQ1, DNAJC6, C6orf105, NALP1, or RGS13 and/or agonists for one or more of RORA, NALP2, PLA2G4A, PKP2, UCHL1, TANC1, ABCA1, VCAN, and/or FAM38B (i.e. the treatment agents described above) will be useful to slow, stop, prevent, or reverse the progression of one or more angiogenic disorders, for example, an ocular angiogenic disorder, for example, a disorder associated with choridal neovascularization, for example, age-related macular degeneration. Examples of these compounds are listed herein.

For example, it is contemplated that an active form of RORA, NALP2, PLA2G4A, PKP2, UCHL1, TANC1, ABCA1, VCAN, or FAM38B can be administered to a subject, such as a mammal, such as a human, using techniques known to those skilled in the art so as to slow down, stop, prevent, or reverse the progression of one or more angiogenic disorders, for example, an ocular angiogenic disorder, for example, a disorder associated with choridal neovascularization, for example, age-related macular degeneration. As another example, it is contemplated that a specific antibody that binds to and reduces the activity of CXCL13, RPS6KA2, MMP7, IL1A, KIAA0888, ENPP2, CRIM1, CXCR4, C5orf26, IGHG3, IGLJ3, SHQ1, DNAJC6, C6orf105, NALP1, or RGS13 can be administered to a subject, such as a mammal, such as a human, using techniques known to those skilled in the art so as to slow down, stop, prevent, or reverse the progression of one or more angiogenic disorders, for example, an ocular angiogenic disorder, for example, a disorder associated with choridal neovascularization, for example, age-related macular degeneration.

### INCORPORATION by REFERENCE

The entire disclosure of each of the publications, patent documents, and database references referred to herein (including sequences, SNPs, and other information identified with reference to database identifiers, for example, in the Ensembl/NCBI databases) is incorporated by reference in its entirety for all purposes to the same extent as if each individual source were individually denoted as being incorporated by reference.

### EQUIVALENTS

The invention may be embodied in other specific forms without departing form the spirit or essential characteristics thereof. The foregoing embodiments are therefore to be considered in all respects illustrative rather than limiting on the invention described herein. Scope of the invention is thus indicated by the appended claims rather than by the foregoing description, and all changes that come within the meaning and range of equivalency of the claims are intended to be embraced therein.

What is claimed is:

1-12. (canceled)

13. A method of determining whether a mammal is at risk of developing, or has, an ocular angiogenic disorder, the method comprising:
measuring the amount of one or more markers in a test sample harvested from the mammal wherein the one or more markers are selected from the group consisting of a CRIM1 gene, a CXCR4 gene, a C5orf26 gene, an IGHG3 gene, an IGLJ3 gene, a SHQ1 gene, a DNAJC6 gene, a C6orf105 gene, a NALP1 gene, a RGS13 gene, an ABCA1 gene, a VCAN gene, a FAM38B gene, a CRIM1 gene product, a CXCR4 gene product, a C5orf26 gene product, an IGHG3 gene product, an IGLJ3 gene product, a SHQ1 gene product, a DNAJC6 gene product, a C6orf105 gene product, a NALP1 gene product, a RGS13 gene product, an ABCA1 gene product, a VCAN gene product, and a FAM38B gene product, wherein when the measured marker is a CRIM1 gene, a CXCR4 gene, a C5orf26 gene, an IGHG3 gene, an IGLJ3 gene, a SHQ1 gene, a DNAJC6 gene, a C6orf105 gene, a NALP1 gene, a RGS13 gene, a CRIM1 gene product, a CXCR4 gene product, a C5orf26 gene product, an IGHG3 gene product, an IGLJ3 gene product, a SHQ1 gene product, a DNAJC6 gene product, a C6orf105 gene product, a NALP1 gene product, or a RGS13 gene product, an amount of the marker in the sample greater than its corresponding control value is indicative that the mammal is at risk of developing, or has, the ocular angiogenic disorder and when the measured marker is an ABCA1 gene, a VCAN gene, a a FAM38B gene, an ABCA1 gene product, a VCAN gene product, or a FAM38B gene product, an amount of the marker in the sample less than its corresponding control value is indicative that the mammal is at risk of developing, or has, the ocular angiogenic disorder.

14. The method of claim 13, wherein the test sample is a tissue or body fluid sample.

15. The method of claim 14, wherein the body fluid sample is selected from the group consisting of blood, serum and plasma.

16. The method of claim 14, wherein the tissue is choroid or retina.

17. The method of claim 13, wherein the marker is a gene product and is a nucleic acid.

18. The method of claim 17, wherein the nucleic acid is an mRNA.

19. The method of claim 17, wherein the nucleic acid is measured by a hybridization assay.

20. The method of claim 13, wherein the marker is a gene product and is a protein.

21. The method of claim 20, wherein the protein is measured by an immunoassay.

22. The method of claim 13, wherein the ocular angiogenic disorder is age-related macular degeneration.

23. The method of claim 13, wherein the mammal is a human.

24. The method of claim 13, wherein when two or more measured markers are different from corresponding control values, it is indicative that the mammal is at risk of developing or has the ocular angiogenic disorder.

25-29. (canceled)

30. A kit comprising
(a) an agent for determining the amount of one or more of a CRIM1 gene, a CXCR4 gene, a C5orf26 gene, an IGHG3 gene, an IGLJ3 gene, a SHQ1 gene, a DNAJC6 gene, a C6orf105 gene, a NALP1 gene, a RGS13 gene, an ABCA1 gene, a VCAN gene, a FAM38B gene, a CRIM1 gene product, a CXCR4 gene product, a C5orf26 gene product, an IGHG3 gene product, an IGLJ3 gene product, a SHQ1 gene product, a DNAJC6 gene product, a C6orf105 gene product, a NALP1 gene product, a RGS13 gene product, an ABCA1 gene product, a VCAN gene product, and a FAM38B gene product in a test sample; and
(b) instructions on how to determine the amount of the one or more genes or gene products in the sample to determine if a mammal is at risk of developing, or has, an ocular angiogenic disorder.

31. The kit of claim 30, wherein the ocular angiogenic disorder is age-related macular degeneration.

32. The kit of claim 31, wherein the age-related macular degeneration is a dry form of age-related macular degeneration or a neovascular form of age-related macular degeneration.

33. The kit of claim 30, wherein the ocular disorder is a disorder associated with choroidal neovascularization.

34. The kit of claim 33, wherein the ocular disorder associated with choroidal neovascularization is selected from the group consisting of age-related macular degeneration, pathologic myopia, angioid streaks, choroidal ruptures, ocular histoplasmosis syndrome, multifocal choroiditis, idiosyncratic macular degeneration, and idiopathic choroidal neovascularization.

35. The method of claim 13, wherein the ocular angiogenic disorder is an ocular disorder associated with choroidal neovascularization.

36. The method of claim 35, wherein the ocular disorder associated with choroidal neovascularization is selected from the group consisting of age-related macular degeneration, pathologic myopia, angioid streaks, choroidal ruptures, ocular histoplasmosis syndrome, multifocal choroiditis, idiosyncratic macular degeneration, and idiopathic choroidal neovascularization.

37. (canceled)

38. The method of claim 22, wherein the age-related macular degeneration is a dry form of age-related macular degeneration or a neovascular form of age-related macular degeneration.

39. (canceled)
